SEQUENCE LISTING (141 sequences)

SEQ ID NO: 1 - protein, 526 aa, Homo sapiens [sequence omitted]

SEQ ID NO: 2 - DNA, 1581 bp, Homo sapiens
  source 1..1581: mol_type = other DNA; note = nucleic acid sequence of human BTN1A1
atggcagttttcccaagctccggtctccccagatgtctgctcaccctcattctcctccagctgcccaaactggattcagctccctttgacgtgattggacccccggagcccatcctggccgttgtgggtgaggacgccaagctgccctgtcgcctgtctccgaacgcgagcgccgagcacttggagctacgctggttccgaaagaaggtttcgccggccgtgctggtgcatagggacgggcgcgagcaggaagccgagcagatgcccgagtaccgcgggcgggcgacgctggtccaggacggcatcgccaaggggcgcgtggccttgaggatccgtggcgtcagagtctctgacgacggggagtacacgtgctttttcagggaggatggaagctacgaagaagccctggtgcatctgaaggtggctgctctgggctctgaccctcacatcagtatgcaagttcaagagaatggagaaatctgtctggagtgcacctcagtgggatggtacccagagccccaggtgcagtggagaacttccaagggagagaagtttccatctacatcagagtccaggaatcctgatgaagaaggtttgttcactgtggctgcttcagtgatcatcagagacacttctgcgaaaaatgtgtcctgctacatccagaatctccttcttggccaggagaagaaagtagaaatatccataccagcttcctccctcccaaggctgactccctggatagtggctgtggctgtcatcctgatggttctaggacttctcaccattgggtccatatttttcacttggagactatacaacgaaagacccagagagaggaggaatgaattcagctctaaagagagactcctggaagaactcaaatggaaaaaggctaccttgcatgcagttgatgtgactctggacccagacacagctcatccccacctctttctttatgaggattcaaaatctgttcgactggaagattcacgtcagaaactgcctgagaaaacagagagatttgactcctggccctgtgtgttgggccgtgagaccttcacctcaggaaggcattactgggaggtggaggtgggagacaggactgactgggcaatcggcgtgtgtagggagaatgtgatgaagaaaggatttgaccccatgactcctgagaatgggttctgggctgtagagttgtatggaaatgggtactgggccctcactcctctccggacccctctcccattggcagggcccccacgccgggttgggattttcctagactatgaatcaggagacatctccttctacaacatgaatgatggatctgatatctatactttctccaatgtcactttctctggccccctccggcccttcttttgcctatggtctagcggtaaaaagcccctgaccatctgcccaattgctgatgggcctgagagggtcacagtcattgctaatgcccaggacctttctaaggagatcccattgtcccccatgggggaggactctgcccctagggatgcagacactctccattctaagctaatccctacccaacccagccaaggggcaccttaa

SEQ ID NO: 3 - protein, 257 aa, Homo sapiens
  source 1..257: note = ICD domain of human BTN1A1
RLYNERPRERRNEFSSKERLLEELKWKKATLHAVDVTLDPDTAHPHLFLYEDSKSVRLEDSRQKLPEKTERFDSWPCVLGRETFTSGRHYWEVEVGDRTDWAIGVCRENVMKKGFDPMTPENGFWAVELYGNGYWALTPLRTPLPLAGPPRRVGIFLDYESGDISFYNMNDGSDIYTFSNVTFSGPLRPFFCLWSSGKKPLTICPIADGPERVTVIANAQDLSKEIPLSPMGEDSAPRDADTLHSKLIPTQPSQGAP

SEQ ID NO: 4 - protein, 256 aa, Mus musculus
  source 1..256: note = ICD domain of mouse BTN1A1
TWKLYKERSSLRKKEFGSKERLLEELRCKKTVLHEVDVTLDPDTAHPHLFLYEDSKSVRLEDSRQILPDRPERFDSWPCVLGRETFTSGRHYWEVEVGDRTDWAIGVCRENVVKKGFDPMTPDNGFWAVELYGNGYWALTPLRTSLRLAGPPRRVGVFLDYDAGDISFYNMSNGSLIYTFPSISFSGPLRPFFCLWSCGKKPLTICSTANGPEKVTVIANVQDDIPLSPLGEGCTSGDKDTLHSKLIPFSPSQAAP

SEQ ID NO: 5 - protein, 13 aa, synthetic construct
  source 1..13: note = VL CDR1 of BTN1A1 antibody STC43H11-1
QASQSVYNNNRCS

SEQ ID NO: 6 - protein, 7 aa, synthetic construct
  source 1..7: note = VL CDR2 of BTN1A1 antibody STC43H11-1
HTSTLDS

SEQ ID NO: 7 - protein, 13 aa, synthetic construct
  source 1..13: note = VL CDR3 of BTN1A1 antibody STC43H11-1
LGNYDCSRADCAA

SEQ ID NO: 8 - protein, 5 aa, synthetic construct
  source 1..5: note = VH CDR1 of BTN1A1 antibody STC43H11-1
SYAMG

SEQ ID NO: 9 - protein, 16 aa, synthetic construct
  source 1..16: note = VH CDR2 of BTN1A1 antibody STC43H11-1
SIGTGGGTGYASWAKG

SEQ ID NO: 10 - skipped

SEQ ID NO: 11 - protein, 22 aa, synthetic construct
  source 1..22: note = VL FR1 of BTN1A1 antibody STC43H11-1
QVLTQTASPVSAAVGGTVTINC

SEQ ID NO: 12 - protein, 15 aa, synthetic construct
  source 1..15: note = VL FR2 of BTN1A1 antibody STC43H11-1
WFQQKPGQPPKRLIY

SEQ ID NO: 13 - protein, 32 aa, synthetic construct
  source 1..32: note = VL FR3 of BTN1A1 antibody STC43H11-1
GVPSRFKGSGSGTQFTLTISDVQCDDAATYYC

SEQ ID NO: 14 - protein, 10 aa, synthetic construct
  source 1..10: note = VL FR4 of BTN1A1 antibody STC43H11-1
FGGGTEVVVK

SEQ ID NO: 15 - protein, 29 aa, synthetic construct
  source 1..29: note = VH FR1 of BTN1A1 antibody STC43H11-1
QSLEESGGRLVTPGGSLTLTCTVSGIDLS

SEQ ID NO: 16 - protein, 14 aa, synthetic construct
  source 1..14: note = VH FR2 of BTN1A1 antibody STC43H11-1
WVRQAPGEGLEWIG

SEQ ID NO: 17 - protein, 30 aa, synthetic construct
  source 1..30: note = VH FR3 of BTN1A1 antibody STC43H11-1
RFTISKTSTTVDLKMTSLTASDTATYFCAT

SEQ ID NO: 18 - protein, 11 aa, synthetic construct
  source 1..11: note = VH FR4 of BTN1A1 antibody STC43H11-1
WGPGTLVTVSS

SEQ ID NO: 19 - protein, 112 aa, synthetic construct
  source 1..112: note = VL Domain of BTN1A1 antibody STC43H11-1
QVLTQTASPVSAAVGGTVTINCQASQSVYNNNRCSWFQQKPGQPPKRLIYHTSTLDSGVPSRFKGSGSGTQFTLTISDVQCDDAATYYCLGNYDCSRADCAAFGGGTEVVVK

SEQ ID NO: 20 - protein, 108 aa, synthetic construct
  source 1..108: note = VH Domain of BTN1A1 antibody STC43H11-1
QSLEESGGRLVTPGGSLTLTCTVSGIDLSSYAMGWVRQAPGEGLEWIGSIGTGGGTGYASWAKGRFTISKTSTTVDLKMTSLTASDTATYFCATSNLWGPGTLVTVSS

SEQ ID NO: 21 - DNA, 336 bp, synthetic construct
  source 1..336: mol_type = other DNA; note = nucleic acid sequence VL domain of BTN1A1 antibody STC43H11-1
caagtgctgacccagactgcatcccccgtgtctgcggctgttggaggcacagtcaccatcaattgccaggccagtcagagtgtttataataacaaccgctgttcctggtttcagcagaaaccagggcagcctcccaagcgcctgatctatcatacatcgactctagattctggggtcccatcgcggttcaaaggcagtggatctgggacacagttcactctcaccatcagcgacgtgcagtgtgacgatgctgccacttactactgtctaggcaattatgattgtagtcgtgctgattgtgctgctttcggcggagggaccgaggtggtggtcaaa

SEQ ID NO: 22 - DNA, 324 bp, synthetic construct
  source 1..324: mol_type = other DNA; note = nucleic acid sequence VH domain of BTN1A1 antibody STC43H11-1
cagtcgctggaggagtccgggggtcgcctggtaacgcctggaggatccctgacactcacctgcacagtctctggaatcgacctcagtagctatgcaatgggctgggtccgccaggctccaggggaggggctggaatggatcggaagcattggtactggtggtggcacaggctacgcgagctgggcaaaaggccgattcaccatctccaaaacctcgaccacggtggatctgaaaatgaccagtctgacagcttcagacacggccacctatttctgtgccaccagtaacttgtggggcccaggcaccctggtcaccgtctcctca

SEQ ID NO: 23 - protein, 15 aa, synthetic construct
  source 1..15: note = BTN1A1 Peptide 4
EVEVGDRTDWAIGVC

SEQ ID NO: 24 - protein, 15 aa, synthetic construct
  source 1..15: note = BTN1A1 Peptide 4, Mutant 5 (G365A)
EVEVADRTDWAIGVC

SEQ ID NO: 25 - protein, 15 aa, synthetic construct
  source 1..15: note = BTN1A1 Peptide 4, Mutant 6 (D366A)
EVEVGARTDWAIGVC

SEQ ID NO: 26 - protein, 15 aa, synthetic construct
  source 1..15: note = BTN1A1 Peptide 4, Mutant 7 (R367A)
EVEVGDATDWAIGVC

SEQ ID NO: 27 - protein, 15 aa, synthetic construct
  source 1..15: note = BTN1A1 Peptide 4, Mutant 8 (T368A)
EVEVGDRADWAIGVC

SEQ ID NO: 28 - protein, 15 aa, synthetic construct
  source 1..15: note = BTN1A1 Peptide 4, Mutant 9 (D369A)
EVEVGDRTAWAIGVC

SEQ ID NO: 29 - protein, 15 aa, synthetic construct
  source 1..15: note = BTN1A1 Peptide 4, Mutant 10 (W370A)
EVEVGDRTDAAIGVC

SEQ ID NO: 30 - protein, 15 aa, synthetic construct
  source 1..15: note = BTN1A1 Peptide 4, Mutant 11 (A371G)
EVEVGDRTDWGIGVC

SEQ ID NO: 31 - protein, 15 aa, synthetic construct
  source 1..15: note = BTN1A1 Peptide 4, Mutant 12 (I372A)
EVEVGDRTDWAAGVC

SEQ ID NO: 32 - protein, 15 aa, synthetic construct
  source 1..15: note = TRIM11 peptide
EVEVGDRTSWALGVC

SEQ ID NO: 33 - protein, 8 aa, Homo sapiens
  source 1..8: note = residues 365-372 of SEQ ID NO:1
GDRTDWAI

SEQ ID NO: 34 - protein, 7 aa, Homo sapiens
  source 1..7: note = residues 366-372 of SEQ ID NO:1
DRTDWAI

SEQ ID NO: 35 - protein, 15 aa, synthetic construct
  source 1..15: note = Peptide from serine/threonine-protein kinase ICK (CILK) isoform 2
AQPVHGRTDWASKYA

SEQ ID NO: 36 - protein, 15 aa, synthetic construct
  source 1..15: note = Peptide from 1-lactate dehydrogenase B chain (LDHB)
VIKLKGYTNWAIGLS

SEQ ID NO: 37 - protein, 15 aa, synthetic construct
  source 1..15: note = Peptide from MAM and LDL-receptor class A domain-containing protein 1 (MALRD1)
TQDSEDDLDWAIGSR

SEQ ID NO: 38 - protein, 13 aa, synthetic construct
  source 1..13: note = VL CDR1 of BTN1A1 antibody STC43G3-1
QSSQSVYNNNNLS

SEQ ID NO: 39 - protein, 7 aa, synthetic construct
  source 1..7: note = VL CDR2 of BTN1A1 antibody STC43G3-1
RASKLPS

SEQ ID NO: 40 - protein, 10 aa, synthetic construct
  source 1..10: note = VL CDR3 of BTN1A1 antibody STC43G3-1
AGGYSGDINV

SEQ ID NO: 41 - protein, 13 aa, synthetic construct
  source 1..13: note = VL CDR1 of BTN1A1 antibody STC85F1-1
QSSQSVYNNKNLS

SEQ ID NO: 42 - protein, 7 aa, synthetic construct
  source 1..7: note = VL CDR2 of BTN1A1 antibody STC85F1-1
RASTLPS

SEQ ID NO: 43 - protein, 10 aa, synthetic construct
  source 1..10: note = VL CDR3 of BTN1A1 antibody STC85F1-1
AGGYSGDINV

SEQ ID NO: 44 - protein, 5 aa, synthetic construct
  source 1..5: note = VH CDR1 of BTN1A1 antibody STC43G3-1
RYGVN

SEQ ID NO: 45 - protein, 16 aa, synthetic construct
  source 1..16: note = VH CDR2 of BTN1A1 antibody STC43G3-1
YIGTTGNTYYASWVNG

SEQ ID NO: 46 - protein, 10 aa, synthetic construct
  source 1..10: note = VH CDR3 of BTN1A1 antibody STC43G3-1
SVVSPSNSSF

SEQ ID NO: 47 - protein, 5 aa, synthetic construct
  source 1..5: note = VH CDR1 of BTN1A1 antibody STC85F1-1
RYGVN

SEQ ID NO: 48 - protein, 16 aa, synthetic construct
  source 1..16: note = VH CDR2 of BTN1A1 antibody STC85F1-1
YIGTTSNTYYASWAKG

SEQ ID NO: 49 - protein, 10 aa, synthetic construct
  source 1..10: note = VH CDR3 of BTN1A1 antibody STC85F1-1
SVVSPSNSSF

SEQ ID NO: 50 - protein, 23 aa, synthetic construct
  source 1..23: note = VL FR1 of BTN1A1 antibody STC43G3-1
DPVMTQTPSSTSAAVGGTVTINC

SEQ ID NO: 51 - protein, 15 aa, synthetic construct
  source 1..15: note = VL FR2 of BTN1A1 antibody STC43G3-1
WFQQKPGQPPKLLIY

SEQ ID NO: 52 - protein, 32 aa, synthetic construct
  source 1..32: note = VL FR3 of BTN1A1 antibody STC43G3-1
GVPPRFSGSGSGTQFTLTISSVQCDDAATYFC

SEQ ID NO: 53 - protein, 10 aa, synthetic construct
  source 1..10: note = VL FR4 of BTN1A1 antibody STC43G3-1
FGGGTEVVVK

SEQ ID NO: 54 - protein, 23 aa, synthetic construct
  source 1..23: note = VL FR1 of BTN1A1 antibody STC85F1-1
DPVMTQTPSSTSAAVGGTVTINC

SEQ ID NO: 55 - protein, 15 aa, synthetic construct
  source 1..15: note = VL FR2 of BTN1A1 antibody STC85F1-1
WFQQKPGQPPKLLIY

SEQ ID NO: 56 - protein, 32 aa, synthetic construct
  source 1..32: note = VL FR3 of BTN1A1 antibody STC85F1-1
GVPSRFKGSGSGTQFTLTISDLECDDAATYYC

SEQ ID NO: 57 - protein, 10 aa, synthetic construct
  source 1..10: note = VL FR4 of BTN1A1 antibody STC85F1-1
FGGGTEVVVK

SEQ ID NO: 58 - protein, 29 aa, synthetic construct
  source 1..29: note = VH FR1 of BTN1A1 antibody STC43G3-1
QSLEESGGRLVTPGTPLTLTCTVSGIDLS

SEQ ID NO: 59 - protein, 14 aa, synthetic construct
  source 1..14: note = VH FR2 of BTN1A1 antibody STC43G3-1
WVRQAPGKGLEWIG

SEQ ID NO: 60 - protein, 30 aa, synthetic construct
  source 1..30: note = VH FR3 of BTN1A1 antibody STC43G3-1
RFTISKTSTTVELKMTSLTAADTATYFCAR

SEQ ID NO: 61 - protein, 11 aa, synthetic construct
  source 1..11: note = VH FR4 of BTN1A1 antibody STC43G3-1
WGQGTLVTVSS

SEQ ID NO: 62 - protein, 29 aa, synthetic construct
  source 1..29: note = VH FR1 of BTN1A1 antibody STC85F1-1
QSLEESGGRLVTPGGSLTLTCTVSGIDLS

SEQ ID NO: 63 - protein, 14 aa, synthetic construct
  source 1..14: note = VH FR2 of BTN1A1 antibody STC85F1-1
WVRQAPGKGLEWIG

SEQ ID NO: 64 - protein, 31 aa, synthetic construct
  source 1..31: note = VH FR3 of BTN1A1 antibody STC85F1-1
RFTISKTSSTTVDLKMTSLTTEDTATYFCAR

SEQ ID NO: 65 - protein, 11 aa, synthetic construct
  source 1..11: note = VH FR4 of BTN1A1 antibody STC85F1-1
WGQGTLVTVSS

SEQ ID NO: 66 - protein, 110 aa, synthetic construct
  source 1..110: note = VL of BTN1A1 antibody STC43G3-1
DPVMTQTPSSTSAAVGGTVTINCQSSQSVYNNNNLSWFQQKPGQPPKLLIYRASKLPSGVPPRFSGSGSGTQFTLTISSVQCDDAATYFCAGGYSGDINVFGGGTEVVVK

SEQ ID NO: 67 - protein, 110 aa, synthetic construct
  source 1..110: note = VL of BTN1A1 antibody STC85F1-1
DPVMTQTPSSTSAAVGGTVTINCQSSQSVYNNKNLSWFQQKPGQPPKLLIYRASTLPSGVPSRFKGSGSGTQFTLTISDLECDDAATYYCAGGYSGDINVFGGGTEVVVK

SEQ ID NO: 68 - protein, 115 aa, synthetic construct
  source 1..115: note = VH of BTN1A1 antibody STC43G3-1
QSLEESGGRLVTPGTPLTLTCTVSGIDLSRYGVNWVRQAPGKGLEWIGYIGTTGNTYYASWVNGRFTISKTSTTVELKMTSLTAADTATYFCARSVVSPSNSSFWGQGTLVTVSS

SEQ ID NO: 69 - protein, 116 aa, synthetic construct
  source 1..116: note = VH of BTN1A1 antibody STC85F1-1
QSLEESGGRLVTPGGSLTLTCTVSGIDLSRYGVNWVRQAPGKGLEWIGYIGTTSNTYYASWAKGRFTISKTSSTTVDLKMTSLTTEDTATYFCARSVVSPSNSSFWGQGTLVTVSS

SEQ ID NO: 70 - protein, 20 aa, synthetic construct
  source 1..20: note = peptide of ECD1 domain in BTN1A1
RKKVSPAVLVHRDGREQEAE

SEQ ID NO: 71 - protein, 7 aa, synthetic construct
  source 1..7: note = Heavy Chain CDR1 of BTN1A1 antibody STC43H11-1 (Chothia)
GIDLSSY

SEQ ID NO: 72 - protein, 5 aa, synthetic construct
  source 1..5: note = Heavy Chain CDR2 of BTN1A1 antibody STC43H11-1 (Chothia)
GTGGG

SEQ ID NO: 73 - skipped

SEQ ID NO: 74 - protein, 13 aa, synthetic construct
  source 1..13: note = Kappa Light Chain CDR1 of BTN1A1 antibody STC43H11-1 (Chothia)
QASQSVYNNNRCS

SEQ ID NO: 75 - protein, 7 aa, synthetic construct
  source 1..7: note = Kappa Light Chain CDR2 of BTN1A1 antibody STC43H11-1 (Chothia)
HTSTLDS

SEQ ID NO: 76 - protein, 13 aa, synthetic construct
  source 1..13: note = Kappa Light Chain CDR3 of BTN1A1 antibody STC43H11-1 (Chothia)
LGNYDCSRADCAA

SEQ ID NO: 77 - protein, 8 aa, synthetic construct
  source 1..8: note = Heavy Chain CDR1 of BTN1A1 antibody STC43H11-1 (IMGT)
GIDLSSYA

SEQ ID NO: 78 - protein, 8 aa, synthetic construct
  source 1..8: note = Heavy Chain CDR2 of BTN1A1 antibody STC43H11-1 (IMGT)
SIGTGGGT

SEQ ID NO: 79 - protein, 5 aa, synthetic construct
  source 1..5: note = Heavy Chain CDR3 of BTN1A1 antibody STC43H11-1 (IMGT)
ATSNL

SEQ ID NO: 80 - protein, 6 aa, synthetic construct
  source 1..6: note = Kappa Light Chain CDR1 of BTN1A1 antibody STC43H11-1 (IMGT)
QSVYNN

SEQ ID NO: 81 - skipped

SEQ ID NO: 82 - protein, 13 aa, synthetic construct
  source 1..13: note = Kappa Light Chain CDR3 of BTN1A1 antibody STC43H11-1 (IMGT)
LGNYDCSRADCAA

SEQ ID NO: 83 - protein, 6 aa, synthetic construct
  source 1..6: note = Heavy Chain CDR1 of BTN1A1 antibody STC43H11-1 (Contact)
SSYAMG

SEQ ID NO: 84 - protein, 12 aa, synthetic construct
  source 1..12: note = Heavy Chain CDR2 of BTN1A1 antibody STC43H11-1 (Contact)
WIGSIGTGGGTG

SEQ ID NO: 85 - protein, 4 aa, synthetic construct
  source 1..4: note = Heavy Chain CDR3 of BTN1A1 antibody STC43H11-1 (Contact)
ATSN

SEQ ID NO: 86 - protein, 9 aa, synthetic construct
  source 1..9: note = Kappa Light Chain CDR1 of BTN1A1 antibody STC43H11-1 (Contact)
YNNNRCSWF

SEQ ID NO: 87 - protein, 10 aa, synthetic construct
  source 1..10: note = Kappa Light Chain CDR2 of BTN1A1 antibody STC43H11-1 (Contact)
RLIYHTSTLD

SEQ ID NO: 88 - protein, 12 aa, synthetic construct
  source 1..12: note = Kappa Light Chain CDR3 of BTN1A1 antibody STC43H11-1 (Contact)
LGNYDCSRADCA

SEQ ID NO: 89 - DNA, 345 bp, synthetic construct
  source 1..345: mol_type = other DNA; note = nucleic acid sequence VH domain of BTN1A1 antibody STC43G3-1
cagtcgctggaggagtccgggggtcgcctggtcacgcctgggacacccctgacactcacctgcacagtctctggaatcgacctcagtagatatggagtgaactgggtccgccaggctccagggaaggggctggaatggatcggatatattggtactactggtaatacatactacgcgagctgggtgaatggtcgattcaccatctccaaaacctcgaccacggtggagctgaaaatgaccagtctgacagccgcggacacggccacctatttctgtgccagaagtgttgttagtcctagtaatagtagtttctggggccaaggcaccctggtcaccgtctcctca

SEQ ID NO: 90 - DNA, 330 bp, synthetic construct
  source 1..330: mol_type = other DNA; note = nucleic acid sequence VL domain of BTN1A1 antibody STC43G3-1
gaccctgtgatgacccagactccatcttccacgtctgcggctgtgggaggcacagtcaccatcaactgccagtccagtcagagtgtttataataataacaacttatcctggtttcagcagaaaccaggtcagcctcccaagctcctgatctacagggcatccaaactgccatctggggtcccaccgcggttcagcggcagtggatctgggacacagttcactctcaccatcagcagcgtgcagtgtgacgatgctgccacttacttctgtgccggcggttatagtggtgatattaatgttttcggcggagggaccgaggtggtggtcaaa

SEQ ID NO: 91 - protein, 7 aa, synthetic construct
  source 1..7: note = Heavy Chain CDR1 of BTN1A1 antibody STC43G3-1 (Chothia)
GIDLSRY

SEQ ID NO: 92 - protein, 5 aa, synthetic construct
  source 1..5: note = Heavy Chain CDR2 of BTN1A1 antibody STC43G3-1 (Chothia)
GTTGN

SEQ ID NO: 93 - protein, 10 aa, synthetic construct
  source 1..10: note = Heavy Chain CDR3 of BTN1A1 antibody STC43G3-1 (Chothia)
SVVSPSNSSF

SEQ ID NO: 94 - protein, 13 aa, synthetic construct
  source 1..13: note = Kappa Light Chain CDR1 of BTN1A1 antibody STC43G3-1 (Chothia)
QSSQSVYNNNNLS

SEQ ID NO: 95 - protein, 7 aa, synthetic construct
  source 1..7: note = Kappa Light Chain CDR2 of BTN1A1 antibody STC43G3-1 (Chothia)
RASKLPS

SEQ ID NO: 96 - protein, 10 aa, synthetic construct
  source 1..10: note = Kappa Light Chain CDR3 of BTN1A1 antibody STC43G3-1 (Chothia)
AGGYSGDINV

SEQ ID NO: 97 - protein, 8 aa, synthetic construct
  source 1..8: note = Heavy Chain CDR1 of BTN1A1 antibody STC43G3-1 (IMGT)
GIDLSRYG

SEQ ID NO: 98 - protein, 7 aa, synthetic construct
  source 1..7: note = Heavy Chain CDR2 of BTN1A1 antibody STC43G3-1 (IMGT)
IGTTGNT

SEQ ID NO: 99 - protein, 12 aa, synthetic construct
  source 1..12: note = Heavy Chain CDR3 of BTN1A1 antibody STC43G3-1 (IMGT)
ARSVVSPSNSSF

SEQ ID NO: 100 - protein, 6 aa, synthetic construct
  source 1..6: note = Kappa Light Chain CDR1 of BTN1A1 antibody STC43G3-1 (IMGT)
QSVYNN

SEQ ID NO: 101 - skipped

SEQ ID NO: 102 - protein, 10 aa, synthetic construct
  source 1..10: note = Kappa Light Chain CDR3 of BTN1A1 antibody STC43G3-1 (IMGT)
AGGYSGDINV

SEQ ID NO: 103 - protein, 6 aa, synthetic construct
  source 1..6: note = Heavy Chain CDR1 of BTN1A1 antibody STC43G3-1 (Contact)
SRYGVN

SEQ ID NO: 104 - protein, 12 aa, synthetic construct
  source 1..12: note = Heavy Chain CDR2 of BTN1A1 antibody STC43G3-1 (Contact)
WIGYIGTTGNTY

SEQ ID NO: 105 - protein, 11 aa, synthetic construct
  source 1..11: note = Heavy Chain CDR3 of BTN1A1 antibody STC43G3-1 (Contact)
ARSVVSPSNSS

SEQ ID NO: 106 - protein, 9 aa, synthetic construct
  source 1..9: note = Kappa Light Chain CDR1 of BTN1A1 antibody STC43G3-1 (Contact)
YNNNNLSWF

SEQ ID NO: 107 - protein, 10 aa, synthetic construct
  source 1..10: note = Kappa Light Chain CDR2 of BTN1A1 antibody STC43G3-1 (Contact)
LLIYRASKLP

SEQ ID NO: 108 - protein, 9 aa, synthetic construct
  source 1..9: note = Kappa Light Chain CDR3 of BTN1A1 antibody STC43G3-1 (Contact)
AGGYSGDIN

SEQ ID NO: 109 - DNA, 348 bp, synthetic construct
  source 1..348: mol_type = other DNA; note = nucleic acid sequence VH domain of BTN1A1 antibody STC85F1-1
cagtcgctggaggagtccgggggtcgcctggtcacgcctggaggatccctgacactcacctgcacagtctctggaatcgacctcagtagatatggagtgaactgggtccgccaggctccagggaaggggctggaatggatcggatatattggtactacttctaacacatactacgcgagctgggcgaaaggccgattcaccatctccaaaacctcgtcgaccacggtggatctgaaaatgaccagtctgacaaccgaggacacggccacctatttctgtgccagaagtgttgttagtcctagtaatagtagtttctggggccaaggcaccctggtcaccgtctcctca

SEQ ID NO: 110 - DNA, 330 bp, synthetic construct
  source 1..330: mol_type = other DNA; note = nucleic acid sequence VL domain of BTN1A1 antibody STC85F1-1
gaccctgtgatgacccagactccatcttccacgtctgcggctgtgggaggcacagtcaccatcaactgccagtccagtcagagtgtttataataacaaaaacttatcctggtttcagcagaaaccaggtcagcctcccaagctcctgatctacagggcatccactctgccatctggggtcccatcgcggttcaaaggcagtggatctgggacacagttcactctcaccatcagtgacctggagtgtgacgatgctgccacttactactgtgccggcggttatagtggtgatattaatgttttcggcggagggaccgaggtggtggtcaaa

SEQ ID NO: 111 - protein, 7 aa, synthetic construct
  source 1..7: note = Heavy Chain CDR1 of BTN1A1 antibody STC85F1-1 (Chothia)
GIDLSRY

SEQ ID NO: 112 - protein, 5 aa, synthetic construct
  source 1..5: note = Heavy Chain CDR2 of BTN1A1 antibody STC85F1-1 (Chothia)
GTTSN

SEQ ID NO: 113 - protein, 10 aa, synthetic construct
  source 1..10: note = Heavy Chain CDR3 of BTN1A1 antibody STC85F1-1 (Chothia)
SVVSPSNSSF

SEQ ID NO: 114 - protein, 13 aa, synthetic construct
  source 1..13: note = Kappa Light Chain CDR1 of BTN1A1 antibody STC85F1-1 (Chothia)
QSSQSVYNNKNLS

SEQ ID NO: 115 - protein, 7 aa, synthetic construct
  source 1..7: note = Kappa Light Chain CDR2 of BTN1A1 antibody STC85F1-1 (Chothia)
RASTLPS

SEQ ID NO: 116 - protein, 10 aa, synthetic construct
  source 1..10: note = Kappa Light Chain CDR3 of BTN1A1 antibody STC85F1-1 (Chothia)
AGGYSGDINV

SEQ ID NO: 117 - protein, 8 aa, synthetic construct
  source 1..8: note = Heavy Chain CDR1 of BTN1A1 antibody STC85F1-1 (IMGT)
GIDLSRYG

SEQ ID NO: 118 - protein, 7 aa, synthetic construct
  source 1..7: note = Heavy Chain CDR2 of BTN1A1 antibody STC85F1-1 (IMGT)
IGTTSNT

SEQ ID NO: 119 - protein, 12 aa, synthetic construct
  source 1..12: note = Heavy Chain CDR3 of BTN1A1 antibody STC85F1-1 (IMGT)
ARSVVSPSNSSF

SEQ ID NO: 120 - protein, 8 aa, synthetic construct
  source 1..8: note = Kappa Light Chain CDR1 of BTN1A1 antibody STC85F1-1 (IMGT)
QSVYNNKN

SEQ ID NO: 121 - skipped

SEQ ID NO: 122 - protein, 10 aa, synthetic construct
  source 1..10: note = Kappa Light Chain CDR3 of BTN1A1 antibody STC85F1-1 (IMGT)
AGGYSGDINV

SEQ ID NO: 123 - protein, 6 aa, synthetic construct
  source 1..6: note = Heavy Chain CDR1 of BTN1A1 antibody STC85F1-1 (Contact)
SRYGVN

SEQ ID NO: 124 - protein, 12 aa, synthetic construct
  source 1..12: note = Heavy Chain CDR2 of BTN1A1 antibody STC85F1-1 (Contact)
WIGYIGTTSNTY

SEQ ID NO: 125 - protein, 11 aa, synthetic construct
  source 1..11: note = Heavy Chain CDR3 of BTN1A1 antibody STC85F1-1 (Contact)
ARSVVSPSNSS

SEQ ID NO: 126 - protein, 9 aa, synthetic construct
  source 1..9: note = Kappa Light Chain CDR1 of BTN1A1 antibody STC85F1-1 (Contact)
YNNKNLSWF

SEQ ID NO: 127 - protein, 10 aa, synthetic construct
  source 1..10: note = Kappa Light Chain CDR2 of BTN1A1 antibody STC85F1-1 (Contact)
LLIYRASTLP

SEQ ID NO: 128 - protein, 9 aa, synthetic construct
  source 1..9: note = Kappa Light Chain CDR3 of BTN1A1 antibody STC85F1-1 (Contact)
AGGYSGDIN

SEQ ID NO: 129 - protein, 19 aa, synthetic construct
  source 1..19: note = N terminal signal peptide
METGLRWLLLVAVLKGVQC

SEQ ID NO: 130 - protein, 22 aa, synthetic construct
  source 1..22: note = N terminal signal peptide
MDTRAPTQLLGLLLLWLPGAIC

SEQ ID NO: 131 - protein, 220 aa, Mus musculus
  source 1..220: note = ECD Domain of mouse BTN1A1
AAPFDVTAPQEPVLALVGSDAELTCGFSPNASSEYMELLWFRQTRSTAVLLYRDGQEQEGQQMTEYRGRATLATAGLLDGRATLLIRDVRVSDQGEYRCLFKDNDDFEEAAVYLKVAAVGSDPQISMTVQENGEMELECTSSGWYPEPQVQWRTGNREMLPSTSESKKHNEEGLFTVAVSMMIRDSSIKNMSCCIQNILLGQGKEVEISLPAPFVPRLTP

SEQ ID NO: 132 - protein, 215 aa, Homo sapiens
  source 1..215: note = ECD Domain of human BTN1A1
APFDVIGPPEPILAWGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLPR

SEQ ID NO: 133 - protein, 216 aa, Oryctolagus cuniculus
  source 1..216: note = ECD Domain of rabbit BTN1A1
PFEVLRPPEPILAWGGDAELPCHLPVNASAQRTELRWFRHTFSPAVLVLLAGREQEDEQMPEYRGRASLVPAVLAGGRAALRIRAVRASDDGEYRCIFRKDSSYQEAAVSLKVAALGSDPHIHMEVQENGEIRLECTSVGWYPEPRVQWRTSRGEKLASTSESRSADEEGLFTVATSWIRDTSLGNMSCCIQNPLLGQEKEAGVSIPAPFFPRLPP

SEQ ID NO: 134 - protein, 134 aa, synthetic construct
  source 1..134: note = STC43G3-1 VH with a signal peptide
METGLRWLLLVAVLKGVQCQSLEESGGRLVTPGTPLTLTCTVSGIDLSRYGVNWVRQAPGKGLEWIGYIGTTGNTYYASWVNGRFTISKTSTTVELKMTSLTAADTATYFCARSVVSPSNSSFWGQGTLVTVSS

SEQ ID NO: 135 - protein, 132 aa, synthetic construct
  source 1..132: note = STC43G3-1 VL with a signal peptide
MDTRAPTQLLGLLLLWLPGAICDPVMTQTPSSTSAAVGGTVTINCQSSQSVYNNNNLSWFQQKPGQPPKLLIYRASKLPSGVPPRFSGSGSGTQFTLTISSVQCDDAATYFCAGGYSGDINVFGGGTEVVVK

SEQ ID NO: 136 - protein, 135 aa, synthetic construct
  source 1..135: note = STC85F1-1 VH with a signal peptide
METGLRWLLLVAVLKGVQCQSLEESGGRLVTPGGSLTLTCTVSGIDLSRYGVNWVRQAPGKGLEWIGYIGTTSNTYYASWAKGRFTISKTSSTTVDLKMTSLTTEDTATYFCARSVVSPSNSSFWGQGTLVTVSS

SEQ ID NO: 137 - protein, 132 aa, synthetic construct
  source 1..132: note = STC85F1-1 VL with a signal peptide
MDTRAPTQLLGLLLLWLPGAICDPVMTQTPSSTSAAVGGTVTINCQSSQSVYNNKNLSWFQQKPGQPPKLLIYRASTLPSGVPSRFKGSGSGTQFTLTISDLECDDAATYYCAGGYSGDINVFGGGTEVVVK

SEQ ID NO: 138 - DNA, 396 bp, synthetic construct
  source 1..396: mol_type = other DNA; note = STC43G3-1 with a signal peptide
atggacacgagggcccccactcagctgctggggctcctgctgctctggctcccaggtgccatatgtgaccctgtgatgacccagactccatcttccacgtctgcggctgtgggaggcacagtcaccatcaactgccagtccagtcagagtgtttataataataacaacttatcctggtttcagcagaaaccaggtcagcctcccaagctcctgatctacagggcatccaaactgccatctggggtcccaccgcggttcagcggcagtggatctgggacacagttcactctcaccatcagcagcgtgcagtgtgacgatgctgccacttacttctgtgccggcggttatagtggtgatattaatgttttcggcggagggaccgaggtggtggtcaaa

SEQ ID NO: 139 - DNA, 396 bp, synthetic construct
  source 1..396: mol_type = other DNA; note = STC85F1-1 VL with a signal peptide
atggacacgagggcccccactcagctgctggggctcctgctgctctggctcccaggtgccatatgtgaccctgtgatgacccagactccatcttccacgtctgcggctgtgggaggcacagtcaccatcaactgccagtccagtcagagtgtttataataacaaaaacttatcctggtttcagcagaaaccaggtcagcctcccaagctcctgatctacagggcatccactctgccatctggggtcccatcgcggttcaaaggcagtggatctgggacacagttcactctcaccatcagtgacctggagtgtgacgatgctgccacttactactgtgccggcggttatagtggtgatattaatgttttcggcggagggaccgaggtggtggtcaaa

SEQ ID NO: 140 - DNA, 402 bp, synthetic construct
  source 1..402: mol_type = other DNA; note = STC43G3-1 VH with signal peptide
atggagactgggctgcgctggcttctcctggtcgctgtgctcaaaggtgtccagtgtcagtcgctggaggagtccgggggtcgcctggtcacgcctgggacacccctgacactcacctgcacagtctctggaatcgacctcagtagatatggagtgaactgggtccgccaggctccagggaaggggctggaatggatcggatatattggtactactggtaatacatactacgcgagctgggtgaatggtcgattcaccatctccaaaacctcgaccacggtggagctgaaaatgaccagtctgacagccgcggacacggccacctatttctgtgccagaagtgttgttagtcctagtaatagtagtttctggggccaaggcaccctggtcaccgtctcctca

SEQ ID NO: 141 - DNA, 405 bp, synthetic construct
  source 1..405: mol_type = other DNA; note = STC85F1-1 VH with a signal peptide
atggagactgggctgcgctggcttctcctggtcgctgtgctcaaaggtgtccagtgtcagtcgctggaggagtccgggggtcgcctggtcacgcctggaggatccctgacactcacctgcacagtctctggaatcgacctcagtagatatggagtgaactgggtccgccaggctccagggaaggggctggaatggatcggatatattggtactacttctaacacatactacgcgagctgggcgaaaggccgattcaccatctccaaaacctcgtcgaccacggtggatctgaaaatgaccagtctgacaaccgaggacacggccacctatttctgtgccagaagtgttgttagtcctagtaatagtagtttctggggccaaggcaccctggtcaccgtctcctca

(19) United States
(12) Patent Application Publication
(10) Pub. No.: US 2025/0189530 A1
(43) Pub. Date: Jun. 12, 2025

(54) BTN1A1 BINDING PROTEINS AND METHODS OF USE THEREOF

(71) Applicant: STCUBE & CO., INC., Seoul (KR)

(72) Inventor: Stephen S. YOO, Centreville, VA (US)

(21) Appl. No.: 18/847,179

(22) PCT Filed: Mar. 15, 2023
(86) PCT No.: PCT/US2023/064478

§ 371 Date: Sep. 13, 2024

Related U.S. Application Data
(60) Provisional application No. 63/320,646, filed on Mar. 16, 2022.

(51) Int. Cl. G01N 33/574; C07K 16/28

(52) U.S. Cl. CPC G01N 33/57492; C07K 16/2803; C07K 2317/33

(57) ABSTRACT

Provided herein are molecules having an antigen binding fragment that immunospecifically binds to BTN1A1, such as anti-BTN1A1 antibodies. These molecules include those having an antigen binding fragment that immunospecifically binds to BTN1A1. Methods of making and using these molecules are also provided, including methods of using them in detecting the presence of BTN1A1 in a sample, such as a bodily fluid or tissue of a patient, for diagnostics and treatment of BTN1A1 mediated diseases or conditions.

### CROSS-REFERENCE TO RELATED APPLICATIONS

This application claims the benefit of priority to U.S. Ser. No. 63/320,646 filed Mar. 16, 2022, the contents of which is incorporated herein by reference in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “13532-025-228_SEQLIST.xml”, was created on Mar. 15, 2023, and is 156,059 bytes in size.

### 1. FIELD

The present invention relates in general to the field of cancer immunology and molecular biology. Provided herein are anti-BTN1A1 antibodies or other molecules having an antigen binding fragment that immunospecifically bind to BTN1A1, as well as the uses thereof.

### 2. SUMMARY

The present disclosure provides proteins that bind to BTN1A1 (e.g., human BTN1A1, SEQ ID NO:1), including binding proteins such as antibodies that bind to BTN1A1. Such binding proteins, including antibodies, can bind to a BTN1A1 polypeptide, a BTN1A1 fragment, and/or a BTN1A1 epitope. Such binding proteins, including antibodies, can be agonists (e.g., induce BTN1A1 ligand-like signaling). In some embodiments, the binding proteins do not compete with BTN1A1 ligand for the interaction with BTN1A1 (e.g., a non-blocking antibody).

Also provided herein are polynucleotides and vectors comprising sequences encoding such antibodies, cells (e.g., host cells) comprising such polynucleotides or vectors, and compositions, reagents, and kits comprising such antibodies. In another aspect, provided herein are methods for detection BTN1A1 from a sample, methods for evaluating a subject for treatment, diagnostic methods and other uses of such anti-BTN1A1 antibodies.

In some embodiments, a binding protein (e.g., an anti-BTN1A1 antibody) comprises six complementarity determining regions (CDRs) or fewer than six CDRs. In other embodiments, a binding protein (e.g., an anti-BTN1A1 antibody) comprises one, two, three, four, five, or six CDRs selected from heavy chain variable region (VH) CDR1, VH CDR2, VH CDR3, light chain variable region (VL) CDR1, VL CDR2, and/or VL CDR3. In certain embodiments, a binding protein (e.g., an anti-BTN1A1 antibody) comprises one, two, three, four, five, or six CDRs selected from VH CDR1, VH CDR2, VH CDR3, VL CDR1, VL CDR2, and/or VL CDR3 of a monoclonal antibody designated as STC43H11-1 as described herein, or a humanized variant thereof. In some embodiments, a binding protein (e.g., an anti-BTN1A1 antibody) further comprises a scaffold region or framework region (FR), including a VH FR1, VH FR2, VH FR3, VH FR4, VL FR1, VL FR2, VL FR3, and/or VL FR4 of a human immunoglobulin amino acid sequence or a variant thereof.

In one aspect, provided herein is an antibody or antigen-binding fragment thereof that (a) binds to an epitope of BTN1A1 recognized by an antibody comprising a light chain variable region having an amino acid sequence of SEQ ID NO:19 and a heavy chain variable region having an amino acid sequence of SEQ ID NO:20; or (b) competes for the binding to BTN1A1 with an antibody comprising a light chain variable region having an amino acid sequence of SEQ ID NO:19 and a heavy chain variable region having an amino acid sequence of SEQ ID NO:20.

In one aspect, provided herein is an antibody or antigen-binding fragment thereof that (a) binds to an epitope of BTN1A1 recognized by an antibody comprising a light chain variable region having an amino acid sequence of SEQ ID NO:66 and a heavy chain variable region having an amino acid sequence of SEQ ID NO:68; or (b) competes for the binding to BTN1A1 with an antibody comprising a light chain variable region having an amino acid sequence of SEQ ID NO:66 and a heavy chain variable region having an amino acid sequence of SEQ ID NO:68.

In one aspect, provided herein is an antibody or antigen-binding fragment thereof that (a) binds to an epitope of BTN1A1 recognized by an antibody comprising a light chain variable region having an amino acid sequence of SEQ ID NO:67 and a heavy chain variable region having an amino acid sequence of SEQ ID NO:69; or (b) competes for the binding to BTN1A1 with an antibody comprising a light chain variable region having an amino acid sequence of SEQ ID NO:67 and a heavy chain variable region having an amino acid sequence of SEQ ID NO:69.

In one aspect, provided herein is an antibody or antigen-binding fragment thereof that binds to BTN1A1, wherein the antibody or antigen-binding fragment thereof comprises: (a) a light chain variable region (VL) comprising VL complementarity determining region 1 (CDR1), VL CDR2, and VL CDR3 of antibody STC43H11-1 as set forth in Table 1; and/or a heavy chain variable region (VH) comprising VH complementarity determining region 1 (CDR1), VH CDR2, and VH CDR3 of antibody STC43H11-1 as set forth in Table 2; (b) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:19; and/or a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:20.

In one aspect, provided herein is an antibody or antigen-binding fragment thereof that binds to BTN1A1, wherein the antibody or antigen-binding fragment thereof comprises: (a) a light chain variable region (VL) comprising VL complementarity determining region 1 (CDR1), VL CDR2, and VL CDR3 of antibody STC43G3-1 as set forth in Table 3; and/or a heavy chain variable region (VH) comprising VH complementarity determining region 1 (CDR1), VH CDR2, and VH CDR3 of antibody STC43G3-1 as set forth in Table 4; (b) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66; and/or a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:68.

In one aspect, provided herein is an antibody or antigen-binding fragment thereof that binds to BTN1A1, wherein the antibody or antigen-binding fragment thereof comprises: (a) a light chain variable region (VL) comprising VL complementarity determining region 1 (CDR1), VL CDR2, and VL CDR3 of antibody STC85F1-1 as set forth in Table 5; and/or a heavy chain variable region (VH) comprising VH complementarity determining region 1 (CDR1), VH CDR2, and VH CDR3 of antibody STC85F1-1 as set forth in Table 6; (b) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:67; and/or a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:69.

In some embodiments, the VH CDR1, VH CDR2, VH CDR3, VL CDR1, VL CDR2, and VL CDR3 amino acid sequences are according to the Kabat numbering system. In some embodiments, the VH CDR1, VH CDR2, VH CDR3, VL CDR1, VL CDR2, and VL CDR3 amino acid sequences are according to the Chothia numbering system. In some embodiments, the VH CDR1, VH CDR2, VH CDR3, VL CDR1, VL CDR2, and VL CDR3 amino acid sequences are according to the ABM numbering system. In some embodiments, the VH CDR1, VH CDR2, VH CDR3, VL CDR1, VL CDR2, and VL CDR3 amino acid sequences are according to the Contact numbering system. In some embodiments, the VH CDR1, VH CDR2, VH CDR3, VL CDR1, VL CDR2, and VL CDR3 amino acid sequences are according to the IMGT numbering system.

In some embodiments, the antibody or antigen-binding fragment thereof comprises: (a) a light chain variable region (VL) further comprising VL framework 1 (FR1), VL FR2, VL FR3, and VL FR4 of any one of antibody STC43H11-1 as set forth in Table 7; and/or (b) a heavy chain variable region (VH) further comprising VH framework 1 (FR1), VH FR2, VH FR3, and VH FR4 of any one of antibody STC43H11-1 as set forth in Table 8.

In some embodiments, the antibody or antigen-binding fragment thereof comprises: (a) a light chain variable region (VL) further comprising VL framework 1 (FR1), VL FR2, VL FR3, and VL FR4 of any one of antibody STC43G3-1 as set forth in Table 7; and/or (b) a heavy chain variable region (VH) further comprising VH framework 1 (FR1), VH FR2, VH FR3, and VH FR4 of any one of antibody STC43G3-1 as set forth in Table 8.

In some embodiments, the antibody or antigen-binding fragment thereof comprises: (a) a light chain variable region (VL) further comprising VL framework 1 (FR1), VL FR2, VL FR3, and VL FR4 of any one of antibody STC85F1-1 as set forth in Table 7; and/or (b) a heavy chain variable region (VH) further comprising VH framework 1 (FR1), VH FR2, VH FR3, and VH FR4 of any one of antibody STC85F1-1 as set forth in Table 8.

In some embodiments, the VL CDR1, VL CDR2, and VL CDR3 comprise amino acid sequences of SEQ ID NOS:5, 6, and 7, respectively, and the VH CDR1, VH CDR2, and VH CDR3 comprise amino acid sequences of SEQ ID NOS:8, 9, and 10, respectively.

In some embodiments, the VL CDR1, VL CDR2, and VL CDR3 comprise amino acid sequences of SEQ ID NOS:74, 75, and 76, respectively, and the VH CDR1, VH CDR2, and VH CDR3 comprise amino acid sequences of SEQ ID NOS:71, 72, and 73, respectively.

In some embodiments, the VL CDR1, VL CDR2, and VL CDR3 comprise amino acid sequences of SEQ ID NOS:80, 81, and 82, respectively, and the VH CDR1, VH CDR2, and VH CDR3 comprise amino acid sequences of SEQ ID NOS:77, 78, and 79, respectively.

In some embodiments, the VL CDR1, VL CDR2, and VL CDR3 comprise amino acid sequences of SEQ ID NOS:86, 87, and 88, respectively, and the VH CDR1, VH CDR2, and VH CDR3 comprise amino acid sequences of SEQ ID NOS:83, 84, and 85, respectively, respectively.

In some embodiments, the antibody or antigen-binding fragment thereof comprises a VL comprising an amino acid sequence having at least 95% sequence identity to SEQ ID NO:19. In some embodiments, the antibody or antigen-binding fragment thereof comprises a VL comprising an amino acid sequence of SEQ ID NO:19.

In some embodiments, the antibody or antigen-binding fragment thereof comprises a VH comprising an amino acid sequence having at least 95% sequence identity to SEQ ID NO:20. In some embodiments, the antibody or antigen-binding fragment thereof comprises a VH comprising an amino acid sequence of SEQ ID NO:20. In some embodiments, the antibody or antigen-binding fragment thereof comprises a VL comprising an amino acid sequence having at least 95% sequence identity to SEQ ID NO:19 and a VH comprising an amino acid sequence having at least 95% sequence identity to SEQ ID NO:20.

In some embodiments, the antibody or antigen-binding fragment thereof comprises: (a) a VL comprising an amino acid sequence of SEQ ID NO:19; and (b) a VH comprising an amino acid sequence of SEQ ID NO:20.

In some embodiments, the VL CDR1, VL CDR2, and VL CDR3 comprise amino acid sequences of SEQ ID NOS:38, 39, and 40, respectively, and the VH CDR1, VH CDR2, and VH CDR3 comprise amino acid sequences of SEQ ID NOS:44, 45, and 46, respectively.

In some embodiments, the VL CDR1, VL CDR2, and VL CDR3 comprise amino acid sequences of SEQ ID NOS:94, 95, and 96, respectively, and the VH CDR1, VH CDR2, and VH CDR3 comprise amino acid sequences of SEQ ID NOS:91, 92, and 93, respectively.

In some embodiments, the VL CDR1, VL CDR2, and VL CDR3 comprise amino acid sequences of SEQ ID NOS:100, 101, and 102, respectively, and the VH CDR1, VH CDR2, and VH CDR3 comprise amino acid sequences of SEQ ID NOS:97, 98, and 99, respectively.

In some embodiments, the VL CDR1, VL CDR2, and VL CDR3 comprise amino acid sequences of SEQ ID NOS:106, 107, and 108, respectively, and the VH CDR1, VH CDR2, and VH CDR3 comprise amino acid sequences of SEQ ID NOS:103, 104, and 105, respectively, respectively.

In some embodiments, the antibody or antigen-binding fragment thereof comprises a VL comprising an amino acid sequence having at least 95% sequence identity to SEQ ID NO:66. In some embodiments, the antibody or antigen-binding fragment thereof comprises a VL comprising an amino acid sequence of SEQ ID NO:66.

In some embodiments, the antibody or antigen-binding fragment thereof comprises a VH comprising an amino acid sequence having at least 95% sequence identity to SEQ ID NO:68. In some embodiments, the antibody or antigen-binding fragment thereof comprises a VH comprising an amino acid sequence of SEQ ID NO:68.

In some embodiments, the antibody or antigen-binding fragment thereof comprises a VL comprising an amino acid sequence having at least 95% sequence identity to SEQ ID NO:66 and a VH comprising an amino acid sequence having at least 95% sequence identity to SEQ ID NO:68. In some embodiments, the antibody or antigen-binding fragment thereof comprises: (a) a VL comprising an amino acid sequence of SEQ ID NO:66; and (b) a VH comprising an amino acid sequence of SEQ ID NO:68.

In some embodiments, the VL CDR1, VL CDR2, and VL CDR3 comprise amino acid sequences of SEQ ID NOS:41, 42, and 43, respectively, and the VH CDR1, VH CDR2, and VH CDR3 comprise amino acid sequences of SEQ ID NOS:47, 48, and 49, respectively.

In some embodiments, the VL CDR1, VL CDR2, and VL CDR3 comprise amino acid sequences of SEQ ID NOS: 114, 115, and 116, respectively, and the VH CDR1, VH CDR2, and VH CDR3 comprise amino acid sequences of SEQ ID NOS: 111, 112, and 113, respectively.

In some embodiments, the VL CDR1, VL CDR2, and VL CDR3 comprise amino acid sequences of SEQ ID NOS:120, 121, and 122, respectively, and the VH CDR1, VH CDR2, and VH CDR3 comprise amino acid sequences of SEQ ID NOS:117, 118, and 119, respectively.

In some embodiments, the VL CDR1, VL CDR2, and VL CDR3 comprise amino acid sequences of SEQ ID NOS:126, 127, and 128, respectively, and the VH CDR1, VH CDR2, and VH CDR3 comprise amino acid sequences of SEQ ID NOS:123, 124, and 125, respectively, respectively

In some embodiments, the antibody or antigen-binding fragment thereof comprises a VL comprising an amino acid sequence having at least 95% sequence identity to SEQ ID NO:67. In some embodiments, the antibody or antigen-binding fragment thereof comprises a VL comprising an amino acid sequence of SEQ ID NO:67.

In some embodiments, the antibody or antigen-binding fragment thereof comprises a VH comprising an amino acid sequence having at least 95% sequence identity to SEQ ID NO:69. In some embodiments, the antibody or antigen-binding fragment thereof comprises a VH comprising an amino acid sequence of SEQ ID NO:69.

In some embodiments, the antibody or antigen-binding fragment thereof comprises a VL comprising an amino acid sequence having at least 95% sequence identity to SEQ ID NO:67 and a VH comprising an amino acid sequence having at least 95% sequence identity to SEQ ID NO:69. In some embodiments, the antibody or antigen-binding fragment thereof comprises: (a) a VL comprising an amino acid sequence of SEQ ID NO:67; and (b) a VH comprising an amino acid sequence of SEQ ID NO:69.

In some embodiments, the antibody or antigen-binding fragment thereof binds to human BTN1A1. In some embodiments, the antibody or antigen-binding fragment thereof binds to mouse BTN1A1.

In some embodiments, when bound to BTN1A1, the antibody or antigen-binding fragment binds to at least one of residues 361-375 within an amino acid sequence of SEQ ID NO:1. In some embodiments, when bound to BTN1A1, the antibody or antigen-binding fragment binds to at least one of residues 365-372 within an amino acid sequence of SEQ ID NO:1. In some embodiments, when bound to BTN1A1, the antibody or antigen-binding fragment binds to at least one of residues 366-372 within an amino acid sequence of SEQ ID NO:1.

In some embodiments, when bound to BTN1A1, the antibody or antigen-binding fragment binds to at least one residue selected from the group consisting of G365, D366, T368, D369, W370, A371, I372, and G373 within an amino acid sequence of SEQ ID NO:1. In some embodiments, when bound to BTN1A1, the antibody or antigen-binding fragment binds to G365 within an amino acid sequence of SEQ ID NO:1. In some embodiments, when bound to BTN1A1, the antibody or antigen-binding fragment binds to D366 within an amino acid sequence of SEQ ID NO:1. In some embodiments, when bound to BTN1A1, the antibody or antigen-binding fragment binds to T368 within an amino acid sequence of SEQ ID NO:1. In some embodiments, when bound to BTN1A1, the antibody or antigen-binding fragment binds to D369 within an amino acid sequence of SEQ ID NO:1. In some embodiments, when bound to BTN1A1, the antibody or antigen-binding fragment binds to W370 within an amino acid sequence of SEQ ID NO:1. In some embodiments, when bound to BTN1A1, the antibody or antigen-binding fragment binds to A371 within an amino acid sequence of SEQ ID NO:1. In some embodiments, when bound to BTN1A1, the antibody or antigen-binding fragment binds to I372 within an amino acid sequence of SEQ ID NO:1. In some embodiments, when bound to BTN1A1, the antibody or antigen-binding fragment binds to G373 within an amino acid sequence of SEQ ID NO:1.

In some embodiments, when bound to BTN1A1, the antibody or antigen-binding fragment binds to D366, D369 and I372 within an amino acid sequence of SEQ ID NO:1. In some embodiments, when bound to BTN1A1, the antibody or antigen-binding fragment binds to G365, T368, D369, A371 and I372 within an amino acid sequence of SEQ ID NO: 1. In some embodiments, when bound to BTN1A1, the antibody or antigen-binding fragment binds to D366, T368, D369, W370, A371, I372, and G373 within an amino acid sequence of SEQ ID NO:1.

In some embodiments, the antibody is a monoclonal antibody. In some embodiments, the antibody is a humanized, human, or chimeric antibody. In some embodiments, the humanized antibody is a deimmunized antibody or a composite human antibody. In some embodiments, the antibody or antigen-binding fragment thereof is a Fab, a Fab′, a F(ab′)2, a Fv, a scFv, a dsFv, a diabody, a triabody, a tetrabody, or a multispecific antibody formed from antibody fragments.

In some embodiments, the antibody or antigen-binding fragment thereof is conjugated to an agent. In some embodiments, the agent is selected from the group consisting of a radioisotope, a metal chelator, an enzyme, a fluorescent compound, a bioluminescent compound, and a chemiluminescent compound.

In one aspect, provided herein is a composition comprising an antibody or antigen-binding fragment thereof provided herein, and a pharmaceutically acceptable carrier.

In one aspect, provided herein is a polynucleotide comprising nucleotide sequences encoding a VH, a VL, or both a VH and a VL of an antibody provided herein.

In one aspect, provided herein is a polynucleotide comprising nucleotide sequences encoding a heavy chain, a light chain, or both a heavy chain and a light chain of an antibody provided herein. In some embodiments, the polynucleotide is operably linked to a promoter.

In one aspect, provided herein is a vector comprising a polynucleotide provided herein.

In one aspect, provided herein is a cell comprising a polynucleotide provided herein. In one aspect, provided herein is a cell comprising a vector provided herein. In one aspect, provided herein is an isolated cell producing an antibody or antigen-binding fragment thereof provided herein.

In one aspect, provided herein is a kit comprising an antibody or antigen-binding fragment thereof provided herein.

In one aspect, provided herein is a method of making an antibody or antigen-binding fragment thereof which specifically binds to an epitope of BTN1A1, comprising culturing a cell provided herein to express the antibody or antigen-binding fragment thereof. In one aspect, provided herein is a method of making an antibody or antigen-binding fragment thereof which specifically binds to an epitope of BTN1A1, comprising expressing a polynucleotide provided herein.

In one aspect, provided herein is a method of detecting a BTN1A1 in a sample, comprising contacting the sample with an anti-BTN1A1 antibody or antigen binding fragment provided herein, and detecting the presence of the anti-BTN1A1 antibody or antigen binding fragment thereof bound to the sample, wherein detection above background of an amount of the anti-BTN1A1 antibody or antigen binding fragment thereof bound to the sample indicates the presence of BTN1A1 in the sample. In some embodiments, the method further comprises quantitating the amount of detected BTN1A1 in the sample.

In some embodiments, the anti-BTN1A1 antibody or antigen binding fragment thereof is conjugated with a detectable agent producing a signal, and the detecting comprises detecting the signal from the sample. In some embodiments, the anti-BTN1A1 antibody or antigen binding fragment thereof is contacted with a secondary binding molecule that specifically binds to the anti-BTN1A1 antibody or antigen binding fragment in the sample, and the detecting comprises detecting the secondary binding molecule from the sample.

In some embodiments, the sample is a cell-containing sample. In some embodiments, the cells in the sample are fixed in situ before contacted with the anti-BTN1A1 antibody or antigen binding fragment thereof. In some embodiments, the sample is a formalin fixed paraffin embedded (FFPE) tissue sample. In some embodiments, the sample is a cancer tissue; optionally wherein the cancer is selected from skin cancer, pancreases cancer, breast cancer, esophagus cancer, and endometrium cancer. In some embodiments, the sample is a bodily fluid sample; optionally wherein the bodily fluid sample is blood, serum or plasma.

In some embodiments, the sample is obtained from a subject, and wherein the method further comprises diagnosing the subject for having or at risk of developing a BTN1A1-mediated disease or condition upon detection above a threshold of an amount of BTN1A1 in the sample. In some embodiments, the BTN1A1-mediated disease or condition is cancer.

In some embodiments, the subject is a mammal. In some embodiments, the subject is human.

In one aspect, provided herein is a kit for performing any one of the methods provided herein.

In one aspect, provided herein is a method of treating a subject with a BTN1A1 targeting agent, comprising a step of detecting BTN1A1 in a sample from the subject. In some embodiments, the step of detecting BTN1A1 in the sample comprises any one of the methods provided herein. In some embodiments, the targeting agent is an antagonistic polypeptide binding to BTN1A1. In some embodiments, the targeting agent is an agonistic polypeptide binding to BTN1A1. In some embodiments, the polypeptide binding to BTN1A1 is an antibody or antigen binding fragment thereof. In some embodiments, the targeting agent is a small molecule compound inhibiting BTN1A1. In some embodiments, the subject has a disease or condition mediated by BTN1A1. In some embodiments, the method eliminates a population of diseased cells expressing BTN1A1. In some embodiments, the subject has cancer.

### 3. BRIEF DESCRIPTION OF THE FIGURES

FIG. 1 shows a general workflow for generating anti-BTN1A1 antibody.

FIG. 2 shows alignment of the ICD domains of human BTN1A1 (SEQ ID NO:3) and mouse BTN1A1 (SEQ ID NO:4), and a peptide (peptide 4 of SEQ ID NO:23) used to immunize animals in the BTN1A1 ICD.

FIGS. 3A-3C show binding of diluted serum sample taken from immunized rabbits to BTN1A1 peptide 4, hBTN1A1-ICD and denatured hBTN1A1-ICD, respectively, by ELISA.

FIGS. 4A-4C show screening of antibodies produced by different hybridoma clones to peptide 4, hBTN1A1-ICD and denatured hBTN1A1-ICD, respectively, by ELISA.

FIGS. 5A and 5B show binding of purified antibody STC43H11-1 to peptide 4 or hBTN1A1-ICD, respectively, by ELISA.

FIGS. 6A-6C show sequencing and isotyping characterization of STC43H11-1.

FIGS. 7A-7F show detection of BTN1A1 protein expressed by HEK293T cells by western blotting with STC43H11-1 at concentration of (1 μg/ml). Also shown are western blotting detection of a Flag peptide and R-actin expressed by the cells as controls.

FIG. 8 shows detection of BTN1A1 protein expressed by HEK293T cells stained by STC43H11-1 or control IgG (upper panel) via an immunohistochemistry assay. HEK293T cells that did not express BTN1A1 were included as a negative control (left column).

FIG. 9A-9C show immunostaining of various tissues samples (histologically normal (HN), bladder, NSCLC, ovary, colon, pancreas) using STC43H11-1 or an anti-PD-L1 antibody.

FIGS. 10A-10E show immunostaining of a tissue microarray (“TMA”) using STC43H11-1 at a concentration of 2 μg/mL (FIG. 10C), 5 μg/mL (FIG. 10D), and 10 μg/mL (FIG. 10D). A negative control was included using staining by rabbit IgG at the concentration of 2 μg/mL (FIG. 10A).

FIG. 11 shows detection of BTN1A1 protein by immunohistochemistry on formalin-fixed paraffin embedded (FFPE) cells expressing BTN1A1. STC43H11-1 was used for immunostaining at a concentration of 2 μg/mL.

FIG. 12 show immunostaining of tissue sample using IgG (negative control), rabbit polyclonal antibody R4-17 (control), STC43H11-1 and E1L3N (PD-L1) antibodies, respectively, in an immunohistochemistry assay on tissue sample (LC-642b TMA). STC43H11-1 was used for immunostaining at a concentration of 2 μg/mL

FIG. 13 show immunostaining of tissue sample using IgG (negative control), rabbit polyclonal antibody R4-17 (control), STC43H11-1 and E1L3N antibodies, respectively, in an immunohistochemistry assay on tissue sample (LC-642b: 6F). STC43H11-1 was used for immunostaining at a concentration of 2 μg/mL.

FIG. 14 show immunostaining of tissue sample using IgG (negative control), rabbit polyclonal antibody R4-17 (control), STC43H11-1 and E1L3N antibodies, respectively, in an immunohistochemistry assay on tissue sample (LC-642b: 6G).

FIG. 15 show immunostaining of tissue sample using IgG (negative control), rabbit polyclonal antibody R4-17 (control), STC43H11-1 and E1L3N antibodies, respectively, in an immunohistochemistry assay on tissue sample (HN TMA: E6). STC43H11-1 was used for immunostaining at a concentration of 2 μg/mL.

FIG. 16 show immunostaining of tissue sample using IgG (negative control), rabbit polyclonal antibody R4-17 (control), STC43H11-1 and E1L3N antibodies, respectively, in an immunohistochemistry assay on tissue sample (HN TMA: B3). STC43H11-1 was used for immunostaining at a concentration of 2 μg/mL.

FIG. 17 shows immunostaining of multiple skin tumor tissue, pancreas cancer tissue and breast cancer tissue using STC43H11-1 in an immunohistochemistry assay. STC43H11-1 was used for immunostaining at a concentration of 2 μg/mL.

FIG. 18 shows immunostaining of esophagus cancer tissue and endometrium cancer tissue using STC43H11-1 in an immunohistochemistry assay.

FIGS. 19A-19D shows immunostaining of mouse 4T1 breast cancer cells expressing (lower panels) or not expressing mBTN1A1 (upper panels) in an immunohistochemistry assay. STC43H11-1 was used for immunostaining at a concentration of 2 μg/mL (FIG. 19B), 5 μg/mL (FIG. 19C) or 10 μg/mL (FIG. 19D). Rabbit IgG was used for immunostaining at a concentration of 10 μg/mL as a negative control (FIG. 19A).

FIG. 20 shows detection of BTN1A1 protein by immunohistochemistry on mouse mammary fat pads that were snap frozen. Mice were either heterologous for mBTN1A1 (hetero) or a BTN1A1 knockout (KO). STC43H11-1 was used for immunostaining at a concentration of 10 μg/mL.

FIG. 21 shows a sensorgram depicting the stages of interaction between STC43H11-1 and variants of peptide 4 containing the mutated alanine (or glycine) residue at specified locations shown in Table 14.

FIG. 22 shows a sensorgram depicting the interaction between STC43H11-1 and mutants of peptide 4, containing alanine mutations. STC43H11-1 was found to bind to peptide 4 and mutant peptide #6 and 10 of peptide 4.

FIG. 23 shows sensorgrams depicting the interaction between STC43H11-1 and peptide 4 or various mutated variants of peptide 4.

FIG. 24 shows sensorgrams depicting the interaction between STC43H11-1 and peptides containing an amino acid sequence that is homologous to the amino acid sequence of peptide 4.

FIG. 25 shows detection of mutated variants of peptide 4 expressed by HEK293T cells by western blot using STC43H11-1 at the concentration of (1 μg/ml). Also shown are western blotting detection of a Flag peptide and R-actin expressed by the cells as controls.

FIG. 26 shows the quantitated percentage of relative binding between STC43H11-1 and mutated variants of peptide 4 as measured by the western blotting assay shown in FIG. 25.

FIG. 27 shows the alignment of the ECD domains of mouse BTN1A1 (SEQ ID NO:131), human BTN1A1 (SEQ ID NO:132), and rabbit BTN1A1 (SEQ ID NO:133).

FIG. 28 shows the alignment of the heavy chains between STC43G3-1 (SEQ ID NO:134) and STC85F1-1 (SEQ ID NO:136), both with an N terminal signal peptide METGLRWLLLVAVLKGVQC (SEQ ID NO:129).

FIG. 29 shows the alignment of the light chains between STC43G3-1 (SEQ ID NO:135) and STC85F1-1 (SEQ ID NO:137), both with an N terminal signal peptide MDTRAPTQLLGLLLLWLPGAIC (SEQ ID NO:130).

FIGS. 30A and 30B show the binding of multiple purified antibodies to ECD1 or hBTN1A1-His, by ELISA.

FIGS. 31A and 31B show the binding of purified STC43G3-1 and STC85F1-1, respectively, to monomer forms of BTN1A1 (denatured hBTN1A1-His and hBTN1A1-His), and the ECD1 peptide, by ELISA.

FIG. 32A shows detection of BTN1A1 protein expressed on HEK293T cells via immunohistochemistry using the STC43G3-1 antibody. HEK293T cells that over express BTN1A1 (lower row) is compared to HEK293T cells that do not over express BTN1A1 (upper row).

FIG. 32B shows magnified images of FIG. 32A. STC43G3-1 antibody shows strong membranous staining of BTN1A1 on the membrane of HEK293T cells.

FIG. 33A shows detection of BTN1A1 protein expressed on HEK293T cells via immunohistochemistry using the STC85F1-1 antibody. HEK293T cells that over express BTN1A1 (lower row) compared to HEK293T cells that do not over express BTN1A1 (upper row).

FIG. 33B shows magnified images of FIG. 33A. STC85F1-1 antibody shows strong membranous staining of BTN1A1 on the membrane of HEK293T cells.

FIG. 34 shows a map of the human BTN1A1 amino acid sequences (SEQ ID NO:1).

### 4. DETAILED DESCRIPTION

The B7 family of co-stimulatory molecules can drive the activation and inhibition of immune cells. A related family of molecules—the buryrophilins—also have immunomodulatory functions similar to B7 family members. Butyrophilin, subfamily 1, member A1 (BTN1A1) is a type I membrane glycoprotein and a major component of milk fat globule membrane, and has structural similarities to the B7 family. BTN1A1 is known to be a major protein regulating the formation of fat droplets in the milk. (Ogg et al. PNAS, 101(27):10084-10089 (2004)). BTN1A1 is also expressed in immune cells, including T cells. Treatment with recombinant BTN1A1 was found to inhibit T cell activation and protect animal models of EAE. (Stefferl et al., J. Immunol. 165(5):2859-65 (2000)).

BTN1A1 is also specifically and highly expressed in cancer cells. The BTN1A1 in cancer cells can be used to aid in the diagnosis of cancer as well as the evaluation of the efficacy of a cancer treatment.

Provided herein are anti-BTN1A1 antibodies and other molecules that can immunospecifically bind to BTN1A1, and methods for use thereof in providing cancer diagnosis, evaluating of a cancer treatment, or modulating the activity of immune cells and in treating cancers.

### 4.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dubel eds., 2d ed. 2010).

### 4.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein, and unless otherwise specified, the term “Butyrophilin, subfamily 1, member A1” or “BTN1A1” refers to BTN1A1 from any vertebrate source, including mammals such as primates (e.g., humans, cynomolgus monkey (cyno)), dogs, and rodents (e.g., mice and rats). Unless otherwise specified, BTN1A1 also includes various BTN1A1 isoforms, related BTN1A1 polypeptides, including SNP variants thereof, as well as different modified forms of BTN1A1, including but not limited to phosphorylated BTN1A1, glycosylated BTN1A1, and ubiquitinated BTN1A1. As used herein, glycosylated BTN1A1 include BTN1A1 with N55, N215, and/or N449 glycosylation.

An exemplary amino acid sequence of human BTN1A1 (BC096314.1 GI: 64654887), is provided below:

(SEQ ID NO: 1)
MAVFPSSGLPRCLLTLILLQLPKLDSAPFDVIGPPEPILAVVGEDAKLP
CRLSPNASAEHLELRWFRKKVSPAVLVHRDGREQEAEQMPEYRGRATLV
QDGIAKGRVALRIRGVRVSDDGEYTCFFREDGSYEEALVHLKVAALGSD
PHISMQVQENGEICLECTSVGWYPEPQVQWRTSKGEKFPSTSESRNPDE
EGLFTVAASVIIRDTSAKNVSCYIQNLLLGQEKKVEISIPASSLPRLTP
WIVAVAVILMVLGLLTIGSIFFTWRLYNERPRERRNEFSSKERLLEELK
WKKATLHAVDVTLDPDTAHPHLFLYEDSKSVRLEDSRQKLPEKTERFDS
WPCVLGRETFTSGRHYWEVEVGDRTDWAIGVCRENVMKKGFDPMTPENG
FWAVELYGNGYWALTPLRTPLPLAGPPRRVGIFLDYESGDISFYNMNDG
SDIYTFSNVTFSGPLRPFFCLWSSGKKPLTICPIADGPERVIVIANAQD
LSKEIPLSPMGEDSAPRDADTLHSKLIPTQPSQGAP

An exemplary encoding nucleic acid sequence of human BTN1A1 (BC096314.1 GI: 64654887), is provided below:

(SEQ ID NO: 2)
ATGGCAGTTTTCCCAAGCTCCGGTCTCCCCAGATGTCTGCTCACCCTCATTCTCCTCC
AGCTGCCCAAACTGGATTCAGCTCCCTTTGACGTGATTGGACCCCCGGAGCCCATCC
TGGCCGTTGTGGGTGAGGACGCCAAGCTGCCCTGTCGCCTGTCTCCGAACGCGAGCG
CCGAGCACTTGGAGCTACGCTGGTTCCGAAAGAAGGTTTCGCCGGCCGTGCTGGTGC
ATAGGGACGGGCGCGAGCAGGAAGCCGAGCAGATGCCCGAGTACCGCGGGCGGGC
GACGCTGGTCCAGGACGGCATCGCCAAGGGGCGCGTGGCCTTGAGGATCCGTGGCG
TCAGAGTCTCTGACGACGGGGAGTACACGTGCTTTTTCAGGGAGGATGGAAGCTAC
GAAGAAGCCCTGGTGCATCTGAAGGTGGCTGCTCTGGGCTCTGACCCTCACATCAGT
ATGCAAGTTCAAGAGAATGGAGAAATCTGTCTGGAGTGCACCTCAGTGGGATGGTA
CCCAGAGCCCCAGGTGCAGTGGAGAACTTCCAAGGGAGAGAAGTTTCCATCTACAT
CAGAGTCCAGGAATCCTGATGAAGAAGGTTTGTTCACTGTGGCTGCTTCAGTGATCA
TCAGAGACACTTCTGCGAAAAATGTGTCCTGCTACATCCAGAATCTCCTTCTTGGCC
AGGAGAAGAAAGTAGAAATATCCATACCAGCTTCCTCCCTCCCAAGGCTGACTCCCT
GGATAGTGGCTGTGGCTGTCATCCTGATGGTTCTAGGACTTCTCACCATTGGGTCCA
TATTTTTCACTTGGAGACTATACAACGAAAGACCCAGAGAGAGGAGGAATGAATTC
AGCTCTAAAGAGAGACTCCTGGAAGAACTCAAATGGAAAAAGGCTACCTTGCATGC
AGTTGATGTGACTCTGGACCCAGACACAGCTCATCCCCACCTCTTTCTTTATGAGGA
TTCAAAATCTGTTCGACTGGAAGATTCACGTCAGAAACTGCCTGAGAAAACAGAGA
GATTTGACTCCTGGCCCTGTGTGTTGGGCCGTGAGACCTTCACCTCAGGAAGGCATT
ACTGGGAGGTGGAGGTGGGAGACAGGACTGACTGGGCAATCGGCGTGTGTAGGGA
GAATGTGATGAAGAAAGGATTTGACCCCATGACTCCTGAGAATGGGTTCTGGGCTGT
AGAGTTGTATGGAAATGGGTACTGGGCCCTCACTCCTCTCCGGACCCCTCTCCCATT
GGCAGGGCCCCCACGCCGGGTTGGGATTTTCCTAGACTATGAATCAGGAGACATCTC
CTTCTACAACATGAATGATGGATCTGATATCTATACTTTCTCCAATGTCACTTTCTCT
GGCCCCCTCCGGCCCTTCTTTTGCCTATGGTCTAGCGGTAAAAAGCCCCTGACCATC
TGCCCAATTGCTGATGGGCCTGAGAGGGTCACAGTCATTGCTAATGCCCAGGACCTT
TCTAAGGAGATCCCATTGTCCCCCATGGGGGAGGACTCTGCCCCTAGGGATGCAGA
CACTCTCCATTCTAAGCTAATCCCTACCCAACCCAGCCAAGGGGCACCTTAA.

A signal peptide is a short amino acid sequence that may control protein secretion and translocation in a living cell. In some embodiments, a signal peptide can be fused to an antibody sequence, such as to the heavy chain (e.g., VH) or light chain (e.g., VL) of an antibody. In some embodiments, a signal peptide is fused to the N terminus of an antibody sequence. In some embodiments, a signal peptide is fused to the N terminus of an antibody VH sequence described herein. In some embodiments, a signal peptide is fused to the N terminus of an antibody VL sequence described herein. In some embodiments, a signal peptide is fused to the N terminus of an antibody VH region. In specific embodiments, the signal peptide has the sequence of METGLRWLLLVAVLKGVQC (SEQ TD NO:129) and is fused to the N terminus of an antibody VH sequence described herein. In specific embodiments, the signal peptide has the sequence of MDTRAPTQLLGLLLLWLPGAIC (SEQ ID NO: 130) and is fused to an antibody VL sequence described herein.

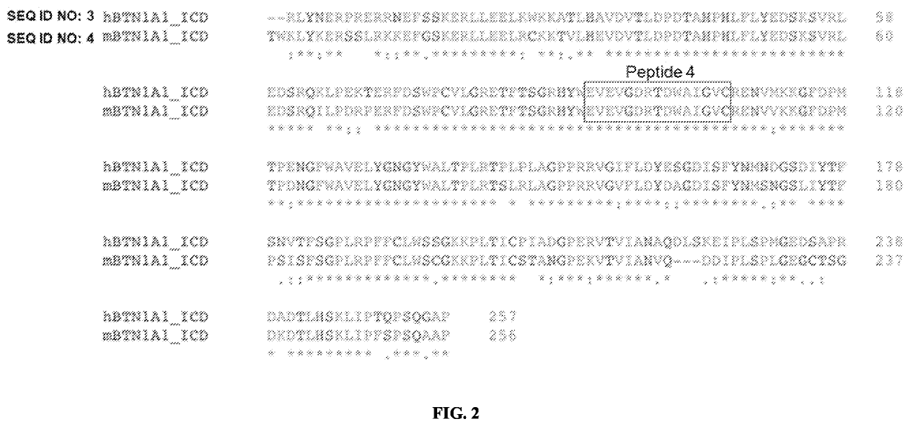

A BTN1A1 polypeptide “intracellular domain” or “ICD” refers to a form of the BTN1A1 polypeptide that is essentially free of the transmembrane and extracellular domains. For example, a BTN1A1 polypeptide ICD may have less than 1% of such transmembrane and/or extracellular domains and can have less than 0.5% of such domains. FIG. 2 shows alignment of two examples of BTN1A1 ICD amino acid sequences originated from human and mouse, respectively. A peptide (peptide 4) having the sequence of EVEVGDRTDWAIGVC (SEQ ID NO:23) in the ICD domain was used as an immunogen to produce anti-BTN1A1 antibodies, such as STC43H11-1.

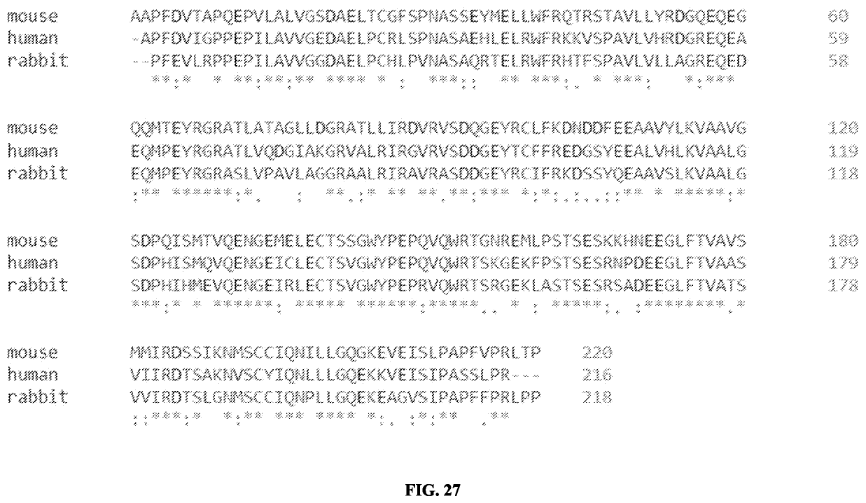

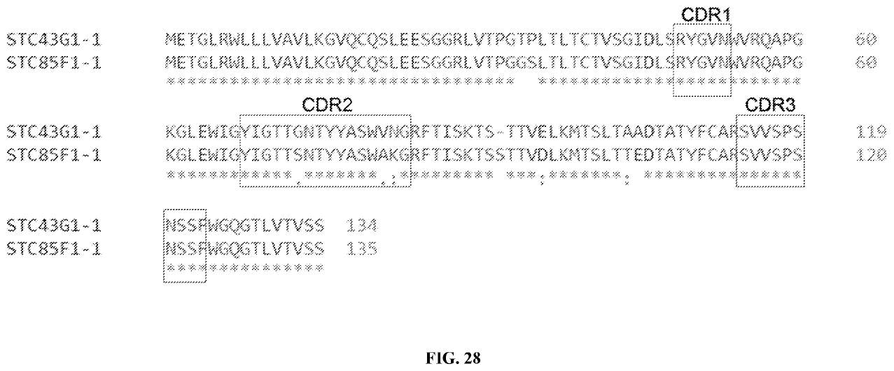

A BTN1A1 polypeptide “extracellular domain” or “ECD” refers to a form of the BTN1A1 polypeptide that is essentially free of the transmembrane and the intracellular domain. For example, a BTN1A1 polypeptide extracellular domain may have less than 1% of such transmembrane and/or intracellular domain and can have less than 0.5% of such domains. Two regions in the BTN1A1 ECD (sometimes referred to as “ECD1” and “ECD2” in this application) were chosen for immunizing animals. FIG. 27 shows the alignment of ECD amino acid sequences among mouse, human, and rabbit. A peptide having the sequence of RKKVSPAVLVHRDGREQEAE (SEQ ID NO:70) in the ECD domain (ECD1) was used as the immunogen to produce anti-BTN1A1 antibodies, such as STC43G3-1 and STC85F1-1.

“Related BTN1A1 polypeptides” include allelic variants (e.g., SNP variants); splice variants; fragments; derivatives; substitution, deletion, and insertion variants; fusion polypeptides; and interspecies homologs, which can retain BTN1A1 activity. As those skilled in the art will appreciate, an anti-BTN1A1 antibody provided herein can bind to a BTN1A1 polypeptide, a BTN1A1 polypeptide fragment, a BTN1A1 antigen, and/or a BTN1A1 epitope. An “epitope” may be part of a larger BTN1A1 antigen, which may be part of a larger BTN1A1 polypeptide fragment, which, in turn, may be part of a larger BTN1A1 polypeptide. BTN1A1 may exist in a native or denatured form. BTN1A1 polypeptides described herein may be isolated from a variety of sources, such as from human tissue types or from another source, or prepared by recombinant or synthetic methods. Orthologs to the BTN1A1 polypeptide are also well known in the art.

The terms “BTN1A1 activity,” “BTN1A1 signaling,” and “BTN1A1 ligand-like signaling” when applied to a binding protein such as an antibody that binds to BTN1A1 of the present disclosure, means that the binding protein (e.g., antibody) mimics or modulates a biological effect induced by the binding of BTN1A1 ligand, and induces a biological response that otherwise would result from BTN1A1 ligand binding to BTN1A1, e.g., in vivo or in vitro. In assessing the binding specificity of anti-BTN1A1 antibody, for example, an antibody or fragment thereof that binds to BTN1A1 (e.g., human BTN1A1), the antibody is deemed to induce a biological response when the response is equal to or greater than 5%, such as equal to or greater than 10%, 15%, 20%, 25%, 30%, 35%, 40%, 45%, 50%, 55% 60% 65%, 70%, 75%, 80%, 85%, 90%, 95%, 100%, 125%, 150%, 175%, or 200% of the activity of a wild type BTN1A1 ligand standard. In one embodiment, the anti-BTN1A1 antibody or the BTN1A1 ligand is immobilized (for example, on a plastic surface or bead). In certain embodiments, the antibody has the following properties: exhibits an efficacy level of equal to or more than 5% of a BTN1A1 ligand standard, with an EC₅₀ of equal to or less than 100 nM, e.g., 90 nM, 80 nM, 70 nM, 60 nM, 50 nM, 40 nM, 30 nM, 20 nM, 10 nM, 5 nM, 2 nM, 1 nM, 0.5 nM, 0.2 nM, or 0.1 nM in (1) human or cynomolgus PBMC assay (see, e.g., Examples 4.2.1 and 4.2.2); or (2) human whole blood sample assay (see, e.g., Example 4.2.1).

The term “binding protein” refers to a protein comprising a portion (e.g., one or more binding regions such as CDRs) that binds to BTN1A1, including human, mouse and/or cynomolgus BTN1A1 and, optionally, a scaffold or framework portion (e.g., one or more scaffold or framework regions) that allows the binding portion to adopt a conformation that promotes binding of the binding protein to a BTN1A1 polypeptide, fragment, or epitope. Examples of such binding proteins include antibodies, such as a human antibody, a humanized antibody, a chimeric antibody, a recombinant antibody, a single chain antibody, a diabody, a triabody, a tetrabody, a Fab fragment, a F(ab′)₂ fragment, an IgD antibody, an IgE antibody, an IgM antibody, an IgG1 antibody, an IgG2 antibody, an IgG3 antibody, or an IgG4 antibody, and fragments thereof. The binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the binding protein as well as wholly synthetic scaffolds comprising, for example, a biocompatible polymer. See, e.g., Korndorfer et al., 2003, Proteins: Structure, Function, and Bioinformatics 53(1):121-29; and Roque et al., 2004, Biotechnol. Prog. 20:639-54. In addition, peptide antibody mimetics (“PAMs”) can be used, as well as scaffolds based on antibody mimetics utilizing fibronectin components as a scaffold. In the context of the present disclosure, a binding protein is said to specifically bind or selectively bind to BTN1A1, for example, when the dissociation constant (K_{D}) is ≤10⁻⁷ M. In some embodiments, the binding proteins (e.g., antibodies) may specifically bind to BTN1A1 with a K_{D} of from about 10⁻⁷ M to about 10⁻¹² M. In certain embodiments, the binding protein (e.g., antibody) may specifically bind to BTN1A1 with high affinity when the K_{D} is ≤10⁻⁸ M or K_{D} is ≤10⁻⁹ M.

In one embodiment, the binding proteins (e.g., antibodies) may specifically bind to purified human BTN1A1 with a K_{D} of from 1×10⁻⁹ M to 10×10⁻⁹ M as measured by Biacore®. In another embodiment, the binding proteins (e.g., antibodies) may specifically bind to purified human BTN1A1 with a K_{D} of from 0.1×10⁻⁹ M to 1×10⁻⁹ M as measured by KinExA™ (Sapidyne, Boise, ID). In yet another embodiment, the binding proteins (e.g., antibodies) specifically bind to human BTN1A1 expressed on cells with a K_{D} of from 0.1×10⁻⁹ M to 10×10⁻⁹ M. In certain embodiments, the binding proteins (e.g., antibodies) specifically bind to human BTN1A1 expressed on cells with a K_{D} of from 0.1×10⁻⁹ M to 1×10⁻⁹ M. In some embodiments, the binding proteins (e.g., antibodies) specifically bind to human BTN1A1 expressed on cells with a K_{D} of 1×10⁻⁹ M to 10×10⁻⁹ M. In certain embodiments, the binding proteins (e.g., antibodies) specifically bind to human BTN1A1 expressed on cells with a K_{D} of about 0.1×10⁻⁹ M, about 0.5×10⁻⁹ M, about 1×10⁻⁹ M, about 5×10⁻⁹ M, about 10×10⁻⁹ M, or any range or interval thereof. In still another embodiment, the binding proteins (e.g., antibodies) may specifically bind to cynomolgus BTN1A1 expressed on cells with a K_{D} of 0.1×10⁻⁹ M to 10×10⁻⁹ M. In certain embodiments, the binding proteins (e.g., antibodies) specifically bind to cynomolgus BTN1A1 expressed on cells with a K_{D} of from 0.1×10⁻⁹ M to 1×10⁻⁹ M. In some embodiments, the binding proteins (e.g., antibodies) specifically bind to cynomolgus BTN1A1 expressed on cells with a K_{D} of 1×10⁻⁹ M to 10×10⁻⁹ M. In certain embodiments, the binding proteins (e.g., antibodies) specifically bind to cynomolgus BTN1A1 expressed on cells with a K_{D} of about 0.1×10⁻⁹ M, about 0.5×10⁻⁹ M, about 1×10⁻⁹ M, about 5×10⁻⁹ M, about 10×10⁻⁹ M, or any range or interval thereof. In still another embodiment, the binding proteins (e.g., antibodies) may specifically bind to mouse BTN1A1 expressed on cells with a K_{D} of 0.1×10⁻⁹ M to 10×10⁻⁹ M. In certain embodiments, the binding proteins (e.g., antibodies) specifically bind to mouse BTN1A1 expressed on cells with a K_{D} of from 0.1×10⁻⁹ M to 1×10⁻⁹ M. In some embodiments, the binding proteins (e.g., antibodies) specifically bind to mouse BTN1A1 expressed on cells with a K_{D} of 1×10⁻⁹ M to 10×10⁻⁹ M. In certain embodiments, the binding proteins (e.g., antibodies) specifically bind to mouse BTN1A1 expressed on cells with a K_{D} of about 0.1×10⁻⁹ M, about 0.5×10⁻⁹ M, about 1×10⁻⁹ M, about 5×10⁻⁹ M, about 10×10⁻⁹ M, or any range or interval thereof

The term “antibody,” “immunoglobulin,” or “Ig” is used interchangeably herein, and is used in the broadest sense and specifically encompasses, for example, individual anti-BTN1A1 monoclonal antibodies (including agonist, antagonist, neutralizing antibodies, full length or intact monoclonal antibodies), anti-BTN1A1 antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain anti-BTN1A1 antibodies, and fragments of anti-BTN1A1 antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a BTN1A1 polypeptide, a BTN1A1 fragment, or a BTN1A1 epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments such as BTN1A1-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments such as BTN1A1-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to a BTN1A1 antigen (e.g., one or more CDRs of an anti-BTN1A1 antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Anti-BTN1A1 antibodies may be agonistic antibodies or antagonistic antibodies. Provided herein are agonistic antibodies to BTN1A1, including antibodies that induce BTN1A1 signaling.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a BTN1A1 epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002). Exemplary methods of producing monoclonal antibodies are provided in the Examples herein.

“Polyclonal antibodies” as used herein refer to an antibody population generated in an immunogenic response to a protein having many epitopes and thus includes a variety of different antibodies directed to the same or different epitopes within the protein. Methods for producing polyclonal antibodies are known in the art (See, e.g., Short Protocols in Molecular Biology (Ausubel et al. eds., 5th ed. 2002)).

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, BTN1A1 fragments or anti-BTN1A1 antibody fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the amino acid sequence of a BTN1A1 polypeptide or an anti-BTN1A1 antibody. In a specific embodiment, a fragment of a BTN1A1 polypeptide or an anti-BTN1A1 antibody retains at least 1, at least 2, at least 3, or more functions of the polypeptide or antibody.

An “antigen” is a predetermined antigen to which an antibody can selectively bind. A target antigen may be a polypeptide, carbohydrate, nucleic acid, lipid, hapten, or other naturally occurring or synthetic compound. In some embodiments, the target antigen is a polypeptide.

The terms “antigen-binding fragment,” “antigen-binding domain,” “antigen-binding region,” and similar terms refer to that portion of an antibody, which comprises the amino acid residues that interact with an antigen and confer on the binding agent its specificity and affinity for the antigen (e.g., the CDRs).

An “epitope” is the site on the surface of an antigen molecule to which a single antibody molecule binds, such as a localized region on the surface of an antigen, such as a BTN1A1 polypeptide or a BTN1A1 polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. In certain embodiments, a BTN1A1 epitope is a three-dimensional surface feature of a BTN1A1 polypeptide. In other embodiments, a BTN1A1 epitope is linear feature of a BTN1A1 polypeptide. Generally an antigen has several or many different epitopes and may react with many different antibodies.

An antibody binds “an epitope,” “essentially the same epitope,” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent, or enzyme labels.

“Epitope mapping” is the process of identifying the binding sites, or epitopes, of antibodies on their target antigens. “Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as BTN1A1, is the affinity of the antibody or functional fragment for that epitope. The ratio of dissociation rate (k_{off}) to association rate (kₒₙ) of an antibody to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent BTN1A1, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The terms “antibodies that specifically bind to BTN1A1,” “antibodies that specifically bind to a BTN1A1 epitope,” and analogous terms are also used interchangeably herein and refer to antibodies that specifically bind to a BTN1A1 polypeptide, such as a BTN1A1 antigen, or fragment, or epitope (e.g., human BTN1A1 such as a human BTN1A1 polypeptide, antigen, or epitope). An antibody that specifically binds to BTN1A1 (e.g., human BTN1A1) may bind to the extracellular domain or a peptide derived from the extracellular domain of BTN1A1. An antibody that specifically binds to a BTN1A1 antigen (e.g., human BTN1A1) may be cross-reactive with related antigens (e.g., cynomolgus BTN1A1). In certain embodiments, an antibody that specifically binds to a BTN1A1 antigen does not cross-react with other antigens. An antibody that specifically binds to a BTN1A1 antigen can be identified, for example, by immunoassays, Biacore®, or other techniques known to those of skill in the art. An antibody binds specifically to a BTN1A1 antigen when it binds to a BTN1A1 antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding antibody specificity. An antibody which “binds an antigen of interest” (e.g., a target antigen such as BTN1A1) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein, for example, as determined by fluorescence activated cell sorting (FACS) analysis or RIA. With regard to the binding of an antibody to a target molecule, the term “specific binding,” “specifically binds to,” or “is specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “anti-BTN1A1 antibody” or “an antibody that binds to BTN1A1” includes an antibody that is capable of binding BTN1A1 with sufficient affinity such that the antibody is useful, for example, as a diagnostic agent in targeting BTN1A1. The term “specific binding,” “specifically binds to,” or “is specific for” a particular polypeptide or an epitope on a particular polypeptide target as used herein refers to binding where a molecule binds to a particular polypeptide or epitope on a particular polypeptide without substantially binding to any other polypeptide or polypeptide epitope. In certain embodiments, an antibody that binds to BTN1A1 has a dissociation constant (K_{D}) of less than or equal to 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, anti-BTN1A1 antibody binds to an epitope of BTN1A1 that is conserved among BTN1A1 from different species (e.g., between human and cynomolgus BTN1A1).

The term “compete” when used in the context of anti-BTN1A1 antibodies (e.g., agonistic antibodies and binding proteins that bind to BTN1A1 and compete for the same epitope or binding site on a target) means competition as determined by an assay in which the antibody (or binding fragment) thereof under study prevents or inhibits the specific binding of a reference molecule (e.g., a reference ligand or reference antigen-binding protein, such as a reference antibody) to a common antigen (e.g., BTN1A1 or a fragment thereof). Numerous types of competitive binding assays can be used to determine if a test antibody competes with a reference antibody for binding to BTN1A1 (e.g., human BTN1A1). Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using 1-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified antigen (e.g., BTN1A1 such as human BTN1A1) bound to a solid surface, or cells bearing either of an unlabeled test antigen-binding protein (e.g., test anti-BTN1A1 antibody) or a labeled reference antigen-binding protein (e.g., reference anti-BTN1A1 antibody). Competitive inhibition may be measured by determining the amount of label bound to the solid surface or cells in the presence of the test antigen-binding protein. Usually the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur. Additional details regarding methods for determining competitive binding are described herein. Usually, when a competing antibody protein is present in excess, it will inhibit specific binding of a reference antibody to a common antigen by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

An “isolated” antibody is substantially free of cellular material or other contaminating proteins from the cell or tissue source and/or other contaminant components from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 25%, 20%, 15%, 10%, 5%, or 1% (by dry weight) of heterologous protein (also referred to herein as a “contaminating protein”). In certain embodiments, when the antibody is recombinantly produced, it is substantially free of culture medium, e.g., culture medium represents less than about 20%, 15%, 10%, 5%, or 1% of the volume of the protein preparation. In certain embodiments, when the antibody is produced by chemical synthesis, it is substantially free of chemical precursors or other chemicals, for example, it is separated from chemical precursors or other chemicals that are involved in the synthesis of the protein. Accordingly such preparations of the antibody have less than about 30%, 25%, 20%, 15%, 10%, 5%, or 1% (by dry weight) of chemical precursors or compounds other than the antibody of interest. Contaminant components can also include, but are not limited to, materials that would interfere with therapeutic uses for the antibody, and may include enzymes, hormones, and other proteinaceous or non-proteinaceous solutes. In certain embodiments, the antibody will be purified (1) to greater than 95% by weight of antibody as determined by the Lowry method (Lowry et al., 1951, J. Bio. Chem. 193: 265-75), such as 96%, 97%, 98%, or 99%, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step. In specific embodiments, antibodies provided herein are isolated.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for and a isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994).

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. Light chain amino acid sequences are well known in the art. A light chain can be a human light chain.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering as in Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described and are contemplated herein, for example, by AbM, Chothia, Contact, IMGT, and AHon.

A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., 1997, J. Biol. Chem. 252:6609-16; Kabat, 1978, Adv. Prot. Chem. 32:1-75). CDR region sequences also have been defined structurally by Chothia as those residues that are not part of the conserved β-sheet framework, and thus are able to adapt different conformations (Chothia and Lesk, 1987, J. Mol. Biol. 196:901-17). Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art.

The term “hypervariable region,” “HVR,” or “HV,” when used herein refers to the regions of an antibody variable region that are hypervariable in sequence and/or form structurally defined loops. Generally, antibodies comprise six hypervariable regions, three in the VH (H1, H2, H3) and three in the VL (L1, L2, L3). A number of hypervariable region delineations are in use and are encompassed herein. The Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. The residues from each of these hypervariable regions or CDRs are noted below.

Recently, a universal numbering system has been developed and widely adopted, ImMunoGeneTics (IMGT) Information System® (Lafranc et al., 2003, Dev. Comp. Immunol. 27(1):55-77). IMGT is an integrated information system specializing in immunoglobulins (IG), T cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). In some embodiments, the CDRs are as defined by the IMGT numbering system. In other embodiments, the CDRs are as defined by the Kabat numbering system. In certain embodiments, the CDRs are as defined by the AbM numbering system. In other embodiments, the CDRs are as defined by the Chothia system. In yet other embodiments, the CDRs are as defined by the Contact numbering system.

| | IMGT | Kabat | AbM | Chothia | Contact |
|---|---|---|---|---|---|
| V_{H} CDR1 | 27-38 | 31-35 | 26-35 | 26-32 | 30-35 |
| V_{H} CDR2 | 56-65 | 50-65 | 50-58 | 53-55 | 47-58 |
| V_{H} CDR3 | 105-117 | 95-102 | 95-102 | 96-101 | 93-101 |
| V_{L} CDR1 | 27-38 | 24-34 | 24-34 | 26-32 | 30-36 |
| V_{L} CDR2 | 56-65 | 50-56 | 50-56 | 50-52 | 46-55 |
| V_{L} CDR3 | 105-117 | 89-97 | 89-97 | 91-96 | 89-96 |

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH. As used herein, the terms “HVR” and “CDR” are used interchangeably.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fe region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human IgG1 Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fe region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “variant” when used in relation to BTN1A1 or to an anti-BTN1A1 antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a BTN1A1 variant may result from one or more (such as, for example, about 1 to about 25, about 1 to about 20, about 1 to about 15, about 1 to about 10, or about 1 to about 5) changes to an amino acid sequence of a native BTN1A1. Also by way of example, a variant of an anti-BTN1A1 antibody may result from one or more (such as, for example, about 1 to about 25, about 1 to about 20, about 1 to about 15, about 1 to about 10, or about 1 to about 5) changes to an amino acid sequence of a native or previously unmodified anti-BTN1A1 antibody. Variants may be naturally occurring, such as allelic or splice variants, or may be artificially constructed. Polypeptide variants may be prepared from the corresponding nucleic acid molecules encoding the variants. In specific embodiments, the BTN1A1 variant or anti-BTN1A1 antibody variant at least retains BTN1A1 or anti-BTN1A1 antibody functional activity, respectively. In specific embodiments, an anti-BTN1A1 antibody variant binds BTN1A1 and/or is antagonistic to BTN1A1 activity. In specific embodiments, an anti-BTN1A1 antibody variant binds BTN1A1 and/or is agonistic to BTN1A1 activity. In certain embodiments, the variant is encoded by a single nucleotide polymorphism (SNP) variant of a nucleic acid molecule that encodes BTN1A1 or anti-BTN1A1 antibody VH or VL regions or subregions, such as one or more CDRs.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Antibody fragments” comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

A “functional fragment,” “binding fragment,” or “antigen-binding fragment” of a diagnostic antibody will exhibit at least one if not some or all of the biological functions attributed to the intact antibody, the function comprising at least binding to the target antigen (e.g., a BTN1A1 binding fragment or fragment that binds to BTN1A1).

The term “fusion protein” as used herein refers to a polypeptide that comprises an amino acid sequence of an antibody and an amino acid sequence of a heterologous polypeptide or protein (e.g., a polypeptide or protein not normally a part of the antibody (e.g., a non-anti-BTN1A1 antigen-binding antibody)). The term “fusion” when used in relation to BTN1A1 or to an anti-BTN1A1 antibody refers to the joining of a peptide or polypeptide, or fragment, variant, and/or derivative thereof, with a heterologous peptide or polypeptide. In certain embodiments, the fusion protein retains the biological activity of the BTN1A1 or anti-BTN1A1 antibody. In certain embodiments, the fusion protein comprises a BTN1A1 antibody VH region, VL region, VH CDR (one, two, or three VH CDRs), and/or VL CDR (one, two, or three VL CDRs), wherein the fusion protein binds to a BTN1A1 epitope, a BTN1A1 fragment, and/or a BTN1A1 polypeptide.

The term “native” when used in connection with biological materials such as nucleic acid molecules, polypeptides, host cells, and the like, refers to those which are found in nature and not manipulated, modified, and/or changed (e.g., isolated, purified, selected) by a human being.

The antibodies provided herein can include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

A “human antibody” is one that possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Brüggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

An “affinity matured” antibody is one with one or more alterations (e.g., amino acid sequence variations, including changes, additions, and/or deletions) in one or more HVRs thereof which result in an improvement in the affinity of the antibody for antigen, compared to a parent antibody which does not possess those alteration(s). Affinity matured antibodies can have nanomolar or even picomolar affinities for the target antigen. Affinity matured antibodies are produced by procedures known in the art. For review, see Hudson and Souriau, 2003, Nature Medicine 9:129-34; Hoogenboom, 2005, Nature Biotechnol. 23:1105-16; Quiroz and Sinclair, 2010, Revista Ingeneria Biomedia 4:39-51.

A “blocking” antibody or an “antagonist” antibody is one which inhibits or reduces biological activity of the antigen it binds. For example, blocking antibodies or antagonist antibodies may substantially or completely inhibit the biological activity of the antigen.

An “agonist” antibody is an antibody that triggers a response, e.g., one that mimics at least one of the functional activities of a polypeptide of interest. An agonist antibody includes an antibody that is a ligand mimetic, for example, wherein a ligand binds to a cell surface receptor and the binding induces cell signaling or activities via an intercellular cell signaling pathway and wherein the antibody induces a similar cell signaling or activation. An “agonist” of BTN1A1 refers to a molecule that is capable of activating or otherwise increasing one or more of the biological activities of BTN1A1, such as in a cell expressing BTN1A1. In some embodiments, an agonist of BTN1A1 (e.g., an agonistic antibody as described herein) may, for example, act by activating or otherwise increasing the activation and/or cell signaling pathways of a cell expressing a BTN1A1 protein, thereby increasing a BTN1A1-mediated biological activity of the cell relative to the BTN1A1-mediated biological activity in the absence of agonist. In some embodiments the antibodies provided herein are agonistic anti-BTN1A1 antibodies, including antibodies that induce BTN1A1 signaling.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using surface plasmon resonance assays by Biacore®, using, for example, a Biacore®TM-2000 or a Biacore®TM-3000, or by biolayer interferometry using, for example, the Octet®QK384 system. An “on-rate” or “rate of association” or “association rate” or “kₒₙ” may also be determined with the same surface plasmon resonance or biolayer interferometry techniques described above using, for example, a Biacore®TM-2000 or a Biacore®TM-3000, or the Octet®QK384 system.

The term “inhibition” or “inhibit,” when used herein, refers to partial (such as, 1%, 2%, 5%, 10^{%}, 20%, 25%, 50%, 75%, 90%, 95%, 99%) or complete (i.e., 100%) inhibition.

The term “attenuate,” “attenuation,” or “attenuated,” when used herein, refers to partial (such as, 1%, 2%, 5%, 10%, 20%, 25%, 50%, 75%, 90%, 95%, 99%) or complete (i.e., 100%) reduction in a property, activity, effect, or value.

“Antibody effector functions” refer to the biological activities attributable to the Fc region (e.g., a native sequence Fc region or amino acid sequence variant Fc region) of an antibody, and vary with the antibody isotype. Examples of antibody effector functions include but are not limited to: C1q binding; CDC; Fe receptor binding; ADCC; phagocytosis; downregulation of cell surface receptors (e.g., B cell receptor); and B cell activation.

“T cell effector functions” refer to the biological activities attributable to various types of T cells, including but not limited to cytotoxic T cells, T helper cells, and memory T cells. Examples of T cell effector functions include: increasing T cell proliferation, secreting cytokines, releasing cytotoxins, expressing membrane-associated molecules, killing target cells, activating macrophages, and activating B cells.

“Antibody-dependent cell-mediated cytotoxicity” or “ADCC” refers to a form of cytotoxicity in which secreted immunoglobulin bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. NK cells, the primary cells for mediating ADCC, express FcγRIII only, whereas monocytes express FcγRI, FcγRII, and FcγRIII. FcR expression on hematopoietic cells is known (see, e.g., Ravetch and Kinet, 1991, Annu. Rev. Immunol. 9:457-92). To assess ADCC activity of a molecule of interest, an in vitro ADCC assay (see, e.g., U.S. Pat. Nos. 5,500,362 and 5,821,337) can be performed. Useful effector cells for such assays include peripheral blood mononuclear cells (PBMC) and Natural Killer (NK) cells. Alternatively or additionally, ADCC activity of the molecule of interest may be assessed in vivo, for example, in an animal model (see, e.g., Clynes et al., 1998, Proc. Natl. Acad. Sci. USA 95:652-56). Antibodies with little or no ADCC activity may be selected for use.

“Antibody-dependent cellular phagocytosis” or “ADCP” refers to the destruction of target cells via monocyte or macrophage-mediated phagocytosis when immunoglobulin bound onto Fc receptors (FcRs) present on certain phagocytotic cells (e.g., neutrophils, monocytes, and macrophages) enable these phagocytotic cells to bind specifically to an antigen-bearing target cell and subsequently kill the target cell. To assess ADCP activity of a molecule of interest, an in vitro ADCP assay (see, e.g., Bracher et al., 2007, J. Immunol. Methods 323:160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248:167-82). Antibodies with little or no ADCP activity may be selected for use.

“Fc receptor” or “FcR” describes a receptor that binds to the Fc region of an antibody. An exemplary FcR is a native sequence human FcR. Moreover, an exemplary FcR is one that binds an IgG antibody (e.g., a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof (see, e.g., Daëron, 1997, Annu. Rev. Immunol. 15:203-34). Various FcRs are known (see, e.g., Ravetch and Kinet, 1991, Annu. Rev. Immunol. 9:457-92; Capel et al., 1994, Immunomethods 4:25-34; and de Haas et al., 1995, J. Lab. Clin. Med. 126:330-41). Other FcRs, including those to be identified in the future, are encompassed by the term “FcR” herein. The term also includes the neonatal receptor, FcRn, which is responsible for the transfer of maternal IgGs to the fetus (see, e.g., Guyer et al., 1976, J. Immunol. 117:587-93; and Kim et al., 1994, Eu. J. Immunol. 24:2429-34). Antibody variants with improved or diminished binding to FcRs have been described (see, e.g., WO 2000/42072; U.S. Pat. Nos. 7,183,387; 7,332,581; and 7,335,742; Shields et al. 2001, J. Biol. Chem. 9(2):6591-604).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay (see, e.g., Gazzano-Santoro et al., 1996, J. Immunol. Methods 202:163) may be performed. Polypeptide variants with altered Fc region amino acid sequences (polypeptides with a variant Fc region) and increased or decreased C1q binding capability have been described (see, e.g., U.S. Pat. No. 6,194,551; WO 1999/51642; Idusogie et al., 2000, J. Immunol. 164: 4178-84). Antibodies with little or no CDC activity may be selected for use.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

In the context of a polypeptide, the term “analog” as used herein refers to a polypeptide that possesses a similar or identical function as a BTN1A1 polypeptide, a fragment of a BTN1A1 polypeptide, or an anti-BTN1A1 antibody but does not necessarily comprise a similar or identical amino acid sequence of a BTN1A1 polypeptide, a fragment of a BTN1A1 polypeptide, or an anti-BTN1A1 antibody, or possess a similar or identical structure of a BTN1A1 polypeptide, a fragment of a BTN1A1 polypeptide, or an anti-BTN1A1 antibody. A polypeptide that has a similar amino acid sequence refers to a polypeptide that satisfies at least one of the followings: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a BTN1A1 polypeptide, a fragment of a BTN1A1 polypeptide, or an anti-BTN1A1 antibody provided herein; (b) a polypeptide encoded by a nucleotide sequence that hybridizes under stringent conditions to a nucleotide sequence encoding a BTN1A1 polypeptide, a fragment of a BTN1A1 polypeptide, or an anti-BTN1A1 antibody (or VH or VL region thereof) described herein at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Sambrook et al., Molecular Cloning: A Laboratory Manual (2001); and Maniatis et al., Molecular Cloning: A Laboratory Manual (1982)); or (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a BTN1A1 polypeptide, a fragment of a BTN1A1 polypeptide, or an anti-BTN1A1 antibody (or VH or VL region thereof) described herein. A polypeptide with similar structure to a BTN1A1 polypeptide, a fragment of a BTN1A1 polypeptide, or an anti-BTN1A1 antibody provided herein refers to a polypeptide that has a similar secondary, tertiary, or quaternary structure of a BTN1A1 polypeptide, a fragment of a BTN1A1 polypeptide, or an anti-BTN1A1 antibody provided herein. The structure of a polypeptide can be determined by methods known to those skilled in the art, including but not limited to, X-ray crystallography, nuclear magnetic resonance, and crystallographic electron microscopy.

In the context of a polypeptide, the term “derivative” as used herein refers to a polypeptide that comprises an amino acid sequence of a BTN1A1 polypeptide, a fragment of a BTN1A1 polypeptide, or an antibody that binds to a BTN1A1 polypeptide which has been altered by the introduction of amino acid residue substitutions, deletions, or additions. The term “derivative” as used herein also refers to a BTN1A1 polypeptide, a fragment of a BTN1A1 polypeptide, or an antibody that binds to a BTN1A1 polypeptide which has been chemically modified, e.g., by the covalent attachment of any type of molecule to the polypeptide. For example, but not by way of limitation, a BTN1A1 polypeptide, a fragment of a BTN1A1 polypeptide, or an anti-BTN1A1 antibody may be chemically modified, e.g., by increase or decrease of glycosylation, acetylation, pegylation, phosphorylation, amidation, derivatization by known protecting/blocking groups, proteolytic cleavage, chemical cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. Further, a derivative of a BTN1A1 polypeptide, a fragment of a BTN1A1 polypeptide, or an anti-BTN1A1 antibody may contain one or more non-classical amino acids. A polypeptide derivative possesses a similar or identical function as a BTN1A1 polypeptide, a fragment of a BTN1A1 polypeptide, or an anti-BTN1A1 antibody provided herein.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “vector” refers to a substance that is used to carry or include a nucleic acid sequence, including for example, a nucleic acid sequence encoding an anti-BTN1A1 antibody as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., an anti-BTN1A1 antibody as described herein), and it is further understood that expression levels can be optimized to obtain sufficient expression using methods well known in the art.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an anti-BTN1A1 antibody of the present disclosure may include a parent hybridoma cell, as well as bacterial and eukaryotic host cells into which nucleic acids encoding the antibodies have been introduced. Suitable host cells are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “recombinant antibody” refers to an antibody that is prepared, expressed, created, or isolated by recombinant means. Recombinant antibodies can be antibodies expressed using a recombinant expression vector transfected into a host cell, antibodies isolated from a recombinant, combinatorial antibody library, antibodies isolated from an animal (e.g., a mouse or cow) that is transgenic and/or transchromosomal for human immunoglobulin genes (see, e.g., Taylor et al., 1992, Nucl. Acids Res. 20:6287-95), or antibodies prepared, expressed, created, or isolated by any other means that involves splicing of immunoglobulin gene sequences to other DNA sequences. Such recombinant antibodies can have variable and constant regions, including those derived from human germline immunoglobulin sequences (See Kabat et al., supra). In certain embodiments, however, such recombinant antibodies may be subjected to in vitro mutagenesis (or, when an animal transgenic for human Ig sequences is used, in vivo somatic mutagenesis), thus the amino acid sequences of the VH and VL regions of the recombinant antibodies are sequences that, while derived from and related to human germline VH and VL sequences, may not naturally exist within the human antibody germline repertoire in vivo.

The term “detectable probe” refers to a composition that provides a detectable signal. The term includes, without limitation, any fluorophore, chromophore, radiolabel, enzyme, antibody or antibody fragment, and the like, that provide a detectable signal via its activity.

The term “detectable agent” refers to a substance that can be used to ascertain the existence or presence of a desired molecule, such as an anti-BTN1A1 antibody as described herein, in a sample or subject. A detectable agent can be a substance that is capable of being visualized or a substance that is otherwise able to be determined and/or measured (e.g., by quantitation).

The term “diagnostic agent” refers to a substance administered to a subject that aids in the diagnosis of a disease, disorder, or condition. Such substances can be used to reveal, pinpoint, and/or define the localization of a disease causing process. In certain embodiments, a diagnostic agent includes a substance that is conjugated to an anti-BTN1A1 antibody as described herein, that when administered to a subject or contacted with a sample from a subject aids in the diagnosis of a BTN1A1-mediated disease.

The term “composition” is intended to encompass a product containing the specified ingredients (e.g., an antibody provided herein) in, optionally, the specified amounts.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain an anti-BTN1A1 antibody, for example, in isolated or purified form, together with a suitable amount of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human.

“Administer” or “administration” refers to the act of injecting or otherwise physically delivering a substance as it exists outside the body (e.g., an anti-BTN1A1 antibody as described herein) into a patient, such as by mucosal, intradermal, intravenous, intramuscular delivery, and/or any other method of physical delivery described herein or known in the art.

The term “effective amount” as used herein refers to the amount of an antibody or pharmaceutical composition provided herein which is sufficient to result in the desired outcome.

The terms “about” and “approximately” mean within 20%, within 15%, within 10%, within 9%, within 8%, within 7%, within 6%, within 5%, within 4%, within 3%, within 2%, within 1%, or less of a given value or range.

“Substantially all” refers to at least about 60%, at least about 65%, at least about 70%, at least about 75%, at least about 80%, at least about 85%, at least about 90%, at least about 95%, at least about 98%, at least about 99%, or about 100%.

The phrase “substantially similar” or “substantially the same” denotes a sufficiently high degree of similarity between two numeric values (e.g., one associated with an antibody of the present disclosure and the other associated with a reference antibody) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference antibody.

The phrase “substantially increased,” “substantially reduced,” or “substantially different,” as used herein, denotes a sufficiently high degree of difference between two numeric values (e.g., one associated with an antibody of the present disclosure and the other associated with a reference antibody) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference antibody.

### 4.3 Compositions and Methods of Making the Same

Provided herein are antibodies that bind to a BTN1A1 polypeptide, a BTN1A1 polypeptide fragment, a BTN1A1 peptide, or a BTN1A1 epitope.

In certain embodiments, the antibodies provided herein bind to human and/or cynomolgus BTN1A1. In one embodiment, the BTN1A1 antibodies bind to human BTN1A1. In one embodiment, the BTN1A1 antibodies bind to cynomolgus BTN1A1. In one embodiment, the BTN1A1 antibodies bind to both human BTN1A1 and cynomolgus BTN1A1. In other embodiments, the antibodies provided herein bind to rodent BTN1A1. In other embodiments, the antibodies provided herein bind to both human and rodent BTN1A1. In other embodiments, the antibodies provided herein bind to human, cynomolgus and rodent BTN1A1.

In some embodiments, the anti-BTN1A1 antibodies bind to the intracellular domain (ICD) of BTN1A1.

In some embodiments, the anti-BTN1A1 antibodies bind to the extracellular domain of BTN1A1. In some embodiments, the anti-BTN1A1 antibodies bind to a region of the extracellular domain identified as ECD1 (amino acid residues 67-86) of BTN1A1. In some embodiments, the anti-BTN1A1 antibodies bind to a region of the extracellular domain identified as ECD2 (amino acid residues 179-197) of BTN1A1.

Also provided are antibodies that competitively block an anti-BTN1A1 antibody provided herein from binding to a BTN1A1 polypeptide.

Also provided are antibodies that compete for binding to a BTN1A1 polypeptide with an anti-BTN1A1 antibody provided herein.

The anti-BTN1A1 antibodies provided herein can also be conjugated or recombinantly fused, e.g., to a diagnostic agent or detectable agent. Further provided are compositions comprising an anti-BTN1A1 antibody.

Also provided herein are isolated nucleic acid molecules encoding an immunoglobulin heavy chain, light chain, VH region, VL region, VH CDR1, VH CDR2, VH CDR3, VL CDR1, VL CDR2, and/or VL CDR3 of anti-BTN1A1 antibodies that bind to a BTN1A1 polypeptide, a BTN1A1 polypeptide fragment, a BTN1A1 peptide, or a BTN1A1 epitope.

Further provided are vectors and host cells comprising nucleic acid molecules encoding anti-BTN1A1 antibodies that bind to a BTN1A1 polypeptide, a BTN1A1 polypeptide fragment, a BTN1A1 peptide, or a BTN1A1 epitope. Also provided are methods of making antibodies that bind to a BTN1A1 polypeptide, a BTN1A1 polypeptide fragment, a BTN1A1 peptide, or a BTN1A1 epitope.

### 4.3.1 Anti-BTN1A1 Antibodies

In one embodiment, the present disclosure provides anti-BTN1A1 antibodies that may find use herein as diagnostic agents. Exemplary antibodies include polyclonal, monoclonal, humanized, human, bispecific, and heteroconjugate antibodies, as well as variants thereof having improved affinity or other properties.

In some embodiments, provided herein are antibodies that bind to BTN1A1, including a BTN1A1 polypeptide, a BTN1A1 polypeptide fragment, a BTN1A1 peptide, or a BTN1A1 epitope. In certain embodiments, the antibodies provided herein bind to human and/or cynomolgus BTN1A1. In other embodiments, the antibodies provided herein bind to rodent BTN1A1 (e.g., a mouse BTN1A1). In one embodiment, an antibody provided herein binds to human BTN1A1. In another embodiment, an antibody provided herein binds to cynomolgus BTN1A1. In another embodiment, an antibody provided herein binds to human BTN1A1 and cynomolgus BTN1A1. In some embodiments, an antibody provided herein binds to both human BTN1A1 and rodent BTN1A1 (e.g., a mouse BTN1A1). In some embodiments, an antibody provided herein binds to both cynomolgus BTN1A1 and rodent BTN1A1 (e.g., a mouse BTN1A1). In some embodiments, an antibody provided herein binds to human BTN1A1, binds to a cynomolgus BTN1A1, and binds to a rodent BTN1A1 (e.g., a mouse BTN1A1). In other embodiments, the anti-BTN1A1 antibodies are humanized antibodies (e.g., comprising human constant regions) that bind BTN1A1, including a BTN1A1 polypeptide, a BTN1A1 polypeptide fragment, a BTN1A1 peptide, or a BTN1A1 epitope.

In certain embodiments, the anti-BTN1A1 antibody comprises a VH region, VL region, VH CDR1, VH CDR2, VH CDR3, VL CDR1, VL CDR2, and/or VL CDR3 of any one of the murine monoclonal antibodies provided herein, such as an amino acid sequence depicted in Tables 1-6. Accordingly, in some embodiments, the isolated antibody or functional fragment thereof provided herein comprises one, two, and/or three heavy chain CDRs and/or one, two, and/or three light chain CDRs from the antibodies STC43H11-1, STC43G3-1, or STC85F1-1, as shown in Tables 1-6.

TABLE 1
VL CDR Sequences of Anti-BTN1A1 Antibody STC43H11-1

| | Region | | | |
|---|---|---|---|---|
| | Definition | CDR1 | CDR2 | CDR3 |
| Kappa | Chothia | QASQSVYNNNRCS | HTSTLDS | LGNYDCSRADCAA |
| light | | (SEQ ID NO: 74) | (SEQ ID NO: 75) | (SEQ ID NO: 76) |
| chain | IMGT | QSVYNN | HTS | LGNYDCSRADCAA |
| | | (SEQ ID NO: 80) | (SEQ ID NO: 81) | (SEQ ID NO: 82) |
| | Kabat | QASQSVYNNNRCS | HTSTLDS | LGNYDCSRADCAA |
| | | (SEQ ID NO: 5) | (SEQ ID NO: 6) | (SEQ ID NO: 7) |
| | Contact | YNNNRCSWF | RLIYHTSTLD | LGNYDCSRADCA |
| | | (SEQ ID NO: 86) | (SEQ ID NO: 87) | (SEQ ID NO: 88) |

TABLE 2
VH CDR Sequences of Anti-BTN1A1 Antibody STC43H11-1

| | Region | | | |
|---|---|---|---|---|
| | Definition | CDR1 | CDR2 | CDR3 |
| Heavy | Chothia | GIDLSSY | GTGGG | SNL |
| chain | | (SEQ ID NO: 71) | (SEQ ID NO: 72) | (SEQ ID NO: 73) |
| | IMGT | GIDLSSYA | SIGTGGGT | ATSNL |
| | | (SEQ ID NO: 77) | (SEQ ID NO: 78) | (SEQ ID NO: 79) |
| | Kabat | SYAMG | SIGTGGGTGYASWAKG | SNL |
| | | (SEQ ID NO: 8) | (SEQ ID NO: 9) | (SEQ ID NO: 10) |
| | Contact | SSYAMG | WIGSIGTGGGTG | ATSN |
| | | (SEQ ID NO: 83) | (SEQ ID NO: 84) | (SEQ ID NO: 85) |

In some embodiments, an antibody provided herein comprises or consists of six CDRs, for example, VH CDR1, VH CDR2, VH CDR3, VL CDR1, VL CDR2, and/or VL CDR3 identified in Tables 1 and 2. In some embodiments, an antibody provided herein can comprise fewer than six CDRs. In some embodiments, the antibody comprises or consists of one, two, three, four, or five CDRs selected from the group consisting of VH CDR1, VH CDR2, VH CDR3, VL CDR1, VL. CDR2, and/or VL. CDR3 identified in Tables 1 and 2. In some embodiments, the antibody comprises or consists of one, two, three, four, or five CDRs selected from the group consisting of VH CDR1, VH CDR2, VH CDR3, VL CDR1, VL. CDR2, and/or VL CDR3 of the monoclonal described herein. Accordingly, in some embodiments, the antibody comprises or consists of one, two, three, four, or five CDRs of anyone of the VH CDR1, VH CDR2, VH CDR3, VL CDR1, VL CDR2, and/or VL CDR3 identified in Tables 1 and 2.

In some embodiments, the antibodies provided herein comprise one or more (e.g., one, two, or three) VH CDRs listed in Table 2. In other embodiments, the antibodies provided herein comprise one or more (e.g., one, two, or three) VL CDRs listed in Table 1. In yet other embodiments, the antibodies provided herein comprise one or more (e.g., one, two, or three) VH CDRs listed in Table 2 and one or more VL CDRs listed in Table 1.

In some embodiments, the antibodies comprise a VH CDR1, VH CDR1, VH CDR3, VL CDR1, VL CDR2, VL CDR3 according to the Kabat numbering system. Accordingly, in some embodiments, the antibodies comprise a VH CDR1 having an amino acid sequence of SEQ ID NO:8. In some embodiments, the antibodies comprise a VH CDR2 having an amino acid sequence of SEQ ID NO:9. In some embodiments, the antibodies comprise a VH CDR3 having an amino acid sequence of SEQ ID NO:10. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 2. In some embodiments, the antibodies comprise a VL CDR1 having an amino acid sequence of any one of SEQ ID NO:5. In another embodiment, the antibodies comprise a VL CDR2 having an amino acid sequence of SEQ ID NO:6. In some embodiments, the antibodies comprise a VL CDR3 having an amino acid sequence of SEQ ID NO:7. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 1.

In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region comprising: (1) a VL CDR1 having an amino acid sequence of SEQ ID NO: 5; (2) a VL CDR2 having an amino acid sequence of SEQ ID NO:6; and (3) a VL CDR3 having an amino acid sequence of SEQ ID NO:7.

In some embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10.

In other embodiments, the antibodies provided herein comprise a VL region comprising: (1) a VL CDR1 having an amino acid sequence of SEQ ID NO:5; (2) a VL CDR2 having an amino acid sequence of SEQ ID NO:6; and (3) a VL CDR3 having an amino acid sequence of SEQ ID NO:7.

Also provided herein are antibodies comprising one or more (e.g., one, two, or three) VH CDRs and one or more (e.g., one, two, or three) VL CDRs listed in Tables 1 and 2. In particular, provided herein is an antibody comprising a VH CDR1 (SEQ ID NO:8) and a VL CDR1 (SEQ ID NO:5). In one embodiment, the antibody comprises a VH CDR1 (SEQ ID NO:8) and a VL CDR2 (SEQ ID NO:6). In other embodiments, the antibody comprises a VH CDR1 (SEQ ID NO:8) and a VL CDR3 (SEQ ID NO:7). In another embodiment, the antibody comprises a VH CDR2 (SEQ ID NO:9) and a VL CDR1 (SEQ ID NO:5). In some embodiments, the antibody comprises a VH CDR2 (SEQ ID NO:9) and a VL CDR2 (SEQ ID NO:6). In one embodiment, the antibody comprises a VH CDR2 (SEQ ID NO:9) and a VL CDR3 (SEQ ID NO:7). In another embodiment, the antibody comprises a VH CDR3 (SEQ ID NO:10) and a VL CDR1 (SEQ ID NO:5). In other embodiments, the antibody comprises a VH CDR3 (SEQ ID NO:10) and a VL CDR2 (SEQ ID NO:6). In some embodiments, the antibody comprises a VH CDR3 (SEQ ID NO:10) and a VL CDR3 (SEQ ID NO:7). In another embodiment, the antibody comprises a VH CDR1 (SEQ ID NO:8), a VH CDR2 (SEQ ID NO:9), and a VL CDR1 (SEQ ID NO:5). In one embodiment, the antibody comprises a VH CDR1 (SEQ ID NO:8), a VH CDR2 (SEQ ID NO:9), and a VL CDR2 (SEQ ID NO:6). In other embodiments, the antibody comprises a VH CDR1 (SEQ ID NO:8), a VH CDR2 (SEQ ID NO:9), and a VL CDR3 (SEQ ID NO:7). In another embodiment, the antibody comprises a VH CDR2 (SEQ ID NO:9), a VH CDR3 (SEQ ID NO:10), and a VL CDR1 (SEQ ID NO:5). In some embodiments, the antibody comprises a VH CDR2 (SEQ ID NO:9), a VH CDR3 (SEQ ID NO:10), and a VL CDR2 (SEQ ID NO:6). In one embodiment, the antibody comprises a VH CDR2 (SEQ ID NO:9), a VH CDR3 (SEQ ID NO:10), and a VL CDR3 (SEQ ID NO:7). In another embodiment, the antibody comprises a VH CDR1 (SEQ ID NO:8), a VH CDR3 (SEQ ID NO:10), and a VL CDR1 (SEQ ID NO:5). In other embodiments, the antibody comprises a VH CDR1 (SEQ ID NO:8), a VH CDR3 (SEQ ID NO:10), and a VL CDR2 (SEQ ID NO:6). In some embodiments, the antibody comprises a VH CDR1 (SEQ ID NO:8), a VH CDR3 (SEQ ID NO:10), and a VL CDR3 (SEQ ID NO:7). In another embodiment, the antibody comprises a VH CDR1 (SEQ ID NO:8), a VL CDR1 (SEQ ID NO:5), and a VL CDR2 (SEQ ID NO:6). In one embodiment, the antibody comprises a VH CDR1 (SEQ ID NO:8), a VL CDR1 (SEQ ID NO:5), and a VL CDR3 (SEQ ID NO:7). In other embodiments, the antibody comprises a VH CDR1 (SEQ ID NO:8), a VL CDR2 (SEQ ID NO:6), and a VL CDR3 (SEQ ID NO:7). In another embodiment, the antibody comprises a VH CDR2 (SEQ ID NO:9), a VL CDR1 (SEQ ID NO:5), and a VL CDR2 (SEQ ID NO:6). In some embodiments, the antibody comprises a VH CDR2 (SEQ ID NO:9), a VL CDR1 (SEQ ID NO:5), and a VL CDR3 (SEQ ID NO:7). In one embodiment, the antibody comprises a VH CDR2 (SEQ ID NO:9), a VL CDR2 (SEQ ID NO:6), and a VL CDR3 (SEQ ID NO:7). In another embodiment, the antibody comprises a VH CDR3 (SEQ ID NO: 10), a VL CDR1 (SEQ ID NO:5), and a VL CDR2 (SEQ ID NO:6). In other embodiments, the antibody comprises a VH CDR3 (SEQ ID NO: 10), a VL CDR1 (SEQ ID NO:5), and a VL CDR3 (SEQ ID NO:7). In some embodiments, the antibody comprises a VH CDR3 (SEQ ID NO:10), a VL CDR2 (SEQ ID NO:6), and a VL CDR3 (SEQ ID NO:7). In another embodiment, the antibody comprises a VH CDR1 (SEQ ID NO:8), a VH CDR2 (SEQ ID NO:9), a VH CDR3 (SEQ ID NO: 10), and a VL CDR1 (SEQ ID NO:5). In one embodiment, the antibody comprises a VH CDR1 (SEQ ID NO:8), a VH CDR2 (SEQ ID NO:9), a VH CDR3 (SEQ ID NO: 10), and a VL CDR2 (SEQ ID NO:6). In other embodiments, the antibody comprises a VH CDR1 (SEQ ID NO:8), a VH CDR2 (SEQ ID NO:9), a VH CDR3 (SEQ ID NO:10), and a VL CDR3 (SEQ ID NO:7). In another embodiment, the antibody comprises a VH CDR1 (SEQ ID NO:8), a VH CDR2 (SEQ ID NO:9), a VL CDR1 (SEQ ID NO:5), and a VL CDR2 (SEQ ID NO:6). In some embodiments, the antibody comprises a VH CDR1 (SEQ ID NO:8), a VH CDR2 (SEQ ID NO:9), a VL CDR1 (SEQ ID NO:5), and a VL CDR3 (SEQ ID NO:7). In one embodiment, the antibody comprises a VH CDR1 (SEQ ID NO:8), a VH CDR2 (SEQ ID NO:9), a VL CDR2 (SEQ ID NO:6), and a VL CDR3 (SEQ ID NO:7). In another embodiment, the antibody comprises a VH CDR1 (SEQ ID NO:8), a VH CDR3 (SEQ ID NO:10), a VL CDR1 (SEQ ID NO:5), and a VL CDR2 (SEQ ID NO:6). In other embodiments, the antibody comprises a VH CDR1 (SEQ ID NO:8), a VH CDR3 (SEQ ID NO:10), a VL CDR1 (SEQ ID NO:5), and a VL CDR3 (SEQ ID NO:7). In some embodiments, the antibody comprises a VH CDR1 (SEQ ID NO:8), a VH CDR3 (SEQ ID NO: 10), a VL CDR2 (SEQ ID NO:6), and a VL CDR3 (SEQ ID NO:7). In another embodiment, the antibody comprises a VH CDR2 (SEQ ID NO:9), a VH CDR3 (SEQ ID NO: 10), a VL CDR1 (SEQ ID NO:5), and a VL CDR2 (SEQ ID NO:6). In one embodiment, the antibody comprises a VH CDR2 (SEQ ID NO:9), a VH CDR3 (SEQ ID NO: 10), a VL CDR1 (SEQ ID NO:5), and a VL CDR3 (SEQ ID NO:7). In other embodiments, the antibody comprises a VH CDR2 (SEQ ID NO:9), a VH CDR3 (SEQ ID NO:10), a VL CDR2 (SEQ ID NO:6), and a VL CDR3 (SEQ ID NO: 7). In another embodiment, the antibody comprises a VH CDR1 (SEQ ID NO:8), a VH CDR2 (SEQ ID NO: 9), a VH CDR3 (SEQ ID NO:10), a VL CDR1 (SEQ ID NO:5), and a VL CDR2 (SEQ ID NO:6). In some embodiments, the antibody comprises a VH CDR1 (SEQ ID NO:8), a VH CDR2 (SEQ ID NO:9), a VH CDR3 (SEQ ID NO:10), a VL CDR1 (SEQ ID NO:5), and a VL CDR3 (SEQ ID NO:7). In one embodiment, the antibody comprises a VH CDR1 (SEQ ID NO:8), a VH CDR2 (SEQ ID NO:9), a VH CDR3 (SEQ ID NO:10), a VL CDR2 (SEQ ID NO:6), and a VL CDR3 (SEQ ID NO:7). In another embodiment, the antibody comprises a VH CDR1 (SEQ ID NO:8), a VH CDR2 (SEQ ID NO:9), a VL CDR1 (SEQ ID NO:5), a VL CDR2 (SEQ ID NO:6), and a VL CDR3 (SEQ ID NO:7). In other embodiments, the antibody comprises a VH CDR1 (SEQ ID NO:8), a VH CDR3 (SEQ ID NO:10), a VL CDR1 (SEQ ID NO:5), a VL CDR2 (SEQ ID NO:6), and a VL CDR3 (SEQ ID NO:7). In some embodiments, the antibody comprises a VH CDR2 (SEQ ID NO:9), a VH CDR3 (SEQ ID NO:10), a VL CDR1 (SEQ ID NO:5), a VL CDR2 (SEQ ID NO:6), and a VL CDR3 (SEQ ID NO:7). In another embodiment, the antibody comprises a VH CDR1 (SEQ ID NO:8), a VL CDR1 (SEQ ID NO:5), a VL CDR2 (SEQ ID NO:6), and a VL CDR3 (SEQ ID NO:7). In one embodiment, the antibody comprises a VH CDR2 (SEQ ID NO:9), a VL CDR1 (SEQ ID NO:5), a VL CDR2 (SEQ ID NO:6), and a VL CDR3 (SEQ ID NO:7). In other embodiments, the antibody comprises a VH CDR3 (SEQ ID NO:10), a VL CDR1 (SEQ ID NO:5), a VL CDR2 (SEQ ID NO:6), and a VL CDR3 (SEQ ID NO:7). In another embodiment, the antibody comprises any combination thereof of the VH CDRs and VL CDRs listed in Tables 1 and 2.

In some embodiments, the antibodies comprise a VH CDR1, VH CDR1, VH CDR3, VL CDR1, VL CDR2, VL CDR3 according to the Chothia numbering system. Accordingly, in some embodiments, the antibodies comprise a VH CDR1 having an amino acid sequence of SEQ ID NO:71. In some embodiments, the antibodies comprise a VH CDR2 having an amino acid sequence of SEQ ID NO:72. In some embodiments, the antibodies comprise a VH CDR3 having an amino acid sequence of SEQ ID NO:73. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 2. In some embodiments, the antibodies comprise a VL CDR1 having an amino acid sequence of any one of SEQ ID NO:74. In another embodiment, the antibodies comprise a VL CDR2 having an amino acid sequence of SEQ ID NO:6. In some embodiments, the antibodies comprise a VL CDR3 having an amino acid sequence of SEQ ID NO:76. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 1.

In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region comprising: (1) a VL CDR1 having an amino acid sequence of SEQ ID NO:74; (2) a VL CDR2 having an amino acid sequence of SEQ ID NO:6; and (3) a VL CDR3 having an amino acid sequence of SEQ ID NO:76.

In some embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73.

In other embodiments, the antibodies provided herein comprise a VL region comprising: (1) a VL CDR1 having an amino acid sequence of SEQ ID NO:74; (2) a VL CDR2 having an amino acid sequence of SEQ ID NO:6; and (3) a VL CDR3 having an amino acid sequence of SEQ ID NO:76.

Also provided herein are antibodies comprising one or more (e.g., one, two, or three) VH CDRs and one or more (e.g., one, two, or three) VL CDRs listed in Tables 1 and 2. In particular, provided herein is an antibody comprising a VH CDR1 (SEQ ID NO:71) and a VL CDR1 (SEQ ID NO:74). In one embodiment, the antibody comprises a VH CDR1 (SEQ ID NO:71) and a VL CDR2 (SEQ ID NO:6). In other embodiments, the antibody comprises a VH CDR1 (SEQ ID NO:71) and a VL CDR3 (SEQ ID NO:76). In another embodiment, the antibody comprises a VH CDR2 (SEQ ID NO:72) and a VL CDR1 (SEQ ID NO:74). In some embodiments, the antibody comprises a VH CDR2 (SEQ ID NO:72) and a VL CDR2 (SEQ ID NO:6). In one embodiment, the antibody comprises a VH CDR2 (SEQ ID NO:72) and a VL CDR3 (SEQ ID NO:76). In another embodiment, the antibody comprises a VH CDR3 (SEQ ID NO:73) and a VL CDR1 (SEQ ID NO:74). In other embodiments, the antibody comprises a VH CDR3 (SEQ ID NO:73) and a VL CDR2 (SEQ ID NO:6). In some embodiments, the antibody comprises a VH CDR3 (SEQ ID NO:73) and a VL CDR3 (SEQ ID NO:76). In another embodiment, the antibody comprises a VH CDR1 (SEQ ID NO:71), a VH CDR2 (SEQ ID NO:72), and a VL CDR1 (SEQ ID NO:74). In one embodiment, the antibody comprises a VH CDR1 (SEQ ID NO:71), a VH CDR2 (SEQ ID NO:72), and a VL CDR2 (SEQ ID NO:6). In other embodiments, the antibody comprises a VH CDR1 (SEQ ID NO:71), a VH CDR2 (SEQ ID NO:72), and a VL CDR3 (SEQ ID NO:76). In another embodiment, the antibody comprises a VH CDR2 (SEQ ID NO:72), a VH CDR3 (SEQ ID NO:73), and a VL CDR1 (SEQ ID NO:74). In some embodiments, the antibody comprises a VH CDR2 (SEQ ID NO:72), a VH CDR3 (SEQ ID NO:73), and a VL CDR2 (SEQ ID NO:6). In one embodiment, the antibody comprises a VH CDR2 (SEQ ID NO:72), a VH CDR3 (SEQ ID NO:73), and a VL CDR3 (SEQ ID NO:76). In another embodiment, the antibody comprises a VH CDR1 (SEQ ID NO:71), a VH CDR3 (SEQ ID NO:73), and a VL CDR1 (SEQ ID NO:74). In other embodiments, the antibody comprises a VH CDR1 (SEQ ID NO:71), a VH CDR3 (SEQ ID NO:73), and a VL CDR2 (SEQ ID NO:6). In some embodiments, the antibody comprises a VH CDR1 (SEQ ID NO:71), a VH CDR3 (SEQ ID NO:73), and a VL CDR3 (SEQ ID NO:76). In another embodiment, the antibody comprises a VH CDR1 (SEQ ID NO:71), a VL CDR1 (SEQ ID NO:74), and a VL CDR2 (SEQ ID NO:6). In one embodiment, the antibody comprises a VH CDR1 (SEQ ID NO:71), a VL CDR1 (SEQ ID NO:74), and a VL CDR3 (SEQ ID NO:76). In other embodiments, the antibody comprises a VH CDR1 (SEQ ID NO:71), a VL CDR2 (SEQ ID NO:6), and a VL CDR3 (SEQ ID NO:76). In another embodiment, the antibody comprises a VH CDR2 (SEQ ID NO:72), a VL CDR1 (SEQ ID NO:74), and a VL CDR2 (SEQ ID NO:6). In some embodiments, the antibody comprises a VH CDR2 (SEQ ID NO:72), a VL CDR1 (SEQ ID NO:74), and a VL CDR3 (SEQ ID NO:76). In one embodiment, the antibody comprises a VH CDR2 (SEQ ID NO:72), a VL CDR2 (SEQ ID NO:6), and a VL CDR3 (SEQ ID NO:76). In another embodiment, the antibody comprises a VH CDR3 (SEQ ID NO:73), a VL CDR1 (SEQ ID NO:74), and a VL CDR2 (SEQ ID NO:6). In other embodiments, the antibody comprises a VH CDR3 (SEQ ID NO:73), a VL CDR1 (SEQ ID NO:74), and a VL CDR3 (SEQ ID NO:76). In some embodiments, the antibody comprises a VH CDR3 (SEQ ID NO:73), a VL CDR2 (SEQ ID NO:6), and a VL CDR3 (SEQ ID NO:76). In another embodiment, the antibody comprises a VH CDR1 (SEQ ID NO:71), a VH CDR2 (SEQ ID NO:72), a VH CDR3 (SEQ ID NO:73), and a VL CDR1 (SEQ ID NO:74). In one embodiment, the antibody comprises a VH CDR1 (SEQ ID NO:71), a VH CDR2 (SEQ ID NO:72), a VH CDR3 (SEQ ID NO:73), and a VL CDR2 (SEQ ID NO:6). In other embodiments, the antibody comprises a VH CDR1 (SEQ ID NO:71), a VH CDR2 (SEQ ID NO:72), a VH CDR3 (SEQ ID NO:73), and a VL CDR3 (SEQ ID NO:76). In another embodiment, the antibody comprises a VH CDR1 (SEQ ID NO:71), a VH CDR2 (SEQ ID NO:72), a VL CDR1 (SEQ ID NO:74), and a VL CDR2 (SEQ ID NO:6). In some embodiments, the antibody comprises a VH CDR1 (SEQ ID NO:71), a VH CDR2 (SEQ ID NO:72), a VL CDR1 (SEQ ID NO:74), and a VL CDR3 (SEQ ID NO:76). In one embodiment, the antibody comprises a VH CDR1 (SEQ ID NO:71), a VH CDR2 (SEQ ID NO:72), a VL CDR2 (SEQ ID NO:6), and a VL CDR3 (SEQ ID NO:76). In another embodiment, the antibody comprises a VH CDR1 (SEQ ID NO:71), a VH CDR3 (SEQ ID NO:73), a VL CDR1 (SEQ ID NO:74), and a VL CDR2 (SEQ ID NO:6). In other embodiments, the antibody comprises a VH CDR1 (SEQ ID NO:71), a VH CDR3 (SEQ ID NO:73), a VL CDR1 (SEQ ID NO:74), and a VL CDR3 (SEQ ID NO:76). In some embodiments, the antibody comprises a VH CDR1 (SEQ ID NO:71), a VH CDR3 (SEQ ID NO:73), a VL CDR2 (SEQ ID NO:6), and a VL CDR3 (SEQ ID NO:76). In another embodiment, the antibody comprises a VH CDR2 (SEQ ID NO:72), a VH CDR3 (SEQ ID NO:73), a VL CDR1 (SEQ ID NO:74), and a VL CDR2 (SEQ ID NO:6). In one embodiment, the antibody comprises a VH CDR2 (SEQ ID NO:72), a VH CDR3 (SEQ ID NO:73), a VL CDR1 (SEQ ID NO:74), and a VL CDR3 (SEQ ID NO:76). In other embodiments, the antibody comprises a VH CDR2 (SEQ ID NO:72), a VH CDR3 (SEQ ID NO:73), a VL CDR2 (SEQ ID NO:6), and a VL CDR3 (SEQ ID NO:76). In another embodiment, the antibody comprises a VH CDR1 (SEQ ID NO:71), a VH CDR2 (SEQ ID NO:72), a VH CDR3 (SEQ ID NO:73), a VL CDR1 (SEQ ID NO:74), and a VL CDR2 (SEQ ID NO:6). In some embodiments, the antibody comprises a VH CDR1 (SEQ ID NO:71), a VH CDR2 (SEQ ID NO:72), a VH CDR3 (SEQ ID NO:73), a VL CDR1 (SEQ ID NO:74), and a VL CDR3 (SEQ ID NO:76). In one embodiment, the antibody comprises a VH CDR1 (SEQ ID NO:71), a VH CDR2 (SEQ ID NO:72), a VH CDR3 (SEQ ID NO:73), a VL CDR2 (SEQ ID NO:6), and a VL CDR3 (SEQ ID NO:76). In another embodiment, the antibody comprises a VH CDR1 (SEQ ID NO:71), a VH CDR2 (SEQ ID NO:72), a VL CDR1 (SEQ ID NO:74), a VL CDR2 (SEQ ID NO:6), and a VL CDR3 (SEQ ID NO:76). In other embodiments, the antibody comprises a VH CDR1 (SEQ ID NO:71), a VH CDR3 (SEQ ID NO:73), a VL CDR1 (SEQ ID NO:74), a VL CDR2 (SEQ ID NO:6), and a VL CDR3 (SEQ ID NO:76). In some embodiments, the antibody comprises a VH CDR2 (SEQ ID NO:72), a VH CDR3 (SEQ ID NO:73), a VL CDR1 (SEQ ID NO:74), a VL CDR2 (SEQ ID NO:6), and a VL CDR3 (SEQ ID NO:76). In another embodiment, the antibody comprises a VH CDR1 (SEQ ID NO:71), a VL CDR1 (SEQ ID NO:74), a VL CDR2 (SEQ ID NO: 6), and a VL CDR3 (SEQ ID NO:76). In one embodiment, the antibody comprises a VH CDR2 (SEQ ID NO:72), a VL CDR1 (SEQ ID NO:74), a VL CDR2 (SEQ ID NO:6), and a VL CDR3 (SEQ ID NO:76). In other embodiments, the antibody comprises a VH CDR3 (SEQ ID NO:73), a VL CDR1 (SEQ ID NO:74), a VL CDR2 (SEQ ID NO:6), and a VL CDR3 (SEQ ID NO:76). In another embodiment, the antibody comprises any combination thereof of the VH CDRs and VL CDRs listed in Tables 1 and 2.

In some embodiments, the antibodies comprise a VH CDR1, VH CDR1, VH CDR3, VL CDR1, VL CDR2, VL CDR3 according to the IMGT numbering system. In some embodiments, the antibodies comprise a VH CDR1 having an amino acid sequence of SEQ ID NO:77. In some embodiments, the antibodies comprise a VH CDR2 having an amino acid sequence of SEQ ID NO:78. In some embodiments, the antibodies comprise a VH CDR3 having an amino acid sequence of SEQ ID NO:79. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 2. In some embodiments, the antibodies comprise a VL CDR1 having an amino acid sequence of any one of SEQ ID NO:80. In another embodiment, the antibodies comprise a VL CDR2 having an amino acid sequence of SEQ ID NO:81. In some embodiments, the antibodies comprise a VL CDR3 having an amino acid sequence of SEQ ID NO:82. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 1.

In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region comprising: (1) a VL CDR1 having an amino acid sequence of SEQ ID NO:80; (2) a VL CDR2 having an amino acid sequence of SEQ ID NO:81; and (3) a VL CDR3 having an amino acid sequence of SEQ ID NO:82.

In some embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79.

In other embodiments, the antibodies provided herein comprise a VL region comprising: (1) a VL CDR1 having an amino acid sequence of SEQ ID NO:80; (2) a VL CDR2 having an amino acid sequence of SEQ ID NO:81; and (3) a VL CDR3 having an amino acid sequence of SEQ ID NO:82.

Also provided herein are antibodies comprising one or more (e.g., one, two, or three) VH CDRs and one or more (e.g., one, two, or three) VL CDRs listed in Tables 1 and 2. In particular, provided herein is an antibody comprising a VH CDR1 (SEQ ID NO:77) and a VL CDR1 (SEQ ID NO:80). In one embodiment, the antibody comprises a VH CDR1 (SEQ ID NO:77) and a VL CDR2 (SEQ ID NO:81). In other embodiments, the antibody comprises a VH CDR1 (SEQ ID NO:77) and a VL CDR3 (SEQ ID NO:82). In another embodiment, the antibody comprises a VH CDR2 (SEQ ID NO:78) and a VL CDR1 (SEQ ID NO:80). In some embodiments, the antibody comprises a VH CDR2 (SEQ ID NO:78) and a VL CDR2 (SEQ ID NO:81). In one embodiment, the antibody comprises a VH CDR2 (SEQ ID NO:78) and a VL CDR3 (SEQ ID NO:82). In another embodiment, the antibody comprises a VH CDR3 (SEQ ID NO:79) and a VL CDR1 (SEQ ID NO:80). In other embodiments, the antibody comprises a VH CDR3 (SEQ ID NO:79) and a VL CDR2 (SEQ ID NO:81). In some embodiments, the antibody comprises a VH CDR3 (SEQ ID NO:79) and a VL CDR3 (SEQ ID NO:82). In another embodiment, the antibody comprises a VH CDR1 (SEQ ID NO:77), a VH CDR2 (SEQ ID NO:78), and a VL CDR1 (SEQ ID NO:80). In one embodiment, the antibody comprises a VH CDR1 (SEQ ID NO:77), a VH CDR2 (SEQ ID NO:78), and a VL CDR2 (SEQ ID NO:81). In other embodiments, the antibody comprises a VH CDR1 (SEQ ID NO:77), a VH CDR2 (SEQ ID NO:78), and a VL CDR3 (SEQ ID NO:82). In another embodiment, the antibody comprises a VH CDR2 (SEQ ID NO:78), a VH CDR3 (SEQ ID NO:79), and a VL CDR1 (SEQ ID NO:80). In some embodiments, the antibody comprises a VH CDR2 (SEQ ID NO:78), a VH CDR3 (SEQ ID NO:79), and a VL CDR2 (SEQ ID NO:81). In one embodiment, the antibody comprises a VH CDR2 (SEQ ID NO:78), a VH CDR3 (SEQ ID NO:79), and a VL CDR3 (SEQ ID NO:82). In another embodiment, the antibody comprises a VH CDR1 (SEQ ID NO:77), a VH CDR3 (SEQ ID NO:79), and a VL CDR1 (SEQ ID NO:80). In other embodiments, the antibody comprises a VH CDR1 (SEQ ID NO:77), a VH CDR3 (SEQ ID NO:79), and a VL CDR2 (SEQ ID NO:81). In some embodiments, the antibody comprises a VH CDR1 (SEQ ID NO:77), a VH CDR3 (SEQ ID NO:79), and a VL CDR3 (SEQ ID NO:82). In another embodiment, the antibody comprises a VH CDR1 (SEQ ID NO:77), a VL CDR1 (SEQ ID NO:80), and a VL CDR2 (SEQ ID NO:81). In one embodiment, the antibody comprises a VH CDR1 (SEQ ID NO:77), a VL CDR1 (SEQ ID NO:80), and a VL CDR3 (SEQ ID NO:82). In other embodiments, the antibody comprises a VH CDR1 (SEQ ID NO:77), a VL CDR2 (SEQ ID NO:81), and a VL CDR3 (SEQ ID NO:82). In another embodiment, the antibody comprises a VH CDR2 (SEQ ID NO:78), a VL CDR1 (SEQ ID NO:80), and a VL CDR2 (SEQ ID NO:81). In some embodiments, the antibody comprises a VH CDR2 (SEQ ID NO:78), a VL CDR1 (SEQ ID NO:80), and a VL CDR3 (SEQ ID NO:82). In one embodiment, the antibody comprises a VH CDR2 (SEQ ID NO:78), a VL CDR2 (SEQ ID NO:81), and a VL CDR3 (SEQ ID NO:82). In another embodiment, the antibody comprises a VH CDR3 (SEQ ID NO:79), a VL CDR1 (SEQ ID NO:80), and a VL CDR2 (SEQ ID NO:81). In other embodiments, the antibody comprises a VH CDR3 (SEQ ID NO:79), a VL CDR1 (SEQ ID NO:80), and a VL CDR3 (SEQ ID NO:82). In some embodiments, the antibody comprises a VH CDR3 (SEQ ID NO:79), a VL CDR2 (SEQ ID NO:81), and a VL CDR3 (SEQ ID NO:82). In another embodiment, the antibody comprises a VH CDR1 (SEQ ID NO:77), a VH CDR2 (SEQ ID NO:78), a VH CDR3 (SEQ ID NO:79), and a VL CDR1 (SEQ ID NO:80). In one embodiment, the antibody comprises a VH CDR1 (SEQ ID NO:77), a VH CDR2 (SEQ ID NO:78), a VH CDR3 (SEQ ID NO:79), and a VL CDR2 (SEQ ID NO:81). In other embodiments, the antibody comprises a VH CDR1 (SEQ ID NO:77), a VH CDR2 (SEQ ID NO:78), a VH CDR3 (SEQ ID NO:79), and a VL CDR3 (SEQ ID NO:82). In another embodiment, the antibody comprises a VH CDR1 (SEQ ID NO:77), a VH CDR2 (SEQ ID NO:78), a VL CDR1 (SEQ ID NO:80), and a VL CDR2 (SEQ ID NO:81). In some embodiments, the antibody comprises a VH CDR1 (SEQ ID NO:77), a VH CDR2 (SEQ ID NO:78), a VL CDR1 (SEQ ID NO:80), and a VL CDR3 (SEQ ID NO:82). In one embodiment, the antibody comprises a VH CDR1 (SEQ ID NO:77), a VH CDR2 (SEQ ID NO:78), a VL CDR2 (SEQ ID NO:81), and a VL CDR3 (SEQ ID NO:82). In another embodiment, the antibody comprises a VH CDR1 (SEQ ID NO:77), a VH CDR3 (SEQ ID NO:79), a VL CDR1 (SEQ ID NO:80), and a VL CDR2 (SEQ ID NO:81). In other embodiments, the antibody comprises a VH CDR1 (SEQ ID NO:77), a VH CDR3 (SEQ ID NO:79), a VL CDR1 (SEQ ID NO:80), and a VL CDR3 (SEQ ID NO:82). In some embodiments, the antibody comprises a VH CDR1 (SEQ ID NO:77), a VH CDR3 (SEQ ID NO:79), a VL CDR2 (SEQ ID NO:81), and a VL CDR3 (SEQ ID NO:82). In another embodiment, the antibody comprises a VH CDR2 (SEQ ID NO:78), a VH CDR3 (SEQ ID NO:79), a VL CDR1 (SEQ ID NO:80), and a VL CDR2 (SEQ ID NO:81). In one embodiment, the antibody comprises a VH CDR2 (SEQ ID NO:78), a VH CDR3 (SEQ ID NO:79), a VL CDR1 (SEQ ID NO:80), and a VL CDR3 (SEQ ID NO:82). In other embodiments, the antibody comprises a VH CDR2 (SEQ ID NO:78), a VH CDR3 (SEQ ID NO:79), a VL CDR2 (SEQ ID NO:81), and a VL CDR3 (SEQ ID NO:82). In another embodiment, the antibody comprises a VH CDR1 (SEQ ID NO:77), a VH CDR2 (SEQ ID NO:78), a VH CDR3 (SEQ ID NO:79), a VL CDR1 (SEQ ID NO:80), and a VL CDR2 (SEQ ID NO:81). In some embodiments, the antibody comprises a VH CDR1 (SEQ ID NO:77), a VH CDR2 (SEQ ID NO:78), a VH CDR3 (SEQ ID NO:79), a VL CDR1 (SEQ ID NO:80), and a VL CDR3 (SEQ ID NO:82). In one embodiment, the antibody comprises a VH CDR1 (SEQ ID NO:77), a VH CDR2 (SEQ ID NO:78), a VH CDR3 (SEQ ID NO:79), a VL CDR2 (SEQ ID NO:81), and a VL CDR3 (SEQ ID NO:82). In another embodiment, the antibody comprises a VH CDR1 (SEQ ID NO:77), a VH CDR2 (SEQ ID NO:78), a VL CDR1 (SEQ ID NO:80), a VL CDR2 (SEQ ID NO:81), and a VL CDR3 (SEQ ID NO:82). In other embodiments, the antibody comprises a VH CDR1 (SEQ ID NO:77), a VH CDR3 (SEQ ID NO:79), a VL CDR1 (SEQ ID NO:80), a VL CDR2 (SEQ ID NO:81), and a VL CDR3 (SEQ ID NO:82). In some embodiments, the antibody comprises a VH CDR2 (SEQ ID NO:78), a VH CDR3 (SEQ ID NO:79), a VL CDR1 (SEQ ID NO:80), a VL CDR2 (SEQ ID NO:81), and a VL CDR3 (SEQ ID NO:82). In another embodiment, the antibody comprises a VH CDR1 (SEQ ID NO:77), a VL CDR1 (SEQ ID NO:80), a VL CDR2 (SEQ ID NO:81), and a VL CDR3 (SEQ ID NO:82). In one embodiment, the antibody comprises a VH CDR2 (SEQ ID NO:78), a VL CDR1 (SEQ ID NO:80), a VL CDR2 (SEQ ID NO:81), and a VL CDR3 (SEQ ID NO:82). In other embodiments, the antibody comprises a VH CDR3 (SEQ ID NO:79), a VL CDR1 (SEQ ID NO:80), a VL CDR2 (SEQ ID NO:81), and a VL CDR3 (SEQ ID NO:82). In another embodiment, the antibody comprises any combination thereof of the VH CDRs and VL CDRs listed in Tables 1 and 2.

In some embodiments, the antibodies comprise a VH CDR1, VH CDR1, VH CDR3, VL CDR1, VL CDR2, VL CDR3 according to the Contact numbering system. In some embodiments, the antibodies comprise a VH CDR1, VH CDR1, VH CDR3, VL CDR1, VL CDR2, VL CDR3 according to the Contact numbering system. In some embodiments, the antibodies comprise a VH CDR1 having an amino acid sequence of SEQ ID NO:83. In some embodiments, the antibodies comprise a VH CDR2 having an amino acid sequence of SEQ ID NO:84. In some embodiments, the antibodies comprise a VH CDR3 having an amino acid sequence of SEQ ID NO:85. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 2. In some embodiments, the antibodies comprise a VL CDR1 having an amino acid sequence of any one of SEQ ID NO:86. In another embodiment, the antibodies comprise a VL CDR2 having an amino acid sequence of SEQ ID NO:87. In some embodiments, the antibodies comprise a VL CDR3 having an amino acid sequence of SEQ ID NO:88. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 1.

In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region comprising: (1) a VL CDR1 having an amino acid sequence of SEQ ID NO:86; (2) a VL CDR2 having an amino acid sequence of SEQ ID NO:87; and (3) a VL CDR3 having an amino acid sequence of SEQ ID NO:88.

In some embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85.

In other embodiments, the antibodies provided herein comprise a VL region comprising: (1) a VL CDR1 having an amino acid sequence of SEQ ID NO:86; (2) a VL CDR2 having an amino acid sequence of SEQ ID NO:87; and (3) a VL CDR3 having an amino acid sequence of SEQ ID NO:88.

Also provided herein are antibodies comprising one or more (e.g., one, two, or three) VH CDRs and one or more (e.g., one, two, or three) VL CDRs listed in Tables 1 and 2. In particular, provided herein is an antibody comprising a VH CDR1 (SEQ ID NO:83) and a VL CDR1 (SEQ ID NO:86). In one embodiment, the antibody comprises a VH CDR1 (SEQ ID NO:83) and a VL CDR2 (SEQ ID NO:87). In other embodiments, the antibody comprises a VH CDR1 (SEQ ID NO:83) and a VL CDR3 (SEQ ID NO:88). In another embodiment, the antibody comprises a VH CDR2 (SEQ ID NO:84) and a VL CDR1 (SEQ ID NO:86). In some embodiments, the antibody comprises a VH CDR2 (SEQ ID NO:84) and a VL CDR2 (SEQ ID NO:87). In one embodiment, the antibody comprises a VH CDR2 (SEQ ID NO:84) and a VL CDR3 (SEQ ID NO:88). In another embodiment, the antibody comprises a VH CDR3 (SEQ ID NO:85) and a VL CDR1 (SEQ ID NO:86). In other embodiments, the antibody comprises a VH CDR3 (SEQ ID NO:85) and a VL CDR2 (SEQ ID NO:87). In some embodiments, the antibody comprises a VH CDR3 (SEQ ID NO:85) and a VL CDR3 (SEQ ID NO:88). In another embodiment, the antibody comprises a VH CDR1 (SEQ ID NO:83), a VH CDR2 (SEQ ID NO:84), and a VL CDR1 (SEQ ID NO:86). In one embodiment, the antibody comprises a VH CDR1 (SEQ ID NO:83), a VH CDR2 (SEQ ID NO:84), and a VL CDR2 (SEQ ID NO:87). In other embodiments, the antibody comprises a VH CDR1 (SEQ ID NO:83), a VH CDR2 (SEQ ID NO:84), and a VL CDR3 (SEQ ID NO:88). In another embodiment, the antibody comprises a VH CDR2 (SEQ ID NO:84), a VH CDR3 (SEQ ID NO:85), and a VL CDR1 (SEQ ID NO:86). In some embodiments, the antibody comprises a VH CDR2 (SEQ ID NO:84), a VH CDR3 (SEQ ID NO:85), and a VL CDR2 (SEQ ID NO:87). In one embodiment, the antibody comprises a VH CDR2 (SEQ ID NO:84), a VH CDR3 (SEQ ID NO:85), and a VL CDR3 (SEQ ID NO:88). In another embodiment, the antibody comprises a VH CDR1 (SEQ ID NO:83), a VH CDR3 (SEQ ID NO:85), and a VL CDR1 (SEQ ID NO:86). In other embodiments, the antibody comprises a VH CDR1 (SEQ ID NO:83), a VH CDR3 (SEQ ID NO:85), and a VL CDR2 (SEQ ID NO:87). In some embodiments, the antibody comprises a VH CDR1 (SEQ ID NO:83), a VH CDR3 (SEQ ID NO:85), and a VL CDR3 (SEQ ID NO:88). In another embodiment, the antibody comprises a VH CDR1 (SEQ ID NO:83), a VL CDR1 (SEQ ID NO:86), and a VL CDR2 (SEQ ID NO:87). In one embodiment, the antibody comprises a VH CDR1 (SEQ ID NO:83), a VL CDR1 (SEQ ID NO:86), and a VL CDR3 (SEQ ID NO:88). In other embodiments, the antibody comprises a VH CDR1 (SEQ ID NO:83), a VL CDR2 (SEQ ID NO:87), and a VL CDR3 (SEQ ID NO:88). In another embodiment, the antibody comprises a VH CDR2 (SEQ ID NO:84), a VL CDR1 (SEQ ID NO:86), and a VL CDR2 (SEQ ID NO:87). In some embodiments, the antibody comprises a VH CDR2 (SEQ ID NO:84), a VL CDR1 (SEQ ID NO:86), and a VL CDR3 (SEQ ID NO:88). In one embodiment, the antibody comprises a VH CDR2 (SEQ ID NO:84), a VL CDR2 (SEQ ID NO:87), and a VL CDR3 (SEQ ID NO:88). In another embodiment, the antibody comprises a VH CDR3 (SEQ ID NO:85), a VL CDR1 (SEQ ID NO:86), and a VL CDR2 (SEQ ID NO:87). In other embodiments, the antibody comprises a VH CDR3 (SEQ ID NO:85), a VL CDR1 (SEQ ID NO:86), and a VL CDR3 (SEQ ID NO:88). In some embodiments, the antibody comprises a VH CDR3 (SEQ ID NO:85), a VL CDR2 (SEQ ID NO:87), and a VL CDR3 (SEQ ID NO:88). In another embodiment, the antibody comprises a VH CDR1 (SEQ ID NO:83), a VH CDR2 (SEQ ID NO:84), a VH CDR3 (SEQ ID NO:85), and a VL CDR1 (SEQ ID NO:86). In one embodiment, the antibody comprises a VH CDR1 (SEQ ID NO:83), a VH CDR2 (SEQ ID NO:84), a VH CDR3 (SEQ ID NO:85), and a VL CDR2 (SEQ ID NO:87). In other embodiments, the antibody comprises a VH CDR1 (SEQ ID NO:83), a VH CDR2 (SEQ ID NO:84), a VH CDR3 (SEQ ID NO:85), and a VL CDR3 (SEQ ID NO:88). In another embodiment, the antibody comprises a VH CDR1 (SEQ ID NO:83), a VH CDR2 (SEQ ID NO:84), a VL CDR1 (SEQ ID NO:86), and a VL CDR2 (SEQ ID NO:87). In some embodiments, the antibody comprises a VH CDR1 (SEQ ID NO:83), a VH CDR2 (SEQ ID NO:84), a VL CDR1 (SEQ ID NO:86), and a VL CDR3 (SEQ ID NO:88). In one embodiment, the antibody comprises a VH CDR1 (SEQ ID NO:83), a VH CDR2 (SEQ ID NO:84), a VL CDR2 (SEQ ID NO:87), and a VL CDR3 (SEQ ID NO:88). In another embodiment, the antibody comprises a VH CDR1 (SEQ ID NO:83), a VH CDR3 (SEQ ID NO:85), a VL CDR1 (SEQ ID NO:86), and a VL CDR2 (SEQ ID NO:87). In other embodiments, the antibody comprises a VH CDR1 (SEQ ID NO:83), a VH CDR3 (SEQ ID NO:85), a VL CDR1 (SEQ ID NO:86), and a VL CDR3 (SEQ ID NO:88). In some embodiments, the antibody comprises a VH CDR1 (SEQ ID NO:83), a VH CDR3 (SEQ ID NO:85), a VL CDR2 (SEQ ID NO:87), and a VL CDR3 (SEQ ID NO:88). In another embodiment, the antibody comprises a VH CDR2 (SEQ ID NO: 84), a VH CDR3 (SEQ ID NO:85), a VL CDR1 (SEQ ID NO:86), and a VL CDR2 (SEQ ID NO:87). In one embodiment, the antibody comprises a VH CDR2 (SEQ ID NO:84), a VH CDR3 (SEQ ID NO:85), a VL CDR1 (SEQ ID NO:86), and a VL CDR3 (SEQ ID NO:88). In other embodiments, the antibody comprises a VH CDR2 (SEQ ID NO:84), a VH CDR3 (SEQ ID NO:85), a VL CDR2 (SEQ ID NO:87), and a VL CDR3 (SEQ ID NO:88). In another embodiment, the antibody comprises a VH CDR1 (SEQ ID NO:83), a VH CDR2 (SEQ ID NO:84), a VH CDR3 (SEQ ID NO:85), a VL CDR1 (SEQ ID NO:86), and a VL CDR2 (SEQ ID NO:87). In some embodiments, the antibody comprises a VH CDR1 (SEQ ID NO:83), a VH CDR2 (SEQ ID NO:84), a VH CDR3 (SEQ ID NO:85), a VL CDR1 (SEQ ID NO:86), and a VL CDR3 (SEQ ID NO:88). In one embodiment, the antibody comprises a VH CDR1 (SEQ ID NO:83), a VH CDR2 (SEQ ID NO:84), a VH CDR3 (SEQ ID NO:85), a VL CDR2 (SEQ ID NO:87), and a VL CDR3 (SEQ ID NO:88). In another embodiment, the antibody comprises a VH CDR1 (SEQ ID NO:83), a VH CDR2 (SEQ ID NO:84), a VL CDR1 (SEQ ID NO:86), a VL CDR2 (SEQ ID NO:87), and a VL CDR3 (SEQ ID NO:88). In other embodiments, the antibody comprises a VH CDR1 (SEQ ID NO:83), a VH CDR3 (SEQ ID NO:85), a VL CDR1 (SEQ ID NO:86), a VL CDR2 (SEQ ID NO:87), and a VL CDR3 (SEQ ID NO:88). In some embodiments, the antibody comprises a VH CDR2 (SEQ ID NO:84), a VH CDR3 (SEQ ID NO:85), a VL CDR1 (SEQ ID NO:86), a VL CDR2 (SEQ ID NO:87), and a VL CDR3 (SEQ ID NO:88). In another embodiment, the antibody comprises a VH CDR1 (SEQ ID NO:83), a VL CDR1 (SEQ ID NO:86), a VL CDR2 (SEQ ID NO:87), and a VL CDR3 (SEQ ID NO:88). In one embodiment, the antibody comprises a VH CDR2 (SEQ ID NO:84), a VL CDR1 (SEQ ID NO:86), a VL CDR2 (SEQ ID NO:87), and a VL CDR3 (SEQ ID NO:88). In other embodiments, the antibody comprises a VH CDR3 (SEQ ID NO:85), a VL CDR1 (SEQ ID NO:86), a VL CDR2 (SEQ ID NO:87), and a VL CDR3 (SEQ ID NO:88). In another embodiment, the antibody comprises any combination thereof of the VH CDRs and VL CDRs listed in Tables 1 and 2.

TABLE 3
VL CDR Sequences of Anti-BTN1A1 Antibody STC43G3-1

| | Region | | | |
|---|---|---|---|---|
| | definition | CDR1 | CDR2 | CDR3 |
| Kappa | Chothia | QSSQSVYNNNNLS | RASKLPS | AGGYSGDINV |
| light | | (SEQ ID NO: 94) | (SEQ ID NO: 95) | (SEQ ID NO: 96) |
| chain | IMGT | QSVYNN | RAS | AGGYSGDINV |
| | | (SEQ ID NO: 100) | (SEQ ID NO: 101) | (SEQ ID NO: 102) |
| | Kabat | QSSQSVYNNNNLS | RASKLPS | AGGYSGDINV |
| | | (SEQ ID NO: 38) | (SEQ ID NO: 39) | (SEQ ID NO: 40) |
| | Contact | YNNNNLSWF | LLIYRASKLP | AGGYSGDIN |
| | | (SEQ ID NO: 106) | (SEQ ID NO: 107) | (SEQ ID NO: 108) |

TABLE 4
VH CDR Sequences of Anti-BTN1A1 Antibody STC43G3-1

| | Region | | | |
|---|---|---|---|---|
| | definition | CDR1 | CDR2 | CDR3 |
| Heavy | Chothia | GIDLSRY | GTTGN | SVVSPSNSSF |
| chain | | (SEQ ID NO: 91) | (SEQ ID NO: 92 | (SEQ ID NO: 93) |
| | IMGT | GIDLSRYG | IGTTGNT | ARSVVSPSNSSF |
| | | (SEQ ID NO: 97) | (SEQ ID NO: 98) | (SEQ ID NO: 99) |
| | Kabat | RYGVN | YIGTTGNTYYASW | SVVSPSNSSF |
| | | (SEQ ID NO: 44) | VNG | (SEQ ID NO: 46) |
| | | | (SEQ ID NO: 45) | |
| | Contact | SRYGVN | WIGYIGTTGNTY | ARSVVSPSNSS |
| | | (SEQ ID NO: 103) | (SEQ ID NO: 104) | (SEQ ID NO: 105) |

In some embodiments, an antibody provided herein comprises or consists of six CDRs, for example, VH CDR1, VH CDR2, VH CDR3, VL CDR1, VL CDR2, and/or VL CDR3 identified in Tables 3 and 4. In some embodiments, an antibody provided herein can comprise fewer than six CDRs. In some embodiments, the antibody comprises or consists of one, two, three, four, or five CDRs selected from the group consisting of VH CDR1, VH CDR2, VH CDR3, VL CDR1, VL CDR2, and/or VL CDR3 identified in Tables 3 and 4. In some embodiments, the antibody comprises or consists of one, two, three, four, or five CDRs selected from the group consisting of VH CDR1, VH CDR2, VH CDR3, VL CDR1, VL CDR2, and/or VL CDR3 of the monoclonal described herein. Accordingly, in some embodiments, the antibody comprises or consists of one, two, three, four, or five CDRs of anyone of the VH CDR1, VH CDR2, VH CDR3, VL CDR1, VL CDR2, and/or VL CDR3 identified in Tables 3 and 4.

In some embodiments, the antibodies provided herein comprise one or more (e.g., one, two, or three) VH CDRs listed in Table 4. In other embodiments, the antibodies provided herein comprise one or more (e.g., one, two, or three) VL CDRs listed in Table 3. In yet other embodiments, the antibodies provided herein comprise one or more (e.g., one, two, or three) VH CDRs listed in Table 4 and one or more VL CDRs listed in Table 3.

In some embodiments, the antibodies comprise a VH CDR1, VH CDR1, VH CDR3, VL CDR1, VL CDR2, VL CDR3 according to the Contact numbering system. In some embodiments, the antibodies comprise a VH CDR1, VH CDR1, VH CDR3, VL CDR1, VL CDR2, VL CDR3 according to the Kabat numbering system. Accordingly, in some embodiments, the antibodies comprise a VH CDR1 having an amino acid sequence of SEQ ID NO:44. In some embodiments, the antibodies comprise a VH CDR2 having an amino acid sequence of SEQ ID NO:45. In some embodiments, the antibodies comprise a VH CDR3 having an amino acid sequence of SEQ ID NO:46. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 4. In some embodiments, the antibodies comprise a VL CDR1 having an amino acid sequence of any one of SEQ ID NO:38. In another embodiment, the antibodies comprise a VL CDR2 having an amino acid sequence of SEQ ID NO:39. In some embodiments, the antibodies comprise a VL CDR3 having an amino acid sequence of SEQ ID NO:40. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 3.

In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region comprising: (1) a VL CDR1 having an amino acid sequence of SEQ ID NO:38; (2) a VL CDR2 having an amino acid sequence of SEQ ID NO:39; and (3) a VL CDR3 having an amino acid sequence of SEQ ID NO:40.

In some embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46.

In other embodiments, the antibodies provided herein comprise a VL region comprising: (1) a VL CDR1 having an amino acid sequence of SEQ ID NO:38; (2) a VL CDR2 having an amino acid sequence of SEQ ID NO:39; and (3) a VL CDR3 having an amino acid sequence of SEQ ID NO:40.

Also provided herein are antibodies comprising one or more (e.g., one, two, or three) VH CDRs and one or more (e.g., one, two, or three) VL CDRs listed in Tables 3 and 4. In particular, provided herein is an antibody comprising a VH CDR1 (SEQ ID NO:44) and a VL CDR1 (SEQ ID NO:38). In one embodiment, the antibody comprises a VH CDR1 (SEQ ID NO:44) and a VL CDR2 (SEQ ID NO:39). In other embodiments, the antibody comprises a VH CDR1 (SEQ ID NO:44) and a VL CDR3 (SEQ ID NO:40). In another embodiment, the antibody comprises a VH CDR2 (SEQ ID NO:45) and a VL CDR1 (SEQ ID NO:38). In some embodiments, the antibody comprises a VH CDR2 (SEQ ID NO:45) and a VL CDR2 (SEQ ID NO:39). In one embodiment, the antibody comprises a VH CDR2 (SEQ ID NO:45) and a VL CDR3 (SEQ ID NO:40). In another embodiment, the antibody comprises a VH CDR3 (SEQ ID NO:46) and a VL CDR1 (SEQ ID NO:38). In other embodiments, the antibody comprises a VH CDR3 (SEQ ID NO:46) and a VL CDR2 (SEQ ID NO:39). In some embodiments, the antibody comprises a VH CDR3 (SEQ ID NO:46) and a VL CDR3 (SEQ ID NO:40). In another embodiment, the antibody comprises a VH CDR1 (SEQ ID NO:44), a VH CDR2 (SEQ ID NO:45), and a VL CDR1 (SEQ ID NO:38). In one embodiment, the antibody comprises a VH CDR1 (SEQ ID NO:44), a VH CDR2 (SEQ ID NO:45), and a VL CDR2 (SEQ ID NO:39). In other embodiments, the antibody comprises a VH CDR1 (SEQ ID NO:44), a VH CDR2 (SEQ ID NO:45), and a VL CDR3 (SEQ ID NO:40). In another embodiment, the antibody comprises a VH CDR2 (SEQ ID NO:45), a VH CDR3 (SEQ ID NO:46), and a VL CDR1 (SEQ ID NO:38). In some embodiments, the antibody comprises a VH CDR2 (SEQ ID NO:45), a VH CDR3 (SEQ ID NO:46), and a VL CDR2 (SEQ ID NO:39). In one embodiment, the antibody comprises a VH CDR2 (SEQ ID NO:45), a VH CDR3 (SEQ ID NO:46), and a VL CDR3 (SEQ ID NO:40). In another embodiment, the antibody comprises a VH CDR1 (SEQ ID NO:44), a VH CDR3 (SEQ ID NO:46), and a VL CDR1 (SEQ ID NO:38). In other embodiments, the antibody comprises a VH CDR1 (SEQ ID NO:44), a VH CDR3 (SEQ ID NO:46), and a VL CDR2 (SEQ ID NO:39). In some embodiments, the antibody comprises a VH CDR1 (SEQ ID NO:44), a VH CDR3 (SEQ ID NO:46), and a VL CDR3 (SEQ ID NO:40). In another embodiment, the antibody comprises a VH CDR1 (SEQ ID NO:44), a VL CDR1 (SEQ ID NO:38), and a VL CDR2 (SEQ ID NO:39). In one embodiment, the antibody comprises a VH CDR1 (SEQ ID NO:44), a VL CDR1 (SEQ ID NO:38), and a VL CDR3 (SEQ ID NO:40). In other embodiments, the antibody comprises a VH CDR1 (SEQ ID NO:44), a VL CDR2 (SEQ ID NO:39), and a VL CDR3 (SEQ ID NO:40). In another embodiment, the antibody comprises a VH CDR2 (SEQ ID NO:45), a VL CDR1 (SEQ ID NO:38), and a VL CDR2 (SEQ ID NO:39). In some embodiments, the antibody comprises a VH CDR2 (SEQ ID NO:45), a VL CDR1 (SEQ ID NO:38), and a VL CDR3 (SEQ ID NO:40). In one embodiment, the antibody comprises a VH CDR2 (SEQ ID NO:45), a VL CDR2 (SEQ ID NO:39), and a VL CDR3 (SEQ ID NO:40). In another embodiment, the antibody comprises a VH CDR3 (SEQ ID NO:46), a VL CDR1 (SEQ ID NO:38), and a VL CDR2 (SEQ ID NO:39). In other embodiments, the antibody comprises a VH CDR3 (SEQ ID NO:46), a VL CDR1 (SEQ ID NO:38), and a VL CDR3 (SEQ ID NO:40). In some embodiments, the antibody comprises a VH CDR3 (SEQ ID NO:46), a VL CDR2 (SEQ ID NO:39), and a VL CDR3 (SEQ ID NO:40). In another embodiment, the antibody comprises a VH CDR1 (SEQ ID NO:44), a VH CDR2 (SEQ ID NO:45), a VH CDR3 (SEQ ID NO:46), and a VL CDR1 (SEQ ID NO:38). In one embodiment, the antibody comprises a VH CDR1 (SEQ ID NO:44), a VH CDR2 (SEQ ID NO:45), a VH CDR3 (SEQ ID NO:46), and a VL CDR2 (SEQ ID NO:39). In other embodiments, the antibody comprises a VH CDR1 (SEQ ID NO:44), a VH CDR2 (SEQ ID NO:45), a VH CDR3 (SEQ ID NO:46), and a VL CDR3 (SEQ ID NO:40). In another embodiment, the antibody comprises a VH CDR1 (SEQ ID NO:44), a VH CDR2 (SEQ ID NO:45), a VL CDR1 (SEQ ID NO:38), and a VL CDR2 (SEQ ID NO:39). In some embodiments, the antibody comprises a VH CDR1 (SEQ ID NO:44), a VH CDR2 (SEQ ID NO:45), a VL CDR1 (SEQ ID NO:38), and a VL CDR3 (SEQ ID NO:40). In one embodiment, the antibody comprises a VH CDR1 (SEQ ID NO:44), a VH CDR2 (SEQ ID NO:45), a VL CDR2 (SEQ ID NO:39), and a VL CDR3 (SEQ ID NO:40). In another embodiment, the antibody comprises a VH CDR1 (SEQ ID NO:44), a VH CDR3 (SEQ ID NO:46), a VL CDR1 (SEQ ID NO:38), and a VL CDR2 (SEQ ID NO:39). In other embodiments, the antibody comprises a VH CDR1 (SEQ ID NO:44), a VH CDR3 (SEQ ID NO:46), a VL CDR1 (SEQ ID NO:38), and a VL CDR3 (SEQ ID NO:40). In some embodiments, the antibody comprises a VH CDR1 (SEQ ID NO:44), a VH CDR3 (SEQ ID NO:46), a VL CDR2 (SEQ ID NO:39), and a VL CDR3 (SEQ ID NO:40). In another embodiment, the antibody comprises a VH CDR2 (SEQ ID NO:45), a VH CDR3 (SEQ ID NO:46), a VL CDR1 (SEQ ID NO:38), and a VL CDR2 (SEQ ID NO:39). In one embodiment, the antibody comprises a VH CDR2 (SEQ ID NO:45), a VH CDR3 (SEQ ID NO:46), a VL CDR1 (SEQ ID NO:38), and a VL CDR3 (SEQ ID NO:40). In other embodiments, the antibody comprises a VH CDR2 (SEQ ID NO:45), a VH CDR3 (SEQ ID NO:46), a VL CDR2 (SEQ ID NO:39), and a VL CDR3 (SEQ ID NO:40). In another embodiment, the antibody comprises a VH CDR1 (SEQ ID NO:44), a VH CDR2 (SEQ ID NO:45), a VH CDR3 (SEQ ID NO:46), a VL CDR1 (SEQ ID NO:38), and a VL CDR2 (SEQ ID NO:39). In some embodiments, the antibody comprises a VH CDR1 (SEQ ID NO:44), a VH CDR2 (SEQ ID NO:45), a VH CDR3 (SEQ ID NO:46), a VL CDR1 (SEQ ID NO:38), and a VL CDR3 (SEQ ID NO:40). In one embodiment, the antibody comprises a VH CDR1 (SEQ ID NO:44), a VH CDR2 (SEQ ID NO:45), a VH CDR3 (SEQ ID NO:46), a VL CDR2 (SEQ ID NO:39), and a VL CDR3 (SEQ ID NO:40). In another embodiment, the antibody comprises a VH CDR1 (SEQ ID NO:44), a VH CDR2 (SEQ ID NO:45), a VL CDR1 (SEQ ID NO:38), a VL CDR2 (SEQ ID NO:39), and a VL CDR3 (SEQ ID NO:40). In other embodiments, the antibody comprises a VH CDR1 (SEQ ID NO:44), a VH CDR3 (SEQ ID NO:46), a VL CDR1 (SEQ ID NO:38), a VL CDR2 (SEQ ID NO:39), and a VL CDR3 (SEQ ID NO:40). In some embodiments, the antibody comprises a VH CDR2 (SEQ ID NO:45), a VH CDR3 (SEQ ID NO:46), a VL CDR1 (SEQ ID NO:38), a VL CDR2 (SEQ ID NO:39), and a VL CDR3 (SEQ ID NO:40). In another embodiment, the antibody comprises a VH CDR1 (SEQ ID NO:44), a VL CDR1 (SEQ ID NO:38), a VL CDR2 (SEQ ID NO:39), and a VL CDR3 (SEQ ID NO:40). In one embodiment, the antibody comprises a VH CDR2 (SEQ ID NO:45), a VL CDR1 (SEQ ID NO:38), a VL CDR2 (SEQ ID NO:39), and a VL CDR3 (SEQ ID NO:40). In other embodiments, the antibody comprises a VH CDR3 (SEQ ID NO:46), a VL CDR1 (SEQ ID NO:38), a VL CDR2 (SEQ ID NO:39), and a VL CDR3 (SEQ ID NO: 40). In another embodiment, the antibody comprises any combination thereof of the VH CDRs and VL CDRs listed in Tables 3 and 4.

In some embodiments, the antibodies comprise a VH CDR1, VH CDR1, VH CDR3, VL CDR1, VL CDR2, VL CDR3 according to the Chothia numbering system. Accordingly, in some embodiments, the antibodies comprise a VH CDR1 having an amino acid sequence of SEQ ID NO:91. In some embodiments, the antibodies comprise a VH CDR2 having an amino acid sequence of SEQ ID NO:92. In some embodiments, the antibodies comprise a VH CDR3 having an amino acid sequence of SEQ ID NO:93. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 4. In some embodiments, the antibodies comprise a VL CDR1 having an amino acid sequence of any one of SEQ ID NO:94. In another embodiment, the antibodies comprise a VL CDR2 having an amino acid sequence of SEQ ID NO:95. In some embodiments, the antibodies comprise a VL CDR3 having an amino acid sequence of SEQ ID NO:96. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 3.

In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region comprising: (1) a VL CDR1 having an amino acid sequence of SEQ ID NO:94; (2) a VL CDR2 having an amino acid sequence of SEQ ID NO:95; and (3) a VL CDR3 having an amino acid sequence of SEQ ID NO:96.

In some embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93.

In other embodiments, the antibodies provided herein comprise a VL region comprising: (1) a VL CDR1 having an amino acid sequence of SEQ ID NO:94; (2) a VL CDR2 having an amino acid sequence of SEQ ID NO:95; and (3) a VL CDR3 having an amino acid sequence of SEQ ID NO:96.

Also provided herein are antibodies comprising one or more (e.g., one, two, or three) VH CDRs and one or more (e.g., one, two, or three) VL CDRs listed in Tables 3 and 4. In particular, provided herein is an antibody comprising a VH CDR1 (SEQ ID NO:91) and a VL CDR1 (SEQ ID NO:94). In one embodiment, the antibody comprises a VH CDR1 (SEQ ID NO:91) and a VL CDR2 (SEQ ID NO:95). In other embodiments, the antibody comprises a VH CDR1 (SEQ ID NO:91) and a VL CDR3 (SEQ ID NO:96). In another embodiment, the antibody comprises a VH CDR2 (SEQ ID NO:92) and a VL CDR1 (SEQ ID NO:94). In some embodiments, the antibody comprises a VH CDR2 (SEQ ID NO:92) and a VL CDR2 (SEQ ID NO:95). In one embodiment, the antibody comprises a VH CDR2 (SEQ ID NO:92) and a VL CDR3 (SEQ ID NO:96). In another embodiment, the antibody comprises a VH CDR3 (SEQ ID NO:93) and a VL CDR1 (SEQ ID NO:94). In other embodiments, the antibody comprises a VH CDR3 (SEQ ID NO:93) and a VL CDR2 (SEQ ID NO:95). In some embodiments, the antibody comprises a VH CDR3 (SEQ ID NO:93) and a VL CDR3 (SEQ ID NO:96). In another embodiment, the antibody comprises a VH CDR1 (SEQ ID NO:91), a VH CDR2 (SEQ ID NO:92), and a VL CDR1 (SEQ ID NO:94). In one embodiment, the antibody comprises a VH CDR1 (SEQ ID NO:91), a VH CDR2 (SEQ ID NO:92), and a VL CDR2 (SEQ ID NO:95). In other embodiments, the antibody comprises a VH CDR1 (SEQ ID NO:91), a VH CDR2 (SEQ ID NO:92), and a VL CDR3 (SEQ ID NO:96). In another embodiment, the antibody comprises a VH CDR2 (SEQ ID NO:92), a VH CDR3 (SEQ ID NO:93), and a VL CDR1 (SEQ ID NO:94). In some embodiments, the antibody comprises a VH CDR2 (SEQ ID NO:92), a VH CDR3 (SEQ ID NO:93), and a VL CDR2 (SEQ ID NO:95). In one embodiment, the antibody comprises a VH CDR2 (SEQ ID NO:92), a VH CDR3 (SEQ ID NO:93), and a VL CDR3 (SEQ ID NO:96). In another embodiment, the antibody comprises a VH CDR1 (SEQ ID NO:91), a VH CDR3 (SEQ ID NO:93), and a VL CDR1 (SEQ ID NO:94). In other embodiments, the antibody comprises a VH CDR1 (SEQ ID NO:91), a VH CDR3 (SEQ ID NO:93), and a VL CDR2 (SEQ ID NO:95). In some embodiments, the antibody comprises a VH CDR1 (SEQ ID NO:91), a VH CDR3 (SEQ ID NO:93), and a VL CDR3 (SEQ ID NO:96). In another embodiment, the antibody comprises a VH CDR1 (SEQ ID NO:91), a VL CDR1 (SEQ ID NO:94), and a VL CDR2 (SEQ ID NO:95). In one embodiment, the antibody comprises a VH CDR1 (SEQ ID NO:91), a VL CDR1 (SEQ ID NO:94), and a VL CDR3 (SEQ ID NO:96). In other embodiments, the antibody comprises a VH CDR1 (SEQ ID NO:91), a VL CDR2 (SEQ ID NO:95), and a VL CDR3 (SEQ ID NO:96). In another embodiment, the antibody comprises a VH CDR2 (SEQ ID NO:92), a VL CDR1 (SEQ ID NO:94), and a VL CDR2 (SEQ ID NO:95). In some embodiments, the antibody comprises a VH CDR2 (SEQ ID NO:92), a VL CDR1 (SEQ ID NO:94), and a VL CDR3 (SEQ ID NO:96). In one embodiment, the antibody comprises a VH CDR2 (SEQ ID NO:92), a VL CDR2 (SEQ ID NO:95), and a VL CDR3 (SEQ ID NO:96). In another embodiment, the antibody comprises a VH CDR3 (SEQ ID NO:93), a VL CDR1 (SEQ ID NO:94), and a VL CDR2 (SEQ ID NO:95). In other embodiments, the antibody comprises a VH CDR3 (SEQ ID NO:93), a VL CDR1 (SEQ ID NO:94), and a VL CDR3 (SEQ ID NO:96). In some embodiments, the antibody comprises a VH CDR3 (SEQ ID NO:93), a VL CDR2 (SEQ ID NO:95), and a VL CDR3 (SEQ ID NO:96). In another embodiment, the antibody comprises a VH CDR1 (SEQ ID NO:91), a VH CDR2 (SEQ ID NO:92), a VH CDR3 (SEQ ID NO:93), and a VL CDR1 (SEQ ID NO:94). In one embodiment, the antibody comprises a VH CDR1 (SEQ ID NO:91), a VH CDR2 (SEQ ID NO:92), a VH CDR3 (SEQ ID NO:93), and a VL CDR2 (SEQ ID NO:95). In other embodiments, the antibody comprises a VH CDR1 (SEQ ID NO:91), a VH CDR2 (SEQ ID NO:92), a VH CDR3 (SEQ ID NO:93), and a VL CDR3 (SEQ ID NO:96). In another embodiment, the antibody comprises a VH CDR1 (SEQ ID NO:91), a VH CDR2 (SEQ ID NO:92), a VL CDR1 (SEQ ID NO:94), and a VL CDR2 (SEQ ID NO:95). In some embodiments, the antibody comprises a VH CDR1 (SEQ ID NO:91), a VH CDR2 (SEQ ID NO:92), a VL CDR1 (SEQ ID NO:94), and a VL CDR3 (SEQ ID NO:96). In one embodiment, the antibody comprises a VH CDR1 (SEQ ID NO:91), a VH CDR2 (SEQ ID NO:92), a VL CDR2 (SEQ ID NO:95), and a VL CDR3 (SEQ ID NO:96). In another embodiment, the antibody comprises a VH CDR1 (SEQ ID NO:91), a VH CDR3 (SEQ ID NO:93), a VL CDR1 (SEQ ID NO:94), and a VL CDR2 (SEQ ID NO:95). In other embodiments, the antibody comprises a VH CDR1 (SEQ ID NO:91), a VH CDR3 (SEQ ID NO:93), a VL CDR1 (SEQ ID NO:94), and a VL CDR3 (SEQ ID NO:96). In some embodiments, the antibody comprises a VH CDR1 (SEQ ID NO:91), a VH CDR3 (SEQ ID NO:93), a VL CDR2 (SEQ ID NO:95), and a VL CDR3 (SEQ ID NO:96). In another embodiment, the antibody comprises a VH CDR2 (SEQ ID NO:92), a VH CDR3 (SEQ ID NO:93), a VL CDR1 (SEQ ID NO:94), and a VL CDR2 (SEQ ID NO:95). In one embodiment, the antibody comprises a VH CDR2 (SEQ ID NO:92), a VH CDR3 (SEQ ID NO:93), a VL CDR1 (SEQ ID NO:94), and a VL CDR3 (SEQ ID NO:96). In other embodiments, the antibody comprises a VH CDR2 (SEQ ID NO:92), a VH CDR3 (SEQ ID NO:93), a VL CDR2 (SEQ ID NO:95), and a VL CDR3 (SEQ ID NO:96). In another embodiment, the antibody comprises a VH CDR1 (SEQ ID NO:91), a VH CDR2 (SEQ ID NO:92), a VH CDR3 (SEQ ID NO:93), a VL CDR1 (SEQ ID NO:94), and a VL CDR2 (SEQ ID NO:95). In some embodiments, the antibody comprises a VH CDR1 (SEQ ID NO:91), a VH CDR2 (SEQ ID NO:92), a VH CDR3 (SEQ ID NO:93), a VL CDR1 (SEQ ID NO:94), and a VL CDR3 (SEQ ID NO:96). In one embodiment, the antibody comprises a VH CDR1 (SEQ ID NO:91), a VH CDR2 (SEQ ID NO:92), a VH CDR3 (SEQ ID NO:93), a VL CDR2 (SEQ ID NO:95), and a VL CDR3 (SEQ ID NO:96). In another embodiment, the antibody comprises a VH CDR1 (SEQ ID NO:91), a VH CDR2 (SEQ ID NO:92), a VL CDR1 (SEQ ID NO:94), a VL CDR2 (SEQ ID NO:95), and a VL CDR3 (SEQ ID NO:96). In other embodiments, the antibody comprises a VH CDR1 (SEQ ID NO:91), a VH CDR3 (SEQ ID NO:93), a VL CDR1 (SEQ ID NO:94), a VL CDR2 (SEQ ID NO:95), and a VL CDR3 (SEQ ID NO:96). In some embodiments, the antibody comprises a VH CDR2 (SEQ ID NO:92), a VH CDR3 (SEQ ID NO:93), a VL CDR1 (SEQ ID NO:94), a VL CDR2 (SEQ ID NO:95), and a VL CDR3 (SEQ ID NO:96). In another embodiment, the antibody comprises a VH CDR1 (SEQ ID NO:91), a VL CDR1 (SEQ ID NO:94), a VL CDR2 (SEQ ID NO:95), and a VL CDR3 (SEQ ID NO:96). In one embodiment, the antibody comprises a VH CDR2 (SEQ ID NO:92), a VL CDR1 (SEQ ID NO:94), a VL CDR2 (SEQ ID NO:95), and a VL CDR3 (SEQ ID NO:96). In other embodiments, the antibody comprises a VH CDR3 (SEQ ID NO:93), a VL CDR1 (SEQ ID NO:94), a VL CDR2 (SEQ ID NO:95), and a VL CDR3 (SEQ ID NO:96). In another embodiment, the antibody comprises any combination thereof of the VH CDRs and VL CDRs listed in Tables 3 and 4.

In some embodiments, the antibodies comprise a VH CDR1, VH CDR1, VH CDR3, VL CDR1, VL CDR2, VL CDR3 according to the IMGT numbering system. Accordingly, in some embodiments, the antibodies comprise a VH CDR1 having an amino acid sequence of SEQ ID NO:97. In some embodiments, the antibodies comprise a VH CDR2 having an amino acid sequence of SEQ ID NO:98. In some embodiments, the antibodies comprise a VH CDR3 having an amino acid sequence of SEQ ID NO:99. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 4. In some embodiments, the antibodies comprise a VL CDR1 having an amino acid sequence of any one of SEQ ID NO:100. In another embodiment, the antibodies comprise a VL CDR2 having an amino acid sequence of SEQ ID NO:101. In some embodiments, the antibodies comprise a VL CDR3 having an amino acid sequence of SEQ ID NO:102. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 3.

In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region comprising: (1) a VL CDR1 having an amino acid sequence of SEQ ID NO:100; (2) a VL CDR2 having an amino acid sequence of SEQ ID NO:101; and (3) a VL CDR3 having an amino acid sequence of SEQ ID NO:102.

In some embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99.

In other embodiments, the antibodies provided herein comprise a VL region comprising: (1) a VL CDR1 having an amino acid sequence of SEQ ID NO:100; (2) a VL CDR2 having an amino acid sequence of SEQ ID NO:101; and (3) a VL CDR3 having an amino acid sequence of SEQ ID NO:102.

Also provided herein are antibodies comprising one or more (e.g., one, two, or three) VH CDRs and one or more (e.g., one, two, or three) VL CDRs listed in Tables 3 and 4. In particular, provided herein is an antibody comprising a VH CDR1 (SEQ ID NO:97) and a VL CDR1 (SEQ ID NO:100). In one embodiment, the antibody comprises a VH CDR1 (SEQ ID NO:97) and a VL CDR2 (SEQ ID NO:101). In other embodiments, the antibody comprises a VH CDR1 (SEQ ID NO:97) and a VL CDR3 (SEQ ID NO:102). In another embodiment, the antibody comprises a VH CDR2 (SEQ ID NO:98 and a VL CDR1 (SEQ ID NO:100). In some embodiments, the antibody comprises a VH CDR2 (SEQ ID NO:98 and a VL CDR2 (SEQ ID NO:101). In one embodiment, the antibody comprises a VH CDR2 (SEQ ID NO:98 and a VL CDR3 (SEQ ID NO:102). In another embodiment, the antibody comprises a VH CDR3 (SEQ ID NO:99) and a VL CDR1 (SEQ ID NO:100). In other embodiments, the antibody comprises a VH CDR3 (SEQ ID NO:99) and a VL CDR2 (SEQ ID NO:101). In some embodiments, the antibody comprises a VH CDR3 (SEQ ID NO:99) and a VL CDR3 (SEQ ID NO:102). In another embodiment, the antibody comprises a VH CDR1 (SEQ ID NO:97), a VH CDR2 (SEQ ID NO:98, and a VL CDR1 (SEQ ID NO:100). In one embodiment, the antibody comprises a VH CDR1 (SEQ ID NO:97), a VH CDR2 (SEQ ID NO:98, and a VL CDR2 (SEQ ID NO:101). In other embodiments, the antibody comprises a VH CDR1 (SEQ ID NO:97), a VH CDR2 (SEQ ID NO:98, and a VL CDR3 (SEQ ID NO:102). In another embodiment, the antibody comprises a VH CDR2 (SEQ ID NO:98, a VH CDR3 (SEQ ID NO:99), and a VL CDR1 (SEQ ID NO:100). In some embodiments, the antibody comprises a VH CDR2 (SEQ ID NO:98, a VH CDR3 (SEQ ID NO:99), and a VL CDR2 (SEQ ID NO:101). In one embodiment, the antibody comprises a VH CDR2 (SEQ ID NO:98, a VH CDR3 (SEQ ID NO:99), and a VL CDR3 (SEQ ID NO:102). In another embodiment, the antibody comprises a VH CDR1 (SEQ ID NO:97), a VH CDR3 (SEQ ID NO:99), and a VL CDR1 (SEQ ID NO:100). In other embodiments, the antibody comprises a VH CDR1 (SEQ ID NO:97), a VH CDR3 (SEQ ID NO:99), and a VL CDR2 (SEQ ID NO:101). In some embodiments, the antibody comprises a VH CDR1 (SEQ ID NO:97), a VH CDR3 (SEQ ID NO:99), and a VL CDR3 (SEQ ID NO:102). In another embodiment, the antibody comprises a VH CDR1 (SEQ ID NO:97), a VL CDR1 (SEQ ID NO:100), and a VL CDR2 (SEQ ID NO:101). In one embodiment, the antibody comprises a VH CDR1 (SEQ ID NO:97), a VL CDR1 (SEQ ID NO:100), and a VL CDR3 (SEQ ID NO:102). In other embodiments, the antibody comprises a VH CDR1 (SEQ ID NO:97), a VL CDR2 (SEQ ID NO:101), and a VL CDR3 (SEQ ID NO:102). In another embodiment, the antibody comprises a VH CDR2 (SEQ ID NO:98, a VL CDR1 (SEQ ID NO:100), and a VL CDR2 (SEQ ID NO:101). In some embodiments, the antibody comprises a VH CDR2 (SEQ ID NO:98, a VL CDR1 (SEQ ID NO:100), and a VL CDR3 (SEQ ID NO:102). In one embodiment, the antibody comprises a VH CDR2 (SEQ ID NO:98, a VL CDR2 (SEQ ID NO:101), and a VL CDR3 (SEQ ID NO:102). In another embodiment, the antibody comprises a VH CDR3 (SEQ ID NO:99), a VL CDR1 (SEQ ID NO:100), and a VL CDR2 (SEQ ID NO:101). In other embodiments, the antibody comprises a VH CDR3 (SEQ ID NO:99), a VL CDR1 (SEQ ID NO: 100), and a VL CDR3 (SEQ ID NO:102). In some embodiments, the antibody comprises a VH CDR3 (SEQ ID NO:99), a VL CDR2 (SEQ ID NO:101), and a VL CDR3 (SEQ ID NO: 102). In another embodiment, the antibody comprises a VH CDR1 (SEQ ID NO:97), a VH CDR2 (SEQ ID NO:98, a VH CDR3 (SEQ ID NO:99), and a VL CDR1 (SEQ ID NO: 100). In one embodiment, the antibody comprises a VH CDR1 (SEQ ID NO:97), a VH CDR2 (SEQ ID NO:98, a VH CDR3 (SEQ ID NO:99), and a VL CDR2 (SEQ ID NO: 101). In other embodiments, the antibody comprises a VH CDR1 (SEQ ID NO:97), a VH CDR2 (SEQ ID NO:98, a VH CDR3 (SEQ ID NO:99), and a VL CDR3 (SEQ ID NO:102). In another embodiment, the antibody comprises a VH CDR1 (SEQ ID NO:97), a VH CDR2 (SEQ ID NO:98, a VL CDR1 (SEQ ID NO:100), and a VL CDR2 (SEQ ID NO:101). In some embodiments, the antibody comprises a VH CDR1 (SEQ ID NO:97), a VH CDR2 (SEQ ID NO:98, a VL CDR1 (SEQ ID NO:100), and a VL CDR3 (SEQ ID NO:102). In one embodiment, the antibody comprises a VH CDR1 (SEQ ID NO:97), a VH CDR2 (SEQ ID NO:98, a VL CDR2 (SEQ ID NO:101), and a VL CDR3 (SEQ ID NO:102). In another embodiment, the antibody comprises a VH CDR1 (SEQ ID NO:97), a VH CDR3 (SEQ ID NO:99), a VL CDR1 (SEQ ID NO:100), and a VL CDR2 (SEQ ID NO:101). In other embodiments, the antibody comprises a VH CDR1 (SEQ ID NO:97), a VH CDR3 (SEQ ID NO:99), a VL CDR1 (SEQ ID NO: 100), and a VL CDR3 (SEQ ID NO: 102). In some embodiments, the antibody comprises a VH CDR1 (SEQ ID NO:97), a VH CDR3 (SEQ ID NO:99), a VL CDR2 (SEQ ID NO:101), and a VL CDR3 (SEQ ID NO:102). In another embodiment, the antibody comprises a VH CDR2 (SEQ ID NO:98, a VH CDR3 (SEQ ID NO:99), a VL CDR1 (SEQ ID NO:100), and a VL CDR2 (SEQ ID NO:101). In one embodiment, the antibody comprises a VH CDR2 (SEQ ID NO:98, a VH CDR3 (SEQ ID NO:99), a VL CDR1 (SEQ ID NO:100), and a VL CDR3 (SEQ ID NO: 102). In other embodiments, the antibody comprises a VH CDR2 (SEQ ID NO:98, a VH CDR3 (SEQ ID NO:99), a VL CDR2 (SEQ ID NO:101), and a VL CDR3 (SEQ ID NO: 102). In another embodiment, the antibody comprises a VH CDR1 (SEQ ID NO:97), a VH CDR2 (SEQ ID NO:98, a VH CDR3 (SEQ ID NO:99), a VL CDR1 (SEQ ID NO:100), and a VL CDR2 (SEQ ID NO:101). In some embodiments, the antibody comprises a VH CDR1 (SEQ ID NO:97), a VH CDR2 (SEQ ID NO:98, a VH CDR3 (SEQ ID NO:99), a VL CDR1 (SEQ ID NO:100), and a VL CDR3 (SEQ ID NO:102). In one embodiment, the antibody comprises a VH CDR1 (SEQ ID NO:97), a VH CDR2 (SEQ ID NO:98, a VH CDR3 (SEQ ID NO:99), a VL CDR2 (SEQ ID NO:101), and a VL CDR3 (SEQ ID NO:102). In another embodiment, the antibody comprises a VH CDR1 (SEQ ID NO:97), a VH CDR2 (SEQ ID NO:98, a VL CDR1 (SEQ ID NO:100), a VL CDR2 (SEQ ID NO:101), and a VL CDR3 (SEQ ID NO: 102). In other embodiments, the antibody comprises a VH CDR1 (SEQ ID NO:97), a VH CDR3 (SEQ ID NO:99), a VL CDR1 (SEQ ID NO:100), a VL CDR2 (SEQ ID NO:101), and a VL CDR3 (SEQ ID NO:102). In some embodiments, the antibody comprises a VH CDR2 (SEQ ID NO:98, a VH CDR3 (SEQ ID NO:99), a VL CDR1 (SEQ ID NO:100), a VL CDR2 (SEQ ID NO:101), and a VL CDR3 (SEQ ID NO:102). In another embodiment, the antibody comprises a VH CDR1 (SEQ ID NO:97), a VL CDR1 (SEQ ID NO:100), a VL CDR2 (SEQ ID NO:101), and a VL CDR3 (SEQ ID NO:102). In one embodiment, the antibody comprises a VH CDR2 (SEQ ID NO:98, a VL CDR1 (SEQ ID NO:100), a VL CDR2 (SEQ ID NO:101), and a VL CDR3 (SEQ ID NO:102). In other embodiments, the antibody comprises a VH CDR3 (SEQ ID NO: 99), a VL CDR1 (SEQ ID NO:100), a VL CDR2 (SEQ ID NO:101), and a VL CDR3 (SEQ ID NO:102). In another embodiment, the antibody comprises any combination thereof of the VH CDRs and VL CDRs listed in Tables 3 and 4.

In some embodiments, the antibodies comprise a VH CDR1, VH CDR1, VH CDR3, VL CDR1, VL CDR2, VL CDR3 according to the Contact numbering system. Accordingly, in some embodiments, the antibodies comprise a VH CDR1 having an amino acid sequence of SEQ ID NO:103. In some embodiments, the antibodies comprise a VH CDR2 having an amino acid sequence of SEQ ID NO: 104. In some embodiments, the antibodies comprise a VH CDR3 having an amino acid sequence of SEQ ID NO:105. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 4. In some embodiments, the antibodies comprise a VL CDR1 having an amino acid sequence of any one of SEQ ID NO:106. In another embodiment, the antibodies comprise a VL CDR2 having an amino acid sequence of SEQ ID NO:107. In some embodiments, the antibodies comprise a VL CDR3 having an amino acid sequence of SEQ ID NO:108. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 3.

In certain embodiments, the antibodies provided herein comprise a VH region comprising: (1) a VH CDR1 having an amino acid sequence of SEQ ID NO:103; (2) a VH CDR2 having an amino acid sequence of SEQ ID NO:104; and (3) a VH CDR3 having an amino acid sequence of SEQ ID NO:105; and a VL region comprising: (1) a VL CDR1 having an amino acid sequence of SEQ ID NO:106; (2) a VL CDR2 having an amino acid sequence of SEQ ID NO:107; and (3) a VL CDR3 having an amino acid sequence of SEQ ID NO:108. In some embodiments, the antibodies provided herein comprise a VH region comprising: (1) a VH CDR1 having an amino acid sequence of SEQ ID NO:103; (2) a VH CDR2 having an amino acid sequence of SEQ ID NO:104; and (3) a VH CDR3 having an amino acid sequence of SEQ ID NO:105.

In other embodiments, the antibodies provided herein comprise a VL region comprising: (1) a VL CDR1 having an amino acid sequence of SEQ ID NO:106; (2) a VL CDR2 having an amino acid sequence of SEQ ID NO:107; and (3) a VL CDR3 having an amino acid sequence of SEQ ID NO:108.

Also provided herein are antibodies comprising one or more (e.g., one, two, or three) VH CDRs and one or more (e.g., one, two, or three) VL CDRs listed in Tables 3 and 4. In particular, provided herein is an antibody comprising a VH CDR1 (SEQ ID NO:103) and a VL CDR1 (SEQ ID NO:106). In one embodiment, the antibody comprises a VH CDR1 (SEQ ID NO:103) and a VL CDR2 (SEQ ID NO:107). In other embodiments, the antibody comprises a VH CDR1 (SEQ ID NO:103) and a VL CDR3 (SEQ ID NO:108). In another embodiment, the antibody comprises a VH CDR2 (SEQ ID NO:104) and a VL CDR1 (SEQ ID NO:106). In some embodiments, the antibody comprises a VH CDR2 (SEQ ID NO:104) and a VL CDR2 (SEQ ID NO:107). In one embodiment, the antibody comprises a VH CDR2 (SEQ ID NO:104) and a VL CDR3 (SEQ ID NO:108). In another embodiment, the antibody comprises a VH CDR3 (SEQ ID NO:105) and a VL CDR1 (SEQ ID NO:106). In other embodiments, the antibody comprises a VH CDR3 (SEQ ID NO: 105) and a VL CDR2 (SEQ ID NO: 107). In some embodiments, the antibody comprises a VH CDR3 (SEQ ID NO: 105) and a VL CDR3 (SEQ ID NO: 108). In another embodiment, the antibody comprises a VH CDR1 (SEQ ID NO: 103), a VH CDR2 (SEQ ID NO:104), and a VL CDR1 (SEQ ID NO:106). In one embodiment, the antibody comprises a VH CDR1 (SEQ ID NO:103), a VH CDR2 (SEQ ID NO:104), and a VL CDR2 (SEQ ID NO:107). In other embodiments, the antibody comprises a VH CDR1 (SEQ ID NO:103), a VH CDR2 (SEQ ID NO:104), and a VL CDR3 (SEQ ID NO: 108). In another embodiment, the antibody comprises a VH CDR2 (SEQ ID NO: 104), a VH CDR3 (SEQ ID NO: 105), and a VL CDR1 (SEQ ID NO:106). In some embodiments, the antibody comprises a VH CDR2 (SEQ ID NO:104), a VH CDR3 (SEQ ID NO:105), and a VL CDR2 (SEQ ID NO:107). In one embodiment, the antibody comprises a VH CDR2 (SEQ ID NO:104), a VH CDR3 (SEQ ID NO:105), and a VL CDR3 (SEQ ID NO:108). In another embodiment, the antibody comprises a VH CDR1 (SEQ ID NO: 103), a VH CDR3 (SEQ ID NO: 105), and a VL CDR1 (SEQ ID NO:106). In other embodiments, the antibody comprises a VH CDR1 (SEQ ID NO:103), a VH CDR3 (SEQ ID NO:105), and a VL CDR2 (SEQ ID NO: 107). In some embodiments, the antibody comprises a VH CDR1 (SEQ ID NO: 103), a VH CDR3 (SEQ ID NO: 105), and a VL CDR3 (SEQ ID NO:108). In another embodiment, the antibody comprises a VH CDR1 (SEQ ID NO:103), a VL CDR1 (SEQ ID NO:106), and a VL CDR2 (SEQ ID NO:107). In one embodiment, the antibody comprises a VH CDR1 (SEQ ID NO:103), a VL CDR1 (SEQ ID NO:106), and a VL CDR3 (SEQ ID NO:108). In other embodiments, the antibody comprises a VH CDR1 (SEQ ID NO: 103), a VL CDR2 (SEQ ID NO: 107), and a VL CDR3 (SEQ ID NO:108). In another embodiment, the antibody comprises a VH CDR2 (SEQ ID NO:104), a VL CDR1 (SEQ ID NO:106), and a VL CDR2 (SEQ ID NO: 107). In some embodiments, the antibody comprises a VH CDR2 (SEQ ID NO: 104), a VL CDR1 (SEQ ID NO: 106), and a VL CDR3 (SEQ ID NO:108). In one embodiment, the antibody comprises a VH CDR2 (SEQ ID NO:104), a VL CDR2 (SEQ ID NO:107), and a VL CDR3 (SEQ ID NO:108). In another embodiment, the antibody comprises a VH CDR3 (SEQ ID NO:105), a VL CDR1 (SEQ ID NO:106), and a VL CDR2 (SEQ ID NO:107). In other embodiments, the antibody comprises a VH CDR3 (SEQ ID NO:105), a VL CDR1 (SEQ ID NO:106), and a VL CDR3 (SEQ ID NO:108). In some embodiments, the antibody comprises a VH CDR3 (SEQ ID NO: 105), a VL CDR2 (SEQ ID NO:107), and a VL CDR3 (SEQ ID NO: 108). In another embodiment, the antibody comprises a VH CDR1 (SEQ ID NO: 103), a VH CDR2 (SEQ ID NO:104), a VH CDR3 (SEQ ID NO:105), and a VL CDR1 (SEQ ID NO: 106). In one embodiment, the antibody comprises a VH CDR1 (SEQ ID NO:103), a VH CDR2 (SEQ ID NO:104), a VH CDR3 (SEQ ID NO:105), and a VL CDR2 (SEQ ID NO:107). In other embodiments, the antibody comprises a VH CDR1 (SEQ ID NO: 103), a VH CDR2 (SEQ ID NO: 104), a VH CDR3 (SEQ ID NO: 105), and a VL CDR3 (SEQ ID NO: 108). In another embodiment, the antibody comprises a VH CDR1 (SEQ ID NO:103), a VH CDR2 (SEQ ID NO:104), a VL CDR1 (SEQ ID NO:106), and a VL CDR2 (SEQ ID NO: 107). In some embodiments, the antibody comprises a VH CDR1 (SEQ ID NO:103), a VH CDR2 (SEQ ID NO:104), a VL CDR1 (SEQ ID NO:106), and a VL CDR3 (SEQ ID NO:108). In one embodiment, the antibody comprises a VH CDR1 (SEQ ID NO:103), a VH CDR2 (SEQ ID NO:104), a VL CDR2 (SEQ ID NO:107), and a VL CDR3 (SEQ ID NO:108). In another embodiment, the antibody comprises a VH CDR1 (SEQ ID NO:103), a VH CDR3 (SEQ ID NO: 105), a VL CDR1 (SEQ ID NO: 106), and a VL CDR2 (SEQ ID NO: 107). In other embodiments, the antibody comprises a VH CDR1 (SEQ ID NO: 103), a VH CDR3 (SEQ ID NO: 105), a VL CDR1 (SEQ ID NO:106), and a VL CDR3 (SEQ ID NO:108). In some embodiments, the antibody comprises a VH CDR1 (SEQ ID NO: 103), a VH CDR3 (SEQ ID NO:105), a VL CDR2 (SEQ ID NO:107), and a VL CDR3 (SEQ ID NO:108). In another embodiment, the antibody comprises a VH CDR2 (SEQ ID NO:104), a VH CDR3 (SEQ ID NO:105), a VL CDR1 (SEQ ID NO:106), and a VL CDR2 (SEQ ID NO:107). In one embodiment, the antibody comprises a VH CDR2 (SEQ ID NO:104), a VH CDR3 (SEQ ID NO:105), a VL CDR1 (SEQ ID NO:106), and a VL CDR3 (SEQ ID NO:108). In other embodiments, the antibody comprises a VH CDR2 (SEQ ID NO: 104), a VH CDR3 (SEQ ID NO:105), a VL CDR2 (SEQ ID NO:107), and a VL CDR3 (SEQ ID NO:108). In another embodiment, the antibody comprises a VH CDR1 (SEQ ID NO: 103), a VH CDR2 (SEQ ID NO:104), a VH CDR3 (SEQ ID NO:105), a VL CDR1 (SEQ ID NO: 106), and a VL CDR2 (SEQ ID NO:107). In some embodiments, the antibody comprises a VH CDR1 (SEQ ID NO:103), a VH CDR2 (SEQ ID NO:104), a VH CDR3 (SEQ ID NO:105), a VL CDR1 (SEQ ID NO:106), and a VL CDR3 (SEQ ID NO: 108). In one embodiment, the antibody comprises a VH CDR1 (SEQ ID NO:103), a VH CDR2 (SEQ ID NO:104), a VH CDR3 (SEQ ID NO:105), a VL CDR2 (SEQ ID NO: 107), and a VL CDR3 (SEQ ID NO:108). In another embodiment, the antibody comprises a VH CDR1 (SEQ TD NO:103), a VH CDR2 (SEQ ID NO 104), a VL CDR3 (SEQ ID NO: 106), a VL CDR2 (SEQ ID NO:107), and a VL CDR3 (SEQ ID NO: 108). In other embodiments, the antibody comprises a VH CDR1 (SEQ ID NO: 103), a VH CDR3 (SEQ ID NO:105), a VL CDR1 (SEQ ID NO: 106), a VL CDR2 (SEQ ID NO: 107), and a VL CDR3 (SEQ ID NO: 108). In some embodiments, the antibody comprises a VH CDR2 (SEQ ID NO: 104), a VH CDR3 (SEQ ID NO:105), a VL CDR (SEQ ID NO:106), a VL CDR2 (SEQ ID NO:107), and a VL CDR3 (SEQ ID NO: 108). In another embodiment, the antibody comprises a VH CDR C (SEQ ID NO:103), a VL CDR2 (SEQ ID NO:106), a VL CDR2 (SEQ ID NO:107), and a VL CDR3 (SEQ ID NO: 108). In one embodiment, the antibody comprises a VH CDR2 (SEQ ID NO:104), a VL CDR1 (SEQ ID NO:106), a VL CDR2 (SEQ ID NO:107), and a VL CDR3 (SEQ ID NO:108). In other embodiments, the antibody comprises a V CDR3 (SEQ ID NO:105), a VL CDR1 (SEQ ID NO:106), a VL CDR2 (SEQ ID NO:107), and a VL CDR3 (SEQ ID NO: 108). In another embodiment, the antibody comprises any combination thereof of the VII CDRs and VL CDRs listed in Tables 3 and 4.

TABLE 5
VL CDR Sequences of Anti-BTN1A1 Antibody STC85F1-1

| | Region | | | |
|---|---|---|---|---|
| | definition | CDR1 | CDR2 | CDR3 |
| Kappa | Chothia | QSSQSVYNNKNLS | RASTLPS | AGGYSGDINV |
| light | | (SEQ ID NO: 114) | (SEQ ID NO: 115) | (SEQ ID NO: 116) |
| chain | IMGT | QSVYNNKN | RSA | AGGYSGDINV |
| | | (SEQ ID NO: 120) | (SEQ ID NO: 121) | (SEQ ID NO: 122) |
| | Kabat | QSSQSVYNNKNL | RASTLPS | AGGYSGDINV |
| | | (SEQ ID NO: 41) | (SEQ ID NO: 42) | (SEQ ID NO: 43 |
| | Contact | YNNKNLSWF | LLIYRASTLP | AGGYSGDIN |
| | | (SEQ ID NO: 126) | (SEQ ID NO: 127) | (SEQ ID NO: 128) |

TABLE 6
VH CDR Sequences of Anti-BTN1A1 Antibody STC85F1-1

| | Region | | | |
|---|---|---|---|---|
| | definition | CDR1 | CDR2 | CDR3 |
| Heavy | Chothia | GIDLSRY | GTTSN | SVVSPSNSSF |
| chain | | (SEQ ID NO: 111) | (SEQ ID NO: 112) | (SEQ ID NO: 113) |
| | IMGT | GIDLSRYG | IGTTSNT | ARSVVSPSNSSF |
| | | (SEQ ID NO: 117) | (SEQ ID NO: 118) | (SEQ ID NO: 119) |
| | Kabat | RYGVN | YIGTTSNTYYASW | SVVSPSNSSF |
| | | (SEQ ID NO: 47) | AKG | (SEQ ID NO: 49) |
| | | | (SEQ ID NO: 48) | |
| | Contact | SRYGVN | WIGYIGTTSNTY | ARSVVSPSNSS |
| | | (SEQ ID NO: 123) | (SEQ ID NO: 124) | (SEQ ID NO: 125) |

In some embodiments, an antibody provided herein comprises or consists of six CDRs, for example, VH CDR1, VH CDR2, VH CDR3, VL CDR1, VL CDR2, and/or VL CDR3 identified in Tables 5 and 6. In some embodiments, an antibody provided herein can comprise fewer than six CDRs. In some embodiments, the antibody comprises or consists of one, two, three, four, or five CDRs selected from the group consisting of VH CDR1, VH CDR2, VH CDR3, VL CDR1, VL CDR2, and/or VL CDR3 identified in Tables 5 and 6. In some embodiments, the antibody comprises or consists of one, two, three, four, or five CDRs selected from the group consisting of VH CDR1, VH CDR2, VH CDR3, VL CDR1, VL CDR2, and/or VL CDR3 of the monoclonal described herein. Accordingly, in some embodiments, the antibody comprises or consists of one, two, three, four, or five CDRs of anyone of the VH CDR1, VH CDR2, VH CDR3, VL CDR1, VL CDR2, and/or VL CDR3 identified in Tables 5 and 6.

In some embodiments, the antibodies provided herein comprise one or more (e.g., one, two, or three) VH CDRs listed in Table 6. In other embodiments, the antibodies provided herein comprise one or more (e.g., one, two, or three) VL CDRs listed in Table 5. In yet other embodiments, the antibodies provided herein comprise one or more (e.g., one, two, or three) VH CDRs listed in Table 6 and one or more VL CDRs listed in Table 5.

In some embodiments, the antibodies comprise a VH CDR1, VH CDR1, VH CDR3, VL CDR1, VL CDR2, VL CDR3 according to the Kabat numbering system. Accordingly, in some embodiments, the antibodies comprise a VH CDR1 having an amino acid sequence of SEQ ID NO:47. In some embodiments, the antibodies comprise a VH CDR2 having an amino acid sequence of SEQ ID NO:48. In some embodiments, the antibodies comprise a VH CDR3 having an amino acid sequence of SEQ ID NO:49. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 6. In some embodiments, the antibodies comprise a VL CDR1 having an amino acid sequence of any one of SEQ ID NO:41. In another embodiment, the antibodies comprise a VL CDR2 having an amino acid sequence of SEQ ID NO:42. In some embodiments, the antibodies comprise a VL CDR3 having an amino acid sequence of SEQ ID NO:43. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 5.

In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region comprising: (1) a VL CDR1 having an amino acid sequence of SEQ ID NO:41; (2) a VL CDR2 having an amino acid sequence of SEQ ID NO:42; and (3) a VL CDR3 having an amino acid sequence of SEQ ID NO:43.

In some embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49.

In other embodiments, the antibodies provided herein comprise a VL region comprising: (1) a VL CDR1 having an amino acid sequence of SEQ ID NO:41; (2) a VL CDR2 having an amino acid sequence of SEQ ID NO:42; and (3) a VL CDR3 having an amino acid sequence of SEQ ID NO:43.

Also provided herein are antibodies comprising one or more (e.g., one, two, or three) VH CDRs and one or more (e.g., one, two, or three) VL CDRs listed in Tables 5 and 6. In particular, provided herein is an antibody comprising a VH CDR1 (SEQ ID NO:47) and a VL CDR1 (SEQ ID NO:41). In one embodiment, the antibody comprises a VH CDR1 (SEQ ID NO:47) and a VL CDR2 (SEQ ID NO:42). In other embodiments, the antibody comprises a VH CDR1 (SEQ ID NO:47) and a VL CDR3 (SEQ ID NO:43). In another embodiment, the antibody comprises a VH CDR2 (SEQ ID NO:48) and a VL CDR1 (SEQ ID NO:41). In some embodiments, the antibody comprises a VH CDR2 (SEQ ID NO:48) and a VL CDR2 (SEQ ID NO:42). In one embodiment, the antibody comprises a VH CDR2 (SEQ ID NO:48) and a VL CDR3 (SEQ ID NO:43). In another embodiment, the antibody comprises a VH CDR3 (SEQ ID NO:49) and a VL CDR1 (SEQ ID NO:41). In other embodiments, the antibody comprises a VH CDR3 (SEQ ID NO:49) and a VL CDR2 (SEQ ID NO:42). In some embodiments, the antibody comprises a VH CDR3 (SEQ ID NO:49) and a VL CDR3 (SEQ ID NO:43). In another embodiment, the antibody comprises a VH CDR1 (SEQ ID NO:47), a VH CDR2 (SEQ ID NO:48), and a VL CDR1 (SEQ ID NO:41). In one embodiment, the antibody comprises a VH CDR1 (SEQ ID NO:47), a VH CDR2 (SEQ ID NO:48), and a VL CDR2 (SEQ ID NO:42). In other embodiments, the antibody comprises a VH CDR1 (SEQ ID NO:47), a VH CDR2 (SEQ ID NO:48), and a VL CDR3 (SEQ ID NO:43). In another embodiment, the antibody comprises a VH CDR2 (SEQ ID NO:48), a VH CDR3 (SEQ ID NO:49), and a VL CDR1 (SEQ ID NO:41). In some embodiments, the antibody comprises a VH CDR2 (SEQ ID NO:48), a VH CDR3 (SEQ ID NO:49), and a VL CDR2 (SEQ ID NO:42). In one embodiment, the antibody comprises a VH CDR2 (SEQ ID NO:48), a VH CDR3 (SEQ ID NO:49), and a VL CDR3 (SEQ ID NO:43). In another embodiment, the antibody comprises a VH CDR1 (SEQ ID NO:47), a VH CDR3 (SEQ ID NO:49), and a VL CDR1 (SEQ ID NO:41). In other embodiments, the antibody comprises a VH CDR1 (SEQ ID NO:47), a VH CDR3 (SEQ ID NO:49), and a VL CDR2 (SEQ ID NO:42). In some embodiments, the antibody comprises a VH CDR1 (SEQ ID NO:47), a VH CDR3 (SEQ ID NO:49), and a VL CDR3 (SEQ ID NO:43). In another embodiment, the antibody comprises a VH CDR1 (SEQ ID NO:47), a VL CDR1 (SEQ ID NO:41), and a VL CDR2 (SEQ ID NO:42). In one embodiment, the antibody comprises a VH CDR1 (SEQ ID NO:47), a VL CDR1 (SEQ ID NO:41), and a VL CDR3 (SEQ ID NO:43). In other embodiments, the antibody comprises a VH CDR1 (SEQ ID NO:47), a VL CDR2 (SEQ ID NO:42), and a VL CDR3 (SEQ ID NO:43). In another embodiment, the antibody comprises a VH CDR2 (SEQ ID NO:48), a VL CDR1 (SEQ ID NO:41), and a VL CDR2 (SEQ ID NO:42). In some embodiments, the antibody comprises a VH CDR2 (SEQ ID NO:48), a VL CDR1 (SEQ ID NO:41), and a VL CDR3 (SEQ ID NO:43). In one embodiment, the antibody comprises a VH CDR2 (SEQ ID NO:48), a VL CDR2 (SEQ ID NO:42), and a VL CDR3 (SEQ ID NO:43). In another embodiment, the antibody comprises a VH CDR3 (SEQ ID NO:49), a VL CDR1 (SEQ ID NO:41), and a VL CDR2 (SEQ ID NO:42). In other embodiments, the antibody comprises a VH CDR3 (SEQ ID NO:49), a VL CDR1 (SEQ ID NO:41), and a VL CDR3 (SEQ ID NO:43). In some embodiments, the antibody comprises a VH CDR3 (SEQ ID NO:49), a VL CDR2 (SEQ ID NO:42), and a VL CDR3 (SEQ ID NO:43). In another embodiment, the antibody comprises a VH CDR1 (SEQ ID NO:47), a VH CDR2 (SEQ ID NO:48), a VH CDR3 (SEQ ID NO:49), and a VL CDR1 (SEQ ID NO:41). In one embodiment, the antibody comprises a VH CDR1 (SEQ ID NO:47), a VH CDR2 (SEQ ID NO:48), a VH CDR3 (SEQ ID NO:49), and a VL CDR2 (SEQ ID NO:42). In other embodiments, the antibody comprises a VH CDR1 (SEQ ID NO:47), a VH CDR2 (SEQ ID NO:48), a VH CDR3 (SEQ ID NO:49), and a VL CDR3 (SEQ ID NO:43). In another embodiment, the antibody comprises a VH CDR1 (SEQ ID NO:47), a VH CDR2 (SEQ ID NO:48), a VL CDR1 (SEQ ID NO:41), and a VL CDR2 (SEQ ID NO:42). In some embodiments, the antibody comprises a VH CDR1 (SEQ ID NO:47), a VH CDR2 (SEQ ID NO:48), a VL CDR1 (SEQ ID NO:41), and a VL CDR3 (SEQ ID NO:43). In one embodiment, the antibody comprises a VH CDR1 (SEQ ID NO:47), a VH CDR2 (SEQ ID NO:48), a VL CDR2 (SEQ ID NO:42), and a VL CDR3 (SEQ ID NO:43). In another embodiment, the antibody comprises a VH CDR1 (SEQ ID NO:47), a VH CDR3 (SEQ ID NO:49), a VL CDR1 (SEQ ID NO:41), and a VL CDR2 (SEQ ID NO:42). In other embodiments, the antibody comprises a VH CDR1 (SEQ ID NO:47), a VH CDR3 (SEQ ID NO:49), a VL CDR1 (SEQ ID NO:41), and a VL CDR3 (SEQ ID NO:43). In some embodiments, the antibody comprises a VH CDR1 (SEQ ID NO:47), a VH CDR3 (SEQ ID NO:49), a VL CDR2 (SEQ ID NO:42), and a VL CDR3 (SEQ ID NO:43). In another embodiment, the antibody comprises a VH CDR2 (SEQ ID NO:48), a VH CDR3 (SEQ ID NO:49), a VL CDR1 (SEQ ID NO:41), and a VL CDR2 (SEQ ID NO:42). In one embodiment, the antibody comprises a VH CDR2 (SEQ ID NO:48), a VH CDR3 (SEQ ID NO:49), a VL CDR1 (SEQ ID NO:41), and a VL CDR3 (SEQ ID NO:43). In other embodiments, the antibody comprises a VH CDR2 (SEQ ID NO:48), a VH CDR3 (SEQ ID NO:49), a VL CDR2 (SEQ ID NO:42), and a VL CDR3 (SEQ ID NO:43). In another embodiment, the antibody comprises a VH CDR1 (SEQ ID NO:47), a VH CDR2 (SEQ ID NO:48), a VH CDR3 (SEQ ID NO:49), a VL CDR1 (SEQ ID NO:41), and a VL CDR2 (SEQ ID NO:42). In some embodiments, the antibody comprises a VH CDR1 (SEQ ID NO:47), a VH CDR2 (SEQ ID NO:48), a VH CDR3 (SEQ ID NO:49), a VL CDR1 (SEQ ID NO:41), and a VL CDR3 (SEQ ID NO:43). In one embodiment, the antibody comprises a VH CDR1 (SEQ ID NO:47), a VH CDR2 (SEQ ID NO:48), a VH CDR3 (SEQ ID NO:49), a VL CDR2 (SEQ ID NO:42), and a VL CDR3 (SEQ ID NO:43). In another embodiment, the antibody comprises a VH CDR1 (SEQ ID NO:47), a VH CDR2 (SEQ ID NO:48), a VL CDR1 (SEQ ID NO:41), a VL CDR2 (SEQ ID NO:42), and a VL CDR3 (SEQ ID NO:43). In other embodiments, the antibody comprises a VH CDR1 (SEQ ID NO:47), a VH CDR3 (SEQ ID NO:49), a VL CDR1 (SEQ ID NO:41), a VL CDR2 (SEQ ID NO:42), and a VL CDR3 (SEQ ID NO:43). In some embodiments, the antibody comprises a VH CDR2 (SEQ ID NO:48), a VH CDR3 (SEQ ID NO:49), a VL CDR1 (SEQ ID NO:41), a VL CDR2 (SEQ ID NO:42), and a VL CDR3 (SEQ ID NO:43). In another embodiment, the antibody comprises a VH CDR1 (SEQ ID NO:47), a VL CDR1 (SEQ ID NO:41), a VL CDR2 (SEQ ID NO:42), and a VL CDR3 (SEQ ID NO:43). In one embodiment, the antibody comprises a VH CDR2 (SEQ ID NO:48), a VL CDR1 (SEQ ID NO:41), a VL CDR2 (SEQ ID NO:42), and a VL CDR3 (SEQ ID NO:43). In other embodiments, the antibody comprises a VH CDR3 (SEQ ID NO:49), a VL CDR1 (SEQ ID NO:41), a VL CDR2 (SEQ ID NO:42), and a VL CDR3 (SEQ ID NO:43). In another embodiment, the antibody comprises any combination thereof of the VH CDRs and VL CDRs listed in Tables 5 and 6.

In some embodiments, the antibodies comprise a VH CDR1, VH CDR1, VH CDR3, VL CDR1, VL CDR2, VL CDR3 according to the Chothia numbering system. Accordingly, in some embodiments, the antibodies comprise a VH CDR1 having an amino acid sequence of SEQ ID NO:111. In some embodiments, the antibodies comprise a VH CDR2 having an amino acid sequence of SEQ ID NO: 112. In some embodiments, the antibodies comprise a VH CDR3 having an amino acid sequence of SEQ ID NO:113. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 6. In some embodiments, the antibodies comprise a VL CDR1 having an amino acid sequence of any one of SEQ ID NO:114. In another embodiment, the antibodies comprise a VL CDR2 having an amino acid sequence of SEQ ID NO: 115. In some embodiments, the antibodies comprise a VL CDR3 having an amino acid sequence of SEQ ID NO:116. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 5.

In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region comprising: (1) a VL CDR1 having an amino acid sequence of SEQ ID NO:114; (2) a VL CDR2 having an amino acid sequence of SEQ ID NO:115; and (3) a VL CDR3 having an amino acid sequence of SEQ ID NO:116.

In some embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113.

In other embodiments, the antibodies provided herein comprise a VL region comprising: (1) a VL CDR1 having an amino acid sequence of SEQ ID NO: 114; (2) a VL CDR2 having an amino acid sequence of SEQ ID NO: 115; and (3) a VL CDR3 having an amino acid sequence of SEQ ID NO: 116.

Also provided herein are antibodies comprising one or more (e.g., one, two, or three) VH CDRs and one or more (e.g., one, two, or three) VL CDRs listed in Tables 5 and 6. In particular, provided herein is an antibody comprising a VH CDR1 (SEQ ID NO:111) and a VL CDR1 (SEQ ID NO:114). In one embodiment, the antibody comprises a VH CDR1 (SEQ ID NO:111) and a VL CDR2 (SEQ ID NO:115). In other embodiments, the antibody comprises a VH CDR1 (SEQ ID NO:111) and a VL CDR3 (SEQ ID NO: 116). In another embodiment, the antibody comprises a VH CDR2 (SEQ ID NO: 112) and a VL CDR1 (SEQ ID NO: 114). In some embodiments, the antibody comprises a VH CDR2 (SEQ ID NO: 112) and a VL CDR2 (SEQ ID NO:115). In one embodiment, the antibody comprises a VH CDR2 (SEQ ID NO: 112) and a VL CDR3 (SEQ ID NO: 116). In another embodiment, the antibody comprises a VH CDR3 (SEQ ID NO:113) and a VL CDR1 (SEQ ID NO: 114). In other embodiments, the antibody comprises a VH CDR3 (SEQ ID NO:113) and a VL CDR2 (SEQ ID NO:115). In some embodiments, the antibody comprises a VH CDR3 (SEQ ID NO: 113) and a VL CDR3 (SEQ ID NO: 116). In another embodiment, the antibody comprises a VH CDR1 (SEQ ID NO:111), a VH CDR2 (SEQ ID NO:112), and a VL CDR1 (SEQ ID NO:114). In one embodiment, the antibody comprises a VH CDR1 (SEQ ID NO:111), a VH CDR2 (SEQ ID NO:112), and a VL CDR2 (SEQ ID NO:115). In other embodiments, the antibody comprises a VH CDR1 (SEQ ID NO:111), a VH CDR2 (SEQ ID NO:112), and a VL CDR3 (SEQ ID NO: 116). In another embodiment, the antibody comprises a VH CDR2 (SEQ ID NO: 112), a VH CDR3 (SEQ ID NO: 113), and a VL CDR1 (SEQ ID NO:114). In some embodiments, the antibody comprises a VH CDR2 (SEQ ID NO: 112), a VH CDR3 (SEQ ID NO: 113), and a VL CDR2 (SEQ ID NO: 115). In one embodiment, the antibody comprises a VH CDR2 (SEQ ID NO:112), a VH CDR3 (SEQ ID NO:113), and a VL CDR3 (SEQ ID NO:116). In another embodiment, the antibody comprises a VH CDR1 (SEQ ID NO:111), a VH CDR3 (SEQ ID NO:113), and a VL CDR1 (SEQ ID NO:114). In other embodiments, the antibody comprises a VH CDR1 (SEQ ID NO:111), a VH CDR3 (SEQ ID NO:113), and a VL CDR2 (SEQ ID NO: 115). In some embodiments, the antibody comprises a VH CDR1 (SEQ ID NO:111), a VH CDR3 (SEQ ID NO: 113), and a VL CDR3 (SEQ ID NO:116). In another embodiment, the antibody comprises a VH CDR1 (SEQ TD NO:111), a VL CDR1 (SEQ ID NO:114), and a VL CDR2 (SEQ ID NO:115). In one embodiment, the antibody comprises a VH CDR1 (SEQ ID NO:111), a VL CDR1 (SEQ ID NO:114), and a VL CDR3 (SEQ ID NO: 116). In other embodiments, the antibody comprises a VH CDR1 (SEQ ID NO:111), a VL CDR2 (SEQ ID NO: 115), and a VL CDR3 (SEQ ID NO: 116). In another embodiment, the antibody comprises a VH CDR2 (SEQ ID NO: 112), a VL CDR1 (SEQ ID NO:114), and a VL CDR2 (SEQ ID NO: 115). In some embodiments, the antibody comprises a VH CDR2 (SEQ ID NO:112), a VL CDR1 (SEQ ID NO: 114), and a VL CDR3 (SEQ ID NO: 116). In one embodiment, the antibody comprises a VH CDR2 (SEQ ID NO: 112), a VL CDR2 (SEQ ID NO: 115), and a VL CDR3 (SEQ ID NO: 116). In another embodiment, the antibody comprises a VH CDR3 (SEQ ID NO: 113), a VL CDR1 (SEQ ID NO:114), and a VL CDR2 (SEQ ID NO:115). In other embodiments, the antibody comprises a VH CDR3 (SEQ ID NO:113), a VL CDR1 (SEQ ID NO: 114), and a VL CDR3 (SEQ ID NO:116). In some embodiments, the antibody comprises a VH CDR3 (SEQ ID NO: 113), a VL CDR2 (SEQ ID NO:115), and a VL CDR3 (SEQ ID NO: 116). In another embodiment, the antibody comprises a VH CDR1 (SEQ ID NO:111), a VH CDR2 (SEQ ID NO:112), a VH CDR3 (SEQ ID NO:113), and a VL CDR1 (SEQ ID NO: 114). In one embodiment, the antibody comprises a VH CDR1 (SEQ ID NO:111), a VH CDR2 (SEQ ID NO:112), a VH CDR3 (SEQ ID NO:113), and a VL CDR2 (SEQ ID NO:115). In other embodiments, the antibody comprises a VH CDR1 (SEQ ID NO:111), a VH CDR2 (SEQ ID NO:112), a VH CDR3 (SEQ ID NO: 113), and a VL CDR3 (SEQ ID NO:116). In another embodiment, the antibody comprises a VH CDR1 (SEQ ID NO:111), a VH CDR2 (SEQ ID NO: 112), a VL CDR1 (SEQ ID NO: 114), and a VL CDR2 (SEQ ID NO:115). In some embodiments, the antibody comprises a VH CDR1 (SEQ ID NO:111), a VH CDR2 (SEQ ID NO:112), a VL CDR1 (SEQ ID NO:114), and a VL CDR3 (SEQ ID NO: 116). In one embodiment, the antibody comprises a VH CDR1 (SEQ ID NO:111), a VH CDR2 (SEQ ID NO:112), a VL CDR2 (SEQ ID NO:115), and a VL CDR3 (SEQ ID NO: 116). In another embodiment, the antibody comprises a VH CDR1 (SEQ ID NO:111), a VH CDR3 (SEQ ID NO: 113), a VL CDR1 (SEQ ID NO: 114), and a VL CDR2 (SEQ ID NO: 115). In other embodiments, the antibody comprises a VH CDR1 (SEQ ID NO:111), a VH CDR3 (SEQ ID NO:113), a VL CDR1 (SEQ ID NO: 114), and a VL CDR3 (SEQ ID NO: 116). In some embodiments, the antibody comprises a VH CDR1 (SEQ ID NO:111), a VH CDR3 (SEQ ID NO: 113), a VL CDR2 (SEQ ID NO: 115), and a VL CDR3 (SEQ ID NO: 116). In another embodiment, the antibody comprises a VH CDR2 (SEQ ID NO:112), a VH CDR3 (SEQ ID NO:113), a VL CDR1 (SEQ ID NO:114), and a VL CDR2 (SEQ ID NO:115). In one embodiment, the antibody comprises a VH CDR2 (SEQ ID NO:112), a VH CDR3 (SEQ ID NO:113), a VL CDR1 (SEQ ID NO:114), and a VL CDR3 (SEQ ID NO:116). In other embodiments, the antibody comprises a VH CDR2 (SEQ ID NO: 112), a VH CDR3 (SEQ ID NO:113), a VL CDR2 (SEQ ID NO:115), and a VL CDR3 (SEQ ID NO:116). In another embodiment, the antibody comprises a VH CDR1 (SEQ ID NO:111), a VH CDR2 (SEQ ID NO:112), a VH CDR3 (SEQ ID NO:113), a VL CDR1 (SEQ ID NO: 114), and a VL CDR2 (SEQ ID NO:115). In some embodiments, the antibody comprises a VH CDR1 (SEQ ID NO:111), a VH CDR2 (SEQ ID NO:112), a VH CDR3 (SEQ ID NO:113), a VL CDR1 (SEQ ID NO: 114), and a VL CDR3 (SEQ ID NO: 116). In one embodiment, the antibody comprises a VH CDR1 (SEQ ID NO: 111), a VH CDR2 (SEQ ID NO: 112), a VH CDR3 (SEQ ID NO: 113), a VL CDR2 (SEQ ID NO: 115), and a VL CDR3 (SEQ ID NO: 116). In another embodiment, the antibody comprises a VH CDR1 (SEQ ID NO: 111), a VH CDR2 (SEQ ID NO: 112), a VL CDR1 (SEQ ID NO:114), a VL CDR2 (SEQ ID NO:115), and a VL CDR3 (SEQ ID NO:116). In other embodiments, the antibody comprises a VH CDR1 (SEQ ID NO: 111), a VH CDR3 (SEQ ID NO: 113), a VL CDR1 (SEQ ID NO: 114), a VL CDR2 (SEQ ID NO: 115), and a VL CDR3 (SEQ ID NO:116). In some embodiments, the antibody comprises a VH CDR2 (SEQ ID NO:112), a VH CDR3 (SEQ ID NO:113), a VL CDR1 (SEQ ID NO:114), a VL CDR2 (SEQ ID NO: 115), and a VL CDR3 (SEQ ID NO: 116). In another embodiment, the antibody comprises a VH CDR1 (SEQ ID NO:111), a VL CDR1 (SEQ ID NO: 114), a VL CDR2 (SEQ ID NO: 115), and a VL CDR3 (SEQ ID NO: 116). In one embodiment, the antibody comprises a VH CDR2 (SEQ ID NO: 112), a VL CDR1 (SEQ ID NO: 114), a VL CDR2 (SEQ ID NO: 115), and a VL CDR3 (SEQ ID NO: 116). In other embodiments, the antibody comprises a VH CDR3 (SEQ ID NO: 113), a VL CDR1 (SEQ ID NO:114), a VL CDR2 (SEQ ID NO: 115), and a VL CDR3 (SEQ ID NO: 116). In another embodiment, the antibody comprises any combination thereof of the VH CDRs and VL CDRs listed in Tables 5 and 6.

In some embodiments, the antibodies comprise a VH CDR1, VH CDR1, VH CDR3, VL CDR1, VL CDR2, VL CDR3 according to the IMGT numbering system. Accordingly, in some embodiments, the antibodies comprise a VH CDR1 having an amino acid sequence of SEQ ID NO:117. In some embodiments, the antibodies comprise a VH CDR2 having an amino acid sequence of SEQ ID NO: 118. In some embodiments, the antibodies comprise a VH CDR3 having an amino acid sequence of SEQ ID NO:119. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 6. In some embodiments, the antibodies comprise a VL CDR1 having an amino acid sequence of any one of SEQ ID NO:120. In another embodiment, the antibodies comprise a VL CDR2 having an amino acid sequence of SEQ ID NO:121. In some embodiments, the antibodies comprise a VL CDR3 having an amino acid sequence of SEQ ID NO:122. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 5.

In certain embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ ID NO:119; and a VL region comprising: (1) a VL CDR1 having an amino acid sequence of SEQ ID NO:120; (2) a VL CDR2 having an amino acid sequence of SEQ ID NO:121; and (3) a VL CDR3 having an amino acid sequence of SEQ ID NO:122.

In some embodiments, the antibodies provided herein comprise a VH region comprising: (1) a VH CDR1 having an amino acid sequence of SEQ ID NO:117; (2) a VH CDR2 having an amino acid sequence of SEQ ID NO:118; and (3) a VH CDR3 having an amino acid sequence of SEQ ID NO:119.

In other embodiments, the antibodies provided herein comprise a VL region comprising: (1) a VL CDR1 having an amino acid sequence of SEQ ID NO:120; (2) a VL CDR2 having an amino acid sequence of SEQ ID NO:121; and (3) a VL CDR3 having an amino acid sequence of SEQ ID NO:122.

Also provided herein are antibodies comprising one or more (e.g., one, two, or three) VH CDRs and one or more (e.g., one, two, or three) VL CDRs listed in Tables 5 and 6. In particular, provided herein is an antibody comprising a VH CDR1 (SEQ ID NO: 117) and a VL CDR1 (SEQ ID NO:120). In one embodiment, the antibody comprises a VH CDR1 (SEQ ID NO:117) and a VL CDR2 (SEQ ID NO:121). In other embodiments, the antibody comprises a VH CDR1 (SEQ ID NO: 117) and a VL CDR3 (SEQ ID NO: 122). In another embodiment, the antibody comprises a VH CDR2 (SEQ ID NO:118) and a VL CDR1 (SEQ ID NO:120). In some embodiments, the antibody comprises a VH CDR2 (SEQ ID NO: 118) and a VL CDR2 (SEQ ID NO:121). In one embodiment, the antibody comprises a VH CDR2 (SEQ ID NO:118) and a VL CDR3 (SEQ ID NO:122). In another embodiment, the antibody comprises a VH CDR3 (SEQ ID NO:119) and a VL CDR1 (SEQ ID NO:120). In other embodiments, the antibody comprises a VH CDR3 (SEQ ID NO: 119) and a VL CDR2 (SEQ ID NO:121). In some embodiments, the antibody comprises a VH CDR3 (SEQ ID NO: 119) and a VL CDR3 (SEQ ID NO: 122). In another embodiment, the antibody comprises a VH CDR1 (SEQ ID NO:117), a VH CDR2 (SEQ ID NO: 118), and a VL CDR1 (SEQ ID NO:120). In one embodiment, the antibody comprises a VH CDR1 (SEQ ID NO:117), a VH CDR2 (SEQ ID NO:118), and a VL CDR2 (SEQ ID NO:121). In other embodiments, the antibody comprises a VH CDR1 (SEQ ID NO:117), a VH CDR2 (SEQ ID NO:118), and a VL CDR3 (SEQ ID NO: 122). In another embodiment, the antibody comprises a VH CDR2 (SEQ ID NO: 118), a VH CDR3 (SEQ ID NO: 119), and a VL CDR1 (SEQ ID NO:120). In some embodiments, the antibody comprises a VH CDR2 (SEQ ID NO:118), a VH CDR3 (SEQ ID NO:119), and a VL CDR2 (SEQ ID NO:121). In one embodiment, the antibody comprises a VH CDR2 (SEQ ID NO: 118), a VH CDR3 (SEQ ID NO:119), and a VL CDR3 (SEQ ID NO:122). In another embodiment, the antibody comprises a VH CDR1 (SEQ ID NO:117), a VH CDR3 (SEQ ID NO:119), and a VL CDR1 (SEQ ID NO:120). In other embodiments, the antibody comprises a VH CDR1 (SEQ ID NO: 117), a VH CDR3 (SEQ ID NO: 119), and a VL CDR2 (SEQ ID NO:121). In some embodiments, the antibody comprises a VH CDR1 (SEQ ID NO:117), a VH CDR3 (SEQ ID NO:119), and a VL CDR3 (SEQ ID NO:122). In another embodiment, the antibody comprises a VH CDR1 (SEQ ID NO: 117), a VL CDR1 (SEQ ID NO:120), and a VL CDR2 (SEQ ID NO:121). In one embodiment, the antibody comprises a VH CDR1 (SEQ ID NO:117), a VL CDR1 (SEQ ID NO:120), and a VL CDR3 (SEQ ID NO:122). In other embodiments, the antibody comprises a VH CDR1 (SEQ ID NO:117), a VL CDR2 (SEQ ID NO:121), and a VL CDR3 (SEQ ID NO:122). In another embodiment, the antibody comprises a VH CDR2 (SEQ ID NO: 118), a VL CDR1 (SEQ ID NO:120), and a VL CDR2 (SEQ ID NO:121). In some embodiments, the antibody comprises a VH CDR2 (SEQ ID NO:118), a VL CDR1 (SEQ ID NO: 120), and a VL CDR3 (SEQ ID NO:122). In one embodiment, the antibody comprises a VH CDR2 (SEQ ID NO:118), a VL CDR2 (SEQ ID NO:121), and a VL CDR3 (SEQ ID NO:122). In another embodiment, the antibody comprises a VH CDR3 (SEQ ID NO: 119), a VL CDR1 (SEQ ID NO:120), and a VL CDR2 (SEQ ID NO:121). In other embodiments, the antibody comprises a VH CDR3 (SEQ ID NO: 119), a VL CDR1 (SEQ ID NO: 120), and a VL CDR3 (SEQ ID NO:122). In some embodiments, the antibody comprises a VH CDR3 (SEQ ID NO: 119), a VL CDR2 (SEQ ID NO:121), and a VL CDR3 (SEQ ID NO: 122). In another embodiment, the antibody comprises a VH CDR1 (SEQ ID NO:117), a VH CDR2 (SEQ ID NO:118), a VH CDR3 (SEQ ID NO: 119), and a VL CDR1 (SEQ ID NO: 120). In one embodiment, the antibody comprises a VH CDR1 (SEQ ID NO:117), a VH CDR2 (SEQ ID NO:118), a VH CDR3 (SEQ ID NO:119), and a VL CDR2 (SEQ ID NO:121). In other embodiments, the antibody comprises a VH CDR1 (SEQ ID NO:117), a VH CDR2 (SEQ ID NO:118), a VH CDR3 (SEQ ID NO:119), and a VL CDR3 (SEQ ID NO: 122). In another embodiment, the antibody comprises a VH CDR1 (SEQ ID NO: 117), a VH CDR2 (SEQ ID NO: 118), a VL CDR1 (SEQ ID NO:120), and a VL CDR2 (SEQ ID NO: 121). In some embodiments, the antibody comprises a VH CDR1 (SEQ ID NO: 117), a VH CDR2 (SEQ ID NO: 118), a VL CDR1 (SEQ ID NO:120), and a VL CDR3 (SEQ ID NO: 122). In one embodiment, the antibody comprises a VH CDR1 (SEQ ID NO:117), a VH CDR2 (SEQ ID NO:118), a VL CDR2 (SEQ ID NO:121), and a VL CDR3 (SEQ ID NO:122). In another embodiment, the antibody comprises a VH CDR1 (SEQ ID NO: 117), a VH CDR3 (SEQ ID NO:119), a VL CDR1 (SEQ ID NO:120), and a VL CDR2 (SEQ ID NO:121). In other embodiments, the antibody comprises a VH CDR1 (SEQ ID NO: 117), a VH CDR3 (SEQ ID NO: 119), a VL CDR1 (SEQ ID NO:120), and a VL CDR3 (SEQ ID NO:122). In some embodiments, the antibody comprises a VH CDR1 (SEQ ID NO: 117), a VH CDR3 (SEQ ID NO: 119), a VL CDR2 (SEQ ID NO:121), and a VL CDR3 (SEQ ID NO:122). In another embodiment, the antibody comprises a VH CDR2 (SEQ ID NO: 118), a VH CDR3 (SEQ ID NO: 119), a VL CDR1 (SEQ ID NO:120), and a VL CDR2 (SEQ ID NO:121). In one embodiment, the antibody comprises a VH CDR2 (SEQ ID NO: 118), a VH CDR3 (SEQ ID NO:119), a VL CDR1 (SEQ ID NO:120), and a VL CDR3 (SEQ ID NO:122). In other embodiments, the antibody comprises a VH CDR2 (SEQ ID NO: 118), a VH CDR3 (SEQ ID NO: 119), a VL CDR2 (SEQ ID NO:121), and a VL CDR3 (SEQ ID NO:122). In another embodiment, the antibody comprises a VH CDR1 (SEQ ID NO: 117), a VH CDR2 (SEQ ID NO: 118), a VH CDR3 (SEQ ID NO: 119), a VL CDR1 (SEQ ID NO:120), and a VL CDR2 (SEQ ID NO:121). In some embodiments, the antibody comprises a VH CDR1 (SEQ ID NO: 117), a VH CDR2 (SEQ ID NO:118), a VH CDR3 (SEQ ID NO:119), a VL CDR1 (SEQ ID NO:120), and a VL CDR3 (SEQ ID NO: 122). In one embodiment, the antibody comprises a VH CDR1 (SEQ ID NO:117), a VH CDR2 (SEQ ID NO:118), a VH CDR3 (SEQ ID NO:119), a VL CDR2 (SEQ ID NO:121), and a VL CDR3 (SEQ ID NO:122). In another embodiment, the antibody comprises a VH CDR1 (SEQ ID NO:117), a VH CDR2 (SEQ ID NO:118), a VL CDR1 (SEQ ID NO:120), a VL CDR2 (SEQ ID NO:121), and a VL CDR3 (SEQ ID NO:122). In other embodiments, the antibody comprises a VH CDR1 (SEQ ID NO: 117), a VH CDR3 (SEQ ID NO: 119), a VL CDR1 (SEQ ID NO:120), a VL CDR2 (SEQ ID NO:121), and a VL CDR3 (SEQ ID NO:122). In some embodiments, the antibody comprises a VH CDR2 (SEQ ID NO: 118), a VH CDR3 (SEQ ID NO:119), a VL CDR1 (SEQ ID NO:120), a VL CDR2 (SEQ ID NO:121), and a VL CDR3 (SEQ ID NO:122). In another embodiment, the antibody comprises a VH CDR1 (SEQ ID NO:117), a VL CDR1 (SEQ ID NO:120), a VL CDR2 (SEQ ID NO:121), and a VL CDR3 (SEQ ID NO: 122). In one embodiment, the antibody comprises a VH CDR2 (SEQ ID NO: 118), a VL CDR1 (SEQ ID NO:120), a VL CDR2 (SEQ ID NO:121), and a VL CDR3 (SEQ ID NO:122). In other embodiments, the antibody comprises a VH CDR3 (SEQ ID NO: 119), a VL CDR1 (SEQ ID NO:120), a VL CDR2 (SEQ ID NO:121), and a VL CDR3 (SEQ ID NO:122). In another embodiment, the antibody comprises any combination thereof of the VH CDRs and VL CDRs listed in Tables 5 and 6.

In some embodiments, the antibodies comprise a VH CDR1, VH CDR1, VH CDR3, VL CDR1, VL CDR2, VL CDR3 according to the Contact numbering system. Accordingly, in some embodiments, the antibodies comprise a VH CDR1 having an amino acid sequence of SEQ ID NO:123. In some embodiments, the antibodies comprise a VH CDR2 having an amino acid sequence of SEQ ID NO: 124. In some embodiments, the antibodies comprise a VH CDR3 having an amino acid sequence of SEQ ID NO: 125. In some embodiments, the antibodies comprise a VH CDR1 and/or a VH CDR2 and/or a VH CDR3 independently selected from any one of the VH CDR1, VH CDR2, VH CDR3 amino acid sequence(s) as depicted in Table 6. In some embodiments, the antibodies comprise a VL CDR1 having an amino acid sequence of any one of SEQ ID NO:126. In another embodiment, the antibodies comprise a VL CDR2 having an amino acid sequence of SEQ ID NO:127. In some embodiments, the antibodies comprise a VL CDR3 having an amino acid sequence of SEQ ID NO:128. In some embodiments, the antibodies comprise a VL CDR1 and/or a VL CDR2 and/or a VL CDR3 independently selected from any one of the VL CDR1, VL CDR2, VL CDR3 amino acid sequences as depicted in Table 5.

In certain embodiments, the antibodies provided herein comprise a VH region comprising: (1) a VH CDR1 having an amino acid sequence of SEQ ID NO:123; (2) a VH CDR2 having an amino acid sequence of SEQ ID NO:124; and (3) a VH CDR3 having an amino acid sequence of SEQ ID NO:125; and a VL region comprising: (1) a VL CDR1 having an amino acid sequence of SEQ ID NO:126; (2) a VL CDR2 having an amino acid sequence of SEQ ID NO:127; and (3) a VL CDR3 having an amino acid sequence of SEQ ID NO:128.

In some embodiments, the antibodies provided herein comprise a VH region comprising: (1) a VH CDR1 having an amino acid sequence of SEQ ID NO:123; (2) a VH CDR2 having an amino acid sequence of SEQ ID NO:124; and (3) a VH CDR3 having an amino acid sequence of SEQ ID NO:125.

In other embodiments, the antibodies provided herein comprise a VL region comprising: (1) a VL CDR1 having an amino acid sequence of SEQ ID NO:126; (2) a VL CDR2 having an amino acid sequence of SEQ ID NO:127; and (3) a VL CDR3 having an amino acid sequence of SEQ ID NO:128.

Also provided herein are antibodies comprising one or more (e.g., one, two, or three) VH CDRs and one or more (e.g., one, two, or three) VL CDRs listed in Tables 5 and 6. In particular, provided herein is an antibody comprising a VH CDR1 (SEQ ID NO:123) and a VL CDR1 (SEQ ID NO:126). In one embodiment, the antibody comprises a VH CDR1 (SEQ ID NO:123) and a VL CDR2 (SEQ ID NO:127). In other embodiments, the antibody comprises a VH CDR1 (SEQ ID NO:123) and a VL CDR3 (SEQ ID NO:128). In another embodiment, the antibody comprises a VH CDR2 (SEQ ID NO:124) and a VL CDR1 (SEQ ID NO:126). In some embodiments, the antibody comprises a VH CDR2 (SEQ ID NO:124) and a VL CDR2 (SEQ ID NO:127). In one embodiment, the antibody comprises a VH CDR2 (SEQ ID NO:124) and a VL CDR3 (SEQ ID NO:128). In another embodiment, the antibody comprises a VH CDR3 (SEQ ID NO:125) and a VL CDR1 (SEQ ID NO:126). In other embodiments, the antibody comprises a VH CDR3 (SEQ ID NO:125) and a VL CDR2 (SEQ ID NO:127). In some embodiments, the antibody comprises a VH CDR3 (SEQ ID NO:125) and a VL CDR3 (SEQ ID NO:128). In another embodiment, the antibody comprises a VH CDR1 (SEQ ID NO:123), a VH CDR2 (SEQ ID NO:124), and a VL CDR1 (SEQ ID NO:126). In one embodiment, the antibody comprises a VH CDR1 (SEQ ID NO:123), a VH CDR2 (SEQ ID NO:124), and a VL CDR2 (SEQ ID NO:127). In other embodiments, the antibody comprises a VH CDR1 (SEQ ID NO:123), a VH CDR2 (SEQ ID NO:124), and a VL CDR3 (SEQ ID NO: 128). In another embodiment, the antibody comprises a VH CDR2 (SEQ ID NO:124), a VH CDR3 (SEQ ID NO:125), and a VL CDR1 (SEQ ID NO:126). In some embodiments, the antibody comprises a VH CDR2 (SEQ ID NO:124), a VH CDR3 (SEQ ID NO:125), and a VL CDR2 (SEQ ID NO:127). In one embodiment, the antibody comprises a VH CDR2 (SEQ ID NO:124), a VH CDR3 (SEQ ID NO:125), and a VL CDR3 (SEQ ID NO:128). In another embodiment, the antibody comprises a VH CDR1 (SEQ ID NO:123), a VH CDR3 (SEQ ID NO:125), and a VL CDR1 (SEQ ID NO:126). In other embodiments, the antibody comprises a VH CDR1 (SEQ ID NO:123), a VH CDR3 (SEQ ID NO:125), and a VL CDR2 (SEQ ID NO: 127). In some embodiments, the antibody comprises a VH CDR1 (SEQ ID NO:123), a VH CDR3 (SEQ ID NO:125), and a VL CDR3 (SEQ ID NO:128). In another embodiment, the antibody comprises a VH CDR1 (SEQ ID NO:123), a VL CDR1 (SEQ ID NO:126), and a VL CDR2 (SEQ ID NO:127). In one embodiment, the antibody comprises a VH CDR1 (SEQ ID NO:123), a VL CDR1 (SEQ ID NO:126), and a VL CDR3 (SEQ ID NO:128). In other embodiments, the antibody comprises a VH CDR1 (SEQ ID NO:123), a VL CDR2 (SEQ ID NO: 127), and a VL CDR3 (SEQ ID NO:128). In another embodiment, the antibody comprises a VH CDR2 (SEQ ID NO:124), a VL CDR1 (SEQ ID NO:126), and a VL CDR2 (SEQ ID NO: 127). In some embodiments, the antibody comprises a VH CDR2 (SEQ ID NO:124), a VL CDR1 (SEQ ID NO:126), and a VL CDR3 (SEQ ID NO:128). In one embodiment, the antibody comprises a VH CDR2 (SEQ ID NO:124), a VL CDR2 (SEQ ID NO:127), and a VL CDR3 (SEQ ID NO:128). In another embodiment, the antibody comprises a VH CDR3 (SEQ ID NO:125), a VL CDR1 (SEQ ID NO:126), and a VL CDR2 (SEQ ID NO: 127). In other embodiments, the antibody comprises a VH CDR3 (SEQ ID NO:125), a VL CDR1 (SEQ ID NO: 126), and a VL CDR3 (SEQ ID NO:128). In some embodiments, the antibody comprises a VH CDR3 (SEQ ID NO: 125), a VL CDR2 (SEQ ID NO:127), and a VL CDR3 (SEQ ID NO: 128). In another embodiment, the antibody comprises a VH CDR1 (SEQ ID NO:123), a VH CDR2 (SEQ ID NO:124), a VH CDR3 (SEQ ID NO:125), and a VL CDR1 (SEQ ID NO: 126). In one embodiment, the antibody comprises a VH CDR1 (SEQ ID NO:123), a VH CDR2 (SEQ ID NO:124), a VH CDR3 (SEQ ID NO:125), and a VL CDR2 (SEQ ID NO:127). In other embodiments, the antibody comprises a VH CDR1 (SEQ ID NO: 123), a VH CDR2 (SEQ ID NO: 124), a VH CDR3 (SEQ ID NO: 125), and a VL CDR3 (SEQ ID NO: 128). In another embodiment, the antibody comprises a VH CDR1 (SEQ ID NO:123), a VH CDR2 (SEQ ID NO:124), a VL CDR1 (SEQ ID NO:126), and a VL CDR2 (SEQ ID NO: 127). In some embodiments, the antibody comprises a VH CDR1 (SEQ ID NO:123), a VH CDR2 (SEQ ID NO: 124), a VL CDR1 (SEQ ID NO: 126), and a VL CDR3 (SEQ ID NO: 128). In one embodiment, the antibody comprises a VH CDR1 (SEQ ID NO:123), a VH CDR2 (SEQ ID NO: 124), a VL CDR2 (SEQ ID NO:127), and a VL CDR3 (SEQ ID NO:128). In another embodiment, the antibody comprises a VH CDR1 (SEQ ID NO:123), a VH CDR3 (SEQ ID NO:125), a VL CDR1 (SEQ ID NO:126), and a VL CDR2 (SEQ ID NO:127). In other embodiments, the antibody comprises a VH CDR1 (SEQ ID NO: 123), a VH CDR3 (SEQ ID NO: 125), a VL CDR1 (SEQ ID NO:126), and a VL CDR3 (SEQ ID NO:128). In some embodiments, the antibody comprises a VH CDR1 (SEQ ID NO: 123), a VH CDR3 (SEQ ID NO:125), a VL CDR2 (SEQ ID NO:127), and a VL CDR3 (SEQ ID NO:128). In another embodiment, the antibody comprises a VH CDR2 (SEQ ID NO:124), a VH CDR3 (SEQ ID NO:125), a VL CDR1 (SEQ ID NO:126), and a VL CDR2 (SEQ ID NO:127). In one embodiment, the antibody comprises a VH CDR2 (SEQ ID NO:124), a VH CDR3 (SEQ ID NO:125), a VL CDR1 (SEQ ID NO:126), and a VL CDR3 (SEQ ID NO:128). In other embodiments, the antibody comprises a VH CDR2 (SEQ ID NO: 124), a VH CDR3 (SEQ ID NO:125), a VL CDR2 (SEQ ID NO:127), and a VL CDR3 (SEQ ID NO:128). In another embodiment, the antibody comprises a VH CDR1 (SEQ ID NO:123), a VH CDR2 (SEQ ID NO:124), a VH CDR3 (SEQ ID NO:125), a VL CDR1 (SEQ ID NO: 126), and a VL CDR2 (SEQ ID NO:127). In some embodiments, the antibody comprises a VH CDR1 (SEQ ID NO:123), a VH CDR2 (SEQ ID NO: 124), a VH CDR3 (SEQ ID NO:125), a VL CDR1 (SEQ ID NO:126), and a VL CDR3 (SEQ ID NO: 128). In one embodiment, the antibody comprises a VH CDR1 (SEQ ID NO: 123), a VH CDR2 (SEQ ID NO: 124), a VH CDR3 (SEQ ID NO:125), a VL CDR2 (SEQ ID NO: 127), and a VL CDR3 (SEQ ID NO:128). In another embodiment, the antibody comprises a VH CDR1 (SEQ ID NO: 123), a VH CDR2 (SEQ ID NO: 124), a VL CDR1 (SEQ ID NO:126), a VL CDR2 (SEQ ID NO:127), and a VL CDR3 (SEQ ID NO:128). In other embodiments, the antibody comprises a VH CDR1 (SEQ ID NO: 123), a VH CDR3 (SEQ ID NO:125), a VL CDR1 (SEQ ID NO: 126), a VL CDR2 (SEQ ID NO: 127), and a VL CDR3 (SEQ ID NO:128). In some embodiments, the antibody comprises a VH CDR2 (SEQ ID NO:124), a VH CDR3 (SEQ ID NO: 125), a VL CDR1 (SEQ ID NO: 126), a VL CDR2 (SEQ ID NO:127), and a VL CDR3 (SEQ ID NO: 128). In another embodiment, the antibody comprises a VH CDR1 (SEQ ID NO:123), a VL CDR1 (SEQ ID NO:126), a VL CDR2 (SEQ ID NO: 127), and a VL CDR3 (SEQ ID NO: 128). In one embodiment, the antibody comprises a VH CDR2 (SEQ ID NO:124), a VL CDR1 (SEQ ID NO: 126), a VL CDR2 (SEQ ID NO: 127), and a VL CDR3 (SEQ ID NO:128). In other embodiments, the antibody comprises a VH CDR3 (SEQ ID NO:125), a VL CDR1 (SEQ ID NO:126), a VL CDR2 (SEQ ID NO:127), and a VL CDR3 (SEQ ID NO:128). In another embodiment, the antibody comprises any combination thereof of the VH CDRs and VL CDRs listed in Tables 5 and 6.

In some embodiments, an antibody provided herein comprises or consists of six CDRs, for example, VH CDR1, VH CDR2, VH CDR3, VL CDR1, VL CDR2, and/or VL CDR3 identified in Tables 1-6. In some embodiments, an antibody provided herein can comprise fewer than six CDRs. In some embodiments, the antibody comprises or consists of one, two, three, four, or five CDRs selected from the group consisting of VH CDR1, VH CDR2, VH CDR3, VL CDR1, VL CDR2, and/or VL CDR3 identified in Tables 1-6. In some embodiments, the antibody comprises or consists of one, two, three, four, or five CDRs selected from the group consisting of VH CDR1, VH CDR2, VH CDR3, VL CDR1, VL CDR2, and/or VL CDR3 of the monoclonal described herein. Accordingly, in some embodiments, the antibody comprises or consists of one, two, three, four, or five CDRs of anyone of the VH CDR1, VH CDR2, VH CDR3, VL CDR1, VL CDR2, and/or VL CDR3 identified in Tables 1-6.

In some embodiments, the antibodies provided herein comprise one or more (e.g., one, two, or three) VH CDRs listed in Tables 2, 4 and 6. In other embodiments, the antibodies provided herein comprise one or more (e.g., one, two, or three) VL CDRs listed in Tables 1, 3, and 5. In yet other embodiments, the antibodies provided herein comprise one or more (e.g., one, two, or three) VH CDRs listed in Table 2 and one or more VL CDRs listed in Table 1. In yet other embodiments, the antibodies provided herein comprise one or more (e.g., one, two, or three) VH CDRs listed in Table 4 and one or more VL CDRs listed in Table 3. In yet other embodiments, the antibodies provided herein comprise one or more (e.g., one, two, or three) VH CDRs listed in Table 6 and one or more VL CDRs listed in Table 5.

In some embodiments, the isolated antibody or functional fragment thereof provided herein further comprises one, two, three, and/or four heavy chain FRs and/or one, two, three, and/or four light chain FRs from the antibodies STC43H11-1, STC43G3-1, or STC85F1-1, as shown in Tables 7 and 8.

TABLE 7
VL FR Amino Acid Sequences

| | VL FR1 | VL FR2 | VL FR3 | VL FR4 |
|---|---|---|---|---|
| Antibody | (SEQ ID NO:) | (SEQ ID NO:) | (SEQ ID NO:) | (SEQ ID NO:) |
| STC43H11-1 | QVLTQTASP | WFQQKPGQPPK | GVPSRFKGSGSGT | FGGGTEVVVK |
| | VSAAVGGTV | RLIY (SEQ ID | QFTLTISDVQCDD | (SEQ ID NO: 14) |
| | TINC (SEQ ID | NO: 12) | AATYYC (SEQ ID | |
| | NO: 11) | | NO: 13) | |
| STC43G3-1 | DPVMTQTPS | WFQQKPGQPPK | GVPPRFSGSGSGT | FGGGTEVVVK |
| | STSAAVGGT | LLIY | QFTLTISSVQCDD | (SEQ ID NO: 53) |
| | VTINC | (SEQ ID NO: 51) | AATYFC | |
| | (SEQ ID | | (SEQ ID NO: 52) | |
| | NO: 50) | | | |
| STC85F1-1 | DPVMTQTPS | WFQQKPGQPPK | GVPSRFKGSGSGT | FGGGTEVVVK |
| | STSAAVGGT | LLIY | QFTLTISDLECDD | (SEQ ID NO: 57) |
| | VTINC | (SEQ ID NO: 55) | AATYYC | |
| | (SEQ ID | | (SEQ ID NO: 56) | |
| | NO: 54) | | | |

TABLE 8
VH FR Amino Acid Sequences

| | VH FR1 | VH FR2 | VH FR3 | VH FR4 |
|---|---|---|---|---|
| Antibody | (SEQ ID NO:) | (SEQ ID NO:) | (SEQ ID NO:) | (SEQ ID NO:) |
| STC43H11-1 | QSLEESGGRL | WVRQAPGEGLE | RFTISKTSTTVDL | WGPGTLVTVSS |
| | VTPGGSLTLT | WIG | KMTSLTASDTAT | (SEQ ID NO: 18) |
| | CTVSGIDLS | (SEQ ID NO: 16) | YFCAT | |
| | (SEQ ID | | (SEQ ID NO: 17) | |
| | NO: 15) | | | |
| STC43G3-1 | QSLEESGGRL | WVRQAPGKGLE | RFTISKTSTTVEL | WGQGTLVTVSS |
| | VTPGTPLTLT | WIG | KMTSLTAADTAT | (SEQ ID NO: 61) |
| | CTVSGIDLS | (SEQ ID NO: 59) | YFCAR | |
| | (SEQ ID | | (SEQ ID NO: 60) | |
| | NO: 58) | | | |
| STC85F1-1 | QSLEESGGRL | WVRQAPGKGLE | RFTISKTSSTTVDL | WGQGTLVTVSS |
| | VTPGGSLTLT | WIG | KMTSLTTEDTAT | (SEQ ID NO: 65) |
| | CTVSGIDLS | (SEQ ID NO: 63) | YFCAR | |
| | (SEQ ID | | (SEQ ID NO: 64) | |
| | NO: 62) | | | |

In certain embodiments, the isolated antibody or functional fragment thereof provided herein further comprises one, two, three, and/or four heavy chain FRs from STC43H11-1, STC43G3-1, or STC85F1-1, as shown in Table 8. In some embodiments, the antibody heavy chain FR(s) is from the antibodies STC43H11-1, STC43G3-1, or STC85F1-1.

In some embodiments, the isolated antibody or functional fragment thereof provided herein further comprises one, two, three, and/or four light chain FRs from the antibody STC43H11-1, STC43G3-1, or STC85F1-1, as shown in Table 7. In some embodiments, the antibody light chain FR(s) is from the antibodies STC43H11-1, STC43G3-1, or STC85F1-1.

In certain embodiments, an antibody or fragment thereof described herein comprises a VH region that comprises: (1) a VH FR1 having an amino acid sequence of SEQ ID NO:15; (2) a VH FR2 having an amino acid sequence of SEQ ID NO:16; (3) a VH FR3 having an amino acid sequence of SEQ ID NO:17; and/or (4) a VH FR4 having an amino acid sequence of SEQ ID NO:18.

Accordingly, in one embodiment, the humanized antibody comprises a VH region that includes a VH FR1 having an amino acid sequence of SEQ ID NO:15. In some embodiments, the humanized antibody comprises a VH region that includes a VH FR2 having an amino acid sequence of SEQ ID NO: 16. In one embodiment, the humanized antibody comprises a VH region that includes a VH FR3 having an amino acid sequence of SEQ ID NO:17. In one embodiment, the humanized antibody comprises a VH region that includes a VH FR3 having an amino acid sequence of SEQ ID NO:18.

In certain embodiments, an antibody or fragment thereof described herein comprises a VL region that comprises: (1) a VL FR1 having an amino acid sequence of SEQ ID NO:11; (2) a VL FR2 having an amino acid sequence of SEQ ID NO:12; (3) a VL FR3 having an amino acid sequence of SEQ ID NO:13; and/or (4) a VL FR4 having an amino acid sequence of SEQ ID NO:14.

Accordingly, in some embodiments, the humanized antibody comprises a VL region that includes a VL FR1 having an amino acid sequence of SEQ ID NO:11. In certain embodiments, the humanized antibody comprises a VL region that includes a VL FR2 having an amino acid sequence of SEQ ID NO:12. In one embodiment, the humanized antibody comprises a VL region that includes a VL FR3 having an amino acid sequence of SEQ ID NO:13. In other embodiments, the humanized antibody comprises a VL region that includes a VL FR4 having an amino acid sequence of SEQ ID NO:14.

In certain embodiments, an antibody or fragment thereof described herein comprises a VH region and a VL region, wherein the VH region comprises: (1) a VH FR1 having an amino acid sequence of SEQ ID NO: 15; (2) a VH FR2 having an amino acid sequence of SEQ ID NO:16; (3) a VH FR3 having an amino acid sequence of SEQ ID NO:17; and/or (4) a VH FR4 having an amino acid sequence of SEQ ID NO:18; and wherein the VL region comprises: (1) a VL FR1 having an amino acid sequence of SEQ ID NO: 11; (2) a VL FR2 having an amino acid sequence of SEQ ID NO:12; (3) a VL FR3 having an amino acid sequence of SEQ ID NO:13; and/or (4) a VL FR4 having an amino acid sequence of SEQ ID NO:14. In some embodiments, the antibody comprises a VH region comprising all four of the above-referenced VH FR1, VH FR2, VH FR3, and VH FR4. In other embodiments, the antibody comprises a VL region comprising all four of the above-referenced VL FR1, VL FR2, VL FR3, and VL FR4. In yet other embodiments, the antibody comprises a VH region comprising all four of the above-referenced VH FR1, VH FR2, VH FR3, and VH FR4, and a VL region comprising all four of the above-referenced VL FR1, VL FR2, VL FR3, and VL FR4.

In some embodiments, an antibody or fragment thereof described comprises a VH region that comprises: (1) a VH FR1 having an amino acid sequence of SEQ ID NO:58; (2) a VH FR2 having an amino acid sequence of SEQ ID NO:59; (3) a VH FR3 having an amino acid sequence of SEQ ID NO:60; and/or (4) a VH FR4 having an amino acid sequence of SEQ ID NO:61.

In some embodiments, the humanized antibody comprises a VH region that includes VH FR1 having an amino acid sequence of SEQ ID NO:58. In some embodiments, the humanized antibody comprises a VH region that includes VH FR1 having an amino acid sequence of SEQ ID NO:59. In some embodiments, the humanized antibody comprises a VH region that includes VH FR1 having an amino acid sequence of SEQ ID NO:60. In some embodiments, the humanized antibody comprises a VH region that includes VH FR1 having an amino acid sequence of SEQ ID NO:61.

In certain embodiments, an antibody or fragment thereof described herein comprises a VL region that comprises: (1) a VL FR1 having an amino acid sequence of SEQ ID NO:50; (2) a VL FR2 having an amino acid sequence of SEQ ID NO:51; (3) a VL FR3 having an amino acid sequence of SEQ ID NO:52; and/or (4) a VL FR4 having an amino acid sequence of SEQ ID NO:53.

In certain embodiments, the humanized antibody comprises a VL region that includes a VL FR1 having an amino acid sequence of SEQ ID NO:50. In certain embodiments, the humanized antibody comprises a VL region that includes a VL FR1 having an amino acid sequence of SEQ ID NO:51. In certain embodiments, the humanized antibody comprises a VL region that includes a VL FR1 having an amino acid sequence of SEQ ID NO:52. In certain embodiments, the humanized antibody comprises a VL region that includes a VL FR1 having an amino acid sequence of SEQ ID NO:53.

In certain embodiments, an antibody or fragment thereof described herein comprises a VH region and a VL region, wherein the VH region comprises: (1) a VH FR1 having an amino acid sequence of SEQ ID NO:58; (2) a VH FR2 having an amino acid sequence of SEQ ID NO:59; (3) a VH FR3 having an amino acid sequence of SEQ ID NO:60; and/or (4) a VH FR4 having an amino acid sequence of SEQ ID NO:61; and wherein the VL region comprises: (1) a VL FR1 having an amino acid sequence of SEQ ID NO:50; (2) a VL FR2 having an amino acid sequence of SEQ ID NO:51; (3) a VL FR3 having an amino acid sequence of SEQ ID NO:52; and/or (4) a VL FR4 having an amino acid sequence of SEQ ID NO:53. In some embodiments, the antibody comprises a VH region comprising all four of the above-referenced VH FR1, VH FR2, VH FR3, and VH FR4. In other embodiments, the antibody comprises a VL region comprising all four of the above-referenced VL FR1, VL FR2, VL FR3, and VL FR4. In yet other embodiments, the antibody comprises a VH region comprising all four of the above-referenced VH FR1, VH FR2, VH FR3, and VH FR4, and a VL region comprising all four of the above-referenced VL FR1, VL FR2, VL FR3, and VL FR4.

In some embodiments, an antibody or fragment thereof described comprises a VH region that comprises: (1) a VH FR1 having an amino acid sequence of SEQ ID NO:62; (2) a VH FR2 having an amino acid sequence of SEQ ID NO:63; (3) a VH FR3 having an amino acid sequence of SEQ ID NO:64; and/or (4) a VH FR4 having an amino acid sequence of SEQ ID NO:65.

In some embodiments, the humanized antibody comprises a VH region that includes VH FR1 having an amino acid sequence of SEQ ID NO:62. In some embodiments, the humanized antibody comprises a VH region that includes VH FR1 having an amino acid sequence of SEQ ID NO:63. In some embodiments, the humanized antibody comprises a VH region that includes VH FR1 having an amino acid sequence of SEQ ID NO:64. In some embodiments, the humanized antibody comprises a VH region that includes VH FR1 having an amino acid sequence of SEQ ID NO:65.

In certain embodiments, an antibody or fragment thereof described herein comprises a VL region that comprises: (1) a VL FR1 having an amino acid sequence of SEQ ID NO:54; (2) a VL FR2 having an amino acid sequence of SEQ ID NO:55; (3) a VL FR3 having an amino acid sequence of SEQ ID NO:56; and/or (4) a VL FR4 having an amino acid sequence of SEQ ID NO:57

In certain embodiments, the humanized antibody comprises a VL region that includes a VL FR1 having an amino acid sequence of SEQ ID NO:54. In certain embodiments, the humanized antibody comprises a VL region that includes a VL FR1 having an amino acid sequence of SEQ ID NO:55. In certain embodiments, the humanized antibody comprises a VL region that includes a VL FR1 having an amino acid sequence of SEQ ID NO:56. In certain embodiments, the humanized antibody comprises a VL region that includes a VL FR1 having an amino acid sequence of SEQ ID NO:57.

In certain embodiments, an antibody or fragment thereof described herein comprises a VH region and a VL region, wherein the VH region comprises: (1) a VH FR1 having an amino acid sequence of SEQ ID NO:62; (2) a VH FR2 having an amino acid sequence of SEQ ID NO:63; (3) a VH FR3 having an amino acid sequence of SEQ ID NO:64; and/or (4) a VH FR4 having an amino acid sequence of SEQ ID NO:65; and wherein the VL region comprises: (1) a VL FR1 having an amino acid sequence of SEQ ID NO:54; (2) a VL FR2 having an amino acid sequence of SEQ ID NO:55; (3) a VL FR3 having an amino acid sequence of SEQ ID NO:56; and/or (4) a VL FR4 having an amino acid sequence of SEQ ID NO:57. In some embodiments, the antibody comprises a VH region comprising all four of the above-referenced VH FR1, VH FR2, VH FR3, and VH FR4. In other embodiments, the antibody comprises a VL region comprising all four of the above-referenced VL FR1, VL FR2, VL FR3, and VL FR4. In yet other embodiments, the antibody comprises a VH region comprising all four of the above-referenced VH FR1, VH FR2, VH FR3, and VH FR4, and a VL region comprising all four of the above-referenced VL FR1, VL FR2, VL FR3, and VL FR4.

In some embodiments, the antibody comprises any combination thereof of the VH FRs (SEQ ID NOS:15-18 and 58-65) and the VL FRs (SEQ ID NOS:11-14 and 50-57) listed in Tables 7 and 8. In some embodiments, the antibody comprises any combination thereof of the VH FRs (SEQ ID NOS:15-18) listed in Table 8 and the VL FRs (SEQ ID NOS: 11-14) listed in Table 7. In some embodiments, the antibody comprises any combination thereof of the VH FRs (SEQ ID NOS:58-61) listed in Table 8 and the VL FRs (SEQ ID NOS:50-53) listed in Table 7. In some embodiments, the antibody comprises any combination thereof of the VH FRs (SEQ ID NOS:62-65) listed in Table 8 and the VL FRs (SEQ ID NOS:54-57) listed in Table 7.

In some embodiments, the antibodies provided herein comprise a VH region or VH domain. In other embodiments, the antibodies provided herein comprise a VL region or VL domain. In certain embodiments, the antibodies provided herein have a combination of (i) a VH domain or VH region; and/or (ii) a VL domain or VL region. The exemplary amino acid sequences for the VL regions and the VH regions of BTN1A1 antibodies STCH11-1, STC43G3-1, and STC85F1-1 are shown in Tables 9 and 10

TABLE 9
VL Domain Amino Acid and Coding Nucleic Acid Sequences

| Antibody | VL (SEQ ID NO:) |
|---|---|
| STC43H11-1 | QVLTQTASPVSAAVGGTVTINCQASQSVYNNNRCSWFQQKPGQPPKRLI |
| VL | YHTSTLDSGVPSRFKGSGSGTQFTLTISDVQCDDAATYYCLGNYDCSRA |
| | DCAAFGGGTEVVVK (SEQ ID NO: 19) |
| STC43G3-1 | DPVMTQTPSSTSAAVGGTVTINCQSSQSVYNNNNLSWFQQKPGQPPKLL |
| VL without | IYRASKLPSGVPPRFSGSGSGTQFTLTISSVQCDDAATYFCAGGYSGDINV |
| signal | FGGGTEVVVK (SEQ ID NO: 66) |
| peptide | |
| STC43G3-1 | MDTRAPTQLLGLLLLWLPGAICDPVMTQTPSSTSAAVGGTVTINCQSSQ |
| VL with a | SVYNNNNLSWFQQKPGQPPKLLIYRASKLPSGVPPRFSGSGSGTQFTLTIS |
| signal | SVQCDDAATYFCAGGYSGDINVFGGGTEVVVK (SEQ ID NO: 135) |
| peptide | |
| STC85F1-1 | DPVMTQTPSSTSAAVGGTVTINCQSSQSVYNNKNLSWFQQKPGQPPKLL |
| VL without | IYRASTLPSGVPSRFKGSGSGTQFTLTISDLECDDAATYYCAGGYSGDIN |
| signal | VFGGGTEVVVK (SEQ ID NO: 67) |
| peptide | |
| STC85F1-1 | MDTRAPTQLLGLLLLWLPGAICDPVMTQTPSSTSAAVGGTVTINCQSSQ |
| VL with a | SVYNNKNLSWFQQKPGQPPKLLIYRASTLPSGVPSRFKGSGSGTQFTLTI |
| signal | SDLECDDAATYYCAGGYSGDINVFGGGTEVVVK (SEQ ID NO: 137) |
| peptide | |

TABLE 10
VH Domain Amino Acid Sequences

| Antibody | VH (SEQ ID NO:) |
|---|---|
| STC43H11-1 | QSLEESGGRLVTPGGSLTLTCTVSGIDLSSYAMGWVRQAPGEGLEWIGSI |
| VH | GTGGGTGYASWAKGRFTISKTSTTVDLKMTSLTASDTATYFCATSNLW |
| | GPGTLVTVSS (SEQ ID NO: 20) |
| STC43G3-1 | QSLEESGGRLVTPGTPLTLTCTVSGIDLSRYGVNWVRQAPGKGLEWIGYI |
| VH without | GTTGNTYYASWVNGRFTISKTSTTVELKMTSLTAADTATYFCARSVVSP |
| signal | SNSSFWGQGTLVTVSS (SEQ ID NO: 68) |
| peptide | |
| STC43G3-1 | METGLRWLLLVAVLKGVQCQSLEESGGRLVTPGTPLTLTCTVSGIDLSR |
| VH with a | YGVNWVRQAPGKGLEWIGYIGTTGNTYYASWVNGRFTISKTSTTVELK |
| signal | MTSLTAADTATYFCARSVVSPSNSSFWGQGTLVTVSS (SEQ ID NO: 134) |
| peptide | |
| STC85F1-1 | QSLEESGGRLVTPGGSLTLTCTVSGIDLSRYGVNWVRQAPGKGLEWIGY |
| VH without | IGTTSNTYYASWAKGRFTISKTSSTTVDLKMTSLTTEDTATYFCARSVVS |
| signal | PSNSSFWGQGTLVTVSS (SEQ ID NO: 69) |
| peptide | |
| STC85F1-1 | METGLRWLLLVAVLKGVQCQSLEESGGRLVTPGGSLTLTCTVSGIDLSR |
| VH with a | YGVNWVRQAPGKGLEWIGYIGTTSNTYYASWAKGRFTISKTSSTTVDL |
| signal | KMTSLTTEDTATYFCARSVVSPSNSSFWGQGTLVTVSS (SEQ ID NO: 136) |
| peptide | |

In yet other embodiments, the antibodies provided herein have a combination of (i) a VH domain or VH region selected from the group consisting SEQ ID NOS:20, 68, 134, 69, and 136 as set forth in Table 10; and/or (ii) a VL domain or VL region selected from the group consisting of SEQ ID NOS: 19, 66, 135, 67, and 137 as set forth in Table 9.

In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:19. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO: 135. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:67. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:137. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:20. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:68. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:134. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:69. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:136.

In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region as set forth in Table 9. In some embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region as set forth in Table 9. In some embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region as set forth in Table 9. In some embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region as set forth in Table 9.

In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region that has an amino acid sequence of SEQ ID NO:19.

In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region that has an amino acid sequence of SEQ ID NO:66.

In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region that has an amino acid sequence of SEQ ID NO:135.

In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region that has an amino acid sequence of SEQ ID NO:67.

In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:8; (2) a VH CDR2 having an amino acid sequence of SEQ ID NO:9; and (3) a VH CDR3 having an amino acid sequence of SEQ ID NO:10; and a VL region that has an amino acid sequence of SEQ ID NO:137.

In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region that has an amino acid sequence of SEQ ID NO:19.

In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region that has an amino acid sequence of SEQ ID NO:66.

In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region that has an amino acid sequence of SEQ ID NO:135.

In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region that has an amino acid sequence of SEQ ID NO:67.

In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:71; (2) a VH CDR2 having an amino acid sequence of SEQ ID NO:72; and (3) a VH CDR3 having an amino acid sequence of SEQ ID NO:73; and a VL region that has an amino acid sequence of SEQ ID NO:137.

In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region that has an amino acid sequence of SEQ ID NO:19.

In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region that has an amino acid sequence of SEQ ID NO:66.

In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region that has an amino acid sequence of SEQ ID NO:135.

In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region that has an amino acid sequence of SEQ ID NO:67.

In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:77; (2) a VH CDR2 having an amino acid sequence of SEQ ID NO:78; and (3) a VH CDR3 having an amino acid sequence of SEQ ID NO:79; and a VL region that has an amino acid sequence of SEQ ID NO:137.

In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region that has an amino acid sequence of SEQ ID NO:19.

In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and a VL region that has an amino acid sequence of SEQ ID NO:66.

In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region that has an amino acid sequence of SEQ ID NO:135.

In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region that has an amino acid sequence of SEQ ID NO:67.

In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:83; (2) a VH CDR2 having an amino acid sequence of SEQ ID NO:84; and (3) a VH CDR3 having an amino acid sequence of SEQ ID NO:85; and a VL region that has an amino acid sequence of SEQ ID NO:137.

In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region as set forth in Table 9. In some embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region as set forth in Table 9. In some embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region as set forth in Table 9. In some embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ ID NO:105; and a VL region as set forth in Table 9.

In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region that has an amino acid sequence of SEQ ID NO:19.

In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region that has an amino acid sequence of SEQ ID NO:66.

In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region that has an amino acid sequence of SEQ ID NO:135.

In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region that has an amino acid sequence of SEQ ID NO:67.

In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:44; (2) a VH CDR2 having an amino acid sequence of SEQ ID NO:45; and (3) a VH CDR3 having an amino acid sequence of SEQ ID NO:46; and a VL region that has an amino acid sequence of SEQ ID NO:137.

In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region that has an amino acid sequence of SEQ ID NO:19.

In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region that has an amino acid sequence of SEQ ID NO:66.

In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region that has an amino acid sequence of SEQ ID NO:135.

In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region that has an amino acid sequence of SEQ ID NO:67.

In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:91; (2) a VH CDR2 having an amino acid sequence of SEQ ID NO:92; and (3) a VH CDR3 having an amino acid sequence of SEQ ID NO:93; and a VL region that has an amino acid sequence of SEQ ID NO:137.

In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region that has an amino acid sequence of SEQ ID NO:19.

In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region that has an amino acid sequence of SEQ ID NO:66.

In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region that has an amino acid sequence of SEQ ID NO:135.

In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region that has an amino acid sequence of SEQ ID NO:67.

In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:97; (2) a VH CDR2 having an amino acid sequence of SEQ ID NO:98; and (3) a VH CDR3 having an amino acid sequence of SEQ ID NO:99; and a VL region that has an amino acid sequence of SEQ ID NO:137.

In certain embodiments, the antibodies provided herein comprise a VH region comprising: (1) a VH CDR1 having an amino acid sequence of SEQ ID NO:103; (2) a VH CDR2 having an amino acid sequence of SEQ ID NO:104; and (3) a VH CDR3 having an amino acid sequence of SEQ ID NO:105; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ ID NO:105; and a VL region that has an amino acid sequence of SEQ ID NO:19.

In certain embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ ID NO:105; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ ID NO:105; and a VL region that has an amino acid sequence of SEQ ID NO:66.

In certain embodiments, the antibodies provided herein comprise a VH region comprising: (1) a VH CDR1 having an amino acid sequence of SEQ ID NO:103; (2) a VH CDR2 having an amino acid sequence of SEQ ID NO: 104; and (3) a VH CDR3 having an amino acid sequence of SEQ ID NO:105; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ TD NO:105; and a VL region that has an amino acid sequence of SEQ ID NO:135.

In certain embodiments, the antibodies provided herein comprise a VH region comprising: (1) a VH CDR1 having an amino acid sequence of SEQ ID NO:103; (2) a VH CDR2 having an amino acid sequence of SEQ ID NO:104; and (3) a VH CDR3 having an amino acid sequence of SEQ ID NO: 105; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ ID NO:105; and a VL region that has an amino acid sequence of SEQ ID NO:67.

In certain embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ ID NO:105; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO: 103; (2) a VH CDR2 having an amino acid sequence of SEQ ID NO:104; and (3) a VH CDR3 having an amino acid sequence of SEQ ID NO:105; and a VL region that has an amino acid sequence of SEQ ID NO:137.

In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region as set forth in Table 9. In some embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region as set forth in Table 9. In some embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ ID NO:119; and a VL region as set forth in Table 9. In some embodiments, the antibodies provided herein comprise a VH region comprising: (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ ID NO:125; and a VL region as set forth in Table 9.

In certain embodiments, the antibodies provided herein comprise a VH region comprising (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region that has an amino acid sequence of SEQ ID NO:19.

In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region that has an amino acid sequence of SEQ ID NO:66.

In certain embodiments, the antibodies provided herein comprise a VH region comprising (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region that has an amino acid sequence of SEQ ID NO:135.

In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region that has an amino acid sequence of SEQ ID NO:67.

In certain embodiments, the antibodies provided herein comprise a VH region comprising (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:47; (2) a VH CDR2 having an amino acid sequence of SEQ ID NO:48; and (3) a VH CDR3 having an amino acid sequence of SEQ ID NO:49; and a VL region that has an amino acid sequence of SEQ ID NO:137.

In certain embodiments, the antibodies provided herein comprise a VH region comprising: (1) a VH CDR1 having an amino acid sequence of SEQ ID NO:111; (2) a VH CDR2 having an amino acid sequence of SEQ ID NO: 112; and (3) a VH CDR3 having an amino acid sequence of SEQ ID NO:113; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region that has an amino acid sequence of SEQ ID NO:19.

In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region that has an amino acid sequence of SEQ ID NO:66.

In certain embodiments, the antibodies provided herein comprise a VH region comprising: (1) a VH CDR1 having an amino acid sequence of SEQ ID NO:111; (2) a VH CDR2 having an amino acid sequence of SEQ ID NO: 112; and (3) a VH CDR3 having an amino acid sequence of SEQ ID NO:113; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region that has an amino acid sequence of SEQ ID NO:135.

In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO: 111; (2) a VH CDR2 having an amino acid sequence of SEQ ID NO:112; and (3) a VH CDR3 having an amino acid sequence of SEQ ID NO: 113; and a VL region that has an amino acid sequence of SEQ ID NO:67.

In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO:111; (2) a VH CDR2 having an amino acid sequence of SEQ ID NO:112; and (3) a VH CDR3 having an amino acid sequence of SEQ ID NO:113; and a VL region that has an amino acid sequence of SEQ ID NO:137.

In certain embodiments, the antibodies provided herein comprise a VH region comprising: (1) a VH CDR1 having an amino acid sequence of SEQ ID NO:117; (2) a VH CDR2 having an amino acid sequence of SEQ ID NO:118; and (3) a VH CDR3 having an amino acid sequence of SEQ ID NO:119; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ ID NO:119; and a VL region that has an amino acid sequence of SEQ ID NO:19.

In certain embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ ID NO:119; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ TD NO: 119; and a VL region that has an amino acid sequence of SEQ ID NO:66.

In certain embodiments, the antibodies provided herein comprise a VH region comprising: (1) a VH CDR1 having an amino acid sequence of SEQ ID NO:117; (2) a VH CDR2 having an amino acid sequence of SEQ ID NO:118; and (3) a VH CDR3 having an amino acid sequence of SEQ ID NO:119; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ ID NO:119; and a VL region that has an amino acid sequence of SEQ ID NO:135.

In certain embodiments, the antibodies provided herein comprise a VH region comprising: ((1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ ID NO:119; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ ID NO: 119; and a VL region that has an amino acid sequence of SEQ ID NO:67.

In certain embodiments, the antibodies provided herein comprise a VH region comprising: (1) a VH CDR1 having an amino acid sequence of SEQ ID NO:117; (2) a VH CDR2 having an amino acid sequence of SEQ ID NO: 118; and (3) a VH CDR3 having an amino acid sequence of SEQ ID NO:119; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO: 117; (2) a VH CDR2 having an amino acid sequence of SEQ ID NO:118; and (3) a VH CDR3 having an amino acid sequence of SEQ TD NO:119; and a VL region that has an amino acid sequence of SEQ ID NO: 137.

In certain embodiments, the antibodies provided herein comprise a VH region comprising: (1) a VH CDR1 having an amino acid sequence of SEQ ID NO:123; (2) a VH CDR2 having an amino acid sequence of SEQ ID NO:124; and (3) a VH CDR3 having an amino acid sequence of SEQ ID NO:125; and a VL region having an amino acid sequence having at least about 75%, at least about 80%, at least about 85%, at least about 90%, at least about 95%, at least about 97%, or at least about 99% sequence identity to SEQ ID NO:19. In certain embodiments, the antibodies provided herein comprise a VH region comprising: (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ ID NO:125; and a VL region that has an amino acid sequence of SEQ ID NO:19.

In certain embodiments, the antibodies provided herein comprise a VH region comprising: (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ ID NO:125; and a VL region having an amino acid sequence having at least about 75%, at least about 80%, at least about 85%, at least about 90%, at least about 95%, at least about 97%, or at least about 99% sequence identity to SEQ ID NO:66. In certain embodiments, the antibodies provided herein comprise a VH region comprising: (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ ID NO:125; and a VL region that has an amino acid sequence of SEQ ID NO:66.

In certain embodiments, the antibodies provided herein comprise a VH region comprising: (1) a VH CDR1 having an amino acid sequence of SEQ ID NO:123; (2) a VH CDR2 having an amino acid sequence of SEQ ID NO: 124; and (3) a VH CDR3 having an amino acid sequence of SEQ ID NO:125; and a VL region having an amino acid sequence having at least about 75%, at least about 80%, at least about 85%, at least about 90%, at least about 95%, at least about 97%, or at least about 99% sequence identity to SEQ ID NO:135. In certain embodiments, the antibodies provided herein comprise a VH region comprising: (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ TD NO:125; and a VL region that has an amino acid sequence of SEQ ID NO:135.

In certain embodiments, the antibodies provided herein comprise a VH region comprising: (1) a VH CDR1 having an amino acid sequence of SEQ ID NO:123; (2) a VH CDR2 having an amino acid sequence of SEQ ID NO:124; and (3) a VH CDR3 having an amino acid sequence of SEQ ID NO:125; and a VL region having an amino acid sequence having at least about 75%, at least about 80%, at least about 85%, at least about 90%, at least about 95%, at least about 97%, or at least about 99% sequence identity to SEQ ID NO:67. In certain embodiments, the antibodies provided herein comprise a VH region comprising (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ ID NO:125; and a VL region that has an amino acid sequence of SEQ ID NO:67.

In certain embodiments, the antibodies provided herein comprise a VH region comprising: (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ ID NO:125; and a VL region having an amino acid sequence having at least about 75%, at least about 80%, at least about 85%, at least about 90%, at least about 95%, at least about 97%, or at least about 99% sequence identity to SEQ ID NO:137. In certain embodiments, the antibodies provided herein comprise a VH region comprising: (1) a VH CDR1 having an amino acid sequence of SEQ ID NO: 123; (2) a VH CDR2 having an amino acid sequence of SEQ ID NO:124; and (3) a VH CDR3 having an amino acid sequence of SEQ ID NO:125; and a VL region that has an amino acid sequence of SEQ ID NO:137.

In other embodiments, the antibodies provided herein comprise a VH as set forth in Table 10; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7. In some embodiments, the antibodies provided herein comprise a VH consisting as set forth in Table 10; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76. In some embodiments, the antibodies provided herein comprise a VH as set forth in Table 10; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82. In some embodiments, the antibodies provided herein comprise a VH as set forth in Table 10; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7. In certain embodiments, the antibodies provided herein comprise a VH region having an amino acid sequence of SEQ ID NO:134; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7. In certain embodiments, the antibodies provided herein comprise a VH region having an amino acid sequence of SEQ ID NO:136; and a VL region comprising: (1) a VL CDR1 having an amino acid sequence selected from the group consisting of SEQ ID NO:5; (2) a VL CDR2 having an amino acid sequence of SEQ ID NO:6; and (3) a VL CDR3 having an amino acid sequence of SEQ ID NO:7.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76. In certain embodiments, the antibodies provided herein comprise a VH region having an amino acid sequence of SEQ ID NO:134; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76. In certain embodiments, the antibodies provided herein comprise a VH region having an amino acid sequence of SEQ ID NO:136; and a VL region comprising: (1) a VL CDR1 having an amino acid sequence selected from the group consisting of SEQ ID NO:74; (2) a VL CDR2 having an amino acid sequence of SEQ ID NO:75; and (3) a VL CDR3 having an amino acid sequence of SEQ ID NO:76.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82. In certain embodiments, the antibodies provided herein comprise a VH region having an amino acid sequence of SEQ ID NO:20; and a VL region comprising: ((1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82. In certain embodiments, the antibodies provided herein comprise a VH region having an amino acid sequence of SEQ ID NO:134; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82. In certain embodiments, the antibodies provided herein comprise a VH region having an amino acid sequence of SEQ ID NO:136; and a VL region comprising: ((1) a VL CDR1 having an amino acid sequence selected from the group consisting of SEQ ID NO:80; (2) a VL CDR2 having an amino acid sequence of SEQ ID NO:81; and (3) a VL CDR3 having an amino acid sequence of SEQ ID NO:82.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:68; and a VL region comprising: ((1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:134; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:136; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:68; and a VL region comprising: ((1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:134; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88. In certain embodiments, the antibodies provided herein comprise a VH region having an amino acid sequence of SEQ ID NO:136; and a VL region comprising: (1) a VL CDR1 having an amino acid sequence selected from the group consisting of SEQ ID NO:86; (2) a VL CDR2 having an amino acid sequence of SEQ ID NO:87; and (3) a VL CDR3 having an amino acid sequence of SEQ ID NO:88.

In other embodiments, the antibodies provided herein comprise a VH as set forth in Table 10; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40. In some embodiments, the antibodies provided herein comprise a VH consisting as set forth in Table 10; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96. In some embodiments, the antibodies provided herein comprise a VH as set forth in Table 10; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102. In some embodiments, the antibodies provided herein comprise a VH as set forth in Table 10; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40. In certain embodiments, the antibodies provided herein comprise a VH region having an amino acid sequence of SEQ ID NO:134; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40. In certain embodiments, the antibodies provided herein comprise a VH region having an amino acid sequence of SEQ ID NO:136; and a VL region comprising: (1) a VL CDR1 having an amino acid sequence selected from the group consisting of SEQ ID NO:38; (2) a VL CDR2 having an amino acid sequence of SEQ ID NO:39; and (3) a VL CDR3 having an amino acid sequence of SEQ ID NO:40.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96. In certain embodiments, the antibodies provided herein comprise a VH region having an amino acid sequence of SEQ ID NO:134; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96. In certain embodiments, the antibodies provided herein comprise a VH region having an amino acid sequence of SEQ ID NO:136; and a VL region comprising: (1) a VL CDR1 having an amino acid sequence selected from the group consisting of SEQ ID NO:94; (2) a VL CDR2 having an amino acid sequence of SEQ ID NO:95; and (3) a VL CDR3 having an amino acid sequence of SEQ ID NO:96.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102. In certain embodiments, the antibodies provided herein comprise a VH region having an amino acid sequence of SEQ ID NO: 134; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102. In certain embodiments, the antibodies provided herein comprise a VH region having an amino acid sequence of SEQ ID NO: 136; and a VL region comprising: (1) a VL CDR1 having an amino acid sequence selected from the group consisting of SEQ ID NO:100; (2) a VL CDR2 having an amino acid sequence of SEQ ID NO:101; and (3) a VL CDR3 having an amino acid sequence of SEQ ID NO:102.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108. In certain embodiments, the antibodies provided herein comprise a VH region having an amino acid sequence of SEQ ID NO: 134; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108. In certain embodiments, the antibodies provided herein comprise a VH region having an amino acid sequence of SEQ ID NO: 136; and a VL region comprising: (1) a VL CDR1 having an amino acid sequence selected from the group consisting of SEQ ID NO:106; (2) a VL CDR2 having an amino acid sequence of SEQ ID NO:107; and (3) a VL CDR3 having an amino acid sequence of SEQ ID NO:108.

In other embodiments, the antibodies provided herein comprise a VH as set forth in Table 10; and a VL region comprising: (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43. In some embodiments, the antibodies provided herein comprise a VH consisting as set forth in Table 10; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116. In some embodiments, the antibodies provided herein comprise a VH as set forth in Table 10; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122. In some embodiments, the antibodies provided herein comprise a VH as set forth in Table 10; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43. In certain embodiments, the antibodies provided herein comprise a VH region having an amino acid sequence of SEQ ID NO:20; and a VL region comprising: ((1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43. In certain embodiments, the antibodies provided herein comprise a VH region having an amino acid sequence of SEQ ID NO:134; and a VL region comprising: ((1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43. In certain embodiments, the antibodies provided herein comprise a VH region having an amino acid sequence of SEQ ID NO:69; and a VL region comprising (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43. In certain embodiments, the antibodies provided herein comprise a VH region having an amino acid sequence of SEQ ID NO:136; and a VL region comprising: ((1) a VL CDR1 having an amino acid sequence selected from the group consisting of SEQ ID NO:41; (2) a VL CDR2 having an amino acid sequence of SEQ ID NO:42; and (3) a VL CDR3 having an amino acid sequence of SEQ ID NO:43.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116. In certain embodiments, the antibodies provided herein comprise a VH region having an amino acid sequence of SEQ ID NO: 134; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:114; (2) a VL CDR2 having an amino acid sequence of SEQ ID NO: 115; and (3) a VL CDR3 having an amino acid sequence of SEQ ID NO:116.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116. In certain embodiments, the antibodies provided herein comprise a VH region having an amino acid sequence of SEQ ID NO: 136; and a VL region comprising: (1) a VL CDR1 having an amino acid sequence selected from the group consisting of SEQ ID NO:114; (2) a VL CDR2 having an amino acid sequence of SEQ ID NO:115; and (3) a VL CDR3 having an amino acid sequence of SEQ ID NO:116.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122. In certain embodiments, the antibodies provided herein comprise a VH region having an amino acid sequence of SEQ ID NO: 134; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122. In certain embodiments, the antibodies provided herein comprise a VH region having an amino acid sequence of SEQ ID NO: 136; and a VL region comprising: (1) a VL CDR1 having an amino acid sequence selected from the group consisting of SEQ ID NO:120; (2) a VL CDR2 having an amino acid sequence of SEQ ID NO:121; and (3) a VL CDR3 having an amino acid sequence of SEQ ID NO:122.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:20; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128. In certain embodiments, the antibodies provided herein comprise a VH region having an amino acid sequence of SEQ ID NO:20; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:68; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128. In certain embodiments, the antibodies provided herein comprise a VH region having an amino acid sequence of SEQ ID NO:68; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:134; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128. In certain embodiments, the antibodies provided herein comprise a VH region having an amino acid sequence of SEQ ID NO: 134; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:69; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128. In certain embodiments, the antibodies provided herein comprise a VH region having an amino acid sequence of SEQ ID NO:69; and a VL region comprising: (1) a VL CDR1 having an amino acid sequence selected from the group consisting of SEQ ID NO:126; (2) a VL CDR2 having an amino acid sequence of SEQ ID NO: 127; and (3) a VL CDR3 having an amino acid sequence of SEQ ID NO:128.

In certain embodiments, the antibodies provided herein comprise a VH region having an amino acid sequence having at least about 75%, at least about 80%, at least about 85%, at least about 90%, at least about 95%, at least about 97%, or at least about 99% sequence identity to SEQ ID NO:136; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128. In certain embodiments, the antibodies provided herein comprise a VH region having an amino acid sequence of SEQ ID NO: 136; and a VL region comprising: (1) a VL CDR1 having an amino acid sequence selected from the group consisting of SEQ ID NO:126; (2) a VL CDR2 having an amino acid sequence of SEQ ID NO:127; and (3) a VL CDR3 having an amino acid sequence of SEQ ID NO:128.

In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:19; and a VH region has an amino acid sequence having at least about 75%, at least about 80%, at least about 85%, at least about 90%, at least about 95%, at least about 97%, or at least about 99% sequence identity to SEQ ID NO:20. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:19; and a VH region has an amino acid sequence having at least about 75%, at least about 80%, at least about 85%, at least about 90%, at least about 95%, at least about 97%, or at least about 99% sequence identity to SEQ ID NO:68. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:19; and a VH region has an amino acid sequence having at least about 75%, at least about 80%, at least about 85%, at least about 90%, at least about 95%, at least about 97%, or at least about 99% sequence identity to SEQ ID NO:134. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:19; and a VH region has an amino acid sequence having at least about 75%, at least about 80%, at least about 85%, at least about 90%, at least about 95%, at least about 97%, or at least about 99% sequence identity to SEQ ID NO:69. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:19; and a VH region has an amino acid sequence having at least about 75%, at least about 80%, at least about 85%, at least about 90%, at least about 95%, at least about 97%, or at least about 99% sequence identity to SEQ ID NO:136.

In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66; and a VH region has an amino acid sequence having at least about 75%, at least about 80%, at least about 85%, at least about 90%, at least about 95%, at least about 97%, or at least about 99% sequence identity to SEQ ID NO:20. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66; and a VH region has an amino acid sequence having at least about 75%, at least about 80%, at least about 85%, at least about 90%, at least about 95%, at least about 97%, or at least about 99% sequence identity to SEQ ID NO:68. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66; and a VH region has an amino acid sequence having at least about 75%, at least about 80%, at least about 85%, at least about 90%, at least about 95%, at least about 97%, or at least about 99% sequence identity to SEQ ID NO:134. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66; and a VH region has an amino acid sequence having at least about 75%, at least about 80%, at least about 85%, at least about 90%, at least about 95%, at least about 97%, or at least about 99% sequence identity to SEQ ID NO:69. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66; and a VH region has an amino acid sequence having at least about 75%, at least about 80%, at least about 85%, at least about 90%, at least about 95%, at least about 97%, or at least about 99% sequence identity to SEQ ID NO:136.

In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:135; and a VH region has an amino acid sequence having at least about 75%, at least about 80%, at least about 85%, at least about 90%, at least about 95%, at least about 97%, or at least about 99% sequence identity to SEQ ID NO:20. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:135; and a VH region has an amino acid sequence having at least about 75%, at least about 80%, at least about 85%, at least about 90%, at least about 95%, at least about 97%, or at least about 99% sequence identity to SEQ ID NO:68. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:135; and a VH region has an amino acid sequence having at least about 75%, at least about 80%, at least about 85%, at least about 90%, at least about 95%, at least about 97%, or at least about 99% sequence identity to SEQ ID NO:134. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:135; and a VH region has an amino acid sequence having at least about 75%, at least about 80%, at least about 85%, at least about 90%, at least about 95%, at least about 97%, or at least about 99% sequence identity to SEQ ID NO:69. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:135; and a VH region has an amino acid sequence having at least about 75%, at least about 80%, at least about 85%, at least about 90%, at least about 95%, at least about 97%, or at least about 99% sequence identity to SEQ ID NO:136.

In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:67; and a VH region has an amino acid sequence having at least about 75%, at least about 80%, at least about 85%, at least about 90%, at least about 95%, at least about 97%, or at least about 99% sequence identity to SEQ ID NO:20. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:67; and a VH region has an amino acid sequence having at least about 75%, at least about 80%, at least about 85%, at least about 90%, at least about 95%, at least about 97%, or at least about 99% sequence identity to SEQ ID NO:68. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:67; and a VH region has an amino acid sequence having at least about 75%, at least about 80%, at least about 85%, at least about 90%, at least about 95%, at least about 97%, or at least about 99% sequence identity to SEQ ID NO:134. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:67; and a VH region has an amino acid sequence having at least about 75%, at least about 80%, at least about 85%, at least about 90%, at least about 95%, at least about 97%, or at least about 99% sequence identity to SEQ ID NO:69. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:67; and a VH region has an amino acid sequence having at least about 75%, at least about 80%, at least about 85%, at least about 90%, at least about 95%, at least about 97%, or at least about 99% sequence identity to SEQ ID NO:136.

In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:137; and a VH region has an amino acid sequence having at least about 75%, at least about 80%, at least about 85%, at least about 90%, at least about 95%, at least about 97%, or at least about 99% sequence identity to SEQ ID NO:20. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:137; and a VH region has an amino acid sequence having at least about 75%, at least about 80%, at least about 85%, at least about 90%, at least about 95%, at least about 97%, or at least about 99% sequence identity to SEQ ID NO:68. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:137; and a VH region has an amino acid sequence having at least about 75%, at least about 80%, at least about 85%, at least about 90%, at least about 95%, at least about 97%, or at least about 99% sequence identity to SEQ ID NO:134. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:137; and a VH region has an amino acid sequence having at least about 75%, at least about 80%, at least about 85%, at least about 90%, at least about 95%, at least about 97%, or at least about 99% sequence identity to SEQ ID NO:69. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:137; and a VH region has an amino acid sequence having at least about 75%, at least about 80%, at least about 85%, at least about 90%, at least about 95%, at least about 97%, or at least about 99% sequence identity to SEQ ID NO:136.

In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:19; and a VH set forth in Table 10. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66; and a VH set forth in Table 10. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:135; and a VH set forth in Table 10. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:66; and a VH set forth in Table 10. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:67; and a VH set forth in Table 10. In some embodiments, the antibodies provided herein comprise a VL region has an amino acid sequence having at least about 75%, at least about 80%, at least about 85%, at least about 90%, at least about 95%, at least about 97%, or at least about 99% sequence identity to SEQ ID NO:137; and a VH set forth in Table 10.

In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:20; and a VL set forth in Table 9. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:68; and a VL set forth in Table 9. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:134; and a VL set forth in Table 9. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:69; and a VL set forth in Table 9. In some embodiments, the antibodies provided herein comprise a VH region has an amino acid sequence having at least about 75%, at least about 80%, at least about 85%, at least about 90%, at least about 95%, at least about 97%, or at least about 99% sequence identity to SEQ ID NO:20; and a VL set forth in Table 9. In some embodiments, the antibodies provided herein comprise a VL region has an amino acid sequence having at least about 95% sequence identity to SEQ ID NO:19 and a VH region has an amino acid sequence having at least about 95% sequence identity to SEQ ID NO:136. In some embodiments, an antibody provided herein has a VL and a VH amino acid sequence of BTN1A1. In some embodiments, an antibody comprises a VL amino acid sequence of SEQ ID NO:19, and a VH amino acid sequence of SEQ ID NO:20.

In some embodiments, the antibodies provided herein comprise a VL region has an amino acid sequence having at least about 95% sequence identity to SEQ ID NO:66 and a VH region has an amino acid sequence having at least about 95% sequence identity to SEQ ID NO:68. In some embodiments, an antibody provided herein has a VL and a VH amino acid sequence of BTN1A1. In some embodiments, an antibody comprises a VH amino acid sequence of SEQ ID NO:66, and a VL amino acid sequence of SEQ ID NO:68.

In some embodiments, the antibodies provided herein comprise a VL region has an amino acid sequence having at least about 95% sequence identity to SEQ ID NO:135 and a VH region has an amino acid sequence having at least about 95% sequence identity to SEQ ID NO:134. In some embodiments, an antibody provided herein has a VL and a VH amino acid sequence of BTN1A1. In some embodiments, an antibody comprises a VH amino acid sequence of SEQ ID NO:135, and a VL amino acid sequence of SEQ ID NO:134.

In some embodiments, the antibodies provided herein comprise a VL region has an amino acid sequence having at least about 95% sequence identity to SEQ ID NO:67 and a VH region has an amino acid sequence having at least about 95% sequence identity to SEQ ID NO:69. In some embodiments, an antibody provided herein has a VL and a VH amino acid sequence of BTN1A1. In some embodiments, an antibody comprises a VH amino acid sequence of SEQ ID NO:67, and a VL amino acid sequence of SEQ ID NO:69.

In some embodiments, the antibodies provided herein comprise a VL region has an amino acid sequence having at least about 95% sequence identity to SEQ ID NO: 137 and a VH region has an amino acid sequence having at least about 950% sequence identity to SEQ ID NO:136. In some embodiments, an antibody provided herein has a VL and a VH amino acid sequence of BTN1A1. In some embodiments, an antibody comprises a VH amino acid sequence of SEQ ID NO:137, and a VL amino acid sequence of SEQ ID NO:136.

Also provided herein are isolated nucleic acid molecules encoding an immunoglobulin heavy chain, light chain, VH region, VL region, VII CDR1, VH CDR2, VII CDR3, VL CDR1, VL CDR2, and/or VL CDR3 of anti-BTN1A1 antibodies that bind to a BTN1A1 polypeptide, a BTN1A1 polypeptide fragment, a BTN1A1 peptide, or a BTN1A1 epitope. The exemplary nucleic acid sequences for the VL regions and the VH regions of BTN1A1 antibodies STCH11-1, STC43G3-1, and STC85F1-1 are shown in Tables 11 and 12.

TABLE 11
VL Coding Nucleic Acid Sequences

| Antibody | Nucleotide sequences |
|---|---|
| STC43H11-1 | CAAGTGCTGACCCAGACTGCATCCCCCGTGTCTGCGGCTGTTGGAG |
| VL | GCACAGTCACCATCAATTGCCAGGCCAGTCAGAGTGTTTATAATAA |
| | CAACCGCTGTTCCTGGTTTCAGCAGAAACCAGGGCAGCCTCCCAAG |
| | CGCCTGATCTATCATACATCGACTCTAGATTCTGGGGTCCCATCGCG |
| | GTTCAAAGGCAGTGGATCTGGGACACAGTTCACTCTCACCATCAGC |
| | GACGTGCAGTGTGACGATGCTGCCACTTACTACTGTCTAGGCAATT |
| | ATGATTGTAGTCGTGCTGATTGTGCTGCTTTCGGCGGAGGGACCGA |
| | GGTGGTGGTCAAA |
| | (SEQ ID NO: 21) |
| STC43G3-1 | GACCCTGTGATGACCCAGACTCCATCTTCCACGTCTGCGGCTGTGG |
| VL without | GAGGCACAGTCACCATCAACTGCCAGTCCAGTCAGAGTGTTTATAA |
| signal peptide | TAATAACAACTTATCCTGGTTTCAGCAGAAACCAGGTCAGCCTCCC |
| | AAGCTCCTGATCTACAGGGCATCCAAACTGCCATCTGGGGTCCCAC |
| | CGCGGTTCAGCGGCAGTGGATCTGGGACACAGTTCACTCTCACCAT |
| | CAGCAGCGTGCAGTGTGACGATGCTGCCACTTACTTCTGTGCCGGC |
| | GGTTATAGTGGTGATATTAATGTTTTCGGCGGAGGGACCGAGGTGG |
| | TGGTCAAA |
| | (SEQ ID NO: 90) |
| STC43G3-1 | ATGGACACGAGGGCCCCCACTCAGCTGCTGGGGCTCCTGCTGCTCT |
| VL with a | GGCTCCCAGGTGCCATATGTGACCCTGTGATGACCCAGACTCCATC |
| signal | TTCCACGTCTGCGGCTGTGGGAGGCACAGTCACCATCAACTGCCAG |
| peptide | TCCAGTCAGAGTGTTTATAATAATAACAACTTATCCTGGTTTCAGCA |
| | GAAACCAGGTCAGCCTCCCAAGCTCCTGATCTACAGGGCATCCAAA |
| | CTGCCATCTGGGGTCCCACCGCGGTTCAGCGGCAGTGGATCTGGGA |
| | CACAGTTCACTCTCACCATCAGCAGCGTGCAGTGTGACGATGCTGC |
| | CACTTACTTCTGTGCCGGCGGTTATAGTGGTGATATTAATGTTTTCG |
| | GCGGAGGGACCGAGGTGGTGGTCAAA |
| | (SEQ ID NO: 138) |
| STC85F1-1 | GACCCTGTGATGACCCAGACTCCATCTTCCACGTCTGCGGCTGTGG |
| VL without | GAGGCACAGTCACCATCAACTGCCAGTCCAGTCAGAGTGTTTATAA |
| signal peptide | TAACAAAAACTTATCCTGGTTTCAGCAGAAACCAGGTCAGCCTCCC |
| | AAGCTCCTGATCTACAGGGCATCCACTCTGCCATCTGGGGTCCCATC |
| | GCGGTTCAAAGGCAGTGGATCTGGGACACAGTTCACTCTCACCATC |
| | AGTGACCTGGAGTGTGACGATGCTGCCACTTACTACTGTGCCGGCG |
| | GTTATAGTGGTGATATTAATGTTTTCGGCGGAGGGACCGAGGTGGT |
| | GGTCAAA |
| | (SEQ ID NO: 110) |
| STC85F1-1 | ATGGACACGAGGGCCCCCACTCAGCTGCTGGGGCTCCTGCTGCTCT |
| VL with a | GGCTCCCAGGTGCCATATGTGACCCTGTGATGACCCAGACTCCATC |
| signal | TTCCACGTCTGCGGCTGTGGGAGGCACAGTCACCATCAACTGCCAG |
| peptide | TCCAGTCAGAGTGTTTATAATAACAAAAACTTATCCTGGTTTCAGCA |
| | GAAACCAGGTCAGCCTCCCAAGCTCCTGATCTACAGGGCATCCACT |
| | CTGCCATCTGGGGTCCCATCGCGGTTCAAAGGCAGTGGATCTGGGA |
| | CACAGTTCACTCTCACCATCAGTGACCTGGAGTGTGACGATGCTGC |
| | CACTTACTACTGTGCCGGCGGTTATAGTGGTGATATTAATGTTTTCG |
| | GCGGAGGGACCGAGGTGGTGGTCAAA |
| | (SEQ ID NO: 139) |

TABLE 12
VH Coding Nucleic Acid Sequences

| Antibody | Nucleotide sequences |
|---|---|
| STC43H11-1 | CAGTCGCTGGAGGAGTCCGGGGGTCGCCTGGTAACGCCTGGAGGAT |
| VH | CCCTGACACTCACCTGCACAGTCTCTGGAATCGACCTCAGTAGCTAT |
| | GCAATGGGCTGGGTCCGCCAGGCTCCAGGGGAGGGGCTGGAATGGA |
| | TCGGAAGCATTGGTACTGGTGGTGGCACAGGCTACGCGAGCTGGGC |
| | AAAAGGCCGATTCACCATCTCCAAAACCTCGACCACGGTGGATCTG |
| | AAAATGACCAGTCTGACAGCTTCAGACACGGCCACCTATTTCTGTGC |
| | CACCAGTAACTTGTGGGGCCCAGGCACCCTGGTCACCGTCTCCTCA |
| | (SEQ ID NO: 22) |
| STC43G3-1 | CAGTCGCTGGAGGAGTCCGGGGGTCGCCTGGTCACGCCTGGGACAC |
| VH without | CCCTGACACTCACCTGCACAGTCTCTGGAATCGACCTCAGTAGATAT |
| signal peptide | GGAGTGAACTGGGTCCGCCAGGCTCCAGGGAAGGGGCTGGAATGGA |
| | TCGGATATATTGGTACTACTGGTAATACATACTACGCGAGCTGGGTG |
| | AATGGTCGATTCACCATCTCCAAAACCTCGACCACGGTGGAGCTGAA |
| | AATGACCAGTCTGACAGCCGCGGACACGGCCACCTATTTCTGTGCCA |
| | GAAGTGTTGTTAGTCCTAGTAATAGTAGTTTCTGGGGCCAAGGCACC |
| | CTGGTCACCGTCTCCTCA |
| | (SEQ ID NO: 89) |
| STC43G3-1 | ATGGAGACTGGGCTGCGCTGGCTTCTCCTGGTCGCTGTGCTCAAAGG |
| VH with | TGTCCAGTGTCAGTCGCTGGAGGAGTCCGGGGGTCGCCTGGTCACGC |
| signal peptide | CTGGGACACCCCTGACACTCACCTGCACAGTCTCTGGAATCGACCTC |
| | AGTAGATATGGAGTGAACTGGGTCCGCCAGGCTCCAGGGAAGGGGC |
| | TGGAATGGATCGGATATATTGGTACTACTGGTAATACATACTACGCG |
| | AGCTGGGTGAATGGTCGATTCACCATCTCCAAAACCTCGACCACGGT |
| | GGAGCTGAAAATGACCAGTCTGACAGCCGCGGACACGGCCACCTAT |
| | TTCTGTGCCAGAAGTGTTGTTAGTCCTAGTAATAGTAGTTTCTGGGG |
| | CCAAGGCACCCTGGTCACCGTCTCCTCA |
| | (SEQ ID NO: 140) |
| STC85F1-1 | CAGTCGCTGGAGGAGTCCGGGGGTCGCCTGGTCACGCCTGGAGGAT |
| VH without | CCCTGACACTCACCTGCACAGTCTCTGGAATCGACCTCAGTAGATAT |
| signal peptide | GGAGTGAACTGGGTCCGCCAGGCTCCAGGGAAGGGGCTGGAATGGA |
| | TCGGATATATTGGTACTACTTCTAACACATACTACGCGAGCTGGGCG |
| | AAAGGCCGATTCACCATCTCCAAAACCTCGTCGACCACGGTGGATCT |
| | GAAAATGACCAGTCTGACAACCGAGGACACGGCCACCTATTTCTGT |
| | GCCAGAAGTGTTGTTAGTCCTAGTAATAGTAGTTTCTGGGGCCAAGG |
| | CACCCTGGTCACCGTCTCCTCA |
| | (SEQ ID NO: 109) |
| STC85F1-1 | ATGGAGACTGGGCTGCGCTGGCTTCTCCTGGTCGCTGTGCTCAAAGG |
| VH with a | TGTCCAGTGTCAGTCGCTGGAGGAGTCCGGGGGTCGCCTGGTCACGC |
| signal peptide | CTGGAGGATCCCTGACACTCACCTGCACAGTCTCTGGAATCGACCTC |
| | AGTAGATATGGAGTGAACTGGGTCCGCCAGGCTCCAGGGAAGGGGC |
| | TGGAATGGATCGGATATATTGGTACTACTTCTAACACATACTACGCG |
| | AGCTGGGCGAAAGGCCGATTCACCATCTCCAAAACCTCGTCGACCA |
| | CGGTGGATCTGAAAATGACCAGTCTGACAACCGAGGACACGGCCAC |
| | CTATTTCTGTGCCAGAAGTGTTGTTAGTCCTAGTAATAGTAGTTTCTG |
| | GGGCCAAGGCACCCTGGTCACCGTCTCCTCA |
| | (SEQ ID NO: 141) |

In some embodiments, an antibody provided herein has a VH and a VL amino acid sequence of BTN1A1. In some embodiments, an antibody comprises a VH amino acid sequence of SEQ ID NO:20, and a VL amino acid sequence of SEQ ID NO:19. In some embodiments, an antibody comprises a VH amino acid sequence of SEQ ID NO:68, and a VL amino acid sequence of SEQ ID NO:66. In some embodiments, an antibody comprises a VH amino acid sequence of SEQ ID NO:134, and a VL amino acid sequence of SEQ ID NO:135. In some embodiments, an antibody comprises a VH amino acid sequence of SEQ ID NO:69, and a VL amino acid sequence of SEQ ID NO:67. In some embodiments, an antibody comprises a VH amino acid sequence of SEQ ID NO:136, and a VL amino acid sequence of SEQ ID NO:137.

In yet another aspect, antibodies are provided that compete with one of the exemplified antibodies or functional fragments for binding to BTN1A1. Such antibodies may also bind to the same epitope as one of the herein exemplified antibodies, or an overlapping epitope. Antibodies and fragments that compete with or bind to the same epitope as the exemplified antibodies are expected to show similar functional properties. The exemplified antigen-binding proteins and fragments include those with the VH and VL regions, and CDRs provided herein, including those in Tables 1-6, 9 and 10. Thus, as a specific example, the antibodies that are provided include those that compete with an antibody comprising: (a) 1, 2, 3, 4, 5, or all 6 of the CDRs listed for an antibody listed in Tables 1-6; (b) a VH and a VL selected from the VH and the VL regions listed for an antibody listed in Tables 8 and 9; or (c) two light chains and two heavy chains comprising a VH and a VL as specified for an antibody listed in Tables 8 and 9. In some embodiments, the antibody is STC43H11-1, STC43G3-1, or STC85F1-1.

In another aspect, antibodies or antigen-binding fragments thereof provided herein bind to a region, including an epitope, of human BTN1A1, cynomolgus or mouse BTN1A1. For example, in some embodiments, an antibody provided herein binds to a region of human BTN1A1 (SEQ ID NO:1) comprising amino acid residues 361-375 of human BTN1A1. In some embodiments, an antibody provided herein binds to a region of human BTN1A1 (SEQ ID NO:1) comprising amino acid residues 67-86 of human BTN1A1. In some embodiments, an antibody provided herein binds to a region of human BTN1A1 (SEQ ID NO:1) comprising amino acid residues 179-197 of human BTN1A1.

In still another aspect, antibodies provided herein bind to a specific epitope of human BTN1A1.

In certain embodiments, the antibody or antigen-binding fragment thereof, when bound to BTN1A1, binds to at least one of residues 361-375 (SEQ ID NO:23) within an amino acid sequence of SEQ ID NO:1. In some embodiments, the antibody or antigen-binding fragment thereof, when bound to BTN1A1, binds to at least one of residues 365-372 (SEQ ID NO:33) within an amino acid sequence of SEQ ID NO:1. In some embodiments, the antibody or antigen-binding fragment thereof, when bound to BTN1A1, binds to at least one of residues 366-372 (SEQ ID NO:34) within an amino acid sequence of SEQ ID NO:1.

In some embodiments, the antibody or antigen-binding fragment thereof, when bound to BTN1A1, binds to at least one of residues 67-86 (SEQ ID NO:70) within an amino acid sequence of SEQ ID NO:1.

In particular embodiments, the antibody or antigen-binding fragment thereof, when bound to BTN1A1, binds to at least one residue selected from the group consisting of G365, D366, T368, D369, W370, A371, and I372 within an amino acid sequence of SEQ ID NO:1. In some embodiments, the antibody or antigen-binding fragment thereof, when bound to BTN1A1, binds to at least one residue selected from the group consisting of D366, D369, and I372 within an amino acid sequence of SEQ ID NO:1. In some embodiments, the antibody or antigen-binding fragment thereof, when bound to BTN1A1, binds to at least one residue selected from the group consisting of G365, T368, D369, A371, and I372 within an amino acid sequence of SEQ ID NO:1. In some embodiments, the antibody or antigen-binding fragment thereof, when bound to BTN1A1, binds to at least one residue selected from the group consisting of D366, T368, D369, W370, A371, I372, and G373 within an amino acid sequence of SEQ ID NO: 1.

In some embodiments, the antibody or antigen-binding fragment thereof, when bound to BTN1A1, binds to two or more residues selected from the group consisting of G365, D366, T368, D369, W370, A371, and I372 within an amino acid sequence of SEQ ID NO:1.

In other embodiments, the antibody or antigen-binding fragment thereof, when bound to BTN1A1, binds to three or more residues selected from the group consisting of G365, D366, T368, D369, W370, A371, and I372 within an amino acid sequence of SEQ ID NO:1.

In certain embodiments, the antibody or antigen-binding fragment thereof, when bound to BTN1A1, binds to four or more residues selected from the group consisting of G365, D366, T368, D369, W370, A371, and I372 within an amino acid sequence of SEQ ID NO:1.

In one embodiment, the antibody or antigen-binding fragment thereof, when bound to BTN1A1, binds to five or more residues selected from the group consisting of G365, D366, T368, D369, W370, A371, and I372 within an amino acid sequence of SEQ ID NO:1.

In another embodiment, the antibody or antigen-binding fragment thereof, when bound to BTN1A1, binds to six or more residues selected from the group consisting of G365, D366, T368, D369, W370, A371, and I372 within an amino acid sequence of SEQ ID NO:1.

In yet another embodiment, the antibody or antigen-binding fragment thereof, when bound to BTN1A1, binds to all seven residues selected from the group consisting of G365, D366, T368, D369, W370, A371, and I372 within an amino acid sequence of SEQ ID NO:1.

In another embodiment, the antibody or antigen-binding fragment thereof, when bound to BTN1A1, binds to G365 within an amino acid sequence of SEQ ID NO:1. In another embodiment, the antibody or antigen-binding fragment thereof, when bound to BTN1A1, binds to D366 within an amino acid sequence of SEQ ID NO:1. In a particular embodiment, the antibody or antigen-binding fragment thereof, when bound to BTN1A1, binds to T368 within an amino acid sequence of SEQ ID NO:1. In one specific embodiment, the antibody or antigen-binding fragment thereof, when bound to BTN1A1, binds to D369 within an amino acid sequence of SEQ ID NO:1. In some embodiments, the antibody or antigen-binding fragment thereof, when bound to BTN1A1, binds to W370 within an amino acid sequence of SEQ ID NO:1. In other embodiments, the antibody or antigen-binding fragment thereof, when bound to BTN1A1, binds to A371 within an amino acid sequence of SEQ ID NO:1. In another embodiment, the antibody or antigen-binding fragment thereof, when bound to BTN1A1, binds to I372 within an amino acid sequence of SEQ ID NO:1. Any combination of two, three, four, five, six, seven, or more of the above-referenced amino acid BTN1A1 binding sites is also contemplated.

### 4.3.1.1 Polyclonal Antibodies

The antibodies of the present disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a BTN1A1 polypeptide or a fusion protein thereof. It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly 1C, Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for BTN1A1 antibody titer. If desired, the mammal can be boosted until the antibody titer increases or plateaus. Additionally or alternatively, lymphocytes may be obtained from the immunized animal for fusion and preparation of monoclonal antibodies from hybridoma as described below.

### 4.3.1.2 Monoclonal Antibodies

The antibodies of the present disclosure may alternatively be monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Plückthun, 1992, Immunol. Revs. 130:151-88.

In some embodiments, an antibody that binds a BTN1A1 epitope comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989).

In some embodiments, an antibody that binds a BTN1A1 epitope comprises an amino acid sequence of a VH CDR or an amino acid sequence of a VL CDR encoded by a nucleotide sequence that hybridizes to the complement of a nucleotide sequence encoding any one of the VH CDRs and/or VL CDRs depicted in Tables 1-6 under stringent conditions (e.g., hybridization to filter-bound DNA in 6×SSC at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, e.g., Ausubel et al., supra).

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, BTN1A1 (e.g., a BTN1A1 polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-BTN1A1 antibodies can be obtained by designing a suitable antigen screening procedure to select for the phage clone of interest followed by construction of a full length anti-BTN1A1 antibody clone using VH and/or VL sequences (e.g., the Fv sequences), or various CDR sequences from VH and VL sequences, from the phage clone of interest and suitable constant region (e.g., Fc) sequences described in Kabat et al., supra.

In another embodiment, anti-BTN1A1 antibody is generated by using methods as described in Bowers et al., 2011, Proc Natl Acad Sci USA. 108:20455-60, e.g., the SHM-XHL™ platform (AnaptysBio, San Diego, CA). Briefly, in this approach, a fully human library of IgGs is constructed in a mammalian cell line (e.g., HEK293) as a starting library. Mammalian cells displaying immunoglobulin that binds to a target peptide or epitope are selected (e.g., by FACS sorting), then activation-induced cytidine deaminase (AID)-triggered somatic hypermutation is reproduced in vitro to expand diversity of the initially selected pool of antibodies. After several rounds of affinity maturation by coupling mammalian cell surface display with in vitro somatic hypermutation, high affinity, high specificity anti-BTN1A1 antibodies are generated. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

### 4.3.1.3 Antibody Fragments

The present disclosure provides antibodies and antibody fragments that bind to BTN1A1 In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to a BTN1A1 epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to a BTN1A1 epitope. Exemplary functional fragments include Fab fragments (e.g., an antibody fragment that contains the antigen-binding domain and comprises a light chain and part of a heavy chain bridged by a disulfide bond); Fab′(e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

### 4.3.1.4 Humanized Antibodies

In some embodiments, antibodies provided herein can be humanized antibodies that bind BTN1A1, including human and/or cynomolgus BTN1A1. For example, humanized antibodies of the present disclosure may comprise one or more CDRs as shown in Tables 1-6. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al, 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al, 1993, J. Immunol. 151:2623-32). In some cases, the framework is derived from the consensus sequences of the most abundant human subclasses, V_{L}6 subgroup I (V_{L}6I) and VH subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

### 4.3.1.5 Human Antibodies

Human anti-BTN1A1 antibodies can be constructed by combining Fv clone variable domain sequence(s) selected from human-derived phage display libraries with known human constant domain sequences(s). Alternatively, human monoclonal anti-BTN1A1 antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Brüggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In some instances, however, VH CDR3 and VL CDR3, as well as VH CDR2, VL CDR2, and VL CDR1 of the non-human antibody may be retained.

### 4.3.1.6 Bispecific Antibodies

Bispecific antibodies are monoclonal antibodies that have binding specificities for at least two different antigens. In certain embodiments, bispecific antibodies are human or humanized antibodies. In certain embodiments, one of the binding specificities is for BTN1A1 and the other is for any other antigen. In some embodiments, one of the binding specificities is for BTN1A1, and the other is for another surface antigen expressed on cells expressing BTN1A1 In certain embodiments, bispecific antibodies may bind to two different epitopes of BTN1A1. Bispecific antibodies can be prepared as full length antibodies or antibody fragments (e.g., F(ab′)₂ bispecific antibodies).

Methods for making bispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating bispecific antibodies, see, for example, Bispecific Antibodies (Kontermann ed., 2011).

### 4.3.1.7 Multivalent Antibodies

A multivalent antibody may be internalized (and/or catabolized) faster than a bivalent antibody by a cell expressing an antigen to which the antibodies bind. The antibodies of the present disclosure can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### 4.3.1.8 Fe Engineering

It may be desirable to modify an anti-BTN1A1 antibody provided herein by Fe engineering. In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29(8):2613-24; and Shields et al., 2001, J. Biol. Chem. 276(9): 6591-604). Other Fc variants are provided elsewhere herein.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### 4.3.1.9 Alternative Binding Agents

The present disclosure encompasses non-immunoglobulin binding agents that specifically bind to the same epitope as an anti-BTN1A1 antibody disclosed herein. In some embodiments, a non-immunoglobulin binding agent is identified as an agent that displaces or is displaced by an anti-BTN1A1 antibody of the present disclosure in a competitive binding assay. These alternative binding agents may include, for example, any of the engineered protein scaffolds known in the art. Such scaffolds may comprise one or more CDRs as shown in Tables 1-6. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352: 95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### 4.3.2 Antibody Variants

In some embodiments, amino acid sequence modification(s) of the antibodies that bind to BTN1A1 or described herein are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the anti-BTN1A1 antibodies provided herein, it is contemplated that anti-BTN1A1 antibody variants can be prepared. For example, anti-BTN1A1 antibody variants can be prepared by introducing appropriate nucleotide changes into the encoding DNA, and/or by synthesis of the desired antibody or polypeptide. Those skilled in the art who appreciate that amino acid changes may alter post-translational processes of the anti-BTN1A1 antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by increase or decrease of glycosylation, acetylation, pegylation, phosphorylation, amidation, derivatization by known protecting/blocking groups, chemical cleavage, proteolytic cleavage, linkage to a cellular ligand or other protein, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites. Accordingly, in one embodiment, an antibody or fragment thereof that binds to a BTN1A1 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a murine monoclonal antibody provided herein. In one embodiment, an antibody or fragment thereof that binds to a BTN1A1 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence depicted in Tables 1-10. In yet another embodiment, an antibody or fragment thereof that binds to a BTN1A1 epitope comprises a VH CDR and/or a VL CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VH CDR amino acid sequence depicted in Tables 2, 4, and 6 and/or a VL CDR amino acid sequence depicted in Tables 1, 3, and 5. The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-BTN1A1 antibody variant DNA.

Any cysteine residue not involved in maintaining the proper conformation of the anti-BTN1A1 antibody also may be substituted, for example, with another amino acid, such as alanine or serine, to improve the oxidative stability of the molecule and to prevent aberrant crosslinking. Conversely, cysteine bond(s) may be added to the anti-BTN1A1 antibody to improve its stability (e.g., where the antibody is an antibody fragment such as an Fv fragment).

In some embodiments, an anti-BTN1A1 antibody molecule of the present disclosure is a “de-immunized” antibody. A “de-immunized” anti-BTN1A1 antibody is an antibody derived from a humanized or chimeric anti-BTN1A1 antibody, which has one or more alterations in its amino acid sequence resulting in a reduction of immunogenicity of the antibody, compared to the respective original non-de-immunized antibody. One of the procedures for generating such antibody mutants involves the identification and removal of T cell epitopes of the antibody molecule. In a first step, the immunogenicity of the antibody molecule can be determined by several methods, for example, by in vitro determination of T cell epitopes or in silico prediction of such epitopes, as known in the art. Once the critical residues for T cell epitope function have been identified, mutations can be made to remove immunogenicity and retain antibody activity. For review, see, for example, Jones et al., 2009, Methods in Molecular Biology 525:405-23.

### 4.3.2.1 In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed as Fab, scFv, or V domain fragments either on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be displayed as single-chain variable fusions (scFv) in which the heavy and light chains are connected by a flexible linker. The scFv is fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In a mammalian cell display system (see, e.g., Bowers et al., 2011, Proc Natl Acad Sci USA. 108:20455-60), a fully human library of IgGs is constructed based on germline sequence V-gene segments joined to prerecombined D(J) regions. Full-length V regions for heavy chain and light chain are assembled with human heavy chain and light chain constant regions and transfected into a mammalian cell line (e.g., HEK293). The transfected library is expanded and subjected to several rounds of negative selection against streptavidin (SA)-coupled magnetic beads, followed by a round of positive selection against SA-coupled magnetic beads coated with biotinylated target protein, peptide fragment, or epitope. Positively selected cells are expanded, and then sorted by rounds of FACS to isolate single cell clones displaying antibodies that specifically bind to the target protein, peptide fragment, or epitope. Heavy and light chain pairs from these single cell clones are retransfected with AID for further maturation. Several rounds of mammalian cell display, coupled with AID-triggered somatic hypermutation, generate high specificity, high affinity antibodies.

Diversity may also be introduced into the CDRs or the whole V genes of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. In a specific embodiment, somatic hypermutation is performed by AID-triggered somatic hypermutation, e.g., using the SHM-XEL™ platform (AnaptysBio, San Diego, CA). Random mutations can be introduced throughout the whole V gene using E. coli mutator strains, error-prone replication with DNA polymerases (see, e.g., Hawkins et al., 1992, J. Mol. Biol. 226:889-96), or RNA replicases. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, BTN1A1 can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 4.3.2.2 Modifications of Anti-BTN1A1 Antibodies

Covalent modifications of anti-BTN1A1 antibodies are included within the scope of the present disclosure. Covalent modifications include reacting targeted amino acid residues of an anti-BTN1A1 antibody with an organic derivatizing agent that is capable of reacting with selected side chains or the N- or C-terminal residues of the anti-BTN1A1 antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the anti-BTN1A1 antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An anti-BTN1A1 antibody of the present disclosure may also be modified to form chimeric molecules comprising an anti-BTN1A1 antibody fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are fusion proteins comprising an antibody provided herein that binds to a BTN1A1 antigen and a heterologous polypeptide. In some embodiments, the heterologous polypeptide to which the antibody is fused is useful for targeting the antibody to cells having cell surface-expressed BTN1A1.

Also provided herein are panels of antibodies that bind to a BTN1A1 antigen. In specific embodiments, the panels of antibodies have different association rates, different dissociation rates, different affinities for a BTN1A1 antigen, and/or different specificities for a BTN1A1 antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96-well or 384-well plates, for assays such as ELISAs.

### 4.3.3 Preparation of Anti-BTN1A1 Antibodies

Anti-BTN1A1 antibodies may be produced by culturing cells transformed or transfected with a vector containing anti-BTN1A1 antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-BTN1A1 antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, 1963, J. Am. Chem. Soc. 85:2149-54). In vitro protein synthesis may be performed using manual techniques or by automation. Various portions of the anti-BTN1A1 antibody may be chemically synthesized separately and combined using chemical or enzymatic methods to produce the desired anti-BTN1A1 antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

### 4.3.4 Immunoconjugates

The present disclosure also provides conjugates comprising any one of the anti-BTN1A1 antibodies of the present disclosure covalently bound by a synthetic linker to one or more non-antibody agents.

In some embodiments, antibodies provided herein are conjugated or recombinantly fused, e.g., to a diagnostic or detectable molecule. The conjugated or recombinantly fused antibodies can be useful, for example, for monitoring or prognosing the onset, development, progression, and/or severity of a BTN1A1-mediated disease.

Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

Also provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising an antigen-binding fragment of an antibody provided herein (e.g., a Fab fragment, Fc fragment, Fv fragment, F(ab)₂ fragment, a VH domain, a VH CDR, a VL domain, or a VL CDR) and a heterologous protein, polypeptide, or peptide. In one embodiment, the heterologous protein, polypeptide, or peptide that the antibody is fused to is useful for targeting the antibody to a particular cell type, such as a cell that expresses BTN1A1. For example, an antibody that binds to a cell surface receptor expressed by a particular cell type may be fused or conjugated to a modified antibody provided herein.

Moreover, antibodies provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of anti-BTN1A1 antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described, for example, in U.S. Pat. No. 4,676,980.

Antibodies that bind to BTN1A1 as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37).

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the F-amino group of Lys residues or the thiol group of Cys residues, which results in heterogenous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

### 4.4 Methods of Using the Antibodies and Compositions

### 4.4.1 IHC Methods for Detecting BTN1A1

Immunohistochemistry (IHC) on formalin fixed paraffin embedded (FFPE) tissue is critical step in R&D therapeutic campaigns by identify cells expressing the target proteins of interest and predicting potential toxicities. A robust IHC assay depends on suitable primary antibodies that reliably recognize the target with optimal specificity and sensitivity. FFPE tissues often present over-fixed proteins with altered conformation, which renders repurposing of antibodies validated in non-IHC assays extremely uncertain. During the tissue preparation and preservation process, if a crosslinking agent, such as formalin, is used, formalin fixation may mask epitopes and result in decreased immunoreactivity (see Arnold et al., Biotech Histochem 71:224-230(1996)). Formalin fixation is a time-dependent process in which increased fixation time results in continued formaldehyde group binding to proteins to a point of equilibrium (see Fox et al., J Histochem Cytochem 33:845-853 (1985)). Studies have shown that formalin fixation, especially if prolonged, results in decreased antigenicity (see Battifora and Kopinski, J Histochem Cytochem 34:1095-1100(1986)), which limits the use of formalin-fixed tissues for diagnostic IHC (see Ramos-Vara, Vet Pathol 42:405-426(2005), Webster et al., J Histochem Cytochem. 57(8): 753-761(2009)). Generation of new IHC antibodies, when suitable reagents are not commercially available, requires screening many candidates against relevant controls.

In one aspect, provided herein is a method of detecting BTN1A1 in a formalin-fixed paraffin-embedded (“FFPE”) sample.

In some embodiments, a BTN1A1 detected by a method of this disclosure includes any BTN1A1 variant, isoform, and species homolog, which is naturally expressed by cells or can be expressed on cells transfected with genes or cDNA encoding the polypeptide. Unless noted, preferably the BTN1A1 is a human BTN1A1.

In some embodiments, a sample used in a method of this disclosure comprises cells collected from bodily fluid or tissue. In some embodiments, a sample may comprise any tissue found in an organism. In some embodiments, the organism comprises a vertebrate or non-vertebrate organism. In some embodiments, the vertebrate comprises a human or a non-human primate. In some embodiments, the monkey comprises a cynomolgus monkey (Macaca fascicularis), a rhesus monkey (Macaca mulatta), a marmoset (Callithrix jacchus), a Chimpanzee (Pan troglodytes), a Bonobo (Pan paniscus), a Bornean orangutan (Pongo pygmaeus), a sumatran orangutan (Pongo abelii), a Tapanuli orangutan (Pongo tapanuliensis) or any other known primate species. In some embodiments, the tissue comprises a tumor tissue. In some embodiments, the tumor tissue comprises a benign tumor. In another embodiment, the tumor tissue comprises a premalignant tumor. In other embodiments, the tumor tissue comprises a malignant tumor. A nonexhaustive list of exemplary tumor tissues contemplated by the present disclosure includes a sarcoma, carcinoma, adenocarcinoma, lymphoma, breast tumor, mammary tumor, prostate tumor, head and neck tumor, brain tumor, pituitary tumor, glioblastoma, medulloblastoma, atypical teratoid/rhabdoid tumor, bladder tumor, pancreatic tumor, islet tumor, liver tumor, ovarian tumor, colorectal tumor, lung tumor, bronchial tumor, tracheobronchial tumor, skin tumor, lymphoid tumor, and a gastrointestinal tumor.

In further embodiments, the tissue may comprise brain tissue. The brain tissue may further comprise the striatum region, thalamus region, midbrain region or medulla region of the brain.

In some embodiments, the sample used in a method of this disclosure is obtained from a subject. In some embodiments, the subject is a mammal. In some embodiments, the mammal is a human or a non-primate human. In other embodiments, the mammal is Macaca fascicularis. Samples may be collected by any method known in the art. A person skilled in the art will appreciate that collection procedures will vary according to the sample type and the intended analysis. For example, blood samples may be collected by intravenous route with an evacuated tube system. Brain tissue may be collected by making an incision in the scalp, drilling a hole in the skull and inserting a needle into the brain to obtain brain tissue.

In some embodiments of this disclosure, tissue or cell samples may be fixed or embedded. Fixatives may be needed, for example, to preserve cells and tissues in a reproducible and life-like manner. Fixatives may also stabilize cells and tissues, thereby protecting them from the rigors of processing and staining techniques. For example, samples comprising tissue blocks, sections, or smears may be immersed in a fixative fluid, or in the case of smears, dried.

Many methods of fixing and embedding tissue specimens are known, for example, formalin-fixation and subsequent paraffin embedding (FFPE). Any suitable fixing agent may be used. Examples include ethanol, acetic acid, picric acid, 2-propanol, 3,3′-diaminobenzidine tetrahydrochloride dihydrate, acetoin (mixture of monomer) and dimer, acrolein, crotonaldehyde (cis+trans), formaldehyde, glutaraldehyde, glyoxal, potassium dichromate, potassium permanganate, osmium tetroxide, paraformaldehyde, mercuric chloride, tolylene-2,4-diisocyanate, trichloroacetic acid, tungstic acid. Other examples include formalin (aqueous formaldehyde) and neutral buffered formalin, glutaraldehyde, carbodiimide, imidates, benzoequinone, osmic acid and osmium tetraoxide. Fresh biopsy specimens, cytological preparations (including touch preparations and blood smears), frozen sections, and tissues for IHC analysis may be fixed in organic solvents, including ethanol, acetic acid, methanol and/or acetone sample.

In some embodiments of this disclosure, the method comprises a pre-treating step to increase the reactivity or accessibility of target molecules in the sample, while reducing nonspecific interactions. This process is referred to as antigen retrieval, which is also known as target retrieval, epitope retrieval, target unmasking or antigen unmasking. See, e.g., Shi et al., J Histochem Cytochem, 45(3): 327 (1997).

### 4.4.2 Method of Treatment

In one aspect, provided herein is a method of treating a subject with a BTN1A1 targeting agent, comprising a step of detecting BTN1A1 in a sample from the subject. In some embodiments, a method of treating a subject with a BTN1A1 targeting agent first comprises detecting BTN1A1. Any of the methods of disclosed herein may be used in a method of detecting BTN1A1.

In some embodiments, the BTN1A1 targeting agent comprises any BTN1A1 targeting agent known in the art. As used herein, “targeting agent” refers to any molecule capable of interacting with a target of interest. Thus, in some embodiments, the targeting agent comprises a chimeric antigen receptor (CAR) T-cell or a T cell redirecting antibody. In other embodiments, the targeting agent comprises an antibody or antigen binding fragment thereof. In some embodiments, the antibody or antigen binding fragment thereof may comprise an anti-BTN1A1 antibody or antigen binding fragment thereof. In further embodiments, the antibody may comprise a bispecific or multispecific antibody. The bispecific or multispecific antibody may comprise an anti-BTN1A1 bispecific or multispecific antibody. In other embodiments, the targeting agent may comprise a small molecule capable of binding BTN1A1. In other embodiments, the targeting agent may comprise a BTN1A1 polypeptide.

In some embodiments, the target of interest is a soluble polypeptide. In other embodiments, the target of interest is a membrane-bound polypeptide including a receptor. In some embodiments, the receptor comprises BTN1A1.

In some embodiments, a subject of the present treatment method has a disease, disorder, condition or syndrome. In some embodiments, the disease, disorder, condition or syndrome may be any human disease, disorder, condition or syndrome known in the art. In some embodiments, the targeting agent of the present disclosure may also be used to treat diseases mediated by BTN1A1. In some embodiments, the targeting agent of the present disclosure may also eliminate diseased cells expressing BTN1A1.

In some embodiments, the subject of the present treatment method has a cancer or tumor. In other embodiments, the cancer may comprise anal cancer, bile duct cancer, bladder cancer, bone cancer, breast cancer, cervical cancer, chronic lymphocytic leukemia (CLL), chronic myelogenous leukemia (CML), chronic myeloproliferative neoplasms, colon cancer, colorectal cancer, endometrial cancer, esophageal cancer, fallopian tube cancer, gallbladder cancer, gastric (stomach) cancer, head and neck cancer, liver cancer, hepatocellular carcinoma, Hodgkin lymphoma, laryngeal cancer, leukemia, lung cancer, non-small cell lung cancer, ovarian cancer, pancreatic cancer, melanoma, merkel cell carcinoma (skin cancer), mesothelioma, malignant, paranasal sinus and nasal cavity cancer (head and neck cancer), parathyroid cancer, penile cancer, pharyngeal cancer (head and neck cancer), prostate cancer, rectal cancer, renal cell cancer, skin cancer, small intestine cancer, stomach cancer, testicular cancer, thyroid cancer, urethral cancer, uterine cancer, vaginal cancer, and vulvar cancer.

In further embodiments, examples of breast cancers include but are not limited to invasive ductal carcinoma, invasive lobular carcinoma, ductal carcinoma in situ and lobular carcinoma in situ. The examples of respiratory tract cancers include but are not limited to small cell lung cancer, non-small cell lung cancer, bronchial adenoma and pleuropulmonary blastoma. The examples of brain cancers include but are not limited to brain stem and hypothalamic gliomas, cerebellar and cerebral astrocytomas, medulloblastoma, ependymoma and neuroectodermal and pineal tumors. Male genital neoplasms include but are not limited to prostatic cancers and testicular cancers. Female genital neoplasms include but are not limited to endometrial cancer, cervical cancer, ovarian cancer, vaginal cancer, vulvar cancer and hysteroma. Gastrointestinal tumors include but are not limited to anal cancer, colon cancer, colorectal cancer, esophageal cancer, gallbladder cancer, stomach cancer, pancreatic cancer, rectal cancer, small intestine cancer and salivary gland cancer. Urethral tumors include but are not limited to bladder cancer, penile cancer, renal carcinoma, renal pelvic carcinoma, ureteral cancer and urethral cancer. Eye cancers include but are not limited to intraocular melanoma and retinoblastoma. The examples of liver cancers include but are not limited to hepatocellular carcinoma (hepatocellular carcinoma with or without fibrolamellar variation), cholangiocarcinoma (intrahepatic cholangiocarcinoma) and combined hepatocellular-cholangiocarcinoma. Skin cancers include but are not limited to squamous-cell carcinoma, Kaposi's sarcoma, malignant melanoma, Merkel cell carcinoma and non-melanoma skin cancers. Head and neck cancers include but are not limited to laryngeal/hypopharyngeal/nasopharyngeal/oropharyngeal carcinomas, as well as lip and oral cancers. Lymphomas include but are not limited to AIDS-associated lymphoma, non-Hodgkin's lymphoma, cutaneous T-cell lymphoma, Hodgkin's disease and central nervous system lymphoma. Sarcomas include but are not limited to soft tissue sarcoma, osteosarcoma, malignant fibrous histiocytoma, lymphosarcoma and rhabdomyosarcoma.

In some embodiments, the cancer may comprise a hematological cancer. Hematologic cancers are cancers of the blood or bone marrow. In some embodiments, the hematological (or hematogenous) cancer of the present disclosure may comprise leukemia (acute leukemia, acute lymphocytic leukemia, acute myelocytic leukemia, acute myelogenous leukemia, myeloblastic leukemia, promyeiocytic leukemia, myelomonocytic leukemia, monocytic leukemia and erythroleukemia), chronic leukemia (chronic myelocytic leukemia, chronic granulocytic leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia B cell lymphoma, plasma cell leukemia, myelodysplasia, secondary leukemia; myeloproliferative syndromes (MPS) or Burkett's lymphoma (Endemic Burkitt's lymphoma or sporadic Burkitt's lymphoma), malignant plasma cell neoplasms, BCMA+high-grade lymphoma, Kahler's disease and myelomatosis, plasma cell leukemia; plasmacytoma, B-cell prolymphocytic leukemia, hairy cell leukemia, follicular lymphoma (including follicular non-Hodgkin's lymphoma types), marginal zone lymphoma (Mucosa-Associated Lymphoid Tissue: MALT 1 MALToma, Monocytoid B cell lymphoma, splenic lymphoma with villous lymphocytes), mantle cell lymphoma; large cell lymphoma (diffuse large cell, diffuse mixed cell, immunoblastic lymphoma, primary mediastinal B cell lymphoma, angiocentric lymphoma pulmonary B cell), small lymphocytic lymphoma (SLL), precursor B-lymphoblastic lymphoma; subacute myeloid leukemia, myeloid sarcoma, chloroma, granulocytic sarcoma, acute promyelocytic leukemia, acute myelomonocytic leukemia) or other B-cell leukemia or lymphoma.

In some embodiments, a subject of the present disclosure has a plasma cell disorder such as heavy-chain disease, primary or immunocyte-associated amyloidosis, and monoclonal gammopathy of undetermined significance (MGUS).

In some embodiments, the subject of the present treatment method has a the disease or disorder. In some embodiments, the disease or disorder may comprise acquired immune deficiency syndrome (AIDS), brain disease, acute flaccid myelitis (AFM), amyotrophic lateral sclerosis (ALS), Alzheimer's disease, amyotrophic lateral sclerosis, arthritis, bone diseases, inflammatory diseases, osteoarthritis (OA), rheumatoid arthritis (RA), asthma, blood disorders, brain disease, dementia, diabetes, enteric disease, liver disease, renal disorders lung disease, skin disease, gastrointestinal disease, ulcerative colitis, inflammatory bowel disease, hypertension, or cardiovascular disorders. In other embodiments, the disease or condition may comprise a disease or disorder of the breasts, respiratory tracts, brains, reproductive organs, alimentary canals, urethrae, eyes, livers, skins, heads and necks, thyroid glands and parathyroid glands.

In some embodiments, a subject of the present treatment method has an immunologic disorder and in particular autoimmune disorders In some embodiments, the autoimmune disorder includes, but is not limited to, systemic lupus erythematosus, myasthenia gravis, autoimmune hemolytic anemia, idiopathic thrombocytopenia purpura, anti-phospholipid syndrome, Chaga's disease, Grave's disease, Wegener's Granulomatosis, Poly-arteritis Nodosa, Rapidly Progressive Glomerulonephritis, rheumatoid arthritis, systemic lupus E (SLE), Type I diabetes, asthma, atopic dermitus, allergic rhinitis, thrombocytopenic purpura, multiple sclerosis, psoriasis, Sjorgren's syndrome, Hashimoto's thyroiditis, Grave's disease, primary biliary cirrhosis, Wegener's granulomatosis, tuberculosis, and graft versus host disease immune-mediated thrombocytopenia, haemolytic anaemia, bullous pemphigoid, myasthenia gravis, Graves' disease, Addison's disease, pemphigus foliaceus, psoriasis, psoriatic arthritis, and ankylosing spondylitis.

In some embodiments, a subject of the present treatment method has an inflammatory disease. In some embodiments, the inflammatory disease is selected from the group consisting of rheumatoid arthritis, psoriasis, allergies, asthma, autoimmune diseases such as Crohn's, IBD, fibromyalgia, mastocytosis, Celiac disease, and any combination thereof. Additionally, the present method may be useful to treat diabetes, particularly Type 1 diabetes.

### 4.5 Pharmaceutical Compositions

In one aspect, the present disclosure further provides pharmaceutical compositions comprising at least one anti-BTN1A1 antibody of the present disclosure. In some embodiments, a pharmaceutical composition comprises 1) an anti-BTN1A1 antibody, and 2) a pharmaceutically acceptable carrier. In some embodiments, the pharmaceutical composition is administered to a subject for in vivo diagnosis a disease or condition mediated by BTN1A1.

Pharmaceutical compositions comprising an antibody are prepared for storage by mixing the antibody having the desired degree of purity with optional physiologically acceptable carriers, excipients, or stabilizers (see, e.g., Remington, Remington's Pharmaceutical Sciences (18th ed. 1980)) in the form of aqueous solutions or lyophilized or other dried forms.

The antibodies of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody that binds to BTN1A1 as described herein, including, but not limited to, encapsulation in liposomes, microparticles, microcapsules, recombinant cells capable of expressing the antibody, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody that binds to BTN1A1 as described herein) or a composition of the invention (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibodies that bind to BTN1A1 as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 4.6 Kits

Also provided herein are kits comprising an antibody (e.g., an anti-BTN1A1 antibody) provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise. Thus, for example, reference to “a peptide sequence” includes a plurality of such sequences and so forth.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 5. EXAMPLES

The examples in this section (i.e., Section 5) are offered by way of illustration, and not by way of limitation.

### 5.1 Example 1: Generation of Anti-BTN1A1 Antibodies

### 5.1.1 Generation of Anti-BTN1A1 Antibodies

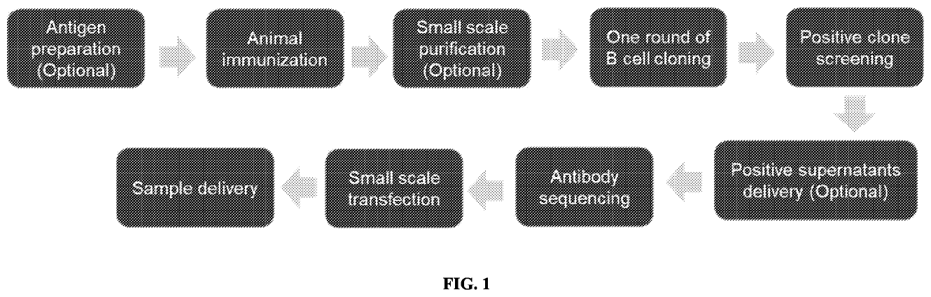

BTN1A1 mAb was generated against a BTN1A1 polypeptide using standard techniques (e.g., by injecting polypeptide comprising BTN1A1 epitopes as immunogens in rats (Aurrand-Lions et al., Immunity, 5(5):391-405(1996)). The procedure for producing the anti-BTN1A1 antibodies as described herein is according to the general workflow shown in FIG. 1 and according to the protocols summarized in Tables 13A to 13E

TABLE 13A
Immunogen Preparation

| | | | | |
|---|---|---|---|---|
| | Price | TAT | Deliver- | QC |
| Steps | ($) | (Weeks) | ables | Standard |

Phase I: Immunogen preparation (SC2032-PF)

| | | | | |
|---|---|---|---|---|
| Peptide synthesis | 250 | 3-4 | 2 mg free | MS&HPLC |
| EVEVGDRTDWAIGVC (Peptide 4) | | | peptide | |
| (SEQ ID NO: 23) | | | | |
| Conjugation with carrier protein | 161 | 1 | / | / |
| Peptide synthesis | | | | |
| RKKVSPAVLVHRDGREQEAE | | | | |
| (ECD1 peptide) (SEQ ID NO: 70) | | | | |

TABLE 13B
Animal Immunization
Phase II: Animal immunization (SC2033-PF)

| | | | | |
|---|---|---|---|---|
| Option 1: Animal | 3750 | 4 | Antiserum report | Indirect ELISA |
| immunization with Phoenix | | | after the 4^{th} | |
| protocol (4 rabbits/peptide | | | immunization | |
| with four injections) | | | | |
| (facility without | | | | |
| accreditation*) | | | | |
| Option 2: Animal | 3168 | 4-6 | Antiserum report | Indirect ELISA |
| immunization with | | | after the 3^{rd} | |
| conventional protocol (2 | | | immunization | |
| rabbits/peptide with three | | | | |
| injections, 6 rabbits total) | | | | |
| (facility without | | | | |
| accreditation* | | | | |

Test samples post the 3rd immunization (ONLY for conventional protocol)

| | | | | |
|---|---|---|---|---|
| Option 1: Small scale antigen- | 964 | 2 | 0.1 mg purified | Indirect ELISA |
| affinity purification with | | | antibody after the | |
| antigen peptide for 4 rabbits | | | 3rd | |
| (0.1 mg total rabbit IgG as | | | immunization/rabbit | |
| negative control) (SC2039-PF) | | | | |
| Option 2: Ammonium Sulfate- | 486 | 1 | 1 mg AS-precipitated | Indirect ELISA |
| precipitation on antisera for 6 | | | total protein after the | |
| rabbits (0.1 mg total rabbit IgG | | | 3rd | |
| as negative control) (SC2033- | | | immunization/rabbit | |
| PF) | | | | |

*For the pricing for AAALAC and OLAW-accredited facility please find in Phase II of V. Project Procedure

TABLE 13C
B cell cloning and screening
Phase III: B cell cloning and screening (SC2034-PF)

| | | | | |
|---|---|---|---|---|
| Option 1: One round of B | 13000 | 2-3 | Report only | Indirect |
| cell cloning & report only | | | | ELISA |
| Option 2: One round of B | 16000 | 4-5 | 1 ml | Indirect |
| cell cloning & supernatants | | | supernatants/clone, | ELISA |
| | | | up to 20 clones | |
| Screening with one | 804/material | / | / | / |
| additional material for one | | | | |
| round of cloning (Optional) | | | | |

TABLE 13D
Antibody Sequencing and Small-Scale Transfection
Phase IV: Antibody sequencing and small scale transfection

| | | | | |
|---|---|---|---|---|
| Antibody sequencing | 4800 | 1-2 | Sequencing reports for up to 5 | Sequencing |
| (SC2040-PF) | | | positive clones | and alignment |
| | | | | report |
| Small scale | 1100 | 2 | 5 ml supernatants/clone from up | Indirect ELISA |
| recombinant | | | to five clones | |
| supernatants | | | | |
| preparation | | | | |
| (SC2038-PF) | | | | |
| Small scale | 1200 | 2 | 0.1 mg recombinant | Indirect ELISA |
| recombinant | | | antibody/clone from up to five | |
| antibody purification | | | clones | |
| (SC2039-PF) | | | | |
| (Optional) | | | | |

Additional deliverables

| | | | | |
|---|---|---|---|---|
| Option 1: Additional | 1125/clone | 3-4 | Sequencing report for one clone | Indirect ELISA |
| antibody sequence | | | 5 ml supernatants/clone from up | |
| and supernatant | | | to five clones | |
| delivery (>10 clones) | | | | |
| (SC2040-PF + | | | | |
| SC2038-PF) | | | | |
| Option 2: Additional | 1366/clone | 6 | Sequencing report for one clone | Indirect ELISA |
| antibody sequence | | | 0.1 mg recombinant | |
| and small-scale | | | antibody/clone | |
| purified antibody | | | | |
| delivery (>10 clones) | | | | |
| (SC2040-PF + | | | | |
| SC2038- | | | | |
| PF + SC2039-PF | | | | |

TABLE 13E
Recombinant Antibody Production and Large Scale Polyclonal Antibody Purification
Phase V: Recombinant antibody production (SC2038-PF + SC2039-PF) (Optional)

| | | | | |
|---|---|---|---|---|
| rAb production | 1670/clone | 4-6 | 3~5 mg | Indirect ELISA |
| | | | rAb/clone | |

Phase VI: Large scale polyclonal antibody purification (SC2038-PF + SC2039-PF)(Optional)

| | | | | |
|---|---|---|---|---|
| Polyclonal antibody | 321/rabbit | 3-4 | Purified | Indirect ELISA |
| purification (4 rabbits) | | | polyclonal | |
| | | | antibody | |

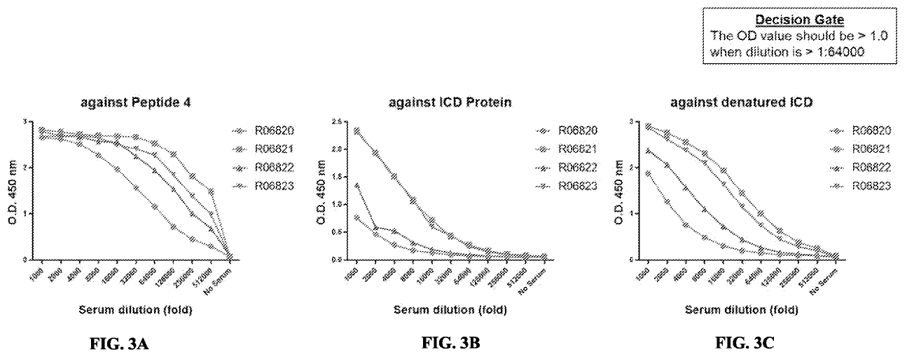

Particularly, a BTN1A1 peptide (peptide 4 of SEQ ID NO:23) was selected from the BTN1A1 ICD (see FIG. 2) and prepared. Peptide 4 was coupled to KLH carrier protein (keyhole limpet hemocyanin, Pierce) was used to immunize New Zealand rabbits. Post-immunization blood samples were taken from the rabbits (#R06820, #R6821, #R06822, #R06823) after 4^{th}, 5^{th}, 6^{th} and 7^{th} immunizations, respectively. Serum samples were subjected to serial dilutions (1:1,000 to 1:512,000) and screened for binding with BTN1A1 peptide 4, BTN1A1 ICD, or denatured BTN1A1 ICD by ELISA. Quantified results were plotted in FIGS. 3A to 3C.

Further, a BTN1A1 peptide (ECD1 peptide of SEQ ID NO:70) was selected from the human BTN1A1 sequence (SEQ ID NO: 1) and prepared. The ECD1 peptide was coupled to KLH carrier protein (keyhole limpet hemocyanin, Pierce) and was used to immunize New Zealand rabbits. Post-immunization blood samples were taken from the rabbits (#R10608, #R10609, #R10610, and #R10611) after 4^{th}, 5^{th}, 6^{th}, and 7^{th} immunizations, respectively. Serum samples were subjected to serial dilutions (1:1,000 to 1:512,000) and screened for binding with BTN1A1 ECD1 or denatured BTN1A1 ECD.

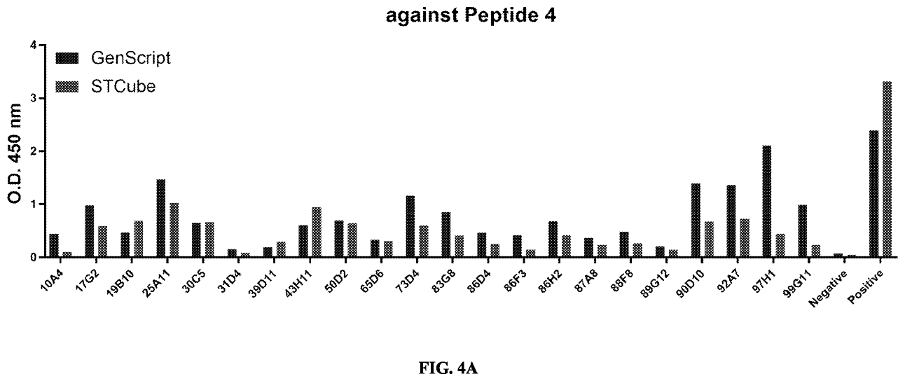

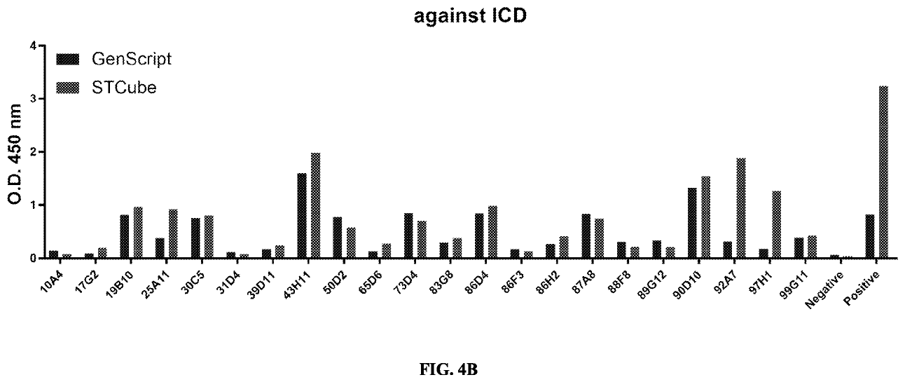

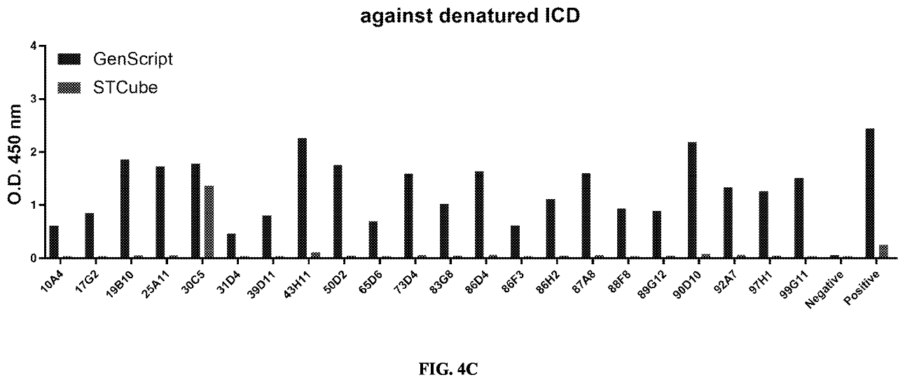

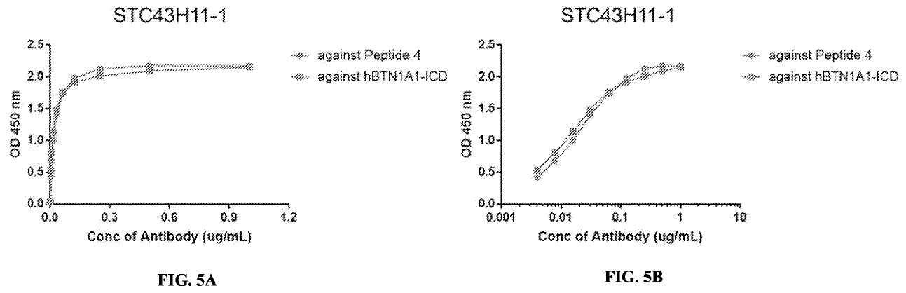

Hybridoma clones producing the anti-BTN1A1 antibodies were generated using methods known in the art. The clones were screened by ELISA for binding with BTN1A1 peptide 4, hBTN1A1 ICD, and denatured hBTN1A1 ICD. Quantified results were summarized in Table 14 and plotted in FIGS. 4A-4C. Clone 43H11 was selected.

TABLE 14
Selection of BTN1A1 Antibody Clones via ELISA

| | Peptide 4 | | ICD | | Denatured ICD | |
|---|---|---|---|---|---|---|
| | GenScript | STCube | GenScript | STCube | GenScript | STCube |
| 10A4 | 0.449 | 0.098 | 0.139 | 0.077 | 0.613 | 0.042 |
| 17G2 | 0.979 | 0.588 | 0.093 | 0.198 | 0.848 | 0.042 |
| 19B10 | 0.472 | 0.687 | 0.813 | 0.969 | 1.861 | 0.053 |
| 25A11 | 1.466 | 1.023 | 0.386 | 0.926 | 1.728 | 0.054 |
| 30C5 | 0.654 | 0.665 | 0.759 | 0.801 | 1.783 | 1.368 |
| 31D4 | 0.154 | 0.084 | 0.116 | 0.074 | 0.466 | 0.043 |
| 39D11 | 0.195 | 0.299 | 0.17 | 0.245 | 0.8 | 0.043 |
| 43H11 | 0.609 | 0.945 | 1.601 | 1.98 | 2.267 | 0.111 |
| 50D2 | 0.696 | 0.645 | 0.775 | 0.579 | 1.752 | 0.047 |
| 65D6 | 0.328 | 0.306 | 0.124 | 0.278 | 0.696 | 0.042 |
| 73D4 | 1.161 | 0.601 | 0.852 | 0.706 | 1.591 | 0.061 |
| 83G8 | 0.85 | 0.412 | 0.292 | 0.382 | 1.024 | 0.047 |
| 86D4 | 0.466 | 0.253 | 0.85 | 0.989 | 1.64 | 0.068 |
| 86F3 | 0.418 | 0.152 | 0.17 | 0.127 | 0.621 | 0.043 |
| 86H2 | 0.684 | 0.418 | 0.269 | 0.419 | 1.122 | 0.048 |
| 87A8 | 0.363 | 0.236 | 0.833 | 0.744 | 1.607 | 0.058 |
| 88F8 | 0.484 | 0.268 | 0.306 | 0.218 | 0.938 | 0.042 |
| 89G12 | 0.204 | 0.143 | 0.336 | 0.207 | 0.892 | 0.045 |
| 90D10 | 1.399 | 0.678 | 1.325 | 1.54 | 2.186 | 0.081 |
| 92A7 | 1.362 | 0.729 | 0.313 | 1.886 | 1.336 | 0.064 |
| 97H1 | 2.104 | 0.449 | 0.179 | 1.263 | 1.257 | 0.044 |
| 99G11 | 0.992 | 0.231 | 0.388 | 0.429 | 1.507 | 0.041 |
| Nega- | 0.074 | 0.048 | 0.063 | 0.04 | 0.062 | 0.041 |
| tive | | | | | | |
| Posi- | 2.39 | 3.314 | 0.821 | 3.245 | 2.439 | 0.253 |
| tive | | | | | | |

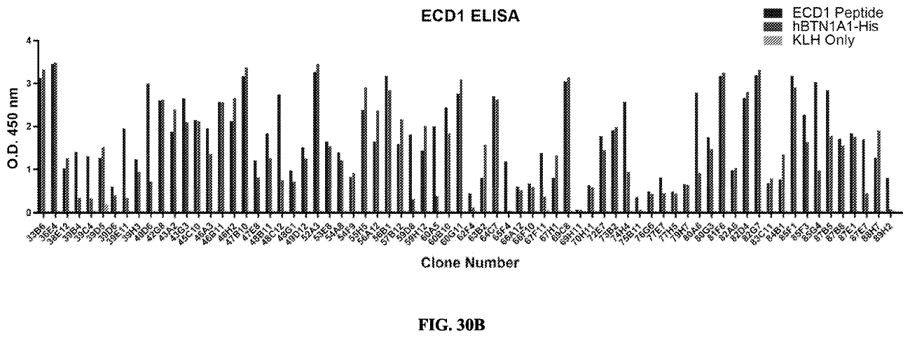

Additional hybridoma clones producing the anti-BTN1A1 antibodies were generated using similar methods known in the art. The clones were screened by ELISA for binding with BTN1A1 ECD1, hBTN1A1-His, and KLH only. Quantified results were summarized in FIGS. 30A and 30B. Clones 43G3-1 and 85F1-1 were selected.

### 5.1.2 Characterization of Antibody STC43H11-1

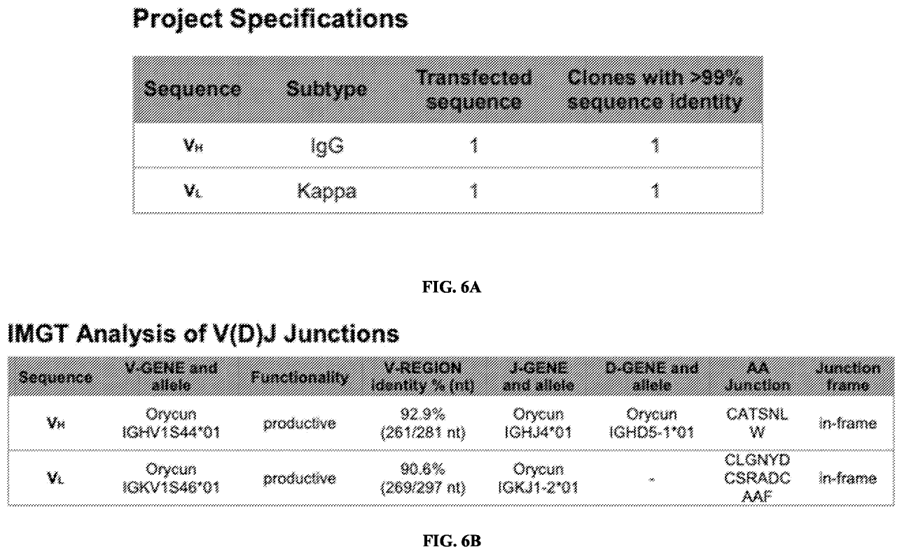

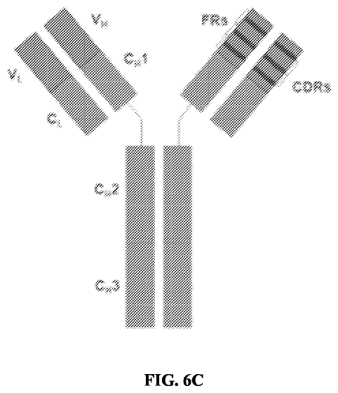

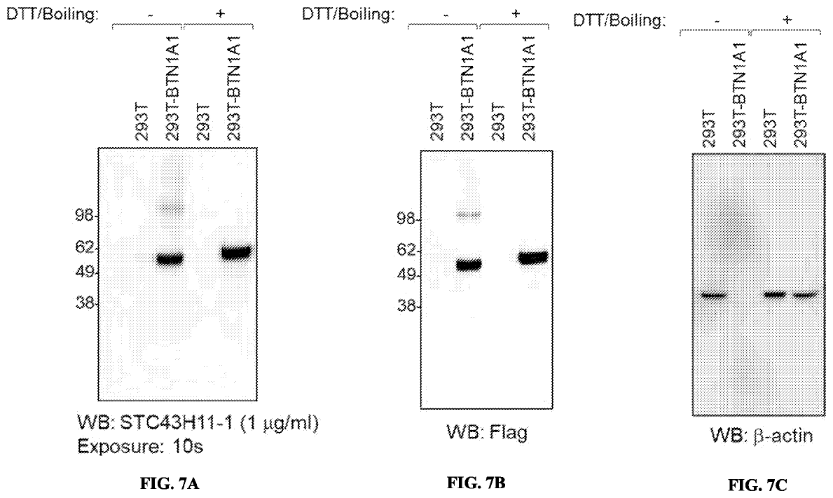

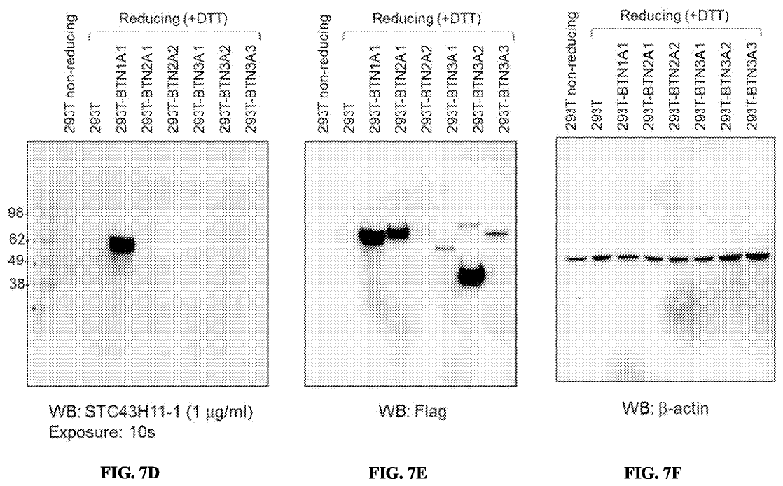

Antibodies produced by clone #43H11 (STC43H11-1) were purified and subjected to further characterizations. First, specific binding of STC43H11-1 to BTN1A1 was assessed using peptide 4, hBTN1A1 ICD and denatured hBTN1A1 ICD via ELISA. Quantified results were summarized in Tables 15, 16 and plotted in FIGS. 6A-6B.

TABLE 15
Interaction between peptide 4, hBTN1A1 ICD
or hBTN1A1 ICD denatured and STC43H11-1

| | | | | | hBTN1A1 ICD | |
|---|---|---|---|---|---|---|
| | BTN1A1 peptide | | hBTN1A1 ICD | | denatured | |
| | Stock | 10X | Stock | 10X | Stock | 10X |
| Clone No. | solution | dilution | solution | dilution | solution | dilution |
| STC43H11-1 | 2.875 | 2.834 | 2.927 | 2.959 | 2.866 | 2.897 |

TABLE 16
Interaction between peptide 4 or hBTN1A1
ICD and purified STC43H11-1

| Antibody | | | | | | |
|---|---|---|---|---|---|---|
| (ug/mL) | against Peptide 4 | | | against hBTN1A1-ICD | | |
| 1 | 2.169 | 2.18 | 2.165 | 2.159 | 2.14 | 2.151 |
| 0.5 | 2.196 | 2.177 | 2.154 | 2.078 | 2.069 | 2.117 |
| 0.25 | 2.13 | 2.11 | 2.124 | 2.004 | 2.009 | 2.029 |
| 0.125 | 2.013 | 1.98 | 1.959 | 1.903 | 1.915 | 1.938 |
| 0.063 | 1.752 | 1.724 | 1.726 | 1.762 | 1.725 | 1.789 |
| 0.031 | 1.408 | 1.401 | 1.405 | 1.479 | 1.487 | 1.491 |
| 0.016 | 0.994 | 1.017 | 0.996 | 1.15 | 1.124 | 1.149 |
| 0.008 | 0.672 | 0.684 | 0.683 | 0.823 | 0.789 | 0.808 |
| 0.004 | 0.424 | 0.428 | 0.416 | 0.543 | 0.519 | 0.54 |
| 0 | 0.042 | 0.042 | 0.043 | 0.041 | 0.043 | 0.046 |

Next, The STC43H11-1 antibody were subjected to sequencing and isotyping analysis. Particularly, total RNA was isolated from B cells using the RNA-easy Isolation Reagent kit (Vazyme, Cat. No. R701-01-AA). Total RNA was then reverse-transcribed into cDNA using either isotype-specific anti-sense primers or universal primers following the technical manual of PrimeScript™ 1st Strand cDNA Synthesis Kit. Antibody fragments of heavy chain and light chain were amplified using specific primers according to the standard operating procedure (SOP) of GenScript. Amplified antibody fragments were cloned into a standard cloning vector separately. Colony PCR was performed to screen for clones with inserts of correct sizes. The consensus sequence was obtained. The antibody molecule had a IgG heavy chain and Kappa light chain (see FIGS. 6A-6C).

### 5.1.3 Characterization of Antibodies STC43G3-1 and STC85F1-1

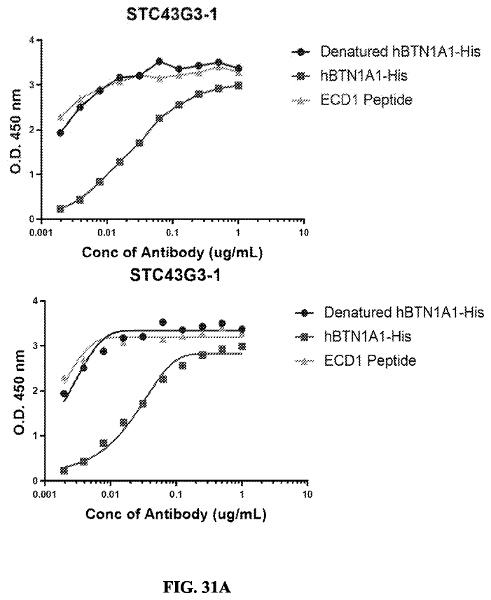

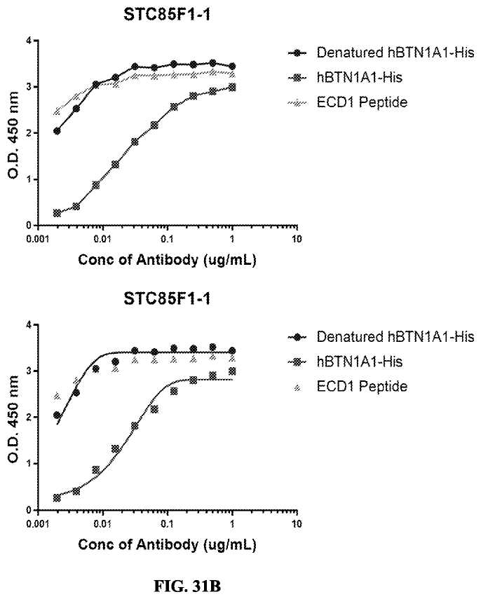

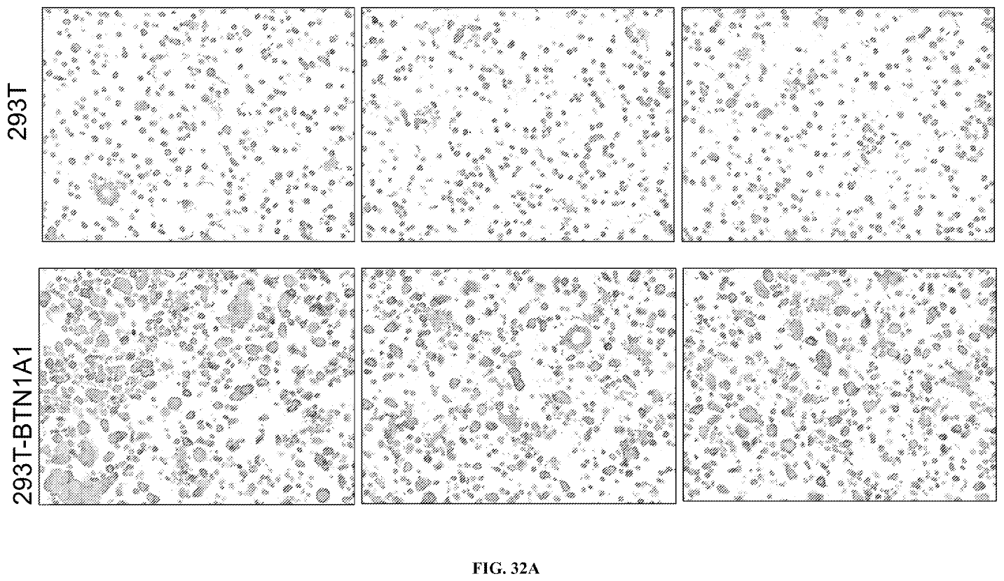

Antibodies produced by clone #43G3-1 (STC43G3-1) and #85F1-1 (STC85F1-1) were purified and subjected to further characterizations. First, specific binding of STC43G3-1 and STC85F1-1 to BTN1A1 was assessed using denatured human BTN1A1-His, human BTN1A1-His, and ECD1 peptide via ELISA. Quantified results were plotted in FIGS. 31A-31B.

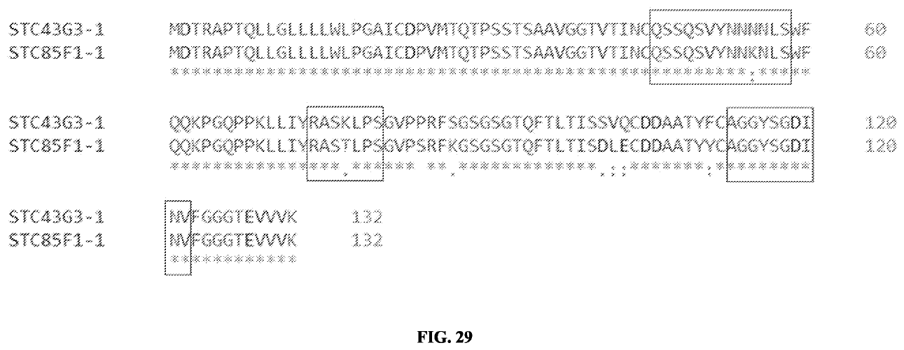

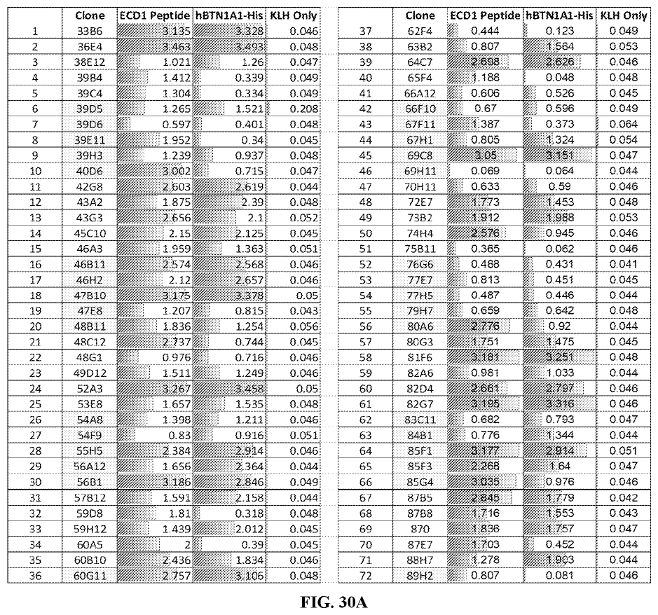

Next, the STC43G3-1 and STC85F1-1 antibodies were subjected to sequencing and isotyping analysis. Particularly, total RNA was isolated from B cells using the RNA-easy Isolation Reagent kit (Vazyme, Cat. No. R701-01-AA). Total RNA was then reverse-transcribed into cDNA using either isotype-specific anti-sense primers or universal primers following the technical manual of PrimeScrip™ 1st Strand cDNA Synthesis Kit. Antibody fragments of heavy chain and light chain were amplified using specific primers according to the standard operating procedure (SOP) of GenScript. Amplified antibody fragments were cloned into a standard cloning vector separately. Colony PCR was performed to screen for clones with inserts of correct sizes. The consensus sequence was obtained. The antibody molecule had a IgG heavy chain and Kappa light chain (see FIGS. 28 and 29).

### 5.1.4 Immunohistochemistry Methods for Detecting BTN1A1

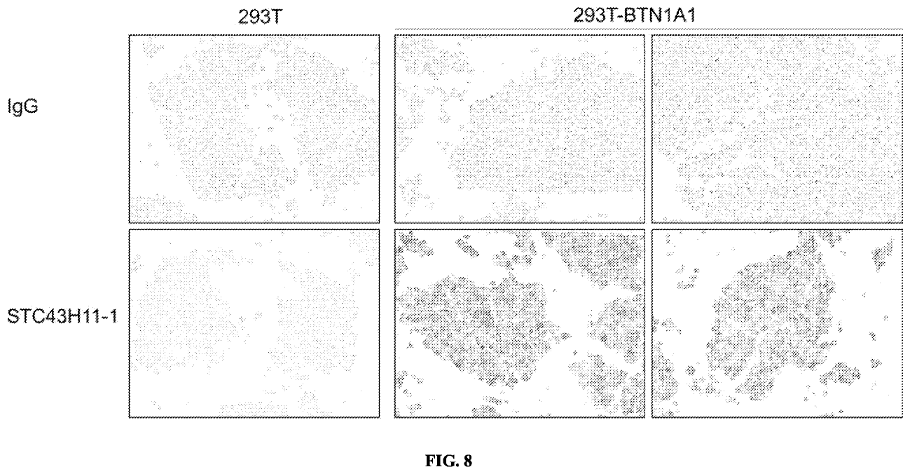

The IHC protocol contained the following steps: 1) prepare the slides by baking said slides in a slide warmer at 60° C. for 60 minutes (the slides contain tissue as described herein); 2) perform antigen retrieval using the Dako system with Low pH Retrieval Buffer; 3) incubate the slides in Vector Labs Bloxall perosidase blocking solution for 10 minutes and then wash in 1×Dako buffer for 5 minutes; 4) incubate the slides in 2.5% horse serum for 20 minutes; 5) dilute the primary antibody to a desired concentration (e.g. Rabbit IgG—5 μg/mL, STC43H11-1, STC43G3-1, or STC85F1-1—5 μg/mL, GenScript culture supplement 50 μL) in 2.5% horse serum and then incubate the slides in the diluted primary antibody for 60 minutes; wash the slides in 1×Dako buffer for 5 minutes; 5) incubate the slides in Vector Labs Amplifier antibody for 15 minutes and then wash slides in 1×Dako buffer for 5 minutes; 6) incubate the slides in Vector Labs Anti-goat IgG HRP-conjugated secondary antibody for 30 minutes and then wash slides in 1×Dako buffer two times; 7) prepare chromogen solution by combining equal parts from Vector Labs kit and then incubate the slides in the chromogen solution for 6 minutes, stopping after the desired color appears; wash the slides in 1×Dako buffer two times; 8) rinse the slides in deionized water, incubate the slides in hematoxylin for two minutes, rinse in water for 5 minutes two times, dip slides in 1×Dako buffer for 30 seconds, and then rinse slides in water; and 9) run slides through ethanol gradient to xylene; mount the slides and let them dry overnight. Antibodies STC43H11-1, STC43G3-1, and STC85F1-1 were used for immunostaining in various immunohistochemical or immunocytochemical assays, and the results were shown in FIG. 8 to FIG. 20 and FIGS. 32 and 33. The protocol for performing immunostaining in various immunohistochemical or immunocytochemical assays is described herein.

FIG. 8 shows detection of BTN1A1 protein expressed by HEK293T cells stained by STC43H11-1 or control IgG (upper panel) via an immunohistochemistry assay. HEK293T cells that did not express BTN1A1 were included as a negative control (left column).

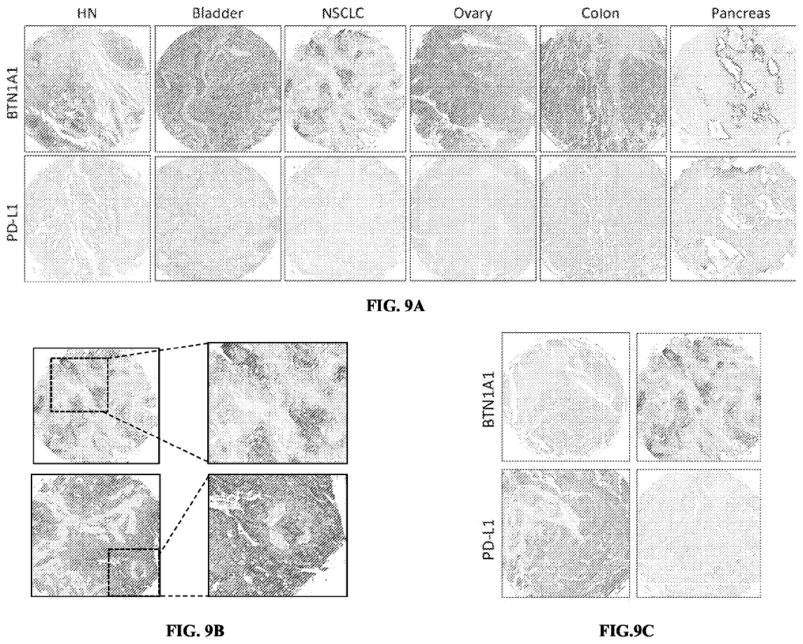

FIG. 9A-9C show immunostaining of various tissues samples (histologically normal (HN), bladder, NSCLC, ovary, colon, pancreas) using STC43H11-1 or an anti-PD-L1 antibody.

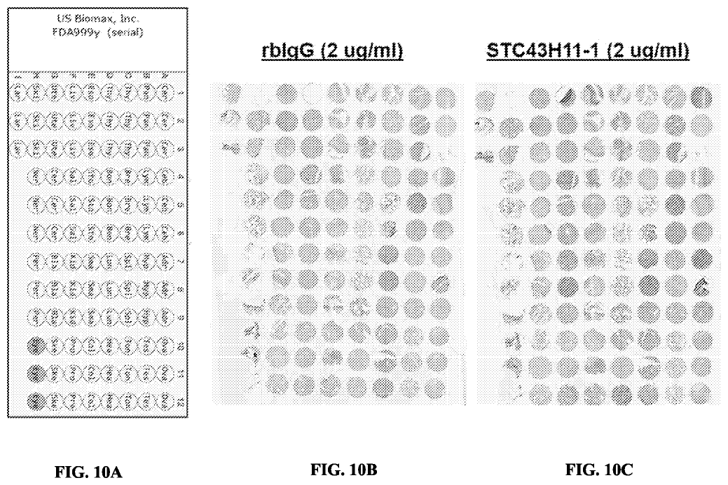

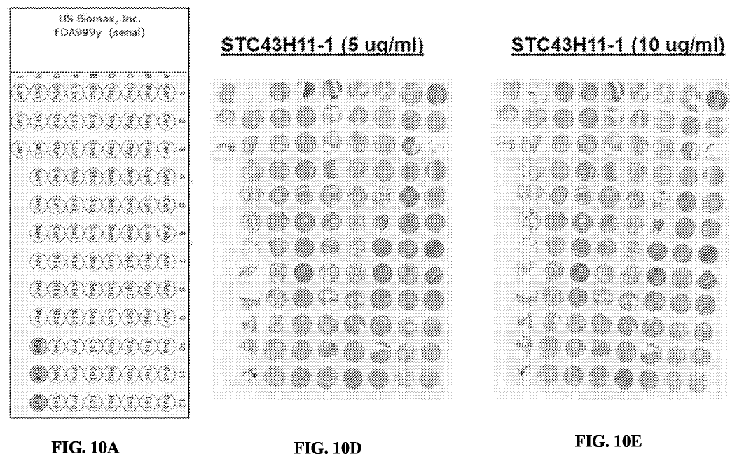

FIGS. 10A-10E show immunostaining of a tissue microarray (“TMA”) using STC43H11-1 at a concentration of 2 μg/mL (FIG. 10C), 5 μg/mL (FIG. 10D), and 10 μg/mL (FIG. 10D). A negative control was included using staining by rabbit IgG at the concentration of 2 μg/mL (FIG. 10A).

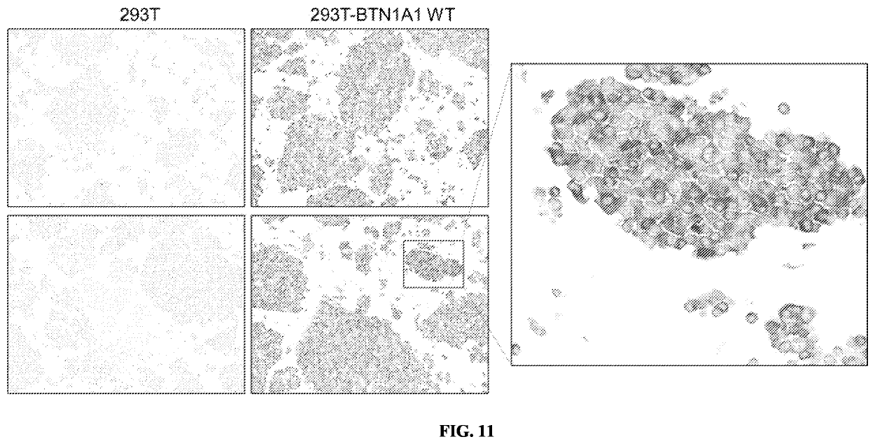

FIG. 11 shows detection of BTN1A1 protein by immunohistochemistry on formalin-fixed paraffin embedded (FFPE) cells expressing BTN1A1. STC43H11-1 was used for immunostaining at a concentration of 2 μg/mL.

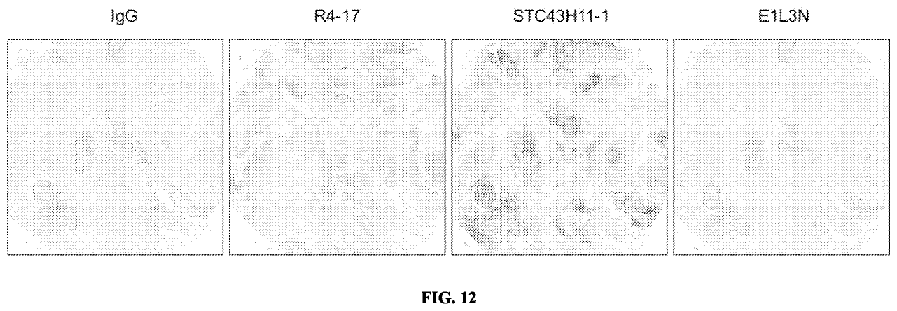

FIG. 12 show immunostaining of tissue sample using IgG (negative control), rabbit polyclonal antibody R4-17 (control), STC43H11-1 and E1L3N (PD-L1) antibodies, respectively, in an immunohistochemistry assay on tissue sample (LC-642b TMA). STC43H11-1 was used for immunostaining at a concentration of 2 μg/mL

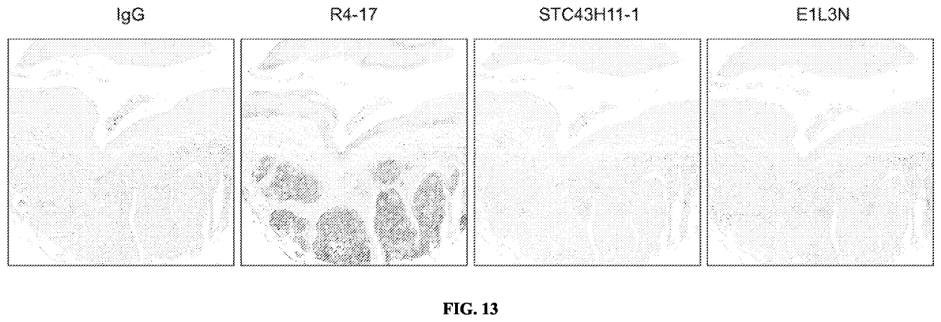

FIG. 13 show immunostaining of tissue sample using IgG (negative control), rabbit polyclonal antibody R4-17 (control), STC43H11-1 and E1L3N antibodies, respectively, in an immunohistochemistry assay on tissue sample (LC-642b: 6F). STC43H11-1 was used for immunostaining at a concentration of 2 μg/mL.

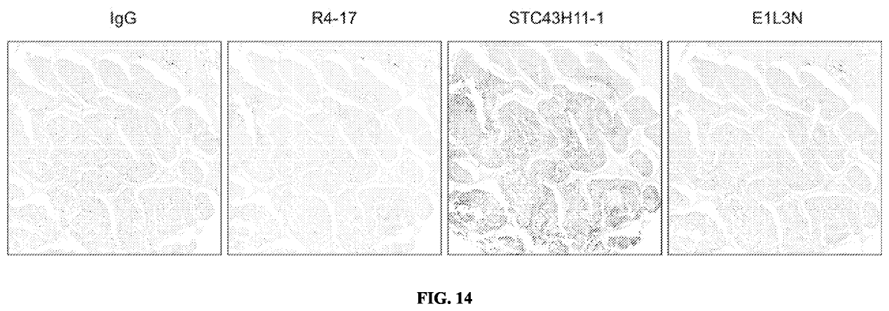

FIG. 14 show immunostaining of tissue sample using IgG (negative control), rabbit polyclonal antibody R4-17 (control), STC43H11-1 and E1L3N antibodies, respectively, in an immunohistochemistry assay on tissue sample (LC-642b: 6G).

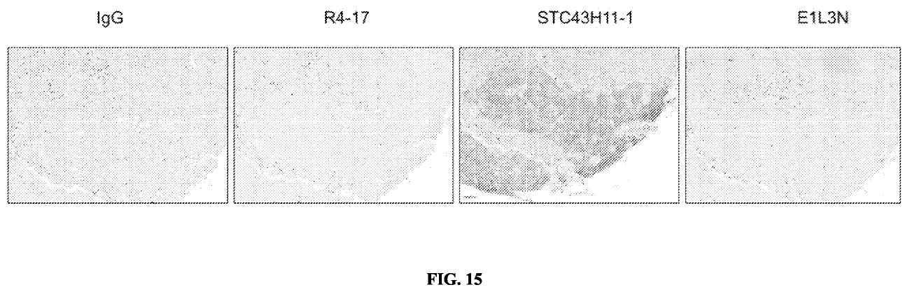

FIG. 15 show immunostaining of tissue sample using IgG (negative control), rabbit polyclonal antibody R4-17 (control), STC43H11-1 and E1L3N antibodies, respectively, in an immunohistochemistry assay on tissue sample (HN TMA: E6). STC43H11-1 was used for immunostaining at a concentration of 2 μg/mL.

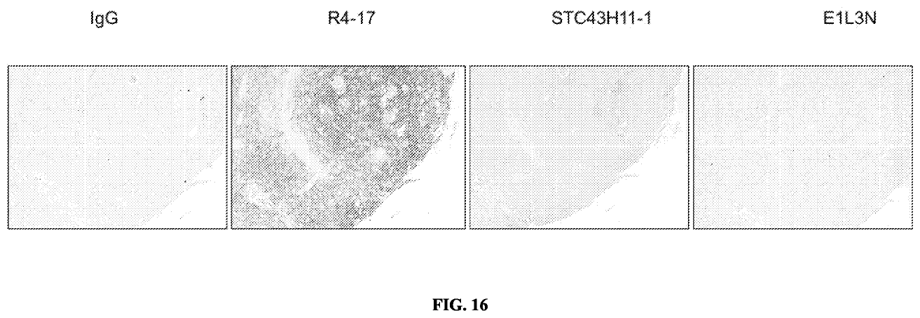

FIG. 16 show immunostaining of tissue sample using IgG (negative control), rabbit polyclonal antibody R4-17 (control), STC43H11-1 and E1L3N antibodies, respectively, in an immunohistochemistry assay on tissue sample (HN TMA: B3). STC43H11-1 was used for immunostaining at a concentration of 2 μg/mL.

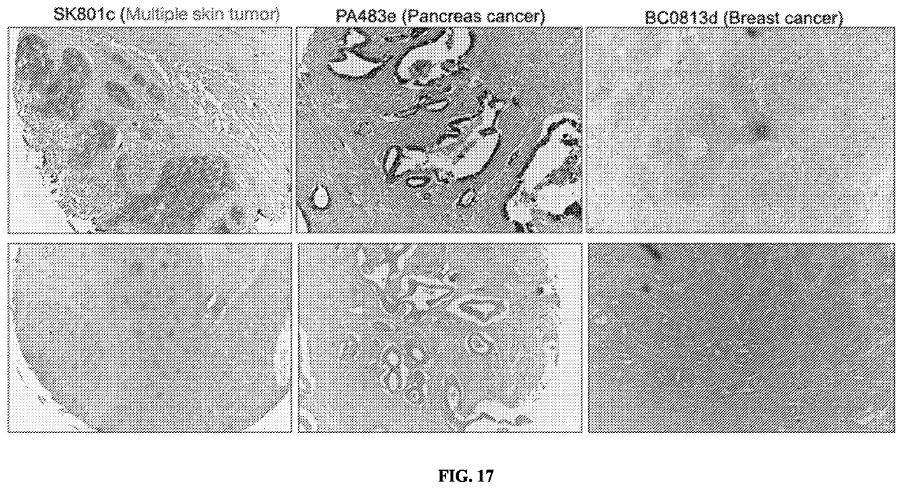

FIG. 17 shows immunostaining of multiple skin tumor tissue, pancreas cancer tissue and breast cancer tissue using STC43H11-1 in an immunohistochemistry assay. STC43H11-1 was used for immunostaining at a concentration of 2 μg/mL.

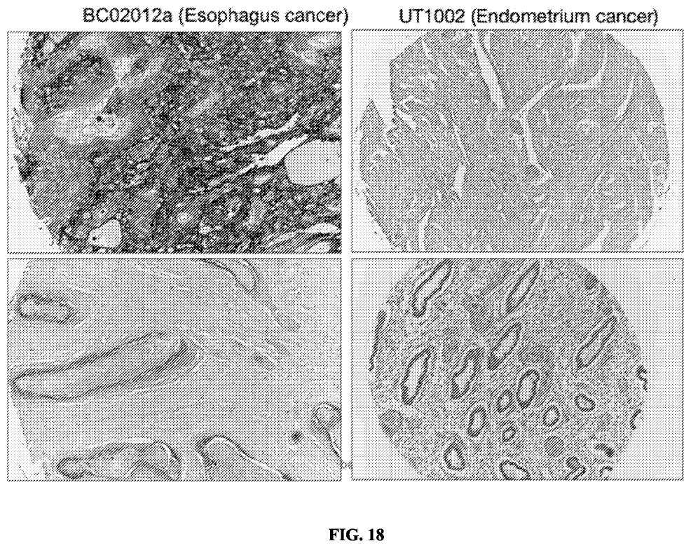

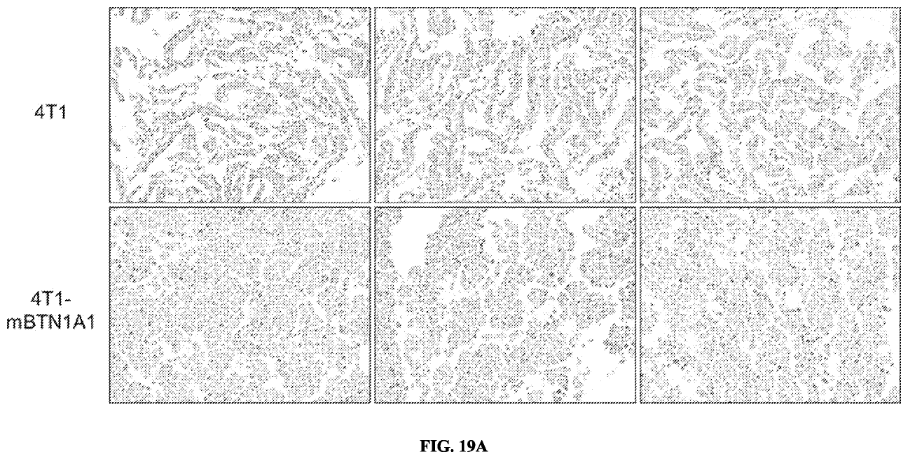

FIG. 18 shows immunostaining of esophagus cancer tissue and endometrium cancer tissue using STC43H11-1 in an immunohistochemistry assay.

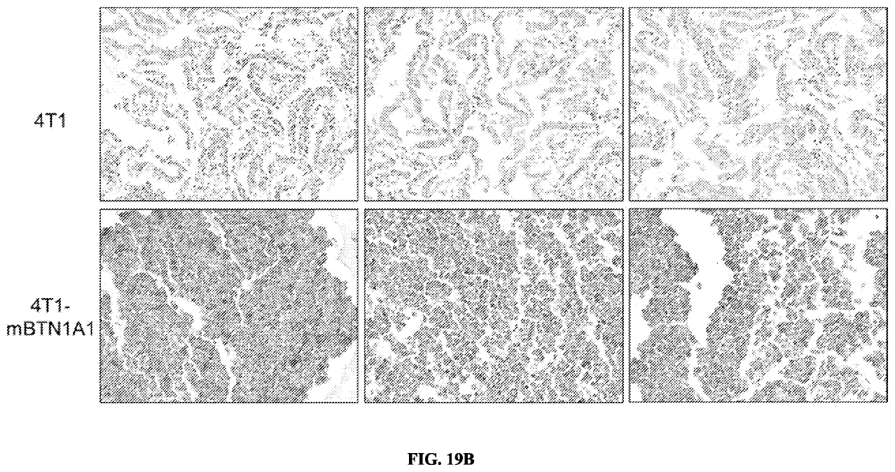

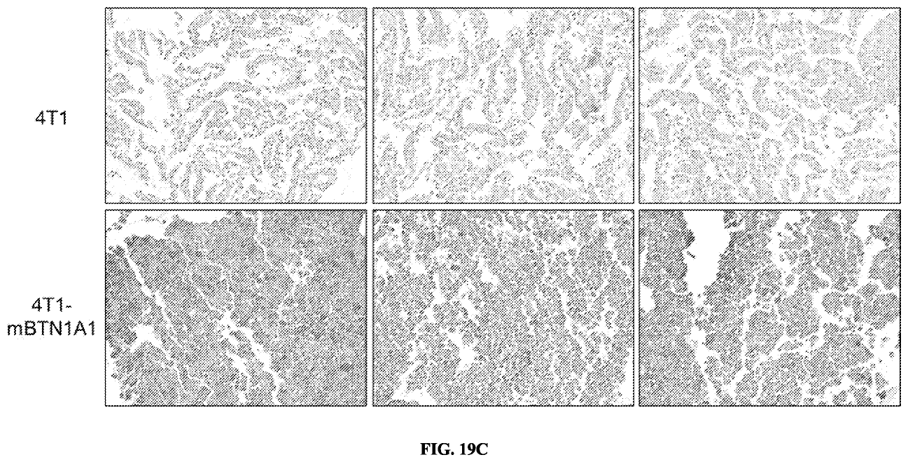

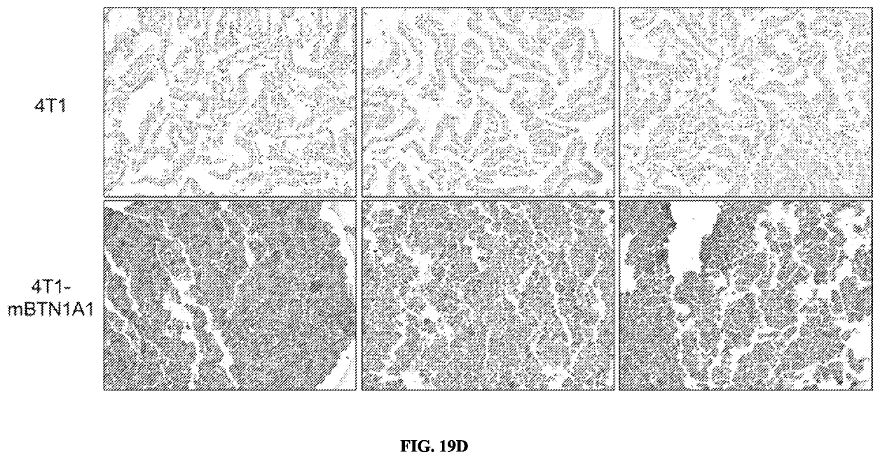

FIGS. 19A-19D shows immunostaining of mouse 4T1 breast cancer cells expressing (lower panels) or not expressing mBTN1A1 (upper panels) in an immunohistochemistry assay. STC43H11-1 was used for immunostaining at a concentration of 2 μg/mL (FIG. 19B), 5 μg/mL (FIG. 19C) or 10 μg/mL (FIG. 19D). Rabbit IgG was used for immunostaining at a concentration of 10 μg/mL as a negative control (FIG. 19A).

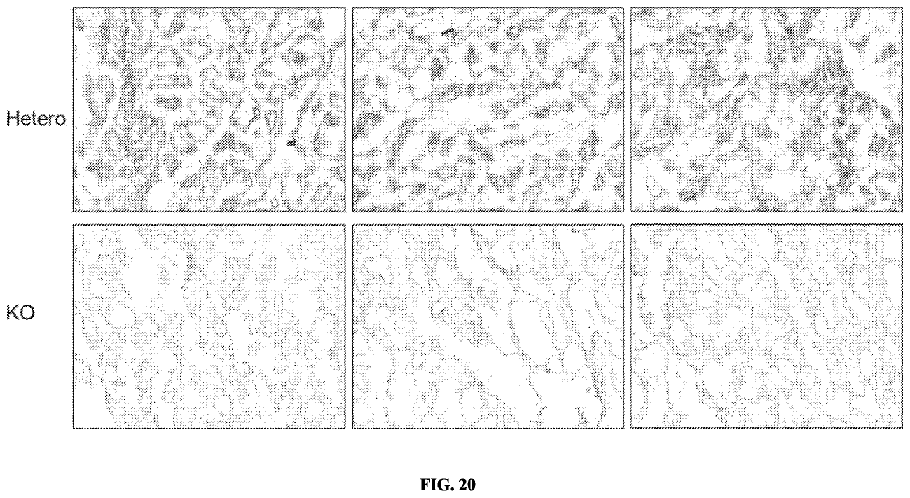

FIG. 20 shows detection of BTN1A1 protein by immunohistochemistry on mouse mammary fat pads that were snap frozen. Mice were either heterologous for mBTN1A1 (hetero) or a BTN1A1 knockout (KO). STC43H11-1 was used for immunostaining at a concentration of 10 μg/mL.

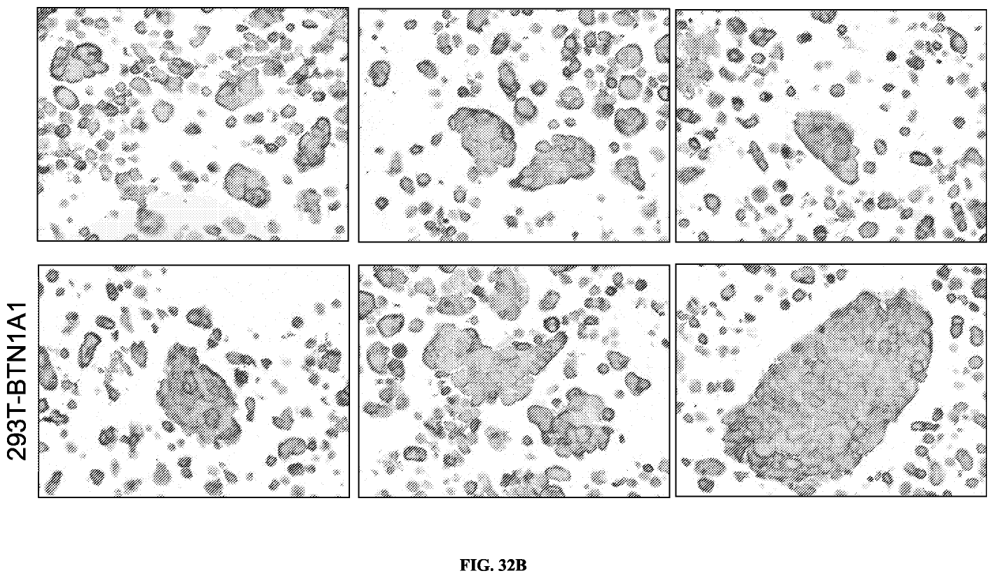

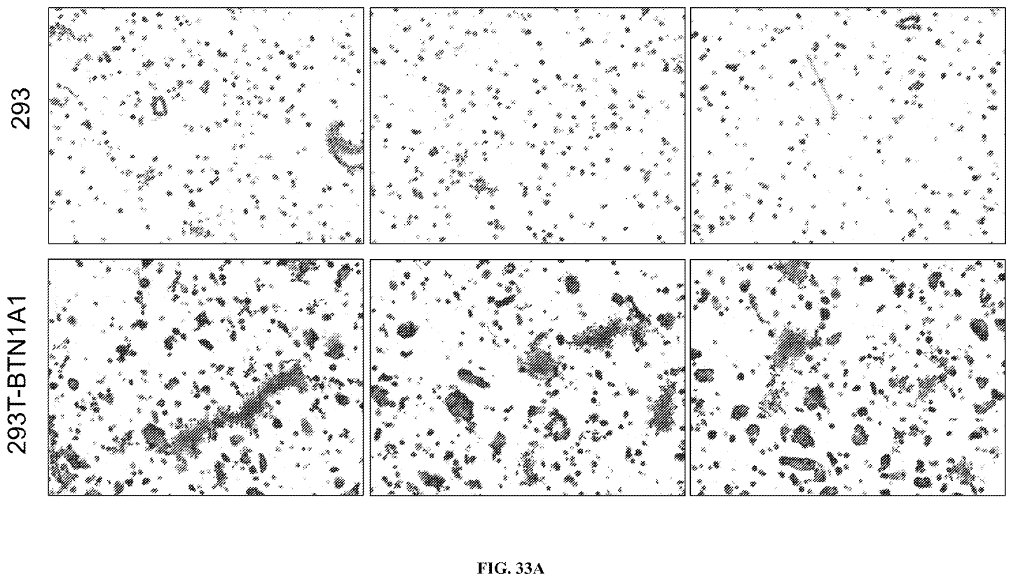

FIGS. 32A and 32B show immunostaining of 293T cells expressing (lower panels) or not expressing BTN1A1 (upper panels) in an immunohistochemistry assay. STC43G3-1 was used for immunostaining at a concentration of #μg/mL. Rabbit IgG was used for immunostaining at a concentration of 10 μg/mL as a negative control. These data show that STC43G3-1 binds to BTN1A1.

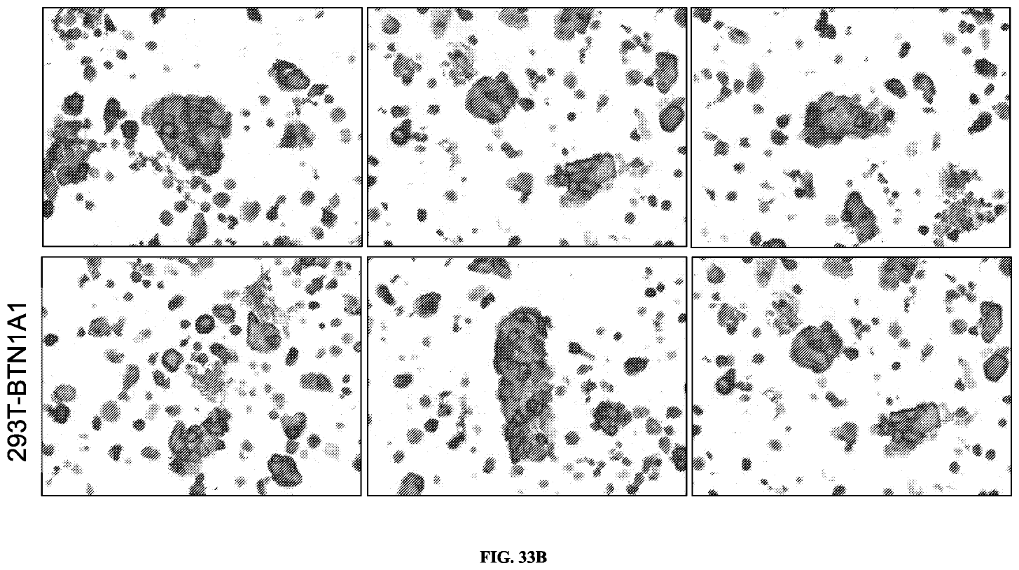

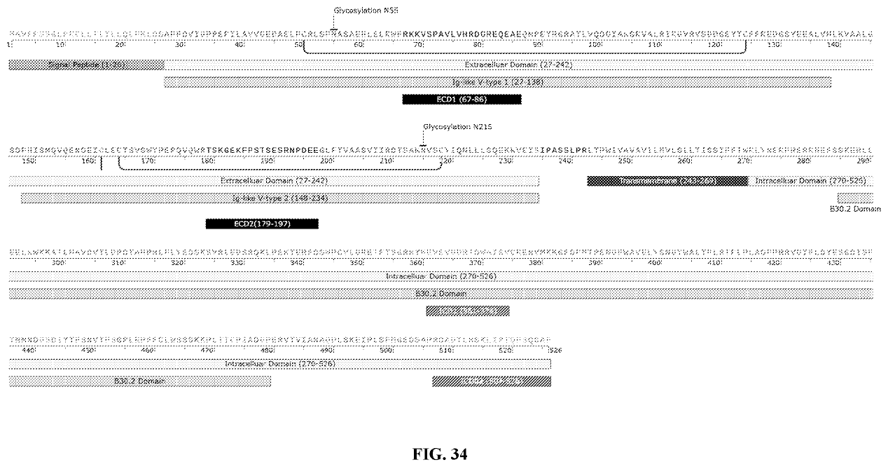

FIGS. 33A and 33B show immunostaining of 293T cells expressing (lower panels) or not expressing BTN1A1 (upper panels) in an immunohistochemistry assay. STC85F1-1 was used for immunostaining at a concentration of #g/mL. Rabbit IgG was used for immunostaining at a concentration of 10 μg/mL as a negative control. These data show that STC85F1-1 binds to BTN1A1.

### 5.1.5 Epitope Mapping

The BTN1A1 epitope recognized by STC43H11-1 was determined by introducing alanine mutations into peptide 4. Table 17 summarizes assay conditions and Table 18 summarizes the positions of alanine (or glycine) mutations introduced into peptide 4.

TABLE 17
Epitope Mapping Assay Conditions

| Conditions | |
|---|---|
| Assay buffer | PBS + 1 mg/ml BSA |
| Biosensor | Protein A biosensor |
| Ab proteins | Ab proteins (STC43H11-1) 3 |
| capture | ug/ml |
| Association | BTN1A1-Pep4 ala mutant |
| | proteins (100 nM) |
| Assay | 1. Baseline: 60 s |
| condition | 2. Loading 1: 700 s (Ab proteins) |
| | 3. Washing: 300 s |
| | 4. Association: 300 s |
| | 5. Dissociation: 900 s |
| | Shake speed: 800 rpm |

TABLE 18
Positions of Alanine Mutations in Peptide 4

| | Corresponding | |
|---|---|---|
| Alanine Mutation | Sequence | |
| BTN1A1 Peptide 4 | EVEVGDRTDWAIGVC | SEQ ID NO: 23 |
| (bold underline: | | |
| epitope) | | |
| Mutant 5 (G365A) | EVEVADRTDWAIGVC | SEQ ID NO: 24 |
| Mutant 6 (D366A) | EVEVGARTDWAIGVC | SEQ ID NO: 25 |
| Mutant 7 (R367A) | EVEVGDATDWAIGVC | SEQ ID NO: 26 |
| Mutant 8 (T368A) | EVEVGDRADWAIGVC | SEQ ID NO: 27 |
| Mutant 9 (D369A) | EVEVGDRTAWAIGVC | SEQ ID NO: 28 |
| Mutant 10 (W370A) | EVEVGDRTDAAIGVC | SEQ ID NO: 29 |
| Mutant 11 (A371G) | EVEVGDRTDWGIGVC | SEQ ID NO: 30 |
| Mutant 12 (1372A) | EVEVGDRTDWAAGVC | SEQ ID NO: 31 |
| TRIM11 peptide | EVEVGDRTSWALGVC | SEQ ID NO: 32 |

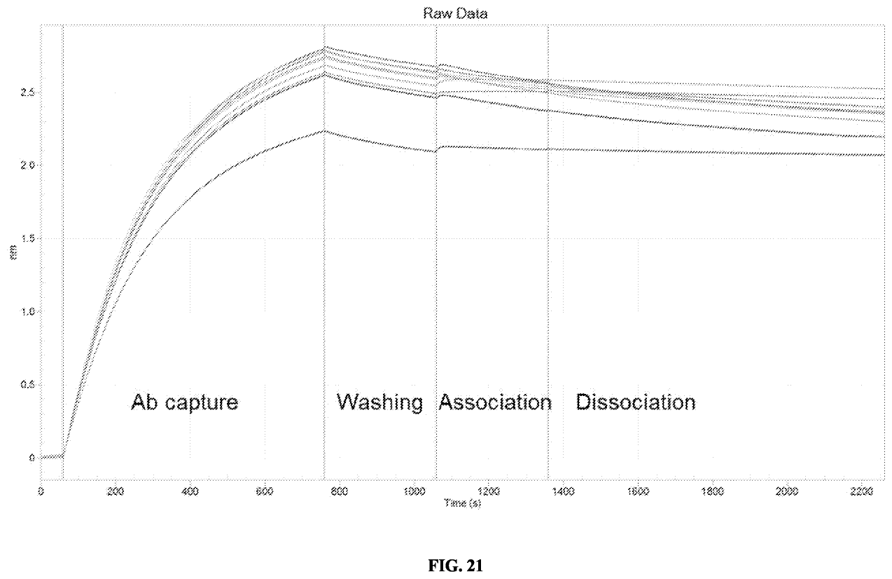

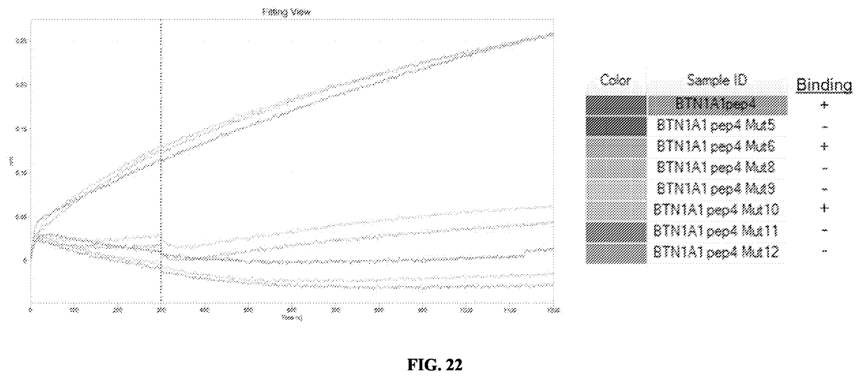

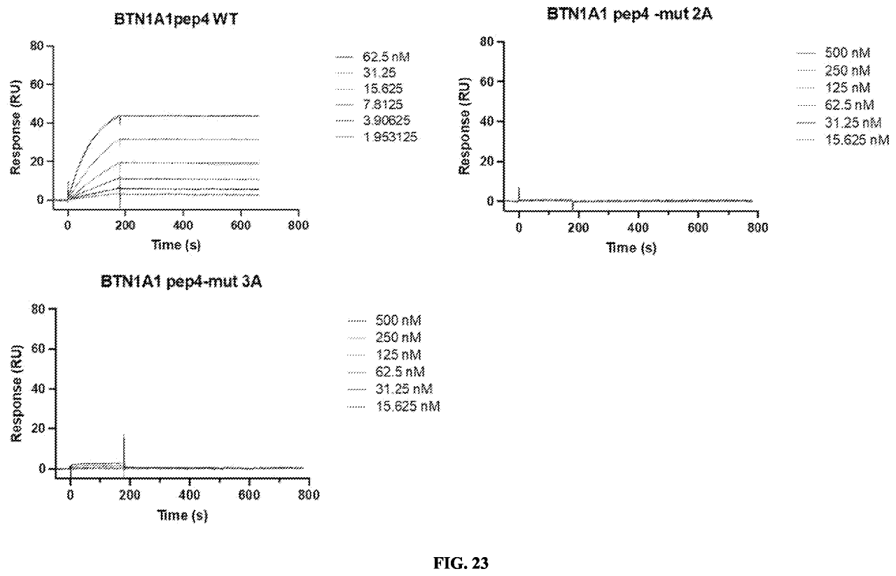

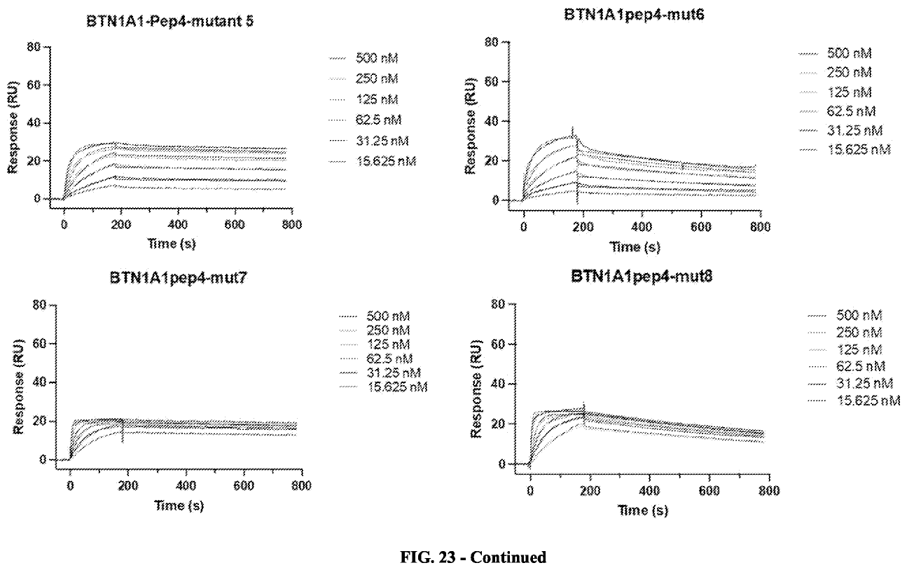

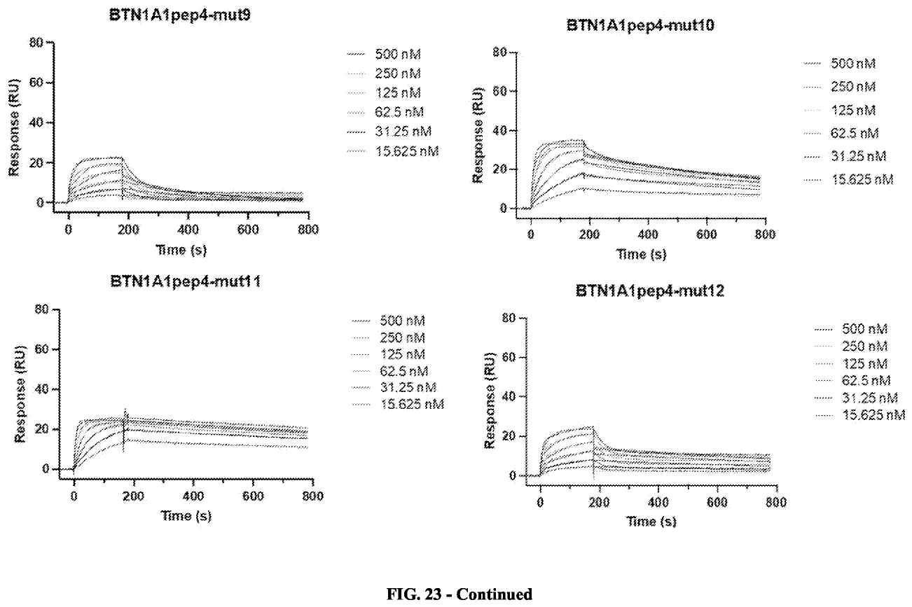

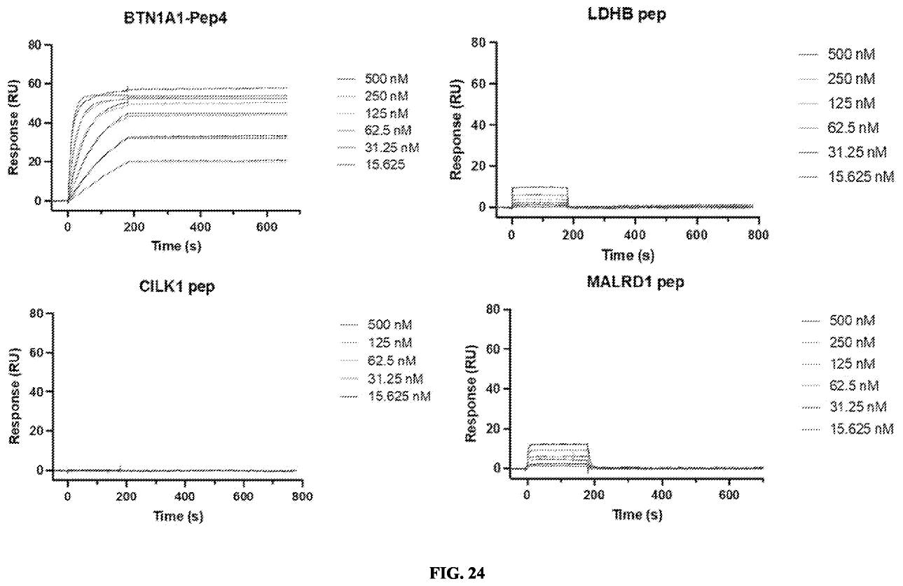

FIG. 21 shows the results of an STC43H11-1:peptide 4 mutant binding assay, wherein the STC43H11-1 captures the mutant and association and disassociation were measured. FIGS. 22 and 23 show that STC43H11-1 bound to peptide 4 and variant peptides 6 and 10. As shown, the mutation at each of G365, T368, D369, A371 and I372 (peptide 4 mutant #5, 8, 9, 11, 12) significantly reduced binding of the peptide 4 variant to STC43H11-1, indicating the epitope include G₃₆₅--T₃₆₈D₃₆₉-A₃₇₁I₃₇₂ in peptide 4.

In a separate assay, mutations at positions D366, D369, W370, and I372 (Pep 4 mutant 46, 9, 10, 12) significantly reduced binding of the peptide 4 variant to STC43H11-1, indicating the epitope include D₃₆₆--D₃₆₉W370-I372 in peptide 4.

TABLE 19

| | | | | Fold |
|---|---|---|---|---|
| Peptides | ka (1/Ms) | kd (1/s) | KD (M) | change |
| BTN1A1-pep4- WT | 1.92E+05 | 1.62E−05 | 8.44E−11 | 1 |
| BTN1A1-pep4- Mutant 5 | 8.48E+04 | 1.46E−04 | 1.72E−09 | 20 |
| BTN1A1-pep4- Mutant 6 | 6.09E+04 | 8.20E−04 | 1.35E−08 | 160 |
| BTN1A1-pep4- Mutant 7 | 4.53E+05 | 1.53E−04 | 3.39E−10 | 4 |
| BTN1A1-pep4- Mutant 8 | 4.64E+05 | 8.22E−04 | 1.77E−09 | 21 |
| BTN1A1-pep4- Mutant 9 | 2.58E+05 | 1.01E−02 | 3.91E−08 | 463 |
| BTN1A1-pep4- Mutant 10 | 2.15E+05 | 1.06E−03 | 4.95E−09 | 59 |
| BTN1A1-pep4- Mutant 11 | 3.06E+05 | 4.25E−04 | 1.39E−09 | 16 |
| BTN1A1-pep4- Mutant 12 | 7.36E+04 | 7.78E−04 | 1.06E−08 | 126 |
| TRIM11 pep | 1.62E+05 | 1.00E−01 | 6.21E−07 | 7,358 |

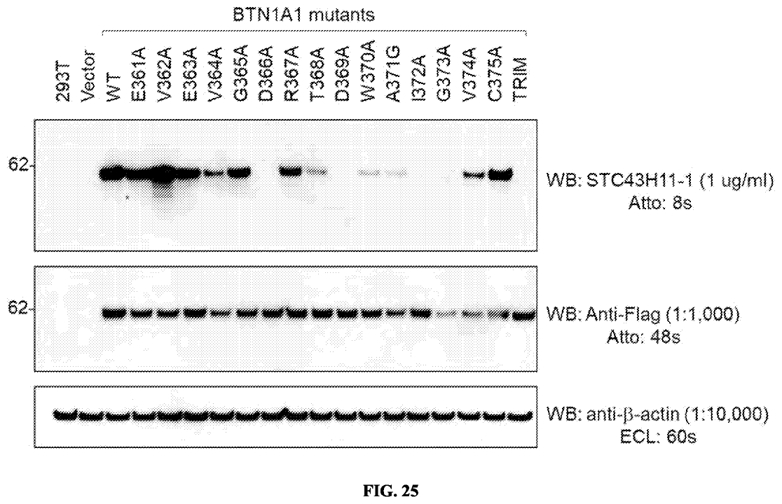

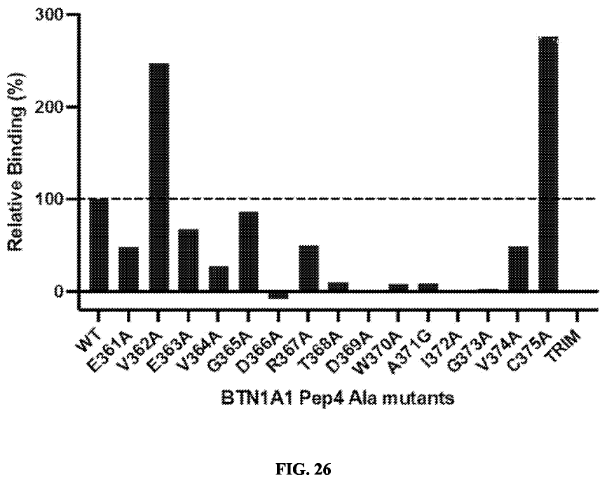

In a separate assay, binding of STC43H11-1 to mutated peptide 4 variants expressed by HEK293T cells was assessed using western blotting. As shown in FIG. 25, mutations at D366, T368, D369, W370, A371, I372 and G373 each significantly reduced binding of the peptide 4 variant to STC43H11-1. The western blotting results were quantitated by calculating the Relative binding (%) as follows, which are summarized in Table 20 and plotted in FIG. 26.

$$Relative binding ( \% ) = \frac{Adjusted volume ( Int ) of STC 43 H 11 - 1}{Adjusted volume ( Int ) of anti - Flag} \times 100$$

These results indicate that the epitope of STC43H11-1 include D₃₆₆-T₃₆₈D₃₆₉W₃₇₀A₃₇₁I₃₇₂G₃₇₃ in peptide 4.

TABLE 20

| | Flag | STC43H11-1 | Normalized | % binding |
|---|---|---|---|---|
| Mutants | Adj. Vol. (Int) | Adj. Vol. (Int) | Fold | Rel. Quant. |
| WT | 8,417,612.78 | 4,021,571.00 | 0.477756711 | 100 |
| E361A | 5,845,971.63 | 1,347,687.00 | 0.230532593 | 48.253136 |
| V362A | 5,946,538.07 | 7,027,958.80 | 1.181857195 | 247.37637 |
| E363A | 8,095,168.76 | 2,614,950.00 | 0.323026002 | 67.613075 |
| V364A | 3,740,534.25 | 479,549.60 | 0.128203505 | 26.834475 |
| G365A | 5,461,070.90 | 2,263,576.80 | 0.414493209 | 86.758218 |
| D366A | 5,577,699.72 | −227,887.00 | −0.04085681 | −8.551802 |
| R367A | 6,174,076.55 | 1,478,171.20 | 0.239415755 | 50.112484 |
| T368A | 7,549,447.82 | 374,347.40 | 0.049586064 | 10.378936 |
| D369A | 6,409,927.52 | 5,466.20 | 0.000852771 | 0.1784948 |
| W370A | 5,607,022.91 | 220,896.40 | 0.039396379 | 8.2461174 |
| A371G | 3,472,263.19 | 156,127.00 | 0.044964045 | 9.4114943 |
| I372A | 6,599,785.74 | 4,009.60 | 0.000607535 | 0.1271641 |
| G373A | 1,849,754.42 | 19,865.00 | 0.010739263 | 2.2478519 |
| V374A | 2,834,780.89 | 672,000.40 | 0.2370555 | 49.618455 |
| C375A | 3,629,511.99 | 4,798,028.60 | 1.321948685 | 276.69913 |
| TRIM | 7,497,998.53 | −7,614.40 | −0.00101552 | −0.212561 |

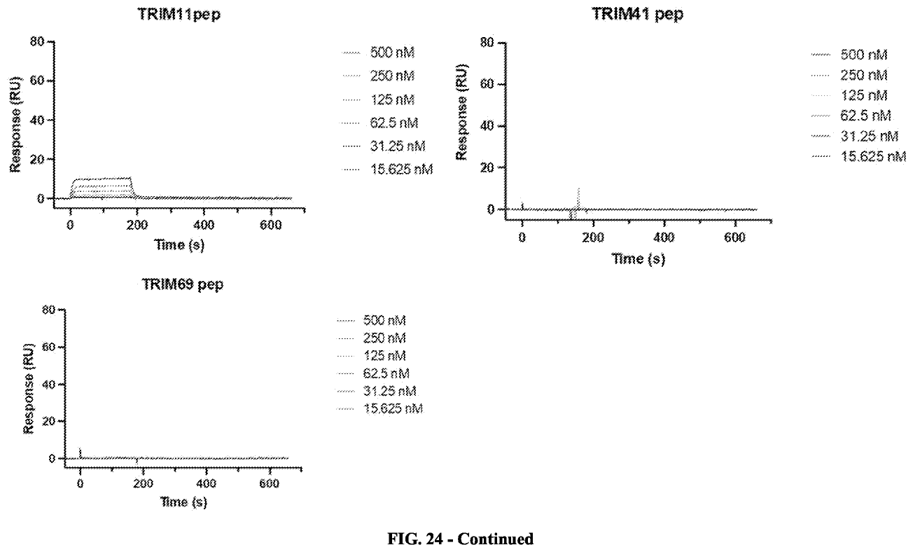

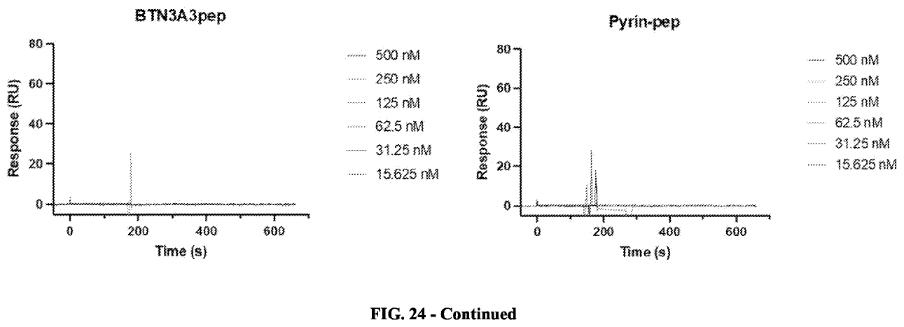

Next, peptides containing homologous sequences to peptide 4 were used in binding assays with STC43H11-1 to demonstrate the specificity of STC43H11-1 for binding BTN1A1, including AQPVHGRTDWASKYA (SEQ ID NO:35) from serine/threonine-protein kinase ICK (CILK) isoform 2 NP_001362326.1, VIKLKGYTNWAIGLS (SEQ ID NO:36) from 1-lactate dehydrogenase B chain (LDHB) isoform X1 XP_006719137, and TQDSEDDLDWAIGSR (SEQ ID NO:37) from MAM and LDL-receptor class A domain-containing protein 1 (MALRD1) isoform X1 XP_011517755.1. As shown in FIG. 24, STC43H11-1 specifically bound to peptide 4 but did not any other proteins tested, including CILK1, MALRD1 or LDHB.

What is claimed:

1. An antibody or antigen-binding fragment thereof that
(a) binds to an epitope of BTN1A1 recognized by an antibody comprising
(i) a light chain variable region having an amino acid sequence of SEQ ID NO: 19 and a heavy chain variable region having an amino acid sequence of SEQ ID NO:20;
(ii) a light chain variable region having an amino acid sequence of SEQ ID NO: 66 and a heavy chain variable region having an amino acid sequence of SEQ ID NO:68; or
(iii) a light chain variable region having an amino acid sequence of SEQ ID NO: 67 and a heavy chain variable region having an amino acid sequence of SEQ ID NO:69; or
(b) competes for the binding to BTN1A1 with an antibody comprising
(i) a light chain variable region having an amino acid sequence of SEQ ID NO:19 and a heavy chain variable region having an amino acid sequence of SEQ ID NO:20;
(ii) a light chain variable region having an amino acid sequence of SEQ ID NO: 66 and a heavy chain variable region having an amino acid sequence of SEQ ID NO: 68; or
(iii) a light chain variable region having an amino acid sequence of SEQ ID NO:67 and a heavy chain variable region having an amino acid sequence of SEQ ID NO:69.

2. An antibody or antigen-binding fragment thereof that binds to BTN1A1, wherein the antibody or antigen-binding fragment thereof comprises:
(a) a light chain variable region (VL) comprising VL complementarity determining region 1 (CDR1), VL CDR2, and VL CDR3 of antibody STC43H11-1 as set forth in Table 1; and/or a heavy chain variable region (VH) comprising VH complementarity determining region 1 (CDR1), VH CDR2, and VH CDR3 of antibody STC43H11-1 as set forth in Table 2;
(b) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:19; and/or a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:20;
(c) a VL comprising VL CDR1, VL CDR2, and VL CDR3 of antibody STC43G3-1 as set forth in Table 3; and/or a VH comprising VH CDR1, VH CDR2, and VH CDR3 of antibody STC43G3-1 as set forth in Table 4;
(d) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66; and/or a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:68;
(e) a VL comprising VL CDR1, VL CDR2, and VL CDR3 of antibody STC85F1-1 as set forth in Table 5; and/or a VH comprising VH CDR1, VH CDR2, and VH CDR3 of antibody STC85F1-1 as set forth in Table 6; or
(f) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:67; and/or a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:69.

3. The antibody or antigen-binding fragment thereof of claim 2, wherein
(a) the VH CDR1, VH CDR2, VH CDR3, VL CDR1, VL CDR2, and VL CDR3 amino acid sequences are according to the Kabat numbering system;
(b) the VH CDR1, VH CDR2, VH CDR3, VL CDR1, VL CDR2, and VL CDR3 amino acid sequences are according to the Chothia numbering system;
(c) the VH CDR1, VH CDR2, VH CDR3, VL CDR1, VL CDR2, and VL CDR3 amino acid sequences are according to the ABM numbering system;
(d) the VH CDR1, VH CDR2, VH CDR3, VL CDR1, VL CDR2, and VL CDR3 amino acid sequences are according to the Contact numbering system; or
(e) the VH CDR1, VH CDR2, VH CDR3, VL CDR1, VL CDR2, and VL CDR3 amino acid sequences are according to the IMGT numbering system.

4. The antibody or antigen-binding fragment thereof of claim 2, wherein the antibody or antigen-binding fragment thereof comprises:
(a) a VL further comprising VL framework 1 (FR1), VL FR2, VL FR3, and VL FR4 of any one of antibody STC43H11-1 as set forth in Table 7; and/or a VH further comprising VH framework 1 (FR1), VH FR2, VH FR3, and VH FR4 of any one of antibody STC43H11-1 as set forth in Table 8;
(b) a VL further comprising VL framework 1 (FR1), VL FR2, VL FR3, and VL FR4 of any one of antibody STC43G3-1 as set forth in Table 7; and/or a VH further comprising VH framework 1 (FR1), VH FR2, VH FR3, and VH FR4 of any one of antibody STC43G3-1 as set forth in Table 8; or
(c) a VL further comprising VL framework 1 (FR1), VL FR2, VL FR3, and VL FR4 of any one of antibody STC85F1-1 as set forth in Table 7; and/or a VH further comprising VH framework 1 (FR1), VH FR2, VH FR3, and VH FR4 of any one of antibody STC85F1-1 as set forth in Table 8.

5. The antibody or antigen-binding fragment thereof of claim 2, wherein
(a) the VL CDR1, VL CDR2, and VL CDR3 comprise amino acid sequences of SEQ ID NOS:5, 6, and 7, respectively, and the VH CDR1, VH CDR2, and VH CDR3 comprise amino acid sequences of SEQ ID NOS:8, 9, and 10, respectively;
(b) the VL CDR1, VL CDR2, and VL CDR3 comprise amino acid sequences of SEQ ID NOS:74, 75, and 76, respectively, and the VH CDR1, VH CDR2, and VH CDR3 comprise amino acid sequences of SEQ ID NOS:71, 72, and 73, respectively;
(c) the VL CDR1, VL CDR2, and VL CDR3 comprise amino acid sequences of SEQ ID NOS:80, 81, and 82, respectively, and the VH CDR1, VH CDR2, and VH CDR3 comprise amino acid sequences of SEQ ID NOS:77, 78, and 79, respectively; or
(d) the VL CDR1, VL CDR2, and VL CDR3 comprise amino acid sequences of SEQ ID NOS:86, 87, and 88, respectively, and the VH CDR1, VH CDR2, and VH CDR3 comprise amino acid sequences of SEQ ID NOS:83, 84, and 85, respectively, respectively

6. The antibody or antigen-binding fragment thereof of claim 2, wherein the antibody or antigen-binding fragment thereof comprises a VL comprising an amino acid sequence having at least 95% sequence identity to SEQ ID NO:19.

7. The antibody or antigen-binding fragment thereof of claim 2, wherein the antibody or antigen-binding fragment thereof comprises a VL comprising an amino acid sequence of SEQ ID NO:19.

8. The antibody or antigen-binding fragment thereof of claim 2, wherein the antibody or antigen-binding fragment thereof comprises a VH comprising an amino acid sequence having at least 95% sequence identity to SEQ ID NO:20.

9. The antibody or antigen-binding fragment thereof of claim 2, wherein the antibody or antigen-binding fragment thereof comprises a VH comprising an amino acid sequence of SEQ ID NO:20.

10. The antibody or antigen-binding fragment thereof of claim 2, wherein the antibody or antigen-binding fragment thereof comprises a VL comprising an amino acid sequence having at least 95% sequence identity to SEQ ID NO: 19 and a VH comprising an amino acid sequence having at least 95% sequence identity to SEQ ID NO:20.

11. The antibody or antigen-binding fragment thereof of claim 2, wherein the antibody or antigen-binding fragment thereof comprises:
(a) a VL comprising an amino acid sequence of SEQ ID NO: 19; and
(b) a VH comprising an amino acid sequence of SEQ ID NO:20.

12. The antibody or antigen-binding fragment thereof of claim 2, wherein
(a) the VL CDR1, VL CDR2, and VL CDR3 comprise amino acid sequences of SEQ ID NOS:38, 39, and 40, respectively, and the VH CDR1, VH CDR2, and VH CDR3 comprise amino acid sequences of SEQ ID NOS:44, 45, and 46, respectively;
(b) the VL CDR1, VL CDR2, and VL CDR3 comprise amino acid sequences of SEQ ID NOS:94, 95, and 96, respectively, and the VH CDR1, VH CDR2, and VH CDR3 comprise amino acid sequences of SEQ ID NOS:91, 92, and 93, respectively;
(c) the VL CDR1, VL CDR2, and VL CDR3 comprise amino acid sequences of SEQ ID NOS:100, 101, and 102, respectively, and the VH CDR1, VH CDR2, and VH CDR3 comprise amino acid sequences of SEQ ID NOS:97, 98, and 99, respectively; or
(d) the VL CDR1, VL CDR2, and VL CDR3 comprise amino acid sequences of SEQ ID NOS:106, 107, and 108, respectively, and the VH CDR1, VH CDR2, and VH CDR3 comprise amino acid sequences of SEQ ID NOS:103, 104, and 105, respectively, respectively

13. The antibody or antigen-binding fragment thereof of claim 2, wherein the antibody or antigen-binding fragment thereof comprises a VL comprising an amino acid sequence having at least 95% sequence identity to SEQ ID NO:66.

14. The antibody or antigen-binding fragment thereof of claim 2, wherein the antibody or antigen-binding fragment thereof comprises a VL comprising an amino acid sequence of SEQ ID NO:66.

15. The antibody or antigen-binding fragment thereof of claim 2, wherein the antibody or antigen-binding fragment thereof comprises a VH comprising an amino acid sequence having at least 95% sequence identity to SEQ ID NO:68.

16. The antibody or antigen-binding fragment thereof of claim 2, wherein the antibody or antigen-binding fragment thereof comprises a VH comprising an amino acid sequence of SEQ ID NO:68.

17. The antibody or antigen-binding fragment thereof of claim 2, wherein the antibody or antigen-binding fragment thereof comprises a VL comprising an amino acid sequence having at least 95% sequence identity to SEQ ID NO:66 and a VH comprising an amino acid sequence having at least 95% sequence identity to SEQ ID NO:68.

18. The antibody or antigen-binding fragment thereof of claim 2, wherein the antibody or antigen-binding fragment thereof comprises:
(a) a VL comprising an amino acid sequence of SEQ ID NO:66; and
(b) a VH comprising an amino acid sequence of SEQ ID NO:68.

19. The antibody or antigen-binding fragment thereof of claim 2, wherein
(a) the VL CDR1, VL CDR2, and VL CDR3 comprise amino acid sequences of SEQ ID NOS:41, 42, and 43, respectively, and the VH CDR1, VH CDR2, and VH CDR3 comprise amino acid sequences of SEQ ID NOS:47, 48, and 49, respectively;
(b) the VL CDR1, VL CDR2, and VL CDR3 comprise amino acid sequences of SEQ ID NOS:114, 115, and 116, respectively, and the VH CDR1, VH CDR2, and VH CDR3 comprise amino acid sequences of SEQ ID NOS:111, 112, and 113, respectively;
(c) the VL CDR1, VL CDR2, and VL CDR3 comprise amino acid sequences of SEQ ID NOS:120, 121, and 122, respectively, and the VH CDR1, VH CDR2, and VH CDR3 comprise amino acid sequences of SEQ ID NOS:117, 118, and 119, respectively; or
(d) the VL CDR1, VL CDR2, and VL CDR3 comprise amino acid sequences of SEQ ID NOS:126, 127, and 128, respectively, and the VH CDR1, VH CDR2, and VH CDR3 comprise amino acid sequences of SEQ ID NOS:123, 124, and 125, respectively, respectively

20. The antibody or antigen-binding fragment thereof of claim 2, wherein the antibody or antigen-binding fragment thereof comprises a VL comprising an amino acid sequence having at least 95% sequence identity to SEQ ID NO:67.

21. The antibody or antigen-binding fragment thereof of claim 2, wherein the antibody or antigen-binding fragment thereof comprises a VL comprising an amino acid sequence of SEQ ID NO:67.

22. The antibody or antigen-binding fragment thereof of claim 2, wherein the antibody or antigen-binding fragment thereof comprises a VH comprising an amino acid sequence having at least 95% sequence identity to SEQ ID NO:69.

23. The antibody or antigen-binding fragment thereof of claim 2, wherein the antibody or antigen-binding fragment thereof comprises a VH comprising an amino acid sequence of SEQ ID NO:69.

24. The antibody or antigen-binding fragment thereof of claim 2, wherein the antibody or antigen-binding fragment thereof comprises a VL comprising an amino acid sequence having at least 95% sequence identity to SEQ ID NO:67 and a VH comprising an amino acid sequence having at least 95% sequence identity to SEQ ID NO:69.

25. The antibody or antigen-binding fragment thereof of claim 2, wherein the antibody or antigen-binding fragment thereof comprises:
(a) a VL comprising an amino acid sequence of SEQ ID NO:67; and
(b) a VH comprising an amino acid sequence of SEQ ID NO:69.

26. The antibody or antigen-binding fragment thereof of any one of claims 1-25, wherein the antibody or antigen-binding fragment thereof binds to human BTN1A1.

27. The antibody or antigen-binding fragment thereof of any one of claims 1-25, wherein the antibody or antigen-binding fragment thereof binds to mouse BTN1A1.

28. The antibody or antigen-binding fragment thereof of any one of claims 1-27, wherein, when bound to BTN1A1, the antibody or antigen-binding fragment binds to at least one of residues 361-375 within an amino acid sequence of SEQ ID NO:1.

29. The antibody or antigen-binding fragment thereof of any one of claims 1-27, wherein, when bound to BTN1A1, the antibody or antigen-binding fragment binds to at least one of residues 365-372 within an amino acid sequence of SEQ ID NO:1.

30. The antibody or antigen-binding fragment thereof of any one of claims 1-27, wherein, when bound to BTN1A1, the antibody or antigen-binding fragment binds to at least one of residues 366-372 within an amino acid sequence of SEQ ID NO:1.

31. The antibody or antigen-binding fragment thereof of any one of claims 1-27, wherein, when bound to BTN1A1, the antibody or antigen-binding fragment binds to at least one residue selected from the group consisting of G365, D366, T368, D369, W370, A371, I372, and G373 within an amino acid sequence of SEQ ID NO:1.

32. The antibody or antigen-binding fragment thereof of claim 31, wherein, when bound to BTN1A1, the antibody or antigen-binding fragment binds to G365 within an amino acid sequence of SEQ ID NO:1.

33. The antibody or antigen-binding fragment thereof of claim 31, wherein, when bound to BTN1A1, the antibody or antigen-binding fragment binds to D366 within an amino acid sequence of SEQ ID NO:1.

34. The antibody or antigen-binding fragment thereof of claim 31, wherein, when bound to BTN1A1, the antibody or antigen-binding fragment binds to T368 within an amino acid sequence of SEQ ID NO:1.

35. The antibody or antigen-binding fragment thereof of claim 31, wherein, when bound to BTN1A1, the antibody or antigen-binding fragment binds to D369 within an amino acid sequence of SEQ ID NO:1.

36. The antibody or antigen-binding fragment thereof of claim 31, wherein, when bound to BTN1A1, the antibody or antigen-binding fragment binds to W370 within an amino acid sequence of SEQ ID NO:1.

37. The antibody or antigen-binding fragment thereof of claim 31, wherein, when bound to BTN1A1, the antibody or antigen-binding fragment binds to A371 within an amino acid sequence of SEQ ID NO:1.

38. The antibody or antigen-binding fragment thereof of claim 31, wherein, when bound to BTN1A1, the antibody or antigen-binding fragment binds to I372 within an amino acid sequence of SEQ ID NO: 1.

39. The antibody or antigen-binding fragment thereof of claim 31, wherein, when bound to BTN1A1, the antibody or antigen-binding fragment binds to G373 within an amino acid sequence of SEQ ID NO:1.

40. The antibody or antigen-binding fragment thereof of claim 31, wherein, when bound to BTN1A1, the antibody or antigen-binding fragment binds to D366, D369 and I372 within an amino acid sequence of SEQ ID NO:1.

41. The antibody or antigen-binding fragment thereof of claim 31, wherein, when bound to BTN1A1, the antibody or antigen-binding fragment binds to G365, T368, D369, A371 and I372 within an amino acid sequence of SEQ ID NO:1.

42. The antibody or antigen-binding fragment thereof of claim 31, wherein, when bound to BTN1A1, the antibody or antigen-binding fragment binds to D366, T368, D369, W370, A371, I372, and G373 within an amino acid sequence of SEQ ID NO:1.

43. The antibody or antigen-binding fragment thereof of any one of claims 1-42, wherein the antibody is a monoclonal antibody.

44. The antibody or antigen-binding fragment thereof of any one of claims 1-43, wherein the antibody is a humanized, human, or chimeric antibody.

45. The antibody or antigen-binding fragment thereof of claim 44, wherein the humanized antibody is a deimmunized antibody or a composite human antibody.

46. The antibody or antigen-binding fragment thereof of any one of claims 1-45, wherein the antibody or antigen-binding fragment thereof is a Fab, a Fab′, a F(ab′)₂, a Fv, a scFv, a dsFv, a diabody, a triabody, a tetrabody, or a multispecific antibody formed from antibody fragments.

47. The antibody or antigen-binding fragment thereof of any one of claims 1-46, wherein the antibody or antigen-binding fragment thereof is conjugated to an agent.

48. The antibody or antigen-binding fragment thereof of claim 47, wherein the agent is selected from the group consisting of a radioisotope, a metal chelator, an enzyme, a fluorescent compound, a bioluminescent compound, and a chemiluminescent compound.

49. A composition comprising the antibody or antigen-binding fragment thereof of any one of claims 1-48, and a pharmaceutically acceptable carrier.

50. A polynucleotide comprising nucleotide sequences encoding a VH, a VL, or both a VH and a VL of the antibody of any one of claims 1-48.

51. A polynucleotide comprising nucleotide sequences encoding a heavy chain, a light chain, or both a heavy chain and a light chain of the antibody of any one of claims 1-48.

52. The polynucleotide of claim 50 or 51, wherein the polynucleotide is operably linked to a promoter.

53. A vector comprising the polynucleotide of any one of claims 50 to 52.

54. A cell comprising the polynucleotide of any one of claims 50 to 52.

55. A cell comprising the vector of claim 53.

56. An isolated cell producing the antibody or antigen-binding fragment thereof of any one of claims 1-48.

57. A kit comprising the antibody or antigen-binding fragment thereof of any one of claims 1-48.

58. A method of making an antibody or antigen-binding fragment thereof which specifically binds to an epitope of BTN1A1, comprising culturing the cell of any one of claims 54 to 56 to express the antibody or antigen-binding fragment thereof.

59. A method of making an antibody or antigen-binding fragment thereof which specifically binds to an epitope of BTN1A1, comprising expressing the polynucleotide of any one of claims 50 to 52.

60. A method of detecting a BTN1A1 in a sample, comprising contacting the sample with the anti-BTN1A1 antibody or antigen binding fragment thereof of any one of claims 1 to 48, and detecting the presence of the anti-BTN1A1 antibody or antigen binding fragment thereof bound to the sample, wherein detection above background of an amount of the anti-BTN1A1 antibody or antigen binding fragment thereof bound to the sample indicates the presence of BTN1A1 in the sample.

61. The method of claim 60, wherein the method further comprises quantitating the amount of detected BTN1A1 in the sample.

62. The method of claim 60 or 61, wherein the anti-BTN1A1 antibody or antigen binding fragment thereof is conjugated with a detectable agent producing a signal, and the detecting comprises detecting the signal from the sample.

63. The method of claim 60 or 61, wherein the anti-BTN1A1 antibody or antigen binding fragment thereof is contacted with a secondary binding molecule that specifically binds to the anti-BTN1A1 antibody or antigen binding fragment in the sample, and the detecting comprises detecting the secondary binding molecule from the sample.

64. The method of any one of claim 60 to 63, wherein the sample is a cell-containing sample.

65. The method of claim 64, wherein the cells in the sample are fixed in situ before contacted with the anti-BTN1A1 antibody or antigen binding fragment thereof.

66. The method of claim 64 or 65, wherein the sample is a formalin fixed paraffin embedded (FFPE) tissue sample.

67. The method of any one of claims 60 to 66, wherein the sample is a cancer tissue; optionally wherein the cancer is selected from skin cancer, pancreases cancer, breast cancer, esophagus cancer, and endometrium cancer.

68. The method of any one of claims 60 to 66, wherein the sample is a bodily fluid sample; optionally wherein the bodily fluid sample is blood, serum or plasma.

69. The method of any one of claims 60 to 68, wherein the sample is obtained from a subject, and wherein the method further comprises diagnosing the subject for having or at risk of developing a BTN1A1-mediated disease or condition upon detection above a threshold of an amount of BTN1A1 in the sample.

70. The method of claim 69, wherein the BTN1A1-mediated disease or condition is cancer.

71. The method of claim 69 or 70, wherein the subject is a mammal.

72. The method of any one of claims 69 to 71, wherein the subject is human.

73. A kit for performing the method of any one of claims 60 to 72.

74. A method of treating a subject with a BTN1A1 targeting agent, comprising a step of detecting BTN1A1 in a sample from the subject.

75. The method of claim 74, wherein the step of detecting BTN1A1 in the sample comprises the method of any of claims 60 to 72.

76. The method of claim 74 or 75, wherein the targeting agent is an antagonistic polypeptide binding to BTN1A1.

77. The method of claim 74 or 75, wherein the targeting agent is an agonistic polypeptide binding to BTN1A1.

78. The method of claim 76 or 77, wherein the polypeptide binding to BTN1A1 is an antibody or antigen binding fragment thereof.

79. The method of claim 74 or 75, wherein the targeting agent is a small molecule compound inhibiting BTN1A1.

80. The method of any of claims 74 to 79, wherein the subject has a disease or condition mediated by BTN1A1.

81. The method of any of claims 74 to 80, wherein the method eliminates a population of diseased cells expressing BTN1A1.

82. The method of any of claims 74 to 81, wherein the subject has cancer.
